(19) United States
(12) Patent Application Publication
(10) Pub. No.: US 2009/0280543 A1
(43) Pub. Date: Nov. 12, 2009

(54) REGIOSELECTIVE GLYCOSYLATION

(75) Inventors: Eng-Kiat Lim, York (GB); Markus Wies, Ellerstadt (DE)

Correspondence Address: SPECKMAN LAW GROUP PLLC, 1201 THIRD AVENUE, SUITE 330, SEATTLE, WA, 98101, US

(73) Assignee: THE UNIVERSITY OF YORK, York (GB)

(21) Appl. No.: 12/067,526

(22) PCT Filed: Sep. 21, 2006
(86) PCT No.: PCT/GB06/03510

§ 371 Date: Dec. 3, 2008

(30) Foreign Application Priority Data
Sep. 21, 2005 (GB) 0519231.5

(51) Int. Cl. C12P 17/06; C12P 5/00; C12P 7/22; C07C 39/12

(52) U.S. Cl. 435/125; 435/166; 435/156; 568/729

(57) ABSTRACT

We describe a screening method for the identification of glycosyltransferase polypeptides that regioselectively modify aglycones and the use of said glycosyltransferase polypeptides to modify aglycones.

### REFERENCE TO RELATED APPLICATIONS

This application is the US national phase entry of International Patent Application No. PCT/GB2006/003510, filed Sep. 21, 2006, which claims priority to UK Patent Application No. 0519231.5, filed Sep. 21, 2005.

### FIELD OF THE INVENTION

The invention relates to the regioselective modification of aglycones by glycosyltransferase polypeptides.

### BACKGROUND OF THE INVENTION

Carbohydrates are ubiquitous throughout nature and play important biological roles. For example, carbohydrates are involved in intercellular recognition in mammalian cells and in plants are a major component of the plant cell wall. A class of enzyme involved in carbohydrate metabolism are the glycosyltransferase (GTase) enzymes. GTases are enzymes that transfer sugar residues from an activated nucleotide sugar to monomeric and polymeric acceptor molecules called aglycones (e.g. other sugars, proteins and peptides, lipids and other organic substrates). These glycosylated molecules take part in diverse metabolic pathways and processes. The transfer of a sugar moiety can alter the acceptor's bioactivity, solubility or transport properties within a cell. Examples of GTases include glucosyltransferases, fucosyltransferases, sialyltransferases and galatosyltransferases.

The chemical synthesis of glycosides requires glycosyl activation and involves multiple steps of protection/deprotection to control regioselectivity that can often reduce yield of the final product.^{[1-3]} Glycosyltransferases (GTases) offer a potential solution to this problem,^{[}4; 5] since the enzymes use unprotected aglycones in aqueous solution and their catalytic activity is chemo-, regio- and enantio-selective. However to date, the availability of characterized enzymes has been limited and their use as biocatalysts constrained by the need to supply activated sugars for the synthesis of the glycosides. Recently, a large multigene family of GTases has been identified in Arabidopsis thaliana and expressed as recombinant enzymes in Escherichia coli.^{[6]} The need to add activated sugars has been successfully overcome by the use of recombinant GTases in a whole-cell biocatalysis system.^{[15-20]}.

### SUMMARY OF THE INVENTION

In this disclosure we apply the whole-cell biocatalysis system in a format that would enable us to screen a library, consisting of multiple GTase, simultaneously. Thus, single colonies of E. coli expressing an individual GTases were cultured in 96-well titer plates. The screen of catalytic activity needed to be independent of aglycone if the method was to be generic. Therefore, we used a calorimetric detection system for D-glucose^{[21; 22]} experimentally released from glucosides formed during the biocatalysis. We disclose a rapid assessment of GTases to detect those with a high potential for development into whole-cell biocatalysts. This provides the foundation for their subsequent detailed analysis and choice of enzyme to use or improve for the synthesis of aromatic glucosides.

In our co-pending application, (currently unpublished PCT/GB2005/003324) we disclose a method for the screening for GTase polypeptide activity with respect to acceptor molecules. The present disclosure describes the regioselective modification of compounds identified by the screening method disclosed in PCT/GB2005/003324 and an improvement to the screening method.

According to an aspect of the invention there is provided the use of a glycosyltransferase in the regioselective modification of an aglycone with a sugar moiety selected from the group consisting of:
i) a glycosyltransferase encoded by a nucleic acid molecule comprising a nucleic acid sequence as represented in Table 2 (SEQ ID NO: 7, 8, 10, 12, 14, 18, 22-27, 29-32, 35, 39, 40, 53, 55, 66, 81, 84, 91, 97 or 99);
ii) a glycosyltransferase encoded by a nucleic acid molecule that hybridises under stringent hybridisation conditions to a nucleic acid molecule in (i) and which regioselectively modifies an aglycone with a sugar moiety.

An aglycone is a non-sugar containing compound that remains after the replacement of a glycosyl group from a glycoside by a hydrogen atom.

In a preferred embodiment of the invention said glycosyltransferase is encoded by a nucleic acid molecule consisting of a nucleic acid sequence as represented in Table 2 (SEQ ID NO: 7, 8, 10, 12, 14, 18, 22-27, 29-32, 35, 39, 40, 53, 55, 66, 81, 84, 91, 97 or 99).

In a preferred embodiment of the invention said nucleic acid molecule comprises a nucleic acid sequence which has about 50% homology to the nucleic acid sequence represented in Table 2 (SEQ ID NO: 7, 8, 10, 12, 14, 18, 22-27, 29-32, 35, 39, 40, 53, 55, 66, 81, 84, 91, 97 or 99).

Preferably said homology is at least 50%, 60%, 70%, 80%, 90%, or at least 99% identity with the nucleic acid sequence represented in Table 2 (SEQ ID NO: 7, 8, 10, 12, 14, 18, 22-27, 29-32, 35, 39, 40, 53, 55, 66, 81, 84, 91, 97 or 99) and which encodes a polypeptide which regioselectively modifies an aglycone with a sugar moiety.

Hybridization of a nucleic acid molecule occurs when two complementary nucleic acid molecules undergo an amount of hydrogen bonding to each other. The stringency of hybridization can vary according to the environmental conditions surrounding the nucleic acids, the nature of the hybridization method, and the composition and length of the nucleic acid molecules used. Calculations regarding hybridization conditions required for attaining particular degrees of stringency are discussed in Sambrook et al., Molecular Cloning: A Laboratory Manual (Cold Spring Harbor Laboratory Press, Cold Spring Harbor, N.Y., 2001); and Tijssen, Laboratory Techniques in Biochemistry and Molecular Biology-Hybridization with Nucleic Acid Probes Part I, Chapter 2 (Elsevier, New York, 1993). The Tₘ is the temperature at which 50% of a given strand of a nucleic acid molecule is hybridized to its complementary strand. The following is an exemplary set of hybridization conditions and is not limiting:

Very High Stringency (Allows Sequences that Share at Least 90% Identity to Hybridize)

| | |
|---|---|
| Hybridization: | 5x SSC at 65° C. for 16 hours |
| Wash twice: | 2x SSC at room temperature (RT) for 15 minutes each |
| Wash twice: | 0.5x SSC at 65° C. for 20 minutes each |

High Stringency (Allows Sequences that Share at Least 80% Identity to Hybridize)

| | |
|---|---|
| Hybridization: | 5x-6x SSC at 65° C.-70° C. for 16-20 hours |
| Wash twice: | 2x SSC at RT for 5-20 minutes each |
| Wash twice: | 1x SSC at 55° C.-70° C. for 30 minutes each |

Low Stringency (Allows Sequences that Share at Least 50% Identity to Hybridize)

| | |
|---|---|
| Hybridization: | 6x SSC at RT to 55° C. for 16-20 hours |
| Wash at least twice: | 2x-3x SSC at RT to 55° C. for 20-30 minutes each. |

In a preferred embodiment of the invention said aglycone is an isoflavone, for example daidzein.

In an alternative preferred embodiment of the invention said aglycone is a stilbene, for example trans-resveratrol.

In a preferred embodiment of the invention diadzein is regioselectively glycosylated at a 7-OH position.

In a further preferred embodiment of the invention diadzein is regioselectively glycosylated at a 7-OH and 4-OH position.

In a preferred embodiment of the invention trans-resveratrol is regioselectively glycosylated at a 3-OH position.

In an alternative preferred embodiment of the invention trans-resveratrol is regioselectively glycosylated at a 4-OH position.

According to a further aspect of the invention there is provided a screening method to assay the activity of at least one glycosyltransferase polypeptide comprising the steps of:
i) providing a cell culture medium comprising a cell transfected or transformed with a nucleic acid molecule that encodes a glycosyltransferase polypeptide and an aglycone which is an acceptor for a sugar moiety;
ii) separating said cell from the cell culture medium;
iii) contacting said cell culture medium with an agent that removes the sugar moiety from the aglycone and contacting the aglycone with a substance to which said aglycone is bound to remove residual aglycone in the cell culture medium; and
iv) detecting the presence of the sugar in said cell culture medium.

In a preferred method of the invention said substance is polypyrrolidone.

In a preferred method of the invention said glycosyltransferase is selected from the group consisting of: glucosyltransferase; fucosyltransferase; sialyltransferase; galatosyltransferases; glucuronosyltransferases; rhamnosyltransferases; and mannosyltransferases.

In a preferred method of the invention said glycosyltransferase is a plant glucosyltransferase.

In a further preferred method of the invention said nucleic acid molecule encodes a glucosyltransferase selected from the group consisting of:
i) nucleic acid molecules consisting of a nucleic acid sequence as represented in Table 1 (SEQ ID NO: 1-107);
ii) nucleic acid molecules that hybridise under stringent hybridisation conditions to the nucleic acid molecules in (i) and which encode a polypeptide with glucosyltransferase activity;
iii) a nucleic acid molecule that is degenerate as a result of the genetic code to the sequences as defined in (i) and (ii) above.

In a preferred method of the invention said nucleic acid molecule consists of a nucleic acid sequence as represented in Table 1 (SEQ ID NO: 1-107).

In an alternative preferred method of the invention said glycosyltransferase is a mammalian glycosyltransferase. Preferably said mammalian glycosyltransferase is human.

In a preferred method of the invention said cell is a prokaryotic cell. Preferably said prokaryotic cell is Eschercheria coli.

In an alternative preferred method of the invention said cell is a eukaryotic cell.

In a preferred method of the invention said eukaryotic cell is selected from the group consisting of: a yeast cell; an insect cell; a mammalian cell or a plant cell.

In a preferred method of the invention said nucleic acid molecule is part of a vector adapted for the expression of said glycosyltransferase.

Typically said adaptation includes, by example and not by way of limitation, the provision of transcription control sequences (promoter sequences) that mediate cell specific expression. These promoter sequences may be cell specific, inducible or constitutive.

Promoter is an art recognised term and, for the sake of clarity, includes the following features which are provided by example only. Enhancer elements are cis acting nucleic acid sequences often found 5′ to the transcription initiation site of a gene (enhancers can also be found 3′ to a gene sequence or even located in intronic sequences and is therefore position independent). Enhancers function to increase the rate of transcription of the gene to which the enhancer is linked. Enhancer activity is responsive to trans acting transcription factors that have been shown to bind specifically to enhancer elements. The binding/activity of transcription factors (please see Eukaryotic Transcription Factors, by David S Latchman, Academic Press Ltd, San Diego) is responsive to a number of environmental cues that include, by example and not by way of limitation, intermediary metabolites (e.g. sugars), environmental effectors (e.g. light, heat). Promoter elements also include so called TATA box and RNA polymerase initiation selection (RIS) sequences that function to select a site of transcription initiation. These sequences also bind polypeptides that function, inter alia, to facilitate transcription initiation selection by RNA polymerase.

Adaptations also include the provision of selectable markers and autonomous replication sequences that both facilitate the maintenance of said vector in either the eukaryotic cell or prokaryotic host. Vectors that are maintained autonomously are referred to as episomal vectors. Episomal vectors are desirable since these molecules can incorporate large DNA fragments (30-50 kb DNA). Episomal vectors of this type are described in WO98/07876.

Adaptations which facilitate the expression of vector encoded genes include the provision of transcription termination/polyadenylation sequences. This also includes the provision of internal ribosome entry sites (IRES) that function to maximise expression of vector encoded genes arranged in bicistronic or multi-cistronic expression cassettes.

These adaptations are well known in the art. There is a significant amount of published literature with respect to expression vector construction and recombinant DNA techniques in general. Please see, Sambrook et al (1989) Molecular Cloning: A Laboratory Manual, Cold Spring Harbour Laboratory, Cold Spring Harbour, N.Y. and references therein; Marston, F (1987) DNA Cloning Techniques: A Practical Approach Vol III IRL Press, Oxford UK; DNA Cloning: F M Ausubel et al, Current Protocols in Molecular Biology, John Wiley & Sons, Inc (1994).

The invention features polypeptide sequences having at least 75% identity with the polypeptide sequences as herein disclosed, or fragments and functionally equivalent polypeptides thereof. In one embodiment, the polypeptides have at least 85% identity, more preferably at least 90% identity, even more preferably at least 95% identity, still more preferably at least 97% identity, and most preferably at least 99% identity with the amino acid sequences illustrated herein and which retain or has enhanced glycosyltransferase activity.

In a preferred method of the invention said test substrate is selected from the group consisting of; other sugars, proteins, peptides, lipids and other organic substrates, for example intermediate metabolites (e.g. phenylpropanoid derivatives, coumarins, flavonoids, isoflavones, for example diadzein, stilbenes, for example trans-resveratrol).

In a preferred method of the invention said cell is further transformed or transfected with a nucleic acid molecule that encodes a polypeptide or peptide substrate for said glycosyltransferase.

In a preferred method of the invention said preparation further includes a test agent wherein said agent is a potential modulator of said glycosyltransferase.

In a preferred method of the invention said agent is an antagonist of said glycosyltransferase.

Antagonistic agents are agents that, either directly or indirectly, inhibit the activity of a glycosyltransferase. Amongst these are preferably nucleotide analogues that are known to be potential inhibitors of glycosyltransferases, please see U.S. Pat. No. 5,770,407.

In a further preferred method of the invention said first agent is an enzyme that cleaves the sugar from the aglycone, for example a glucosidase.

Cleavage of a sugar moiety prior to detection may be accomplished either chemically or enzymatically (e.g. a glycosidase). The detection of the sugar moiety may be conducted by methods well known in the art.

In a further preferred method of the invention said method comprises a plurality of glycosyltransferases.

In a preferred method of the invention said cell culture medium includes an exogenous source of sugar.

Test formats that allow the simultaneous or near simultaneous assaying of a plurality of glycosyltransferases are known in the art and include the use of multiwell plates comprising assay reactants. Systems are available for the collation of signals from multiple assays.

In a preferred method of the invention said assay further comprises the steps of:
i) collating the signal generated during detection of said sugar from said plurality of glycosyltransferases;
ii) converting the collated signals into a data analysable form; and optionally
iii) providing an output for the analysed data.

According to a further aspect of the invention there is provided a modified aglycone formed by the method according to the invention.

The screening of large numbers of aglycones and/or agents requires preparing arrays of cells for the handling and the administration of substrates/agents. Standard multiwell micro titre plates with formats such as 6, 12, 48, 96 and 384 wells are typically used for compatibility with automated loading and robotic handling systems. Typically, high throughput screens use homogeneous mixtures of agents with an indicator compound that is either converted or modified resulting in the production of a signal. The signal is measured by suitable means (for example detection of fluorescence emission, optical density, or radioactivity) followed by integration of the signals from each well containing the cells, substrate/agent and indicator compound. The present invention utilises the detection of a sugar in cell culture medium and this detection may be the result of the direct detection of the sugar or an indirect measure of the concentration of cleaved sugar from a modified substrate.

### BRIEF DESCRIPTION OF THE FIGURES

An embodiment of the invention will now be described by example only and with reference to the following figures:

FIG. 1: Design of the rapid screening method. This method consists of three stages: aglycone biotransformation (stage 1), cleavage of the glucoside (stage 2), and detection of

the released D-glucose in a coupled enzymatic assay (stage 3);

FIG. 2: Screening of a GT-library against the aglycone scopoletin. a) The readings at A_{405 nm} for D-glucose detection are presented in a colored code format. b) The correlation of the colorimetric detection at A_{405 nm} and the HPLC analysis. HPLC quantifications of glucosides are normalized on the strongest peak and annotated in percentage. c) Examples of RP-HPLC chromatographs of active and non-active GTs in whole-cell biocatalysis are illustrated;

FIG. 3: Screening of a GT-library against the aglycone daidzein. a) The readings at A_{405 nm} for D-glucose detection are presented in a colored code format. b) Examples of RP-HPLC trace of active and non-active GTs in whole-cell biocatalysis are illustrated. c) The regioselectivity of the active GTs towards daidzein, defined by the percentage of a regiospecific glucoside in the total amount of monoglucosides formed;

FIG. 4: Screening of a GT-library against the aglycone trans-resveratrol. a) The readings at A_{405 nm} for D-glucose detection are presented in a colored code format. b) Examples of RP HPLC trace of active and non active GTs in whole cell biocatalysis are illustrated. c) The regioselectivity of the active GTs towards trans-resveratrol, defined by the percentage of a regiospecific glucoside in the total amount of monoglucosides formed;

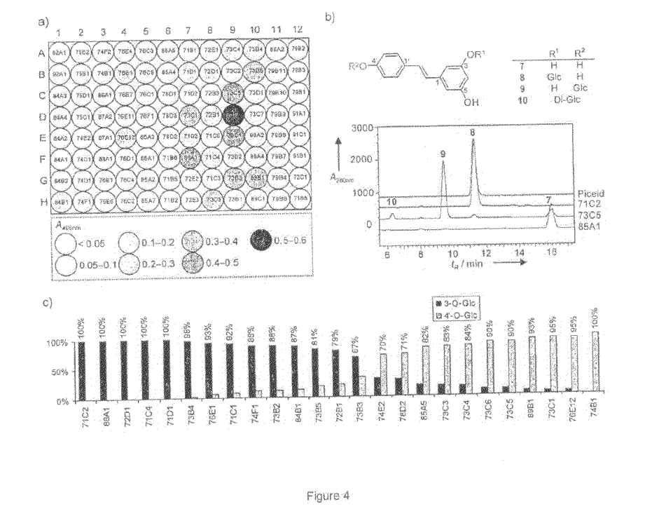

FIG. 5: Investigation of ecsulin hydrolysis. Neither a) autohydrolysis in MES buffer nor b) hydrolysis in bacterial culture of esculin (12) was detected. Samples at 24 h, 44 h incubation and additionally a standard of the aglycone esculetin (11) are illustrated;

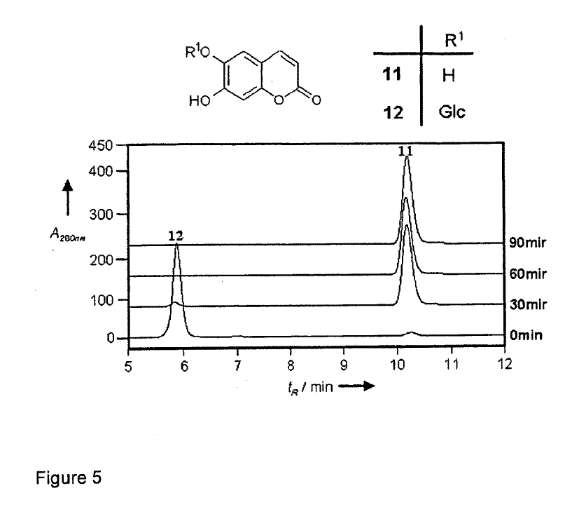

FIG. 6: Cleavage of esculin by □-glucosidase. Samples of the cleavage reaction for the glucoside esculin (12) were analysed by RP-HPLC at 0, 30, 60 and 90 min incubation time;

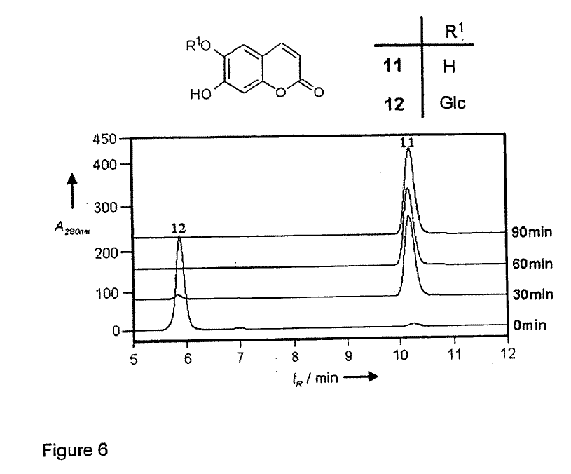

FIG. 7: Removal of different aglycones through adsorbtion by PVPP. The removal of a) trans-resveratrol (100%), b) esculetin (70%), c) daidzein (81%), and d) scopoletin (92%) by PVPP was analyzed by RP-HPLC. The efficiency was defined as the ratio of compounds removed by PVPP over that in the untreated samples;

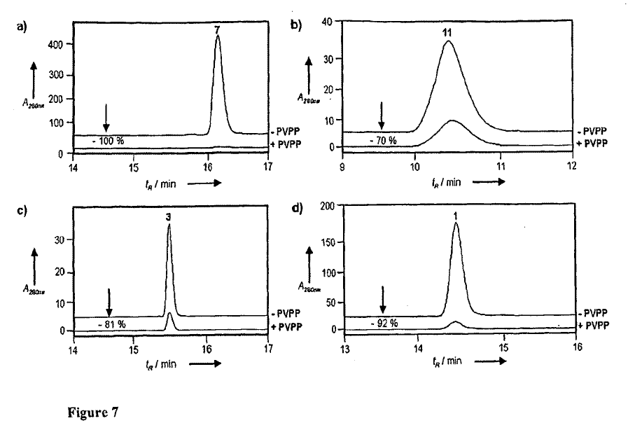

FIG. 8: Lack of D-glucose adsorption by PVPP. The HPAEC chromatograph of D-glucose (13) samples treated with and without PVPP are illustrated demonstrating that no significant loss of D-glucose occurred by filtration through PVPP;

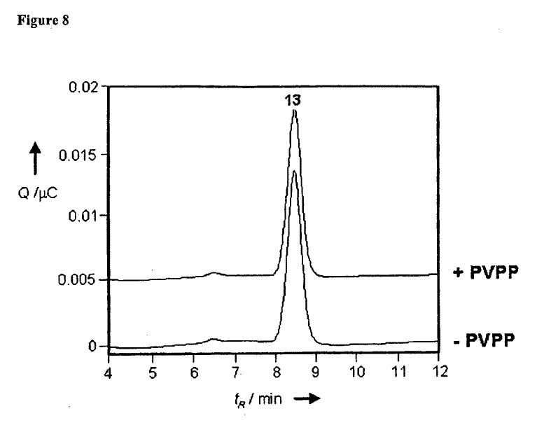

FIG. 9: The correlation of the colorimetric detection at A_{405 nm} and HPLC analysis. HPLC quantifications of glucosides are normalized on the strongest peak and annotated in percentage: a) daidzein glucosides and b) trans-resveratrol glucosides;

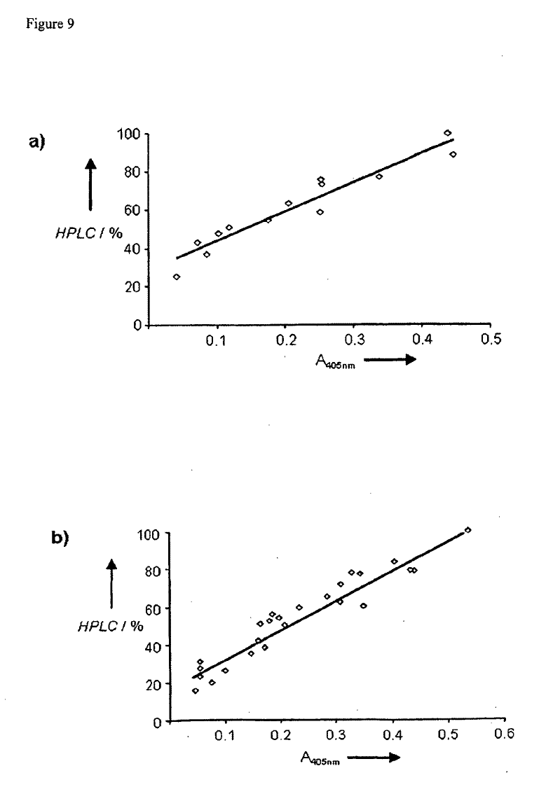

FIG. 10: ¹H-NMR spectral data for daidzein and trans-resveratrol mono-glucosides;

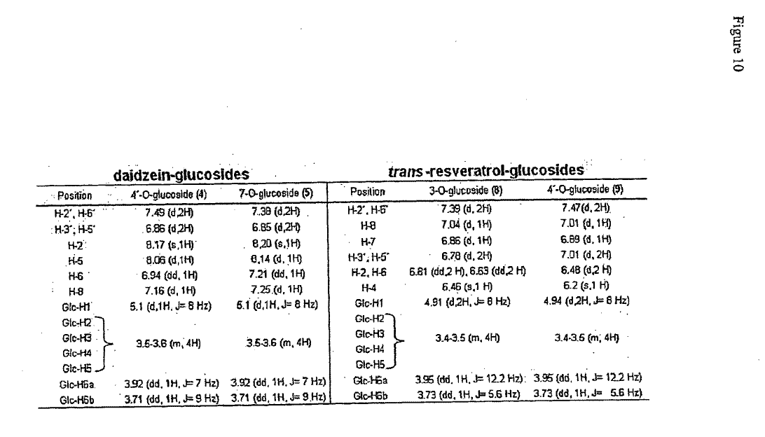

FIG. 11: MS analysis of daidzein glucosides. a) 4′-O-glucoside (4) (m/z: 415.11 [M⁻H]), b) 7-O-glucoside (5) (m/z: 415.10 [M⁻-H]), daidzein (3) (m/z: 253.03 [M⁻-H]), c) daidzein di-glucoside (6) (m/z: 577.10 [M⁻-H]), other peaks annotated are derived fragments; and

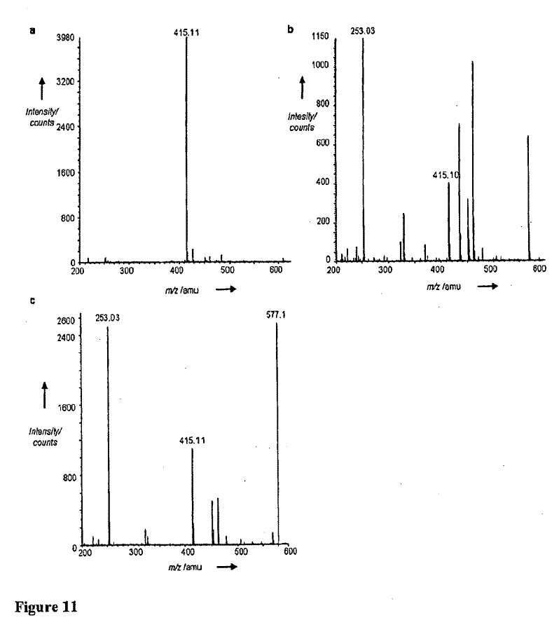

FIG. 12: MS analysis of trans-resveratrol glucosides. a) 4′-O-glucoside (8) (m/z: 389.13 [M⁻-H]), trans-resveratrol (7) (m/z: 227.08 [M⁻-H]) b) 3-O-glucoside (9) (m/z: 389.13 [M⁻-H]), c) trans-resveratrol di-glucoside (10) (m/z: 551.18 [M⁻-H]), other peaks annotated are derived fragments.

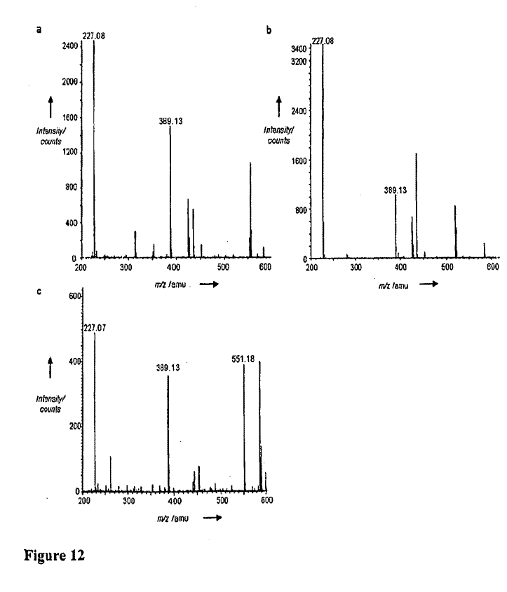

Table 1 shows the coding sequences of 107 Arabidopsis glycosyltransferases; and

Table 2 is a selection of coding sequences of Arabidopsis glycosyltransferases that show regioselective modification of diadzein or trans-resveratrol.

### DETAILED DESCRIPTION

Throughout the description and claims of this specification, the words “comprise” and “contain” and variations of the words, for example “comprising” and “comprises”, means “including but not limited to”, and is not intended to (and does not) exclude other moieties, additives, components, integers or steps.

Throughout the description and claims of this specification, the singular encompasses the plural unless the context otherwise requires. In particular, where the indefinite article is used, the specification is to be understood as contemplating plurality as well as singularity, unless the context requires otherwise.

Features, integers, characteristics, compounds, chemical moieties or groups described in conjunction with a particular aspect, embodiment or example of the invention are to be understood to be applicable to any other aspect, embodiment or example described herein unless incompatible therewith.

### Materials

All reagents were of analytical grade. Scopoletin, daidzein, esculetin, esculin, trans-resveratrol, dadzein-7-O-β-D-glucopyranoside (daidzin), glucose oxidase and almond β-glucosidase were obtained from Sigma-Aldrich (U.K.). Horseradish peroxidase and ABTS™ were purchased from Calbiochem® (U.K.). trans-Resvertarol-3-O-β-D-glucopyranoside (piceid) was obtained from Alexis® Biochemicals (U.K.). MilliQ purified water was used for the preparation of all solutions.

### Analytical Methods

Reverse-phase HPLC (RP-HPLC): RP-HPLC (Agilent 1100 system with Photodiode Array Detector, Agilent, U.K.) analysis was carried out using a Columbus 5-μ C18 column (150×3.20 mm, Phenomenex, U.K.). Glucosides were separated from their respective aglycones using a linear gradient of acetonitrile/0.1% formic acid (v/v) in H₂O: 10-45% (trans-resveratrol/glucosides), 10-50% (daidzein/glucosides) at 0.5 mL/min over 20 min and monitored at 280 nm and 250 nm. Separation of scopoletin/scopolin and esculetin/esculin was carried out using the conditions described previously.^{[11]}

High Performance Anion Exchange Chromatography (HPAEC): HPAEC coupled with integrated amperometric detection (IAD) (Dionex, U.K.) was used to detect D-glucose using a CarboPac™ PA10 column (2×250 mm, Dionex). Seven different monosaccharides including L-Fucose, L-rhamnose, D-galactose, L-arabinose, D-glucose, D-manose and D-xylose were used as references. The D-glucose was separated isocratically at a flow rate 0.35 mL/min with 24 mM NaOH (pH>12.5) over 18 min. The column was then washed with a linear gradient of NaOH from 24 mM to 200 mM over 5 min. The IAD waveform was set following manufacturer's recommendation.

¹H-NMR: Glucosides, produced in a large-scale biocatalysis, were extracted from the culture media into n-butanol, purified using HPLC, re-extracted with n-butanol, dried under vacuum and solubilized in CD₃OD for ¹H-NMR analysis (Bruker AMX 500-MHz ¹H-NMR spectrometer). The data were processed and analyzed using Bruker XWIN-NMR software version 2.6.

ESI-MS: Negative ion electrospray MS and MS/MS data (Applied Biosystems QSTAR Pulsar i hybrid quadropole time-of-flight instrument) were collected and processed using ANALYST QS (Applied Biosystems) software. The mass spectrometer was operated in negative ion mode with an ion spray voltage of −2500 V at 300° C. and the nebulisor and turbo gases set at 70 units. Parent ions were fragmented by collision induced dissociation (CID) and product ions analysed from 50 to 800 amu. The energy fragmentation experiments used collision energy settings of −60 V.

### Development of the Screening Method

For each round of screening, a negative control containing the substrate and E. coli transformed with the vector pGEX-2T was included. In addition, E. coli expressing GT 71 C1 and incubated with scopoletin was used as a positive control. Each stage in the screening method was validated by further controls described as follows.

The lack of autohydrolysis during incubation was confirmed using esculin (12) (esculetin-6-O-glucoside) incubated in 50 mM MES buffer (pH 7.0). Incubation of esculin with E. coli transformed with pGEX-2T vector indicated the glucoside was not hydrolyzed in the presence of the bacterial culture. For these controls, samples were incubated for 44 h at 25° C. as in the standard experimental conditions, and analyzed by RP-HPLC to confirm the lack of aglycone (esculetin, 11) (FIG. S1).

The cDNA library of 96 Arabidopsis thaliana GTs was subcloned into the multiple cloning site of the glutathione-S-transferase (GST) gene fusion vector pGEX-2T (Amersham Biosciences, U.K.) as described previously^{[10]} and transformed into the strain E. coli BL21 (DE3) for use in the screening method.

Stage 1, biotransformation: single colonies of the GT library grown on LB-agar plates overnight were transferred to individual wells in a 96-well bacterial culture plate containing 400 μl 2×YT medium (16 g/L bacto tryptone, 10 g/L yeast extract, 5 g/L NaCl) and 50 μg/mL ampicillin. The plate was covered with an adhesive plate seal (Abgene, U.K.) and incubated at 37° C. (250 rpm). The bacterial growth was monitored at 595 nm by a plate reader. After 4 h, the cultures had reached exponential phase. The plate was centrifuged (4000 g, 10 min), the supernatants discarded and cell pellets were resuspended in isopropyl-D-thiogalactopyranoside (0.1 mM), 2-(N-morpholino)ethanesulfonic acid (50 mM, pH 7.0), ampicillin (50 μg/mL), L-arabinose (10 g/L) and 500 □M of aglycone to a total whole-cell reaction volume of 400 μl/well. The 96-well plate was closed with a gas permeable adhesive plate seal, wrapped in alu foil for light protection and incubated at 25° C. (250 rpm). After 44 h the cultures were centrifuged (4000 g, 15 min) and the supernatants analyzed.

Stage 2, cleavage: supernatants (100 μl) were transferred to a microtiter plate, 1 μl of β-glucosidase (1 U) was added and the plate incubated for 90 min at 37° C.

Stage 3, detection: 50 μl of the reaction mix were transferred to a 96-well filtration plate (Abgene, U.K.), mixed with an equal volume of PVPP aqueous suspension (25 g/L), shaken for 1 h at 25° C. before centrifugation (1000 g, 5 min). To each filtrate, 50 mM 2-morpholino-ethanesulfonic acid buffer (MES) (pH 7.0), ABTS™ (0.1 mM), peroxidase (2 U) and glucose oxidase (2 U) were added to a final volume of 125 μl. The formation of the green dye was monitored at 405 nm at 30 min using a plate reader (Bio-Tec Instruments Inc., U.S.A).

### EXAMPLES

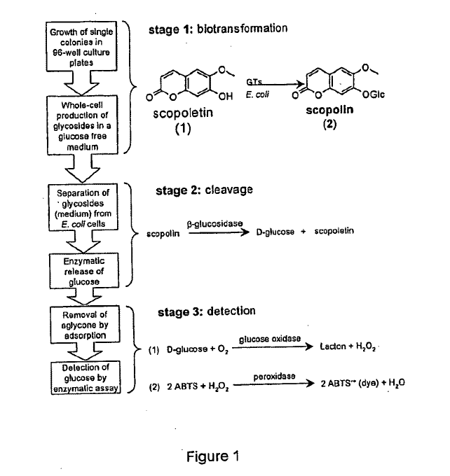

The method, illustrated in scheme 1, was established and optimized for a 96-well plate format using the conversion of the hydroxycoumarin, scopoletin (1) to scopolin (2) as a model system. In vitro catalysis had already demonstrated that the substrate was recognized by multiple recombinant arabidopsis GTs.^{[10]} Cells were cultured in standard media before transfer to D-glucose-minus medium in which L-arabinose was the carbon source. Following induction, addition of substrate and incubation, cells were separated and the media from each well were collected and samples either analyzed directly using reverse-phase (RP) HPLC or treated with □ β-glucosidase, filtered through polyvinyl-polypyrrolidone (PVPP) to remove remaining aglycone and levels of D-glucose detected in an enzymatic assay. FIG. 1 illustrates the GT activities towards scopoletin and demonstrates a linear relationship between the amount of scopolin formed in each reaction and D-glucose detection. The whole-cell biocatalysis and screen identified 45 GTs with activity towards scopoletin, confirming and extending the earlier data from in vitro catalysis. Invariably, a negative in the D-glucose detection assay correlated with a negative result in the RP-HPLC analysis.

The utility of the method to discover novel biocatalysts was investigated using the isoflavone, daidzein (3) and the stilbene, trans-resveratrol (7). Both compounds exist as glucosides, have attracted considerable pharmaceutical interest,^{[23-27]} and chemical synthesis of their different glycosides has been attempted but resulted in poor yields and lack of regioselective discrimination.^{[28-30]} Daidzein, as well as other isoflavones, occurs naturally in legumes as the 7- and 4′-β-O-glucosides (4 daidzin, 5).^{[31]} trans-Resveratrol (7), a naturally occurring hydroxystilbene, is found as glucosides^{[32]} and methoxides.^{[33]} Piceid (3-β-O-glucoside) (8) and resveratroloside (4′-β-O-glucoside) (9) are the most abundant conjugates. Bioactivity of these compounds has been reported in relation to cancer prevention,^{[34-36]} coronary heart disease,^{[37; 38]} antioxidant activity^{[39; 40}] and estrogenic activity.^{[41; 42]} Since neither daidzein nor trans-resveratrol is reported to occur in arabidopsis, they represent non-natural substrates for the GT screen.

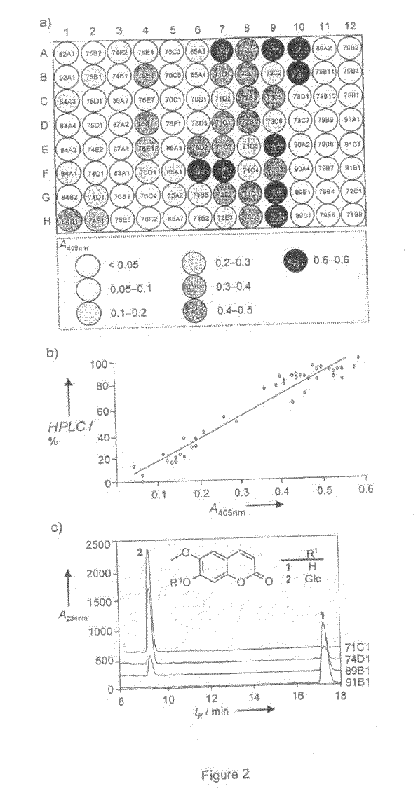

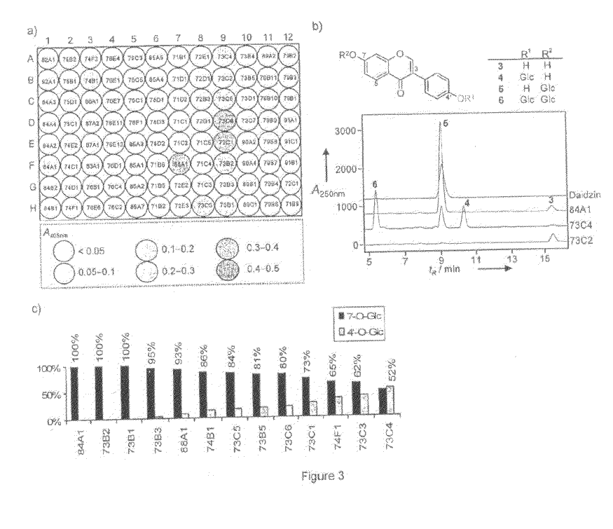

The utility of the screening method and regioselective biocatalysis by the GTs are illustrated in FIGS. 2 and 3. Thirteen GTs recognized daidzein and twenty-five GTs were identified that glycosylated trans-resveratrol. As previously described for scopoletin, RP-HPLC quantification of the glucosides formed in the biocatalysis revealed a linear correlation to D-glucose detection for both substrates (FIG. S5, supporting information). The mono- and di-glucosides of daidzein (4-6) and trans-resveratrol (8-10), eluting earlier than the two aglycones under the RP-HPLC conditions used (FIGS. 2b and 3b), were identified using external standards when available, or by electrospray liquid chromatography-mass spectrometry (LC-MS). ¹H-NMR analysis was used to confirm the structure of the monoglucosides (Table 1, SEQ ID NO: 1-107). From the thirteen GTs that recognized daidzein, three (GTs 84A1, 73B2 and 73B1) were found to be 100% regioselective for the 7-OH; the remaining enzymes glycosylated the 4′-OH and 7-OH positions to varying degrees, and one GT, 73C4, produced the diglucoside in addition to the monoglucosides (FIG. 2b). Similarly, regioselective glycosylation of trans-resveratrol was observed. From the twenty-five enzymes that recognized the substrate, five GTs were specific for the 3-OH position (GTs 71 D1, 71C2, 88A1, 72D1 and 71C4) and one GT 74B1 was specific for the 4′-OH position (FIG. 3b). Only trace levels of a diglucoside were observed under the reaction conditions used. As before, for both daidzein and trans-resveratrol biocatalysis, the D-glucose based detection system did not miss any positive enzyme activities; however in these assays, two false positives in screens of each compound were observed, where an intense absorption was not associated with any product formation.

In conclusion, we have successfully developed a generic screen to determine the activity of recombinant GT libraries towards aromatic compounds in whole-cell biocatalysis. We have demonstrated that the method provides the means to rapidly identify GTs of high utility that can be further developed for use in biotransformations or chemo-enzymatic synthesis of small molecule glycosides. The regio- and enantio-selectivity of GT biocatalysts offers a useful complement to classical chemical approaches.

### REFERENCES

[1.] H. Pellissier, Tetrahedron 2005, 61 2947-2993.
[2.] K. C. Nicolaou, H. J. Mitchell, Angew. Chem. Int. Ed Engl. 2001, 40 1576-1624.
[3.] S. Hanessian, B. L. Lou, Chemical Reviews 2000, 100 4443-4463.
[4.] S. L. Flitsch, Curr. Opin. Chem. Biol. 2000, 4 619-625.
[5.] K. M. Koeller, C. H. Wong, Chemical Reviews 2000, 100 4465-4493.
[6.] Y. Li, S. Baldauf, E. K. Lim, D. J. Bowles, J. Biol. Chem. 2001, 276 4338-4343.
[7.] B. Hou, E. K. Lim, G. S. Higgins, D. J. Bowles, J. Biol. Chem. 2004, 279 47822-47832.
[8.] E. K. Lim, C. J. Doucet, Y. Li, L. Elias, D. Worrall, S. P. Spencer, J. Ross, D. J. Bowles, J. Biol. Chem. 2002, 277 586-592.
[9.] E. K. Lim, C. J. Doucet, B. Hou, R. G. Jackson, S. R. Abrams, D. J. Bowles, Tetrahedron-Asymmetry 2005, 16 143-147.
[10.] E. K. Lim, S. Baldauf, Y. Li, L. Elias, D. Worrall, S. P. Spencer, R. G. Jackson, G. Taguchi, J. Ross, D. J. Bowles, Glycobiology 2003, 13 139-145.
[11.] C. Loutre, D. P. Dixon, M. Brazier, M. Slater, D. J. Cole, R. Edwards, Plant J 2003, 34 485-493.
[12.] B. Poppenberger, F. Berthiller, D. Lucyshyn, T. Sieberer, R. Schuhmacher, R. Krska, K. Kuchler, J. Glossl, C. Luschnig, G. Adam, J. Biol. Chem. 2003, 278 47905-47914.
[13.] T. Hefner, J. Arend, H. Warzecha, K. Siems, J. Stockigt, Bioorg. Med. Chem. 2002, 10 1731-1741.
[14.] B. Messner, O. Thulke, A. R. Schaffner, Planta 2003, 217 138-146.
[15.] V. Kren, J. Thiem, Chemical Society Reviews 1997, 26 463-473.
[16.] S. Koizumi, T. Endo, K. Tabata, A. Ozaki, Nature Biotechnology 1998, 16 847-850.
[17.] E. K. Lim, D. A. Ashford, B. Hou, R. G. Jackson, D. J. Bowles, Biotechnol. Bioeng. 2004, 87 623-631.
[18.] M. G. Willits, M. Giovanni, R. T. Prata, C. M. Kramer, L. De, V, J. C. Steffens, G. Graser, Phytochemistry 2004, 65 31-41.
[19.] S. Koizumi, Trends in Glycoscience and Glycotechnology 2003, 15 65-74.
[20.] J. Arend, H. Warzecha, T. Hefner, J. Stockigt, Biotechnol. Bioeng. 2001, 76 126-131.
[21.] N. P. Groome, J. Clin. Chem. Clin. Biochem. 1980, 18 345-349.
[22.] D. C. Williams, G. F. Huff, W. R. Seitz, Clin. Chem. 1976, 22 372-374.
[23.] G. Galati, P. J. O'Brien, Free Radical Biology and Medicine 2004, 37287-303.
[24.] L. Fremont, Life Sci. 2000, 66 663-673.
[25.] P. Signorelli, R. Ghidoni, The Journal of Nutritional Biochemistry 2005, 16 449-466.
[26.] J. Reynaud, D. Guilet, R. Terreux, M. Lussignol, N. Walchshofer, Nat. Prod. Rep. 2005, 22 504-515.
[27.] K. D. R. Setchell, A. Cassidy, Journal of Nutrition 1999, 129 758S-767S.
[28.] P. W. Needs, G. Williamson, Carbohydr. Res. 2001, 330 511-515.
[29.] F. Orsini, F. Pelizzoni, B. Bellini, G. Miglierini, Carbohydr. Res. 1997, 301 95-109.
[30.] P. T. Lewis, K. Wahala, Tetrahedron Letters 1998, 39 9559-9562.
[31.] Y. Shibuya, S. Tahara, Y. Kimura, J. Miyzutani, Z. Naturforsch. 1991, 46c 513-518.
[32.] D. M. Goldberg, E. Ng, A. Karumanchiri, E. P. Diamandis, G. J. Soleas, Am. J. Enol. Vitic. 1996, 47 415-420.
[33.] P. Langcake, C. A. Cornford, R. J. Pryce, Phytochemistry 1979, 18 1025-1027.
[34.] H. Adlercreutz, M. Yaghoob, K. Hoeckerstedt, Acta Oncologica 1992, 350 115-181.
[35.] M. Jang, L. Cai, G. O. Udeani, K. V. Slowing, C. F. Thomas, C. W. Beecher, H. H. Fong, N. R. Farnsworth, A. D. Kinghorn, R. G. Mehta, R. C. Moon, J. M. Pezzuto, Science 1997, 275 218-220.
[36.] D. Ingram, K. Sanders, M. Kolybaba, D. Lopez, Lancet 1998, 350 990-994.
[37.] S. Samman, P. Lyons-Wall, N. Cook, Antioxid. Health Dis 1998, 7 469-481.
[38.] P. Nestel, T. Yamashita, T. Sasahara, S. Pomeroy, A. Dart, P. Komesaroff, A. Owen, A. Abbey, Arterioscler. Throm. Biol. 1997, 17 3392-3398.
[39.] M. Begona-Ruiz-Larrera, A. Moham, G. Paganga, N. Miller, G. Bolwell, C. Rice-Evans, Free Radical Res. 1997, 26 63-70.
[40.] M. J. Tikkanen, K. Wahala, S. Ojala, V. Vihma, H. Adlercreutz, Proc. Natl. Acad. Sci. U.S.A 1998, 95 3106-3110.
[41.] S. D. Garrett, H. A. Lee, M. R. A. Morgan, Nature Biotechnology 1999, 17 1219-1222.
[42.] B. D. Gehm, J. M. McAndrews, P. Y. Chien, J. L. Jameson, Proc. Natl. Acad. Sci. U.S.A 1997, 94 14138-14143.

TABLE 1

SEQ ID NO: 1 >UGT71B1
ATGAAAGTAGAACTTGTGTTCATACCATCGCCGGGCGTTGGCCATATCCGAGCAAC
AACGGCGTTAGCAAAGCTTCTCGTTGCCAGCGACAACCGCCTCTCCGTCACTCTCA
TCGTCATTCCTTCACGAGTCTCCGACGACGCTTCTTCCTCCGTCTACACGAACTCC
GAAGACCGTCTCCGCTACATCCTCCTCCCCGCCCGAGATCAAACTACTGATCTCGT
ATCTTACATCGACAGCCAGAAACCACAAGTAAGAGCCGTCGTGTCCAAGGTCGCTG
GAGATGTTTCAACACGTTCAGACTCACGGCTAGCTGGGATTGTCGTAGACATGTTC
TGCACGTCCATGATAGACATCGCCGATGAGTTTAACCTCTCGGCTTATATCTTCTAC
ACGTCCAACGCTTCTTATCTCGGGCTACAGTTCCACGTTCAATCTCTTTACGACGAG
AAAGAACTCGACGTAAGTGAGTTCAAAGATACGGAGATGAAGTTTGACGTTCCAAC
TCTGACTCAGCCTTTTCCGGCAAAATGTTTGCCTTCAGTGATGCTAAACAAGAAATG
GTTTCCTTACGTTTTGGGTCGAGCTAGAAGTTTTAGAGCAACGAAGGGTATTTTGGT
AAATTCGGTGGCTGACATGGAACCTCAGGCGTTGAGTTTCTTTTCCGGTGGAAATG
GGAATACAAATATCCCTCCGGTGTACGCGGTTGGGCCCATTATGGACTTAGAATCT
AGCGGCGATGAAGAGAAGAGAAAGGAGATTTTACATTGGCTAAAAGAGCAACCGAC
GAAATCTGTAGTGTTTCTCTGTTTTGGGAGCATGGGAGGTTTCAGTGAGGAACAAG
CAAGAGAAATAGCTGTGGCGCTCGAGCGAAGCGGACACAGGTTTCTCTGGTCGCT
TCGCCGCGCTTCTCCTGTTGGAAACAAGTCTAATCCTCCTCCCGGAGAATTCACGA
ACTTAGAGGAGATTCTTCCAAAAGGGTTTTTAGATCGGACGGTGGAGATAGGGAAG
ATCATAAGCTGGGCACCACAAGTAGATGTGTTGAATAGTCCTGCTATAGGAGCGTT
CGTGACACATTGTGGATGGAACTCAATTCTCGAGAGTCTTTGGTTCGGTGTTCCGA
TGGCGGCGTGGCCTATCTATGCTGAGCAACAGTTTAACGCGTTTCATATGGTGGAT
GAGCTTGGTTTAGCGGCGGAGGTAAAGAAGGAGTACCGTAGAGATTTTCTGGTGG
AGGAGCCGGAGATTGTGACGGCTGATGAGATAGAGAGAGGGATCAAGTGTGCGAT
GGAGCAGGATAGCAAGATGAGGAAGAGGGTGATGGAGATGAAGGATAAGCTCCAC
GTGGCGTTGGTGGACGGTGGATCTTCGAACTGTGCTCTAAAGAAGTTTGTTCAAGA
CGTGGTCGATAATGTTCCATAA
SEQ ID NO: 2 >UGT71B2
ATGAAACTGGAGCTGGTGTTCATACCATCACCTGGTGACGGACATCTCCGGCCATT
AGTGGAGGTAGCTAAGCTTCATGTTGACCGTGACGACCATCTCTCCATCACCATCA
TCATCATCCCTCAGATGCATGGATTTAGTAGCAGTAACTCTTCTTCTTACATCGCTT
CTCTCTCCTCTGATTCTGAAGAACGTCTTAGCTACAACGTTCTCTCCGTCCCTGATA
AACCAGACTCCGATGACACCAAACCACATTTTTTCGACTACATTGATAACTTCAAGC
CGCAGGTCAAAGCCACGGTGGAAAAACTTACTGACCCGGGTCCACCAGATTCGCC
GTCGCGTCTTGCTGGATTCGTGGTGGATATGTTTTGCATGATGATGATTGATGTCG
CTAATGAGTTTGGTGTTCCCAGTTACATGTTTTACACATCCAACGCAACGTTTCTTG
GATTGCAAGTTCATGTTGAATACCTTTACGACGTTAAGAACTATGACGTTAGTGACC
TCAAGGACTCGGACACTACTGAGCTGGAAGTTCCTTGTTTGACTCGTCCTTTACCG
GTTAAGTGTTTCCCCTCGGTTCTATTAACCAAGGAGTGGTTACCGGTTATGTTTAGA
CAAACCAGAAGATTCCGAGAAACTAAAGGTATTTTGGTAAATACATTCGCTGAGCTT
GAGCCTCAAGCTATGAAGTTTTTCTCCGGCGTAGATAGTCCTCTGCCTACGGTGTA
CACAGTTGGACCGGTTATGAATCTTAAAATCAACGGTCCAAATTCATCTGACGATAA
GCAATCGGAGATCCTACGGTGGCTAGACGAGCAGCCACGTAAATCCGTTGTTTTCC
TCTGTTTCGGAAGCATGGGAGGTTTCCGTGAGGGCCAAGCTAAAGAAATCGCAATC
GCGCTTGAGCGAAGTGGTCACCGCTTTGTCTGGTCTCTTCGTCGTGCTCAACCAAA
AGGATCGATAGGACCTCCCGAAGAATTTACGAATCTTGAGGAAATTCTCCCGGAAG
GATTCTTGGAACGGACGGCAGAGATAGGAAAGATTGTAGGTTGGGCTCCACAAAG
CGCCATTCTAGCAAATCCTGCGATCGGAGGGTTCGTGTCGCATTGTGGATGGAACT
CGACGCTAGAGAGTCTATGGTTCGGAGTTCCGATGGCTACGTGGCCGCTTTACGC
AGAGCAACAAGTTAACGCGTTCGAGATGGTTGAGGAGCTAGGGCTAGCGGTGGAG
GTCCGAAATAGTTTCCGAGGAGATTTCATGGCGGCGGATGATGAGTTGATGACGG
CAGAGGAGATAGAGAGAGGGATCCGGTGTTTGATGGAGCAGGATAGTGACGTGAG
GAGTAGAGTGAAGGAGATGAGCGAGAAGAGTCACGTAGCTTTAATGGACGGTGGA
TCTTCGCACGTTGCTCTTCTAAAGTTTATTCAAGACGTCACTAAGAATATCTCTTGA
SEQ ID NO: 3 >UGT71B5
ATGAAGATTGAGCTTGTGTTCATACCTTTGCCGGGGATTGGTCATCTCAGGCCAAC
CGTGAAGCTAGCGAAGCAACTCATAGGCAGCGAAAACCGTCTTTCGATCACCATAA
TCATCATCCCTTCAAGATTTGACGCCGGTGATGCATCCGCCTGTATCGCATCTCTCA
CCACGTTGTCTCAAGATGATCGCCTCCATTACGAATCCATATCCGTCGCAAAACAAC
CACCAACCTCCGACCCGGATCCTGTTCCGGCTCAAGTGTACATAGAGAAACAAAAG
ACGAAAGTGAGAGATGCAGTCGCGGCGAGAATCGTCGATCCAACAAGAAAGCTCG
CGGGATTCGTGGTGGACATGTTCTGTTCCTCGATGATCGATGTAGCTAACGAGTTT
GGAGTTCCGTGTTATATGGTATACACATCGAACGCTACGTTTTTAGGAACCATGCTT
CACGTTCAACAAATGTACGATCAAAAGAAGTATGACGTCAGCGAGTTAGAAAACTC
GGTCACCGAGTTGGAGTTTCCGTCTCTGACTCGTCCTTATCCAGTGAAGTGTCTTC
CTCATATCCTCACTTCAAAGGAGTGGTTACCTCTCTCTCTAGCTCAAGCTAGGTGTT
TCCGGAAGATGAAGGGTATTTTGGTAAATACAGTTGCTGAGCTTGAACCTCACGCT
TTGAAAATGTTCAATATTAATGGTGACGATCTTCCTCAAGTTTATCCTGTTGGACCA
GTGTTGCATCTCGAAAACGGCAATGACGATGATGAGAAGCAATCGGAAATTTTGCG
GTGGCTCGACGAGCAACCGTCTAAATCTGTTGTGTTTCTCTGCTTTGGGAGCTTGG
GAGGTTTCACTGAAGAACAAACAAGAGAAACCGCTGTGGCCCTAGATAGAAGCGGT
CAGCGGTTTCTTTGGTGTCTTCGTCACGCATCGCCAAATATAAAAACAGATCGTCCC
AGAGATTACACGAATCTTGAGGAGGTTTTACCGGAGGGGTTCTTGGAACGGACTTT
GGATAGAGGGAAAGTGATTGGATGGGCACCACAAGTGGCGGTACTAGAGAAGCCG
GCGATAGGAGGGTTTGTCACTCACTGCGGTTGGAACTCTATTTTAGAGAGCTTGTG
GTTCGGTGTTCCAATGGTGACGTGGCCGCTATACGCGGAACAGAAGGTTAACGCG
TTTGAGATGGTTGAGGAGCTGGGTTTGGCGGTGGAGATACGGAAGTACTTAAAAG
GAGATTTGTTCGCCGGAGAGATGGAGACGGTTACCGCGGAGGATATAGAGAGAGC
CATTAGGCGTGTGATGGAGCAAGACAGTGACGTTAGGAACAACGTGAAAGAGATG
GCGGAGAAGTGCCACTTCGCGTTAATGGACGGTGGATCTTCGAAGGCGGCTTTGG
AAAAGTTTATTCAAGACGTGATAGAGAATATGGATTAA
SEQ ID NO: 4 >UGT71B6
ATGAAAATAGAGCTAGTATTCATTCCCTCTCCGGCAATTAGTCATCTCATGGCGACG
GTAGAGATGGCGGAGCAACTAGTTGATAAAAACGACAACCTCTCTATCACCGTAAT
CATCATATCTTTTAGTTCTAAAAATACATCCATGATCACCTCTCTTACATCCAACAAC
CGCCTCCGGTACGAAATAATCTCCGGAGGAGATCAACAACCAACGGAGCTCAAAG
CAACTGATTCCCACATCCAAAGTCTAAAGCCACTGGTGAGAGACGCGGTTGCTAAA
CTCGTAGATTCCACTCTACCAGACGCGCCTCGTCTTGCGGGATTCGTTGTTGACAT
GTACTGCACGTCGATGATCGATGTCGCTAACGAATTTGGCGTCCCTAGTTACTTGT
TTTACACCTCTAACGCTGGATTTCTTGGACTTTTGCTTCACATTCAGTTCATGTACGA
TGCAGAGGATATCTATGACATGAGCGAATTAGAAGACTCTGACGTAGAGTTGGTGG
TTCCGAGTTTGACTAGTCCTTATCCGTTGAAATGTCTTCCTTACATTTTCAAATCAAA
AGAGTGGCTCACTTTTTTTGTAACTCAAGCGAGAAGATTCAGAGAAACTAAGGGCA
TTTTGGTAAACACGGTTCCTGACTTGGAACCTCAAGCGTTGACGTTTCTTTCCAATG
GTAACATTCCACGTGCTTACCCAGTAGGACCATTGTTGCATCTCAAAAACGTAAATT
GTGATTACGTGGACAAGAAGCAATCGGAGATTTTACGGTGGCTAGACGAGCAACC
GCCAAGATCTGTAGTGTTCCTCTGTTTCGGGAGCATGGGAGGGTTCAGTGAGGAA
CAAGTGAGAGAAACCGCATTAGCTCTCGATCGAAGCGGCCACCGGTTTCTTTGGTC
TCTCCGTCGTGCATCTCCGAATATATTGAGAGAGCCTCCCGGAGAATTCACAAACC
TAGAGGAGATTCTCCCAGAAGGGTTTTTCGATCGGACGGCTAACAGAGGAAAGGTT
ATCGGATGGGCTGAACAGGTGGCCATATTGGCGAAGCCGGCGATCGGAGGTTTTG
TTTCTCACGGCGGATGGAATTCGACGTTGGAGAGTTTGTGGTTTGGTGTTCCGATG
GCGATTTGGCCGCTTTACGCTGAACAGAAGTTTAACGCTTTCGAGATGGTGGAAGA
GCTTGGTTTGGCTGTGGAGATCAAGAAGCATTGGCGAGGAGATCTTTTGTTGGGG
AGGTCGGAGATTGTGACGGCGGAGGAGATTGAGAAAGGAATCATATGTTTGATGG
AGCAAGACAGTGACGTCAGGAAGAGAGTGAATGAGATCAGCGAGAAGTGCCACGT
GGCTTTAATGGACGGTGGATCGTCAGAAACTGCTTTGAAAAGATTTATTCAAGACGT
AACGGAGAATATTGCTTGGTCGGAAACTGAAAGCTAG
SEQ ID NO: 5 >UGT71B7
ATGAAATTTGAGCTTGTTTTCATCCCCTATCCCGGAATCGGTCATCTCCGATCAACG
GTAGAAATGGCAAAGCTACTAGTGGACCGTGAAACTCGTCTCTCTATCTCCGTTATC
ATCCTTCCTTTCATTTCCGAAGGCGAAGTCGGTGCTTCCGATTACATCGCAGCCCT
CTCCGCCTCATCCAACAACCGCCTCCGCTACGAAGTTATCTCCGCCGTAGATCAAC
CAACCATCGAGATGACGACAATTGAAATCCATATGAAGAACCAAGAACCAAAGGTG
AGAAGCACCGTTGCAAAACTCCTTGAAGACTATTCGTCTAAACCGGACTCGCCGAA
GATCGCTGGCTTTGTTCTAGACATGTTTTGCACTTCGATGGTAGATGTAGCGAACG
AGTTTGGTTTCCCGAGTTATATGTTTTACACCTCCAGTGCCGGGATTCTCTCAGTTA
CATATCATGTTCAAATGTTGTGCGATGAGAACAAGTACGATGTTAGTGAAAATGATT
ATGCAGACTCGGAAGCTGTGTTGAACTTTCCGAGTTTGAGTCGTCCTTATCCGGTG
AAGTGTCTTCCTCACGCTCTGGCAGCTAATATGTGGCTCCCGGTGTTTGTAAACCA
AGCGAGAAAGTTTAGGGAGATGAAAGGTATTTTGGTAAATACTGTTGCTGAGCTTG
AACCTTATGTGTTAAAGTTTCTTTCTAGTAGTGATACTCCTCCTGTTTATCCTGTTGG
ACCATTGTTGCATCTTGAGAACCAACGTGATGATTCTAAGGACGAGAAACGGTTGG
AGATTATACGGTGGTTGGATCAGCAACCACCAAGTTCGGTTGTGTTTCTCTGCTTT
GGGAGCATGGGAGGCTTCGGTGAGGAACAAGTAAGAGAGATCGCAATCGCGTTAG
AGCGAAGTGGGCACCGGTTTCTCTGGTCTCTTCGTCGCGCATCTCCGAATATATTC
AAAGAACTTCCAGGAGAGTTTACTAATCTAGAGGAAGTTCTCCCGGAAGGATTCTTT
GATCGAACGAAAGATATAGGTAAAGTGATTGGATGGGCTCCACAAGTAGCCGTTCT
TGCGAATCCGGCTATAGGAGGTTTCGTAACTCATTGCGGGTGGAATTCTACGCTAG
AGAGTCTTTGGTTTGGTGTTCCAACAGCTGCATGGCCGTTATACGCAGAGCAGAAG
TTCAATGCTTTCTTAATGGTGGAGGAGCTTGGATTGGCGGTGGAGATAAGGAAGTA
TTGGCGAGGTGAACATTTGGCGGGATTACCGACGGCTACTGTGACAGCGGAGGAG
ATAGAGAAAGCAATCATGTGTCTAATGGAACAAGATAGTGACGTGAGGAAAAGAGT
GAAGGATATGAGCGAGAAATGCCATGTGGCTTTAATGGATGGTGGATCGTCGCGTA
CTGCGTTGCAAAAGTTTATTGAAGAGGTTGCGAAGAATATAGTTTCACTAGATAAGG
AATTTGAGCATGTAGCTCTTAAATGA
SEQ ID NO: 6 >UGT71B8
ATGAACAAATTTGCGCTTGTCTTCGTACCATTTCCTATACTTGGTCATCTCAAATCAA
CCGCCGAGATGGCTAAGCTACTAGTGGAGCAAGAAACTCGCCTCTCTATCTCCATT
ATCATCCTTCCTCTTCTTTCCGGAGACGACGTCAGTGCTTCCGCTTATATCTCAGCT
CTTTCCGCCGCATCCAACGACCGCCTTCACTATGAAGTGATCTCGGACGGAGATCA
ACCAACCGTCGGGTTACATGTCGATAACCACATCCCGATGGTGAAACGTACCGTTG
CAAAACTCGTTGATGACTACTCAAGGCGGCCGGACTCGCCGAGGCTCGCTGGTTT
AGTTGTTGACATGTTTTGTATCTCGGTGATAGACGTGGCTAATGAGGTTAGTGTTCC
GTGTTACTTGTTTTACACGTCAAACGTTGGGATTCTTGCTCTTGGGTTACATATTCA
GATGTTGTTTGATAAGAAGGAGTACAGTGTCAGTGAAACTGATTTTGAAGACTCGG
AAGTTGTGTTGGATGTTCCGAGTTTGACTTGTCCTTATCCGGTGAAGTGTCTTCCTT
ATGGTTTGGCAACGAAAGAGTGGCTTCCTATGTATCTAAATCAAGGTAGAAGATTCA
GAGAGATGAAAGGTATTTTGGTAAATACTTTTGCTGAGCTTGAACCTTATGCGTTGG
AGTCTCTTCACTCTAGTGGTGATACTCCTCGTGCTTATCCAGTGGGACCATTGTTGC
ATCTCGAGAACCATGTTGACGGTTCTAAAGACGAGAAGGGTTCGGACATTTTACGG
TGGTTAGATGAACAACCACCTAAATCGGTAGTGTTCCTCTGCTTTGGAAGCATAGG
AGGCTTTAACGAGGAACAAGCAAGAGAAATGGCCATTGCACTTGAGAGAAGTGGTC
ACCGCTTCTTGTGGTCTCTTCGCCGTGCATCTCGAGATATAGATAAGGAACTTCCC
GGAGAATTCAAGAATCTTGAAGAAATTCTCCCGGAAGGATTCTTTGATCGGACAAA
GGATAAAGGAAAGGTGATCGGATGGGCTCCACAAGTAGCCGTGCTGGCTAAGCCA
GCAATCGGAGGTTTTGTTACTCATTGCGGGTGGAACTCGATACTCGAGAGTCTTTG
GTTCGGTGTTCCTATAGCGCCATGGCCGTTATACGCTGAGCAGAAGTTTAATGCTT
TCGTGATGGTGGAGGAGCTTGGTTTGGCAGTGAAGATAAGAAAGTATTGGCGAGG
CGATCAGTTGGTGGGAACGGCGACGGTCATAGTGACGGCAGAGGAGATAGAGAG
AGGAATCAGATGTTTGATGGAGCAAGATAGTGACGTGAGGAATAGAGTGAAGGAG
ATGAGTAAGAAATGTCACATGGCTTTAAAGGATGGTGGCTCGTCTCAATCTGCTTTG
AAATTATTTATTCAAGACGTTACGAAGTATATTGCTTGA
SEQ ID NO: 7 >UGT71C1
ATGGGGAAGCAAGAAGATGCAGAGCTCGTCATCATACCTTTCCCTTTCTCCGGACA
CATTCTCGCAACAATCGAACTCGCCAAACGTCTCATAAGTCAAGACAATCCTCGGAT
CCACACCATCACCATCCTCTATTGGGGATTACCTTTTATTCCTCAAGCTGACACAAT
CGCTTTCCTCCGATCCCTAGTCAAAAATGAGCCTCGTATCCGTCTCGTTACGTTGC
CCGAAGTCCAAGACCCTCCACCAATGGAACTCTTTGTGGAATTTGCCGAATCTTAC
ATTCTTGAATACGTCAAGAAAATGGTTCCCATCATCAGAGAAGCTCTCTCCACTCTC
TTGTCTTCCCGCGATGAATCGGGTTCAGTTCGTGTGGCTGGATTGGTTCTTGACTT
CTTCTGCGTCCCTATGATCGATGTAGGAAACGAGTTTAATCTCCCTTCTTACATTTT
CTTGACGTGTAGCGCAGGGTTCTTGGGTATGATGAAGTATCTTCCAGAGAGACACC
GCGAAATCAAATCGGAATTCAACCGGAGCTTCAACGAGGAGTTGAATCTCATTCCT
GGTTATGTCAACTCTGTTCCTACTAAGGTTTTGCCGTCAGGTCTATTCATGAAAGAG
ACCTACGAGCCTTGGGTCGAACTAGCAGAGAGGTTTCCTGAAGCTAAGGGTATTTT
GGTTAATTCATACACAGCTCTCGAGCCAAACGGTTTTAAATATTTCGATCGTTGTCC
GGATAACTACCCAACCATTTACCCAATCGGGCCGATATTATGCTCCAACGACCGTC
CGAATTTGGACTCATCGGAACGAGATCGGATCATAACTTGGCTAGATGACCAACCC
GAGTCATCGGTCGTGTTCCTCTGTTTCGGGAGCTTGAAGAATCTCAGCGCTACTCA
GATCAACGAGATAGCTCAAGCCTTAGAGATCGTTGACTGCAAATTCATCTGGTCGT
TTCGAACCAACCCGAAGGAGTACGCGAGCCCTTACGAGGCTCTACCACACGGGTT
CATGGACCGGGTCATGGATCAAGGCATTGTTTGTGGTTGGGCTCCTCAAGTTGAAA
TCCTAGCCCATAAAGCTGTGGGAGGATTCGTATCTCATTGTGGTTGGAACTCGATA
TTGGAGAGTTTGGGTTTCGGCGTTCCAATCGCCACGTGGCCGATGTACGCGGAAC
AACAACTAAACGCGTTCACGATGGTGAAGGAGCTTGGTTTAGCCTTGGAGATGCGG
TTGGATTACGTGTCGGAAGATGGAGATATAGTGAAAGCTGATGAGATCGCAGGAAC
CGTTAGATCTTTAATGGACGGTGTGGATGTGCCGAAGAGTAAAGTGAAGGAGATTG
CTGAGGCGGGAAAAGAAGCTGTGGACGGTGGATCTTCGTTTCTTGCGGTTAAAAG
ATTCATCGGTGACTTGATCGACGGCGTTTCTATAAGTAAGTAG
SEQ ID NO: 8 >UGT71C2
ATGGCGAAGCAGCAAGAAGCAGAGCTCATCTTCATCCCATTTCCAATCCCCGGACA
CATTCTCGCCACAATCGAACTCGCGAAACGTCTCATCAGTCACCAACCTAGTCGGA
TCCACACCATCACCATCCTCCATTGGAGCTTACCTTTTCTTCCTCAATCTGACACTA
TCGCCTTCCTCAAATCCCTAATCGAAACAGAGTCTCGTATCCGTCTCATTACCTTAC
CCGATGTCCAAAACCCTCCACCAATGGAGCTATTTGTGAAAGCTTCCGAATCTTACA
TTCTTGAATACGTCAAGAAAATGGTTCCTTTGGTCAGAAACGCTCTCTCCACTCTCT
TGTCTTCTCGTGATGAATCGGATTCAGTTCATGTCGCCGGATTAGTTCTTGATTTCT
TCTGTGTCCCTTTGATCGATGTCGGAAACGAGTTTAATCTCCCTTCTTACATCTTCT
TGACGTGTAGCGCAAGTTTCTTGGGTATGATGAAGTATCTTCTGGAGAGAAACCGC
GAAACCAAACCGGAACTTAACCGGAGCTCTGACGAGGAAACAATATCAGTTCCTGG
TTTTGTTAACTCCGTTCCGGTTAAAGTTTTGCCACCGGGTTTGTTCACGACTGAGTC
TTACGAAGCTTGGGTCGAAATGGCGGAAAGGTTCCCTGAAGCCAAGGGTATTTTGG
TCAATTCATTTGAATCTCTAGAACGTAACGCTTTTGATTATTTCGATCGTCGTCCGG
ATAATTACCCACCCGTTTACCCAATCGGGCCAATTCTATGCTCCAACGATCGTCCGA
ATTTGGATTTATCGGAACGAGACCGGATCTTGAAATGGCTCGATGACCAACCCGAG
TCATCTGTTGTGTTTCTCTGCTTCGGGAGCTTGAAGAGTCTCGCTGCGTCTCAGAT
TAAAGAGATCGCTCAAGCCTTAGAGCTCGTCGGAATCAGATTCCTCTGGTCGATTC
GAACGGACCCGAAGGAGTACGCGAGCCCGAACGAGATTTTACCGGACGGGTTTAT
GAACCGAGTCATGGGTTTGGGCCTTGTTTGTGGTTGGGCTCCTCAAGTTGAAATTC
TGGCCCATAAAGCAATTGGAGGGTTCGTGTCACACTGCGGTTGGAACTCGATATTG
GAGAGTTTGCGTTTCGGAGTTCCAATTGCCACGTGGCCAATGTACGCGGAACAACA
ACTAAACGCGTTCACGATTGTGAAGGAGCTTGGTTTGGCGTTGGAGATGCGGTTG
GATTACGTGTCGGAATATGGAGAAATCGTGAAAGCTGATGAAATCGCAGGAGCCGT
ACGATCTTTGATGGACGGTGAGGATGTGCCGAGGAGGAAACTGAAGGAGATTGCG
GAGGCGGGAAAAGAGGCTGTGATGGACGGTGGATCTTCGTTTGTTGCGGTTAAAA
GATTCATAGATGGGCTTTGA
SEQ ID NO: 9 >UGT71C3
ATGAAAGCAGAAGCAGAGATCATCTTCGTTACATATCCATCCCCTGGTCATCTTCTT
GTCTCCATTGAATTCGCTAAATCTCTCATCAAACGTGATGATCGCATCCACACCATC
ACCATCCTCTACTGGGCTTTACCTCTCGCTCCTCAAGCCCACCTTTTCGCTAAGTCC
CTCGTTGCTTCACAGCCTCGAATCCGTCTCCTTGCGTTGCCTGATGTTCAAAACCCT
CCACCATTGGAACTCTTCTTTAAAGCTCCCGAAGCTTATATTCTTGAGTCCACCAAG
AAAACAGTTCCTTTAGTCAGAGACGCTCTCTCCACTCTAGTTTCTTCACGTAAAGAA
TCCGGTTCGGTTCGTGTAGTCGGTTTGGTTATCGATTTTTTTTGTGTTCCAATGATC
GAAGTGGCAAACGAGCTTAACCTTCCTTCTTACATCTTCCTAACGTGTAACGCTGG
GTTTTTAAGTATGATGAAGTATCTCCCTGAGAGACATCGCATAACCACTTCTGAGCT
AGATTTAAGCTCCGGCAACGTAGAACATCCAATTCCTGGCTACGTCTGCTCCGTGC
CGACGAAGGTTTTGCCTCCAGGTCTATTCGTGAGAGAGTCCTACGAGGCTTGGGT
CGAGATTGCAGAGAAGTTCCCTGGAGCCAAGGGCATTTTGGTAAACTCAGTCACAT
GTCTTGAGCAGAATGCATTTGATTACTTCGCTCGTCTTGATGAGAACTATCCTCCGG
TTTACCCGGTCGGACCGGTTCTTAGTTTGAAGGATCGTCCGTCTCCAAATCTGGAC
GCATCGGACCGGGATCGGATCATGAGATGGCTCGAGGACCAGCCGGAGTCGTCAA
TTGTGTATATCTGCTTCGGAAGCCTCGGAATCATTGGCAAGCTGCAGATTGAAGAG
ATAGCTGAAGCCTTGGAACTCACCGGCCACAGGTTTCTTTGGTCAATACGTACAAA
TCCGACGGAGAAAGCGAGCCCGTACGATCTGTTGCCGGAGGGATTTCTCGATCGG
ACGGCCAGTAAGGGATTGGTGTGTGATTGGGCCCCGCAAGTAGAAGTTCTGGCCC
ATAAAGCGCTCGGAGGATTCGTGTCTCACTGCGGTTGGAACTCTGTACTGGAGAG
CTTATGGTTCGGTGTTCCGATCGCCACGTGGCCAATGTACGCTGAGCAACAGTTAA
ACGCATTCTCGATGGTGAAGGAGTTAGGGTTAGCCGTGGAGCTGCGTTTAGACTAC
GTTTCGGCGTACGGAGAGATAGTAAAAGCTGAGGAGATCGCGGGAGCCATACGAT
CATTGATGGACGGTGAGGATACGCCGAGGAAGAGAGTGAAGGAGATGGCGGAAG
CGGCGAGGAATGCTTTGATGGACGGAGGATCTTCGTTTGTTGCGGTTAAACGATTT
CTCGACGAGTTGATCGGCGGAGATGTTTAG
SEQ ID NO: 10 >UGT71C4
ATGGTGAAGGAAACAGAGCTAATCTTCATTCCAGTTCCATCCACAGGTCATATTCTC
GTCCATATTGAATTCGCCAAGCGTCTCATCAATCTCGACCATCGGATCCACACCATC
ACTATTCTCAACTTATCCTCACCCTCTTCTCCTCACGCCTCCGTCTTCGCCAGATCT
CTCATCGCTTCCCAGCCCAAAATCCGTCTCCACGACCTTCCCCCTATCCAAGATCCT
CCTCCATTCGATCTTTACCAAAGAGCTCCCGAAGCTTACATAGTAAAACTCATCAAG
AAAAATACTCCTCTGATAAAAGACGCCGTCTCCAGCATCGTCGCGTCGCGTCGTGG
AGGCTCAGATTCGGTTCAAGTCGCCGGTTTGGTTCTCGATTTATTCTGCAATTCATT
GGTAAAAGATGTTGGCAACGAGCTTAATCTTCCTTCTTACATATACCTTACGTGTAA
CGCTAGATACTTGGGGATGATGAAATATATTCCGGATCGGCATCGGAAAATCGCAT
CTGAGTTCGATTTGAGCTCCGGCGATGAAGAATTGCCGGTTCCGGGATTCATAAAC
GCTATTCCGACGAAATTTATGCCGCCTGGATTGTTCAATAAGGAAGCTTACGAGGC
TTACGTAGAGCTAGCGCCGAGATTCGCAGATGCGAAGGGTATTTTGGTTAATTCCT
TCACGGAGCTTGAGCCGCACCCGTTTGACTATTTCTCTCACCTGGAGAAATTCCCT
CCGGTTTACCCGGTCGGACCGATTCTCAGCTTGAAAGATCGAGCGAGTCCGAACG
AAGAAGCAGTCGATCGGGATCAGATCGTTGGGTGGCTCGATGATCAGCCGGAGTC
ATCGGTGGTGTTCCTCTGTTTCGGGAGCAGAGGAAGCGTTGATGAGCCGCAAGTG
AAGGAGATAGCTCGAGCTTTGGAACTCGTCGGCTGCAGATTTCTTTGGTCAATTAG
AACAAGCGGCGACGTCGAGACGAATCCTAACGATGTGTTGCCGGAGGGGTTCATG
GGCCGAGTAGCAGGCCGAGGTTTGGTATGTGGTTGGGCTCCACAAGTGGAAGTGT
TGGCCCATAAAGCAATAGGAGGATTTGTGTCTCACTGTGGTTGGAACTCCACGCTT
GAAAGCTTATGGTTCGGGGTTCCTGTCGCAACGTGGCCGATGTACGCAGAGCAAC
AGCTTAACGCCTTCACGCTGGTGAAAGAGCTTGGGCTTGCGGTGGACCTGCGGAT
GGATTACGTGTCGAGTCGTGGGGGTTTGGTGACTTGTGATGAGATAGCCAGAGCC
GTACGATCTTTGATGGACGGTGGAGATGAGAAGAGAAAAAAGGTTAAGGAGATGG
CTGATGCGGCAAGGAAGGCTTTGATGGATGGAGGATCGTCTTCTTTGGCAACTGCT
CGATTCATCGCAGAATTGTTTGAAGATGGTTCGTCGTGCTAA
SEQ ID NO: 11 >UGT71C5
ATGAAGACAGCAGAGCTCATATTCGTTCCTCTGCCGGAGACCGGCCATCTCTTGTC
AACGATCGAGTTTGGAAAGCGTCTACTCAATCTAGACCGTCGGATTTCTATGATTAC
AATCCTCTCCATGAATCTTCCTTACGCTCCTCACGCCGACGCTTCTCTTGCTTCGCT
AACAGCCTCCGAGCCTGGTATCCGAATCATCAGTCTCCCGGAGATCCACGATCCAC
CTCCGATCAAGCTTCTTGACACTTCCTCCGAGACTTACATCCTCGATTTCATCCATA
AAAACATACCTTGTCTCAGAAAAACCATCCAAGATTTAGTCTCATCATCATCATCTTC
CGGAGGTGGTAGTAGTCATGTCGCCGGCTTGATTCTTGATTTCTTCTGCGTTGGTT
TGATCGACATCGGCCGTGAGGTAAACCTTCCTTCCTATATCTTCATGACTTCCAACT
TTGGTTTCTTAGGGGTTCTACAGTATCTCCCGGAACGACAACGTTTGACTCCGTCG
GAGTTCGATGAGAGCTCCGGCGAGGAAGAGTTACATATTCCGGCGTTTGTGAACC
GTGTTCCCGCCAAGGTTCTGCCGCCAGGTGTGTTCGATAAACTCTCTTACGGGTCT
CTGGTCAAAATCGGCGAGCGATTACATGAAGCCAAGGGTATTTTGGTTAATTCATTT
ACCCAAGTGGAGCCTTATGCTGCTGAACATTTTTCTCAAGGACGAGATTACCCTCA
CGTGTATCCTGTTGGGCCGGTTCTCAACTTAACGGGCCGTACAAATCCGGGTCTAG
CTTCGGCCCAATATAAAGAGATGATGAAGTGGCTTGACGAGCAACCAGACTCGTCG
GTTTTGTTCCTGTGTTTCGGGAGCATGGGAGTCTTCCCTGCACCTCAGATCACAGA
GATTGCTCACGCGCTCGAGCTTATCGGGTGCAGGTTCATCTGGGCGATCCGTACG
AACATGGCGGGAGATGGCGATCCTCAGGAGCCGCTTCCAGAAGGATTTGTCGATC
GAACAATGGGCCGTGGAATTGTGTGTAGTTGGGCTCCACAAGTGGATATCTTGGCC
CACAAGGCAACAGGTGGATTCGTTTCTCACTGCGGGTGGAATTCCGTCCAAGAGA
GTCTATGGTACGGTGTACCTATTGCAACGTGGCCAATGTATGCGGAGCAACAACTG
AACGCATTTGAGATGGTGAAGGAGTTGGGCTTAGCAGTGGAGATAAGGCTTGACTA
CGTGGCGGATGGTGATAGGGTTACTTTGGAGATCGTGTCAGCCGATGAAATAGCC
ACAGCCGTCCGATCATTGATGGATAGTGATAACCCCGTGAGAAAGAAGGTTATAGA
AAAATCTTCAGTGGCGAGGAAAGCTGTTGGTGATGGTGGGTCTTCTACGGTGGCC
ACATGTAATTTTATCAAAGATATTCTTGGGGATCACTTTTGA
SEQ ID NO: 12 >UGT71D1
ATGCGGAATGTAGAGCTCATCTTCATCCCCACACCAACCGTTGGTCATCTTGTTCC
GTTTCTTGAATTTGCTAGGCGTCTCATTGAGCAAGATGATAGGATCCGTATCACAAT
CCTCTTGATGAAACTACAAGGTCAGTCTCATCTAGACACTTATGTTAAATCAATTGC
CTCCTCTCAACCGTTTGTTAGATTCATTGATGTCCCTGAGTTAGAGGAGAAACCTAC
ACTTGGTAGTACACAATCTGTGGAAGCTTATGTGTATGATGTTATTGAGAGAAATAT
CCCTCTTGTGAGGAATATAGTCATGGATATTTTAACTTCTCTTGCATTGGATGGAGT
TAAGGTCAAGGGATTAGTTGTTGACTTTTTCTGTCTCCCTATGATTGACGTTGCTAA
AGATATAAGTCTCCCTTTCTATGTGTTCTTGACTACAAATTCCGGGTTCTTAGCTAT
GATGCAGTATCTAGCAGATCGACATAGTAGAGATACATCGGTTTTTGTAAGAAACTC
GGAAGAAATGTTGTCGATACCTGGATTTGTAAACCCTGTCCCAGCCAATGTTCTGC
CGTCAGCTCTGTTTGTTGAAGATGGTTATGATGCTTACGTTAAGCTGGCCATATTGT
TTACAAAGGCCAATGGAATCCTAGTGAATAGCTCCTTTGATATTGAGCCTTACTCTG
TGAATCATTTTCTTCAAGAACAGAATTATCCTTCTGTTTATGCTGTTGGCCCCATATT
TGACTTGAAAGCCCAGCCTCATCCAGAGCAGGACCTAACCCGTCGTGACGAGTTGA
TGAAATGGCTTGATGATCAACCCGAGGCATCGGTTGTATTCCTTTGTTTTGGGAGT
ATGGCAAGGTTAAGAGGTTCTCTAGTGAAGGAAATAGCTCATGGACTTGAGCTATG
TCAATATAGATTCCTCTGGTCACTCCGTAAAGAAGAGGTGACAAAGGATGATTTGCC
AGAGGGGTTCCTTGACCGTGTCGATGGACGTGGAATGATATGTGGTTGGTCTCCT
CAGGTAGAAATACTGGCCCATAAGGCAGTGGGAGGCTTTGTTTCTCACTGTGGATG
GAACTCAATAGTAGAGAGTTTGTGGTTTGGCGTGCCAATTGTGACATGGCCAATGT
ATGCAGAGCAACAACTCAATGCGTTTCTGATGGTGAAGGAACTGAAGCTAGCTGTG
GAGCTGAAGCTTGATTACAGGGTACATAGTGATGAGATAGTAAACGCAAACGAGAT
AGAGACCGCTATTCGTTATGTAATGGACACGGATAATAATGTTGTGAGGAAACGAG
TGATGGATATCTCGCAGATGATCCAGAGAGCTACGAAGAATGGTGGATCTTCGTTT
GCCGCAATTGAGAAATTCATATATGACGTGATAGGAATTAAGCCCTAG
SEQ ID NO: 13 >UGT71D2
ATGAGGAATGCAGAGCTCATCTTCATCCCAACACCAACTGTTGGTCATCTTGTTCCG
TTTCTTGAATTTGCTAGGCGTCTCATTGAGCAGGATGATAGAATCCGTATCACCTTC
CTCTTGATGAAGCAACAAGGTCAGTCTCATCTGGATTCCTATGTTAAGACAATTTCC
TCGTCTCTGCCGTTTGTTAGATTTATTGATGTCCCTGAGTTAGAGGAGAAACCAACA
CTTGGTACACAGTCTGTGGAAGCCTATGTGTACGATTTTATTGAAACAAATGTCCCT
CTTGTGCAAAATATAATCATGGGTATCCTATCTTCTCCTGCATTTGATGGAGTTACG
GTCAAGGGATTCGTTGCTGATTTTTTCTGTCTCCCGATGATTGATGTTGCAAAAGAT
GCAAGTCTTCCTTTTTATGTGTTCTTGACTTCAAATTCCGGATTCCTAGCTATGATG
CAGTATCTGGCATATGGACATAAGAAAGATACCTCAGTTTTTGCAAGAAACTCTGAA
GAAATGTTGTCAATTCCTGGATTTGTAAACCCTGTCCCAGCCAAAGTACTGCCGTCA
GCTCTGTTTATTGAGGATGGTTATGATGCTGACGTTAAACTGGCTATATTGTTTACA
AAGGCTAATGGAATCCTAGTGAATACCTCCTTTGATATTGAGCCTACCTCTCTGAAT
CATTTTCTTGGAGAAGAGAATTACCCTTCTGTTTATGCTGTTGGCCCCATATTTAAC
CCGAAGGCCCATCCTCATCCAGATCAAGACCTCGCCTGTTGTGACGAGTCGATGAA
ATGGCTTGATGCTCAACCCGAGGCATCAGTTGTATTCCTTTGTTTTGGGAGTATGG
GTAGCTTAAGAGGTCCTCTAGTGAAGGAAATAGCACATGGACTTGAGCTATGTCAG
TATAGATTCCTCTGGTCACTCCGCACAGAAGAAGTGACAAATGATGATCTTTTGCCA
GAGGGATTCATGGACCGTGTCAGTGGACGGGGAATGATATGCGGTTGGTCTCCTC
AGGTGGAAATACTGGCCCATAAAGCAGTGGGAGGTTTTGTTTCTCATTGTGGATGG
AACTCAATAGTAGAGAGTTTATGGTTTGGTGTGCCAATTGTGACATGGCCAATGTAT
GCAGAGCAACAGCTCAATGCGTTTCTGATGGTGAAGGAACTGAAGCTCGCAGTGG
AGCTGAAACTCGATTATAGTGTACATAGTGGTGAGATTGTAAGTGCAAACGAGATA
GAGACAGCGATTTCTTGTGTAATGAACAAGGATAATAATGTTGTGAGGAAACGAGT
GATGGATATCTCGCAGATGATCCAGAGAGCTACGAAGAATGGTGGATCTTCGTTTG
CCGCAATTGAGAAATTCATACATGACGTGATAGGAACCAGGACTTAG
SEQ ID NO: 14 >UGT72B1
ATGGAGGAATCCAAAACACCTCACGTTGCGATCATACCAAGTCCGGGAATGGGTCA
TCTCATACCACTCGTCGAGTTTGCTAAACGACTCGTCCATCTTCACGGCCTCACCG
TTACCTTCGTCATCGCCGGCGAAGGTCCACCATCAAAAGCTCAGAGAACCGTCCTC
GACTCTCTCCCTTCTTCAATCTCCTCCGTCTTTCTCCCTCCTGTTGATCTCACCGAT
CTCTCTTCGTCCACTCGCATCGAATCTCGGATCTCCCTCACCGTGACTCGTTCAAA
CCCGGAGCTCCGGAAAGTCTTCGACTCGTTCGTGGAGGGAGGTCGTTTGCCAACG
GCGCTCGTCGTCGATCTCTTCGGTACGGACGCTTTCGACGTGGCCGTAGAATTTCA
CGTGCCACCGTATATTTTCTACCCAACAACGGCCAACGTCTTGTCGTTTTTTCTCCA
TTTGCCTAAACTAGACGAAACGGTGTCGTGTGAGTTCAGGGAATTAACCGAACCGC
TTATGCTTCCTGGATGTGTACCGGTTGCCGGGAAAGATTTCCTTGACCCGGCCCAA
GACCGGAAAGACGATGCATACAAATGGCTTCTCCATAACACCAAGAGGTACAAAGA
AGCCGAAGGTATTCTTGTGAATACCTTCTTTGAGCTAGAGCCAAATGCTATAAAGGC
CTTGCAAGAACCGGGTCTTGATAAACCACCGGTTTATCCGGTTGGACCGTTGGTTA
ACATTGGTAAGCAAGAGGCTAAGCAAACCGAAGAGTCTGAATGTTTAAAGTGGTTG
GATAACCAGCCGCTCGGTTCGGTTTTATATGTGTCCTTTGGTAGTGGCGGTACCCT
CACATGTGAGCAGCTCAATGAGCTTGCTCTTGGTCTTGCAGATAGTGAGCAACGGT
TTCTTTGGGTCATACGAAGTCCTAGTGGGATCGCTAATTCGTCGTATTTTGATTCAC
ATAGCCAAACAGATCCATTGACATTTTTACCACCGGGATTTTTAGAGCGGACTAAAA
AAAGAGGTTTTGTGATCCCTTTTTGGGCTCCACAAGCCCAAGTCTTGGCGCATCCA
TCCACGGGAGGATTTTTAACTCATTGTGGATGGAATTCGACTCTAGAGAGTGTAGT
AAGCGGTATTCCACTTATAGCATGGCCATTATACGCAGAACAGAAGATGAATGCGG
TTTTGTTGAGTGAAGATATTCGTGCGGCACTTAGGCCGCGTGCCGGGGACGATGG
GTTAGTTAGAAGAGAAGAGGTGGCTAGAGTGGTAAAAGGATTGATGGAAGGTGAA
GAAGGCAAAGGAGTGAGGAACAAGATGAAGGAGTTGAAGGAAGCAGCTTGTAGGG
TGTTGAAGGATGATGGGACTTCGACAAAAGCACTTAGTCTTGTGGCCTTAAAGTGG
AAAGCCCACAAAAAAGAGTTAGAGCAAAATGGCAACCACTAA
SEQ ID NO: 15 >UGT72B2
ATGCAAAAAATGGCAGATGGAAACACTCCACATGTAGCAATCATACCAAGTCCCGG
TATAGGTCACCTCATCCCACTCGTCGAGTTAGCAAAGCGACTCCTTGACAATCACG
GTTTCACCGTCACTTTCATCATCCCCGGCGATTCTCCTCCGTCTAAGGCTCAAAGAT
CCGTTCTCAACTCTCTCCCTTCCTCCATAGCCTCCGTCTTCCTCCCTCCCGCCGATC
TTTCCGACGTTCCTTCGACAGCTCGAATCGAAACTCGGATATCGCTCACCGTGACT
CGTTCCAACCCGGCGCTCCGGGAGCTTTTTGGCTCGTTATCGGCGGAGAAACGTC
TCCCGGCGGTTCTCGTCGTCGATCTATTTGGTACGGATGCGTTCGACGTGGCTGC
TGAGTTCCACGTGTCGCCATACATTTTCTATGCATCAAATGCCAACGTCCTCACGTT
TCTGCTTCACTTGCCGAAGCTAGACGAAACGGTGTCGTGTGAGTTTAGGGAATTAA
CCGAACCGGTTATTATTCCCGGTTGTGTCCCCATAACCGGTAAGGATTTCGTCGAT
CCGTGTCAAGACCGAAAAGATGAATCATACAAATGGCTTCTACACAACGTCAAGAG
ATTCAAAGAAGCTGAAGGGATTCTAGTGAATTCCTTCGTCGATTTAGAGCCAAACAC
TATAAAGATTGTACAAGAACCGGCTCCTGATAAACCACCGGTTTACCTGATTGGGC
CGTTGGTTAACTCGGGTTCACACGATGCTGACGTGAACGATGAGTACAAATGTTTA
AATTGGCTAGACAACCAACCATTCGGGTCGGTTCTATACGTATCCTTTGGAAGCGG
CGGAACACTCACGTTTGAGCAGTTCATTGAGCTGGCTCTTGGCCTAGCGGAGAGT
GGAAAACGGTTTCTTTGGGTCATACGAAGTCCGAGTGGGATAGCTAGTTCATCGTA
TTTCAATCCACAAAGCCGAAATGATCCATTTTCGTTTTTACCACAAGGCTTCTTAGAC
CGAACCAAAGAAAAAGGTCTAGTGGTTGGGTCATGGGCTCCACAGGCTCAAATTCT
GACTCATACATCTATAGGTGGATTTTTAACTCATTGTGGATGGAATTCGAGTCTAGA
AAGTATTGTAAACGGTGTACCGCTCATAGCATGGCCGTTATACGCGGAGCAAAAGA
TGAACGCATTGCTACTCGTGGATGTTGGTGCGGCTCTAAGAGCACGACTGGGTGA
AGACGGGGTCGTAGGAAGGGAAGAAGTGGCGAGAGTGGTAAAAGGATTGATAGAA
GGAGAAGAAGGGAATGCGGTAAGGAAAAAAATGAAAGAGTTGAAAGAAGGATCTGT
TAGAGTCTTAAGGGACGATGGATTCTCTACCAAATCGCTTAATGAAGTTTCGTTGAA
GTGGAAAGCCCACCAACGAAAGATCGACCAAGAACAGGAATCATTTCTATGA
SEQ ID NO: 16 >UGT72B3
ATGAGCATAGATATTTTTCAAGAAATAAGAATAAAGAAAATTCTACTCTTAATGGCGG
AAGCAAACACTCCACACATAGCAATCATGCCGAGTCCCGGTATGGGTCACCTTATC
CCATTCGTCGAGTTAGCAAAGCGACTCGTTCAGCACGACTGTTTCACCGTCACAAT
GATCATCTCCGGTGAAACTTCGCCGTCTAAGGCACAAAGATCCGTTCTCAACTCTC
TCCCTTCCTCCATAGCCTCCGTATTTCTCCCTCCCGCCGATCTTTCCGATGTTCCCT
CCACAGCGCGAATCGAAACTCGGGCCATGCTCACCATGACTCGTTCCAATCCGGC
GCTCCGGGAGCTTTTTGGCTCTTTATCAACGAAGAAAAGTCTCCCGGCGGTTCTCG
TCGTCGATATGTTTGGTGCGGATGCGTTCGACGTGGCCGTTGACTTCCACGTGTCA
CCATACATTTTCTATGCATCCAATGCAAACGTCTTGTCGTTTTTTCTTCACTTGCCGA
AACTAGACAAAACGGTGTCGTGTGAGTTTAGGTACTTAACCGAACCGCTTAAGATTC
CCGGCTGTGTCCCGATAACCGGTAAGGACTTTCTTGATACGGTTCAAGACCGAAAC
GACGACGCATACAAATTGCTTCTCCATAACACCAAGAGGTACAAAGAAGCTAAAGG
GATTCTAGTGAATTCCTTCGTTGATTTAGAGTCGAATGCAATAAAGGCCTTACAAGA
ACCGGCTCCTGATAAACCAACGGTATACCCGATTGGGCCGCTGGTTAACACAAGTT
CATCTAATGTTAACTTGGAAGACAAGTTCGGATGTTTAAGTTGGCTAGACAACCAAC
CATTCGGCTCGGTTCTATACATATCATTTGGAAGCGGCGGAACACTTACATGTGAG
CAGTTTAATGAGCTTGCTATTGGTCTTGCGGAGAGCGGAAAACGGTTTATTTGGGT
CATACGAAGTCCAAGCGAGATAGTTAGTTCGTCGTATTTCAATCCACACAGCGAGA
CAGACCCCTTTTCGTTTTTACCAATTGGGTTCTTAGACCGAACCAAAGAGAAAGGTT
TGGTGGTTCCATCATGGGCTCCACAGGTTCAAATCCTGGCTCATCCATCCACATGC
GGGTTTTTAACACACTGTGGATGGAATTCGACCTTAGAAAGCATTGTAAACGGTGTA
CCACTCATAGCGTGGCCTTTATTCGCGGAGCAAAAGATGAATACATTGCTACTCGT
GGAGGATGTTGGAGCGGCTCTAAGAATCCATGCGGGTGAAGATGGGATTGTACGG
AGGGAAGAAGTGGTGAGAGTGGTGAAGGCACTGATGGAAGGTGAAGAGGGAAAA
GCCATAGGAAATAAAGTGAAGGAGTTGAAAGAAGGAGTTGTTAGAGTCTTGGGTGA
CGATGGATTGTCCAGCAAGTCATTTGGTGAAGTTTTGTTAAAGTGGAAAACGCACC
AGCGAGATATCAACCAAGAGACGTCCCACTAA
SEQ ID NO: 17 >UGT72C1
ATGGAACTTCACGGAGCTCTAGTGGCTAGTCCGGGCATGGGACATGCCGTACCCA
TCTTAGAACTCGGTAAACATCTCCTGAACCACCACGGGTTCGACCGTGTCACTGTC
TTCCTAGTCACAGACGATGTCTCACGTTCGAAATCCCTAATTGGAAAAACGTTGATG
GAAGAAGATCCAAAATTTGTGATCAGGTTTATTCCACTCGATGTTTCGGGTCAAGAT
CTGAGTGGTTCACTATTGACTAAACTAGCAGAGATGATGAGGAAGGCATTACCAGA
GATCAAGTCTTCAGTCATGGAGTTAGAACCGCGGCCTAGGGTTTTCGTAGTTGACT
TGTTGGGCACGGAAGCTTTAGAGGTGGCTAAGGAGCTTGGGATCATGAGAAAACA
TGTTCTGGTTACTACCAGTGCTTGGTTTCTAGCTTTTACGGTTTATATGGCGAGTCT
TGACAAACAGGAGTTGTATAAGCAGTTGAGTAGCATAGGAGCATTGCTTATACCCG
GATGCAGCCCGGTTAAGTTTGAGCGGGCTCAAGATCCGAGAAAATATATTCGGGAA
CTCGCTGAGTCTCAGCGTATTGGGGATGAGGTGATAACCGCAGATGGGGTGTTTG
TGAATACGTGGCACAGTCTGGAGCAAGTGACCATCGGGTCTTTCTTGGATCCAGAG
AATCTCGGTCGGGTTATGAGAGGAGTGCCGGTTTATCCTGTTGGACCGCTGGTTA
GACCAGCAGAACCAGGTTTGAAACATGGCGTGCTGGACTGGCTTGACTTACAACCC
AAAGAGTCAGTGGTTTATGTTCTTTTGGGAGTGGTGGGGGCACTAACCTTCGAGCA
GACAAACGAGCTGGCTTACGGTTTGGAGCTGACTGGCCACAGATTTGTTTGGGTAG
TCAGACCACCGGCTGAAGACGACCCATCGGCATCAATGTTCGACAAGACCAAGAAT
GAGACAGAACCTCTCGATTTCTTACCCAACGGGTTTCTAGACCGAACCAAAGACAT
CGGTTTGGTGGTCCGTACATGGGCACCACAAGAAGAGATTCTGGCACACAAGTCAA
CAGGAGGGTTTGTGACTCACTGCGGATGGAACTCAGTTTTGGAGAGTATTGTGAAT
GGTGTGCCAATGGTAGCTTGGCCGTTGTACTCAGAGCAGAAGATGAACGCGAGGA
TGGTTTCTGGGGAGCTAAAGATTGCGTTGCAGATTAATGTTGCAGATGGGATTGTA
AAGAAGGAGGTGATAGCTGAAATGGTGAAGAGAGTGATGGATGAAGAAGAAGGAA
AAGAGATGAGAAAGAATGTTAAGGAACTGAAGAAGACAGCAGAAGAAGCTCTCAAC
ATGACTCACATTCCATCTGCTTACTTCACCTAA
SEQ ID NO: 18 >UGT72D1
ATGGACCAGCCTCACGCGCTTCTAGTGGCTAGCCCTGGCTTGGGTCACCTCATCC
CTATCCTGGAGCTCGGCAACCGTCTCTCCTCCGTCCTAAACATCCACGTCACCATT
CTCGCGGTCACCTCCGGCTCCTCTTCACCGACAGAAACCGAAGCCATACATGCAG
CCGCGGCTAGAACAATCTGTCAAATTACGGAAATTCCCTCGGTGGATGTAGACAAC
CTCGTGGAGCCAGATGCTACAATTTTCACTAAGATGGTGGTGAAGATGCGAGCCAT
GAAGCCCGCGGTACGAGATGCCGTGAAATTAATGAAACGAAAACCAACGGTCATGA
TTGTTGACTTTTTGGGTACGGAACTGATGTCCGTAGCCGATGACGTAGGCATGACG
GCTAAATACGTTTACGTTCCAACTCATGCGTGGTTCTTGGCAGTCATGGTGTACTTG
CCGGTGTTAGATACGGTAGTGGAAGGTGAGTATGTTGATATTAAGGAGCCTTTGAA
GATACCGGGTTGTAAACCGGTCGGACCGAAGGAGCTGATGGAAACGATGTTAGAC
CGGTCGGGCCAGCAATATAAAGAGTGTGTACGAGCTGGCTTAGAGGTACCTATGA
GCGATGGTGTTTTGGTAAATACTTGGGAGGAGTTACAAGGAAACACTCTCGCTGCG
CTTAGAGAGGACGAAGAATTGAGCCGGGTCATGAAAGTACCGGTTTATCCTATTGG
GCCAATTGTTAGGACTAACCAGCATGTAGACAAACCCAATAGTATATTCGAGTGGCT
AGACGAGCAACGGGAAAGGTCAGTGGTGTTTGTGTGTTTAGGGAGCGGTGGAACG
TTGACGTTTGAGCAAACAGTGGAACTCGCTTTGGGTTTAGAGTTAAGTGGTCAAAG
GTTCGTTTGGGTTCTACGTAGGCCCGCTTCATATCTCGGGGCGATCTCCAGCGATG
ATGAACAGGTAAGTGCCAGTCTACCTGAAGGTTTCTTGGACCGCACGCGTGGTGT
GGGGATTGTGGTTACGCAATGGGCACCACAAGTTGAGATCTTGAGCCATAGATCGA
TCGGTGGGTTCTTGTCTCACTGCGGTTGGAGTTCGGCTTTGGAAAGTTTGACTAAA
GGAGTTCCGATCATCGCTTGGCCTCTTTATGCGGAGCAGTGGATGAATGCCACGTT
ATTGACTGAGGAGATCGGTGTGGCCGTTCGTACATCGGAGTTACCGTCGGAGAGA
GTCATCGGAAGGGAAGAAGTGGCATCTCTGGTGAGAAAGATTATGGCGGAAGAGG
ATGAAGAAGGACAGAAAATTAGGGCTAAAGCTGAGGAGGTGAGGGTTAGCTCCGA
ACGAGCTTGGAGTAAAGACGGGTCATCTTATAATTCTCTATTCGAATGGGCAAAAC
GATGTTATCTTGTACCGTGA
SEQ ID NO: 19 >UGT72E1
ATGAAGATTACAAAACCACATGTGGCCATGTTCGCTAGCCCCGGAATGGGCCACAT
CATCCCGGTGATCGAGCTCGGAAAACGCTTAGCTGGTTCCCACGGCTTCGATGTCA
CCATTTTCGTCCTTGAAACCGACGCAGCCTCAGCTCAATCTCAATTCCTTAACTCAC
CAGGCTGCGACGCGGCCCTTGTTGATATCGTTGGCCTCCCAACGCCCGATATCTC
CGGTTTAGTCGACCCATCAGCCTTTTTTGGGATCAAGCTCTTGGTCATGATGCGTG
AGACCATTCCTACCATCCGGTCAAAGATAGAGGAGATGCAACACAAACCAACGGCT
CTGATCGTAGACTTGTTTGGTTTGGACGCGATACCGCTCGGTGGTGAGTTCAACAT
GTTGACTTATATCTTCATCGCTTCAAACGCACGTTTTCTCGCGGTGGCTTTGTTTTT
CCCAACGTTGGACAAAGACATGGAAGAAGAGCACATAATCAAGAAGCAACCTATGG
TTATGCCTGGATGTGAACCGGTTCGGTTTGAAGATACACTTGAAACATTCCTTGACC
CAAACAGCCAACTCTACCGGGAATTTGTTCCTTTCGGTTCGGTTTTCCCAACGTGT
GATGGTATTATTGTGAATACATGGGATGATATGGAGCCCAAAACTTTGAAATCTCTT
CAAGACCCAAAGCTCTTGGGTCGAATTGCTGGTGTACCGGTTTATCCAATTGGTCC
TTTGTCTAGACCGGTTGATCCATCTAAAACTAATCATCCGGTTTTGGATTGGTTAAA
CAAACAGCCGGACGAGTCGGTACTTTACATTTCATTTGGAAGCGGTGGCTCTCTCT
CGGCTAAACAACTAACCGAATTGGCTTGGGGACTTGAGATGAGTCAGCAACGGTTC
GTTTGGGTGGTTCGACCCCCGGTGGACGGTTCAGCTTGCAGTGCATATTTATCCG
CTAACAGTGGTAAAATACGAGACGGTACACCTGATTATCTCCCGGAAGGTTTTGTTA
GCCGGACTCATGAGAGAGGCTTTATGGTCTCTTCTTGGGCTCCCCAAGCGGAGAT
CTTGGCCCACCAAGCCGTAGGTGGGTTTCTAACTCACTGCGGTTGGAATTCGATTC
TCGAGAGCGTCGTTGGTGGCGTTCCGATGATCGCGTGGCCACTTTTTGCGGAGCA
GATGATGAACGCGACACTCCTCAACGAAGAGCTTGGCGTTGCCGTCCGCTCTAAG
AAACTACCGTCGGAGGGAGTGATTACGAGGGCGGAGATCGAGGCGTTGGTGAGAA
AGATCATGGTGGAGGAGGAAGGTGCTGAGATGAGAAAGAAGATAAAGAAGCTGAA
AGAGACCGCTGCCGAATCGCTGAGTTGCGACGGTGGAGTGGCGCATGAATCGTTG
TCAAGAATCGCCGACGAGAGCGAGCATCTTTTGGAGCGTGTCAGGTGCATGGCAC
GTGGTGCCTAG
SEQ ID NO: 20 >UGT72E2
ATGCATATCACAAAACCACACGCCGCCATGTTTTCCAGTCCCGGAATGGGCCATGT
CATCCCGGTGATCGAGCTTGGAAAGCGTCTCTCCGCTAACAACGGCTTCCACGTCA
CCGTCTTCGTCCTCGAAACCGACGCAGCCTCCGCTCAATCCAAGTTCCTAAACTCA
ACCGGCGTCGACATCGTCAAACTTCCATCGCCGGACATTTATGGTTTAGTGGACCC
CGACGACCATGTAGTGACCAAGATCGGAGTCATTATGCGTGCAGCAGTTCCAGCC
CTCCGATCCAAGATCGCTGCCATGCATCAAAAGCCAACGGCTCTGATCGTTGACTT
GTTTGGCACAGATGCGTTATGTCTCGCAAAGGAATTTAACATGTTGAGTTATGTGTT
TATCCCTACCAACGCACGTTTTCTCGGAGTTTCGATTTATTATCCAAATTTGGACAA
AGATATCAAGGAAGAGCACACAGTGCAAAGAAACCCACTCGCTATACCGGGGTGTG
AACCGGTTAGGTTCGAAGATACTCTGGATGCATATCTGGTTCCCGACGAACCGGTG
TACCGGGATTTTGTTCGTCATGGTCTGGCTTACCCAAAAGCCGATGGAATTTTGGT
AAATACATGGGAAGAGATGGAGCCCAAATCATTGAAGTCCCTTCTAAACCCAAAGC
TCTTGGGCCGGGTTGCTCGTGTACCGGTCTATCCAATCGGTCCCTTATGCAGACCG
ATACAATCATCCGAAACCGATCACCCGGTTTTGGATTGGTTAAACGAACAACCGAAC
GAGTCGGTTCTCTATATCTCCTTCGGGAGTGGTGGTTGTCTATCGGCGAAACAGTT
AACTGAATTGGCGTGGGGACTCGAGCAGAGCCAGCAACGGTTCGTATGGGTGGTT
CGACCACCGGTCGACGGTTCGTGTTGTAGCGAGTATGTCTCGGCTAACGGTGGTG
GAACCGAAGACAACACGCCAGAGTATCTACCGGAAGGGTTCGTGAGTCGTACTAG
TGATAGAGGTTTCGTGGTCCCCTCATGGGCCCCACAAGCTGAAATCCTGTCCCATC
GGGCCGTTGGTGGGTTTTTGACCCATTGCGGTTGGAGCTCGACGTTGGAAAGCGT
CGTTGGCGGCGTTCCGATGATCGCATGGCCACTTTTTGCCGAGCAGAATATGAATG
CGGCGTTGCTCAGCGACGAACTGGGAATCGCAGTCAGATTGGATGATCCAAAGGA
GGATATTTCTAGGTGGAAGATTGAGGCGTTGGTGAGGAAGGTTATGACTGAGAAG
GAAGGTGAAGCGATGAGAAGGAAAGTGAAGAAGTTGAGAGACTCGGCGGAGATGT
CACTGAGCATTGACGGTGGTGGTTTGGCGCACGAGTCGCTTTGCAGAGTCACCAA
GGAGTGTCAACGGTTTTTGGAACGTGTCGTGGACTTGTCACGTGGTGCTTAG
SEQ ID NO: 21 >UGT72E3
ATGCATATCACAAAACCACACGCCGCCATGTTTTCCAGTCCCGGAATGGGCCATGT
CCTCCCGGTGATCGAGCTAGCTAAGCGTCTCTCCGCTAACCACGGCTTCCACGTCA
CCGTCTTCGTCCTTGAAACTGACGCAGCCTCCGTTCAGTCCAAGCTCCTTAACTCA
ACCGGTGTTGACATCGTCAACCTTCCATCGCCCGACATTTCTGGCTTGGTAGACCC
CAACGCCCATGTGGTGACCAAGATCGGAGTCATTATGCGTGAAGCTGTTCCAACCC
TCCGATCCAAGATCGTTGCCATGCATCAAAACCCAACGGCTCTGATCATTGACTTGT
TTGGCACAGATGCGTTATGTCTTGCAGCGGAGTTAAACATGTTGACTTATGTCTTTA
TCGCTTCCAACGCGCGTTATCTCGGAGTTTCGATATATTATCCAACTTTGGACGAAG
TTATCAAAGAAGAGCACACAGTGCAACGAAAACCGCTCACTATACCGGGGTGTGAA
CCGGTTAGATTTGAAGATATTATGGATGCATATCTGGTTCCGGACGAACCGGTGTA
CCACGATTTGGTTCGTCACTGTCTGGCCTACCCAAAAGCGGATGGAATCTTGGTGA
ATACATGGGAAGAGATGGAGCCCAAATCATTAAAGTCCCTTCAAGACCCGAAACTTT
TGGGCCGGGTCGCTCGTGTACCGGTTTATCCGGTTGGTCCGTTATGCAGACCGAT
ACAATCATCCACGACCGATCACCCGGTTTTTGATTGGTTAAACAAACAACCAAACGA
GTCGGTTCTCTACATTTCCTTCGGGAGTGGTGGTTCTCTAACGGCTCAACAGTTAA
CCGAATTGGCGTGGGGGCTCGAGGAGAGCCAGCAACGGTTTATATGGGTGGTTCG
ACCGCCCGTTGACGGCTCGTCTTGCAGTGATTATTTCTCGGCTAAAGGCGGTGTAA
CCAAAGACAACACGCCAGAGTATCTACCAGAAGGGTTCGTGACTCGTACTTGCGAT
AGAGGTTTCATGATCCCATCATGGGCACCGCAAGCTGAAATCCTAGCCCATCAGGC
CGTTGGTGGGTTTTTAACACATTGTGGTTGGAGCTCGACGTTGGAAAGCGTCCTTT
GCGGCGTTCCAATGATAGCGTGGCCGCTTTTCGCCGAGCAGAATATGAACGCGGC
GTTGCTTAGCGATGAACTGGGAATCTCTGTTAGAGTGGATGATCCAAAGGAGGCGA
TTTCTAGGTCGAAGATTGAGGCGATGGTGAGGAAGGTTATGGCTGAGGACGAAGG
TGAAGAGATGAGAAGGAAAGTGAAGAAGTTGAGAGACACGGCGGAGATGTCACTT
AGTATTCACGGTGGTGGTTCGGCGCATGAGTCGCTTTGCAGAGTCACGAAGGAGT
GTCAACGGTTTTTGGAATGTGTCGGGGACTTGGGACGTGGTGCTTAG
SEQ ID NO: 22 >UGT73B1
ATGGGAACTCCTGTCGAAGTCTCTAAGCTCCATTTCTTGCTCTTCCCTTTCATGGCT
CATGGCCATATGATACCAACTCTAGACATGGCTAAGCTCTTTGCCACCAAAGGAGC
TAAATCCACTATCCTCACTACACCTCTCAATGCCAAGCTCTTCTTCGAGAAACCCAT
CAAATCATTCAACCAAGACAACCCGGGACTCGAAGACATCACCATCCAGATCCTTAA
TTTCCCTTGCACAGAGCTTGGTTTGCCTGATGGCTGTGAGAATACTGATTTCATCTT
CTCCACACCTGACCTAAACGTAGGTGACTTGAGTCAAAAGTTTTTACTCGCAATGAA
ATATTTCGAAGAGCCACTAGAGGAGCTCCTCGTGACAATGAGACCAGACTGTCTTG
TCGGTAACATGTTCTTCCCTTGGTCCACTAAAGTTGCTGAGAAGTTCGGAGTACCG
AGACTTGTGTTCCACGGCACAGGCTACTTCTCTTTATGTGCTTCTCATTGCATAAGG
CTCCCTAAGAATGTGGCAACAAGTTCTGAGCCCTTTGTGATTCCTGATCTCCCGGG
AGACATTTTGATTACAGAGGAACAGGTCATGGAGACAGAAGAAGAGTCTGTAATGG
GGAGGTTTATGAAGGCAATAAGAGACTCAGAGAGAGATAGCTTTGGCGTGTTGGT
GAACAGCTTCTACGAGCTTGAACAGGCTTACTCAGATTATTTCAAGAGCTTTGTGGC
GAAAAGAGCGTGGCATATCGGTCCGCTTTCCTTAGGAAATAGAAAGTTCGAGGAGA
AAGCAGAAAGAGGCAAAAAGGCAAGCATTGATGAGCATGAATGTTTGAAATGGCTC
GACTCCAAGAAATGTGATTCAGTGATTTACATGGCCTTTGGAACCATGTCTAGCTTT
AAAAACGAGCAGCTGATAGAGATTGCAGCTGGTTTAGATATGTCAGGACATGATTTT
GTCTGGGTGGTTAACAGAAAAGGCAGCCAAGTTGAGAAGGAAGATTGGTTACCAG
AGGGGTTTGAAGAGAAGACCAAGGGAAAAGGATTGATAATCCGAGGGTGGGCGCC
ACAAGTGCTGATACTTGAGCACAAAGCAATTGGCGGATTTTTGACGCATTGTGGAT
GGAACTCGTTATTAGAAGGGGTGGCAGCGGGCCTGCCAATGGTGACATGGCCCGT
GGGAGCCGAGCAGTTCTACAACGAGAAATTGGTGACACAAGTGTTGAAAACAGGA
GTGAGTGTGGGAGTGAAGAAGATGATGCAAGTAGTTGGAGACTTCATTAGCAGAGA
GAAAGTGGAGGGAGCGGTGAGGGAAGTGATGGTTGGAGAAGAGAGGAGGAAACG
GGCCAAGGAGTTAGCAGAAATGGCGAAAAATGCGGTGAAAGAAGGAGGATCTTCA
GATCTAGAGGTAGATAGGTTGATGGAAGAGCTTACGTTAGTTAAACTGCAAAAAGA
GAAGGTATAA
SEQ ID NO: 23 >UGT73B2
ATGGGTAGTGATCATCATCATCGAAAGCTCCACGTTATGTTCTTCCCTTTCATGGCT
TATGGTCACATGATACCAACTCTAGACATGGCTAAGCTTTTCTCTAGCAGAGGAGC
CAAATCCACAATCCTCACCACATCTCTCAACTCCAAGATCCTCCAAAAACCCATCGA
CACATTCAAGAATCTGAATCCGGGTCTCGAAATCGACATCCAGATCTTCAATTTCCC
TTGCGTGGAGCTGGGGTTACCAGAAGGATGTGAAAACGTTGATTTCTTCACTTCAA
ACAACAATGATGATAAAAACGAGATGATCGTGAAATTCTTTTTCTCGACAAGGTTTTT
CAAAGACCAGCTTGAGAAACTCCTCGGGACAACGAGACCAGACTGTCTTATCGCCG
ACATGTTCTTCCCCTGGGCTACTGAAGCTGCTGGGAAGTTCAATGTGCCAAGACTT
GTGTTCCACGGCACTGGCTACTTCTCTTTATGCGCTGGTTATTGCATCGGAGTGCA
TAAACCACAGAAGAGAGTGGCTTCAAGCTCTGAGCCATTTGTGATTCCCGAGCTCC
CTGGGAACATTGTGATAACTGAAGAACAGATCATAGATGGCGATGGAGAATCCGAC
ATGGGAAAGTTTATGACTGAAGTTAGGGAATCGGAAGTGAAGAGCTCAGGAGTTGT
TTTGAATAGTTTCTACGAGCTAGAACATGATTACGCCGATTTTTACAAAAGTTGTGTA
CAAAAGAGAGCGTGGCATATCGGTCCGCTATCGGTTTACAACAGGGGATTTGAGG
AGAAGGCTGAGAGAGGAAAGAAAGCGAACATTGATGAGGCTGAATGCCTCAAATG
GCTTGACTCCAAGAAACCAAATTCAGTCATTTATGTTTCCTTTGGGAGCGTGGCTTT
CTTCAAGAATGAACAGTTATTCGAGATCGCTGCAGGGTTAGAAGCTTCCGGTACAA
GTTTCATTTGGGTTGTTAGGAAAACCAAAGTGATAGAGAAGAATGGTTACCAGAAG
GGTTCGAAGAGAGGGTGAAAGGGAAAGGTATGATAATAAGAGGATGGGCACCACA
GGTGCTGATACTTGACCACCAAGCAACCGGTGGGTTTGTGACCCATTGCGGCTGG
AACTCGCTTCTTGAAGGAGTGGCTGCAGGGCTACCAATGGTGACATGGCCTGTAG
GAGCGGAGCAATTCTACAATGAGAAATTGGTTACGCAAGTGCTCAGAACAGGAGTG
AGCGTGGGAGCGAGCAAGCATATGAAAGTTATGATGGGAGATTTCATTAGCAGAGA
GAAAGTGGATAAAGCGGTGAGGGAGGTTTTGGCTGGGGAAGCAGCAGAGGAGAG
GCGGAGACGGGCAAAGAAGCTAGCGGCGATGGCTAAAGCTGCCGTGGAAGAAGG
AGGGTCTTCCTTCAACGATCTAAACAGCTTCATGGAAGAGTTTAGTTCATAA
SEQ ID NO: 24 >UGT73B3
ATGAGTAGTGATCCTCATCGTAAGCTCCATGTTGTGTTCTTCCCTTTCATGGCTTAT
GGTCACATGATACCAACTCTAGACATGGCTAAGCTTTTCTCTAGCAGAGGAGCCAA
ATCTACAATCCTCACCACACCTCTCAACTCCAAGATCTTCCAAAAACCCATCGAAAG
ATTCAAGAACCTGAATCCGAGTTTCGAAATCGACATCCAGATCTTCGATTTCCCTTG
CGTGGATCTCGGGTTACCAGAAGGATGCGAAAACGTCGATTTCTTCACCTCAAACA
ACAATGATGATAGACAGTATCTGACCTTGAAGTTCTTTAAGTCGACAAGGTTTTTCA
AAGATCAGCTTGAGAAGCTCCTCGAGACAACGAGACCAGACTGTCTTATCGCCGAC
ATGTTCTTCCCCTGGGCTACGGAAGCTGCTGAGAAGTTCAATGTGCCAAGACTTGT
GTTCCACGGTACTGGCTACTTTTCTTTATGCTCTGAATATTGCATCAGAGTGCATAA
CCCACAAAACATAGTAGCTTCAAGGTACGAGCCATTTGTGATTCCTGATCTCCCGG
GGAACATAGTGATAACTCAAGAACAGATAGCAGACCGTGACGAAGAAAGCGAGATG
GGGAAGTTTATGATTGAGGTCAAAGAATCTGATGTGAAGAGCTCAGGTGTTATTGT
AAACAGCTTCTACGAGCTTGAACCTGATTACGCCGACTTTTACAAGAGTGTTGTACT
GAAGAGAGCGTGGCATATCGGTCCGCTTTCGGTTTACAACAGAGGATTTGAGGAG
AAGGCTGAGAGAGGAAAGAAAGCAAGCATTAATGAGGTTGAATGCCTCAAATGGCT
TGACTCCAAGAAACCAGATTCAGTCATTTACATTTCTTTTGGGAGCGTGGCTTGCTT
CAAGAACGAGCAGCTATTCGAGATCGCTGCAGGATTAGAAACTTCTGGAGCAAATT
TCATCTGGGTTGTTAGGAAAAACATAGGTATTGAAAAAGAAGAATGGTTACCAGAAG
GGTTCGAAGAGAGGGTGAAAGGAAAAGGGATGATTATAAGAGGATGGGCACCACA
GGTGCTCATACTTGATCATCAAGCAACTTGTGGGTTTGTGACCCATTGCGGCTGGA
ACTCGCTTCTGGAAGGAGTGGCTGCAGGGCTACCAATGGTGACATGGCCTGTAGC
AGCGGAGCAATTCTACAATGAGAAATTGGTTACGCAAGTGCTCAGAACAGGAGTGA
GCGTGGGAGCGAAAAAGAATGTAAGAACTACGGGAGATTTCATTAGCAGAGAGAAA
GTGGTTAAAGCGGTGAGGGAGGTGTTGGTTGGGGAAGAGGCGGATGAGAGGCGG
GAGAGGGCAAAGAAGTTGGCAGAGATGGCTAAAGCTGCCGTGGAAGGAGGGTCTT
CTTTCAACGATCTAAACAGCTTCATAGAAGAGTTTACCTCGTAA
SEQ ID NO: 25 >UGT73B4
ATGAACAGAGAGCAAATTCATATTTTGTTCTTCCCCTTCATGGCTCATGGCCACATG
ATTCCACTCTTAGACATGGCCAAGCTTTTCGCTAGAAGAGGAGCCAAATCAACTCTC
CTCACAACCCCAATAAATGCTAAGATCTTGGAGAAACCCATTGAAGCATTCAAAGTT
CAAAATCCTGATCTCGAAATCGGAATCAAGATCCTCAATTTCCCTTGTGTAGAGCTT
GGATTGCCAGAAGGATGCGAGAACCGTGACTTCATTAACTCATACCAAAAATCTGA
CTCATTTGACTTGTTCTTGAAGTTTCTTTTCTCTACCAAGTATATGAAACAGCAGTTG
GAGAGTTTCATTGAAACAACCAAACCGAGTGCTCTTGTAGCCGATATGTTCTTCCCT
TGGGCAACAGAATCCGCGGAGAAGATCGGTGTTCCAAGACTTGTGTTCCACGGCA
CATCATCCTTTGCCTTGTGTTGTTCGTATAACATGAGGATTCATAAGCCACACAAGA
AAGTCGCTTCGAGTTCTACTCCATTTGTAATCCCTGGTCTCCCTGGAGACATAGTTA
TTACAGAAGACCAAGCCAATGTCACCAACGAAGAAACTCCATTCGGAAAGTTTTGG
AAAGAAGTCAGGGAATCAGAGACCAGTAGCTTTGGTGTTTTGGTGAATAGCTTCTA
CGAGCTGGAATCATCTTATGCTGATTTTTACCGTAGTTTTGTGGCGAAAAAAGCGTG
GCATATAGGTCCACTTTCACTATCCAACAGAGGGATTGCAGAGAAAGCCGGAAGAG
GGAAAAAGGCAAACATTGATGAGCAAGAATGCCTCAAATGGCTTGACTCTAAGACA
CCTGGCTCAGTAGTTTACTTGTCCTTTGGTAGCGGAACCGGCTTACCCAACGAACA
GCTGTTAGAGATTGCTTTCGGCCTTGAAGGCTCTGGACAAAATTTCATTTGGGTGG
TTAGCAAAAATGAAAACCAAGGTGAAAATGAAGATTGGTTGCCTAAAGGGTTTGAAG
AGAGGAATAAAGGAAAAGGGCTGATAATACGCGGATGGGCCCCGCAAGTGCTGAT
ACTTGACCACAAAGCAATCGGAGGATTTGTGACGCATTGCGGATGGAACTCGACTT
TGGAGGGCATTGCCGCAGGGCTGCCTATGGTGACTTGGCCGATGGGGGCAGAAC
AGTTCTACAACGAGAAGTTATTGACAAAAGTGTTGAGAATAGGAGTGAACGTTGGA
GCTACCGAGTTGGTGAAAAAAGGAAAGTTGATTAGTAGAGCACAAGTGGAGAAGGC
AGTAAGGGAAGTGATTGGTGGTGAGAAGGCAGAGGAAAGGCGGCTAAGGGCTAA
GGAGCTGGGCGAGATGGCTAAAGCCGCTGTGGAAGAAGGAGGGTCTTCTTATAAT
GATGTGAACAAGTTTATGGAAGAGCTGAATGGTAGAAAGTAG
SEQ ID NO: 26 >UGT73B5
ATGAACAGAGAAGTCTCTGAGAGAATTCATATTTTGTTCTTCCCCTTCATGGCTCAA
GGCCACATGATTCCAATTTTGGACATGGCCAAGCTTTTCTCGAGGAGAGGAGCCAA
GTCAACCCTTCTCACAACCCCAATCAACGCTAAGATCTTCGAGAAACCTATTGAAGC
ATTCAAAAATCAAAACCCTGATCTCGAAATCGGAATCAAGATCTTCAATTTCCCTTGT
GTAGAGCTTGGATTGCCTGAAGGATGCGAGAACGCTGACTTTATCAACTCATACCA
AAAATCTGACTCAGGTGACTTGTTCTTGAAGTTTCTTTTCTCTACCAAGTATATGAAA
CAACAGTTGGAGAGTTTCATTGAAACAACCAAACCAAGTGCTCTTGTTGCCGATATG
TTCTTCCCTTGGGCGACAGAATCTGCTGAGAAGCTCGGTGTACCAAGACTTGTGTT
CCACGGTACATCTTTCTTTTCTTTGTGTTGTTCGTATAACATGAGGATTCATAAGCC
ACACAAGAAAGTCGCTACGAGTTCTACTCCTTTTGTAATCCCTGGTCTCCCAGGAG
ACATAGTTATTACAGAAGACCAAGCCAATGTTGCCAAAGAAGAAACGCCAATGGGA
AAGTTTATGAAAGAGGTTAGGGAATCAGAGACCAATAGCTTTGGTGTATTGGTTAAT
AGCTTCTACGAGCTGGAATCAGCTTATGCTGATTTTTATCGTAGTTTTGTGGCGAAA
AGAGCTTGGCATATCGGTCCGCTTTCGCTATCTAACAGAGAGTTAGGAGAGAAAGC
CAGAAGAGGGAAAAAGGCTAACATTGATGAGCAAGAATGCCTAAAATGGCTGGACT
CTAAGACACCTGGTTCAGTAGTTTACTTGTCCTTTGGGAGCGGAACTAATTTCACCA
ACGACCAGCTGTTAGAGATCGCTTTTGGTCTTGAAGGTTCTGGACAAAGTTTCATCT
GGGTGGTTAGGAAAAATGAAAACCAAGGTGACAATGAAGAGTGGTTGCCTGAAGG
GTTTAAAGAGAGGACAACAGGGAAAGGGCTAATAATACCTGGATGGGCGCCGCAA
GTGCTGATACTTGACCATAAAGCAATTGGAGGATTTGTGACTCATTGCGGATGGAA
CTCGGCTATAGAGGGCATTGCCGCGGGGCTGCCTATGGTAACATGGCCAATGGGG
GCAGAACAGTTCTACAATGAGAAGCTATTGACAAAAGTGTTGAGAATAGGAGTGAA
CGTTGGAGCTACCGAGTTGGTGAAAAAAGGAAAGTTGATTAGTAGAGCACAAGTGG
AGAAGGCAGTAAGGGAAGTGATTGGTGGTGAGAAGGCAGAGGAAAGGCGGCTAT
GGGCTAAGAAGCTGGGCGAGATGGCTAAAGCCGCTGTGGAAGAAGGAGGGTCCT
CTTATAATGATGTGAACAAGTTTATGGAAGAGCTGAATGGTAGAAAGTAG
SEQ ID NO: 27 >UGT73C1
ATGGCATCGGAATTTCGTCCTCCTCTTCATTTTGTTCTCTTCCCTTTCATGGCTCAA
GGCCACATGATCCCAATGGTAGATATTGCAAGGCTCCTGGCTCAGCGCGGGGTGA
CTATAACCATTGTCACTACACCTCAAAACGCAGGCCGGTTCAAGAACGTTCTTAGCC
GGGCTATCCAATCCGGCTTGCCCATCAATCTCGTGCAAGTAAAGTTTCCATCTCAA
GAATCGGGTTCACCGGAAGGACAGGAGAATTTGGACTTGCTCGATTCATTGGGGG
CTTCATTAACCTTCTTCAAAGCATTTAGCCTGCTCGAGGAACCAGTCGAGAAGCTCT
TGAAAGAGATTCAACCTAGGCCAAACTGCATAATCGCTGACATGTGTTTGCCTTATA
CAAACAGAATTGCCAAGAATCTTGGTATACCAAAAATCATCTTTCATGGCATGTGTT
GCTTCAATCTTCTTTGTACGCACATAATGCACCAAAACCACGAGTTCTTGGAAACTA
TAGAGTCTGACAAGGAATACTTCCCCATTCCTAATTTCCCTGACAGAGTTGAGTTCA
CAAAATCTCAGCTTCCAATGGTATTAGTTGCTGGAGATTGGAAAGACTTCCTTGACG
GAATGACAGAAGGGGATAACACTTCTTATGGTGTGATTGTTAACACGTTTGAAGAG
CTCGAGCCAGCTTATGTTAGAGACTACAAGAAGGTTAAAGCGGGTAAGATATGGAG
CATCGGACCGGTTTCCTTGTGCAACAAGTTAGGAGAAGACCAAGCTGAGAGGGGA
AACAAGGCGGACATTGATCAAGACGAGTGTATTAAATGGCTTGATTCTAAAGAAGAA
GGGTCGGTGCTATATGTTTGCCTTGGAAGTATATGCAATCTTCCTCTGTCTCAGCTC
AAAGAGCTCGGCTTAGGCCTCGAGGAATCCCAAAGACCTTTCATTTGGGTCATAAG
AGGTTGGGAGAAGTATAACGAGTTACTTGAATGGATCTCAGAGAGCGGTTATAAGG
AAAGAATCAAAGAAAGAGGCCTTCTCATAACAGGATGGTCGCCTCAAATGCTTATCC
TTACACATCCTGCCGTTGGAGGATTCTTGACACATTGTGGATGGAACTCTACTCTTG
AAGGAATCACTTCAGGCGTTCCATTACTCACGTGGCCACTGTTTGGAGACCAATTC
TGCAATGAGAAATTGGCGGTGCAGATACTAAAAGCCGGTGTGAGAGCTGGGGTTG
AAGAGTCCATGAGATGGGGAGAAGAGGAGAAAATAGGAGTACTGGTGGATAAAGA
AGGAGTAAAGAAGGCAGTGGAGGAATTGATGGGTGATAGTAATGATGCTAAGGAG
AGAAGAAAAAGAGTGAAAGAGCTTGGAGAATTAGCTCACAAGGCTGTGGAAGAAG
GAGGCTCTTCTCATTCCAACATCACATTCTTGCTACAAGACATAATGCAATTAGAAC
AACCCAAGAAATGA
SEQ ID NO: 28 >UGT73C2
ATGGCTTTCGAGAAGACCCGCCAATTTCTTCCTCCGCTTCACTTTGTTCTCTTCCCT
TTCATGGCTCAAGGCCACATGATCCCCATGGTGGATATTGCAAGGATCTTGGCTCA
GCGCGGGGTGACTATTACCATTGTCACGACGCCTCACAACGCAGCCAGGTTCAAA
GATGTCCTAAACCGGGCCATCCAGTCAGGCTTGCACATTAGGGTTGAGCATGTGAA
GTTTCCTTTTCAAGAAGCTGGTTTGCAAGAAGGACAAGAGAATGTTGATTTTCTTGA
CTCAATGGAGTTAATGGTACATTTCTTTAAAGCGGTTAACATGCTTGAAAATCCGGT
CATGAAGCTCATGGAAGAGATGAAACCTAAACCAAGCTGCCTAATTTCTGATTTTTG
TTTGCCTTATACAAGCAAAATCGCTAAGAGGTTCAATATCCCAAAGATCGTTTTCCA
TGGCGTGTCTTGCTTTTGTCTTTTGAGTATGCATATTCTACACCGAAACCACAATAT
CTTACATGCTTTAAAGTCGGACAAAGAGTATTTCTTGGTTCCTAGTTTTCCAGATAG
AGTTGAATTTACAAAGCTTCAAGTTACTGTGAAAACAAACTTTAGTGGAGATTGGAA
AGAGATCATGGACGAACAGGTGGATGCTGATGACACGTCCTATGGTGTAATTGTCA
ACACATTTCAGGATTTGGAGTCTGCCTATGTGAAAAACTACACGGAGGCTAGGGCT
GGTAAAGTATGGAGCATCGGTCCGGTTTCCTTGTGCAACAAGGTAGGAGAAGACAA
AGCTGAGAGGGGAAACAAGGCAGCCATTGATCAAGACGAGTGTATTAAATGGCTTG
ATTCTAAAGATGTAGAGTCGGTGCTGTATGTTTGCCTTGGAAGTATATGCAATCTTC
CTCTGGCTCAGCTTAGAGAGCTCGGGCTAGGCCTCGAGGCAACTAAAAGACCATT
CATTTGGGTCATAAGAGGTGGGGGAAAGTATCATGAACTAGCTGAGTGGATCTTAG
AGAGCGGTTTTGAAGAAAGAACCAAAGAGAGAAGCCTTCTCATAAAAGGATGGTCG
CCTCAAATGCTTATCCTTTCACACCCTGCCGTTGGAGGATTCCTGACACATTGTGGA
TGGAACTCAACTTTAGAAGGAATCACCTCAGGGGTTCCATTGATCACTTGGCCATTA
TTTGGAGACCAATTCTGCAACCAGAAACTGATCGTGCAGGTGCTAAAAGCAGGTGT
AAGTGTTGGGGTTGAAGAGGTCATGAAATGGGGAGAAGAGGAGAGTATTGGAGTG
TTAGTGGATAAAGAAGGAGTGAAGAAGGCAGTGGACGAAATAATGGGCGAGAGTG
ATGAAGCAAAAGAGAGAAGAAAAAGAGTCAGAGAGCTTGGAGAATTAGCTCACAAG
GCTGTGGAAGAAGGAGGCTCTTCTCATTCTAATATCATATTTTTGCTACAAGATATA
ATGCAACAAGTAGAATCCAAGAGTTGA
SEQ ID NO: 29 >UGT73C3
ATGGCTACGGAAAAAACCCACCAATTTCATCCTTCTCTTCACTTTGTCCTCTTCCCTT
TCATGGCTCAAGGCCACATGATTCCCATGATTGATATTGCAAGACTCTTGGCTCAG
CGTGGTGTGACCATAACAATTGTCACGACACCTCACAACGCAGCAAGGTTTAAGAA
TGTCCTAAACCGAGCGATCGAGTCTGGCTTGGCCATCAACATACTGCATGTGAAGT
TTCCATATCAAGAGTTTGGTTTGCCAGAAGGAAAAGAGAATATAGATTCGTTAGACT
CAACGGAGTTGATGGTACCTTTCTTCAAAGCGGTGAACTTGCTTGAAGATCCGGTC
ATGAAGCTCATGGAAGAGATGAAACCTAGACCTAGCTGTCTAATTTCTGATTGGTGT
TTGCCTTATACAAGCATAATCGCCAAGAACTTCAATATACCAAAGATAGTTTTCCAC
GGCATGGGTTGCTTTAATCTTTTGTGTATGCATGTTCTACGCAGAAACTTAGAGATC
CTAGAGAATGTAAAGTCGGATGAAGAGTATTTCTTGGTTCCTAGTTTTCCTGATAGA
GTTGAATTTACAAAGCTTCAACTTCCTGTGAAAGCAAATGCAAGTGGAGATTGGAAA
GAGATAATGGATGAAATGGTAAAAGCAGAATACACATCCTATGGTGTGATCGTCAA
CACATTTCAGGAGTTGGAGCCACCTTATGTCAAAGACTACAAAGAGGCAATGGATG
GAAAAGTATGGTCCATTGGACCCGTTTCCTTGTGTAACAAGGCAGGTGCAGACAAA
GCTGAGAGGGGAAGCAAGGCCGCCATTGATCAAGATGAGTGTCTTCAATGGCTTG
ATTCTAAAGAAGAAGGTTCGGTGCTCTATGTTTGCCTTGGAAGTATATGTAATCTTC
CTTTGTCTCAGCTCAAGGAGCTGGGGCTAGGCCTTGAGGAATCTCGAAGATCTTTT
ATTTGGGTCATAAGAGGTTCGGAAAAGTATAAAGAACTATTTGAGTGGATGTTGGA
GAGCGGTTTTGAAGAAAGAATCAAAGAGAGAGGACTTCTCATTAAAGGGTGGGCAC
CTCAAGTCCTTATCCTTTCACATCCTTCCGTTGGAGGATTCCTGACACACTGTGGAT
GGAACTCGACTCTCGAAGGAATCACCTCAGGCATTCCACTGATCACTTGGCCGCTG
TTTGGAGACCAATTCTGCAACCAAAAACTGGTCGTTCAAGTACTAAAAGCCGGTGTA
AGTGCCGGGGTTGAAGAAGTCATGAAATGGGGAGAAGAAGATAAAATAGGAGTGT
TAGTGGATAAAGAAGGAGTGAAAAAGGCTGTGGAAGAATTGATGGGTGATAGTGAT
GATGCAAAAGAGAGGAGAAGAAGAGTCAAAGAGCTTGGAGAATTAGCTCACAAAGC
TGTGGAAAAAGGAGGCTCTTCTCATTCTAACATCACACTCTTGCTACAAGACATAAT
GCAACTAGCACAATTCAAGAATTGA
SEQ ID NO: 30 >UGT73C4
ATGGCTTCCGAAAAATCCCACAAAGTTCATCCTCCTCTTCACTTTATTCTTTTCCCTT
TCATGGCTCAGGGCCACATGATTCCCATGATTGATATAGCAAGGCTCTTGGCTCAG
CGCGGTGCGACAGTAACTATTGTCACGACACGTTATAATGCAGGGAGGTTCGAGAA
TGTCTTAAGTCGTGCCATGGAGTCTGGTTTACCCATCAACATAGTGCATGTGAATTT
TCCATATCAAGAATTTGGTTTGCCAGAAGGAAAAGAGAATATAGATTCGTATGACTC
AATGGAGCTGATGGTACCTTTCTTTCAAGCAGTTAACATGCTCGAAGATCCGGTCAT
GAAGCTCATGGAAGAGATGAAACCTAGACCTAGCTGTATTATTTCTGATTTGCTCTT
GCCTTATACAAGCAAAATCGCAAGGAAATTCAGTATACCAAAGATAGTTTTCCACGG
CACGGGTTGCTTTAATCTTTTGTGTATGCATGTTCTACGCAGAAACCTCGAGATCTT
GAAGAACTTAAAGTCGGATAAAGATTATTTCCTGGTTCCTAGTTTTCCTGATAGAGT
TGAATTTACAAAGCCTCAAGTTCCAGTGGAAACAACTGCAAGTGGAGATTGGAAAG
CGTTCTTGGACGAAATGGTAGAAGCAGAATACACATCCTATGGTGTGATCGTCAAC
ACATTTCAGGAGTTGGAGCCTGCTTATGTCAAAGACTACACGAAGGCTAGGGCTGG
AAAAGTATGGTCCATTGGACCTGTTTCCTTGTGCAACAAGGCAGGTGCTGATAAAG
CTGAGAGGGGAAACCAGGCCGCCATTGATCAAGATGAGTGTCTTCAATGGCTTGAT
TCTAAAGAAGATGGTTCGGTGTTATATGTTTGCCTTGGAAGTATCTGTAATCTACCT
TTGTCTCAGCTCAAGGAGCTGGGGCTAGGCCTTGAAAAATCCCAAAGATCTTTTATT
TGGGTCATAAGAGGTTGGGAAAAGTATAATGAACTATATGAGTGGATGATGGAGAG
CGGTTTTGAAGAAAGAATCAAAGAGAGAGGACTTCTTATTAAAGGGTGGTCACCTC
AAGTCCTTATCCTTTCACATCCTTCCGTTGGAGGATTCCTGACACACTGTGGATGGA
ACTCGACTCTCGAAGGAATCACCTCAGGCATTCCACTGATCACTTGGCCGCTGTTT
GGAGACCAATTCTGCAACCAAAAACTGGTCGTTCAAGTACTAAAAGCCGGTGTAAG
TGCCGGGGTTGAAGAAGTCATGAAATGGGGAGAAGAGGAGAAAATAGGAGTGTTA
GTGGATAAAGAAGGAGTAAAGAAGGCAGTGGAAGAGTTAATGGGTGCGAGTGATG
ATGCAAAAGAGAGGAGAAGAAGAGTCAAAGAGCTTGGAGAATCAGCTCACAAGGCT
GTGGAAGAAGGAGGCTCTTCTCATTCTAACATCACATACTTGCTACAAGACATAATG
CAACAAGTGAAATCCAAGAACTGA
SEQ ID NO: 31 >UGT73C5
ATGGTTTCCGAAACAACCAAATCTTCTCCACTTCACTTTGTTCTCTTCCCTTTCATGG
CTCAAGGCCACATGATTCCCATGGTTGATATTGCAAGGCTCTTGGCTCAGCGTGGT
GTGATCATAACAATTGTCACGACGCCTCACAATGCAGCGAGGTTCAAGAATGTCCT
AAACCGTGCCATTGAGTCTGGCTTGCCCATCAACTTAGTGCAAGTCAAGTTTCCATA
TCTAGAAGCTGGTTTGCAAGAAGGACAAGAGAATATCGATTCTCTTGACACAATGG
AGCGGATGATACCTTTCTTTAAAGCGGTTAACTTTCTCGAAGAACCAGTCCAGAAGC
TCATTGAAGAGATGAACCCTCGACCAAGCTGTCTAATTTCTGATTTTTGTTTGCCTT
ATACAAGCAAAATCGCCAAGAAGTTCAATATCCCAAAGATCCTCTTCCATGGCATGG
GTTGCTTTTGTCTTCTGTGTATGCATGTTTTACGCAAGAACCGTGAGATCTTGGACA
ATTTAAAGTCAGATAAGGAGCTTTTCACTGTTCCTGATTTTCCTGATAGAGTTGAATT
CACAAGAACGCAAGTTCCGGTAGAAACATATGTTCCAGCTGGAGACTGGAAAGATA
TCTTTGATGGTATGGTAGAAGCGAATGAGACATCTTATGGTGTGATCGTCAACTCAT
TTCAAGAGCTCGAGCCTGCTTATGCCAAAGACTACAAGGAGGTAAGGTCCGGTAAA
GCATGGACCATTGGACCCGTTTCCTTGTGCAACAAGGTAGGAGCCGACAAAGCAG
AGAGGGGAAACAAATCAGACATTGATCAAGATGAGTGCCTTAAATGGCTCGATTCT
AAGAAACATGGCTCGGTGCTTTACGTTTGTCTTGGAAGTATCTGTAATCTTCCTTTG
TCTCAACTCAAGGAGCTGGGACTAGGCCTAGAGGAATCCCAAAGACCTTTCATTTG
GGTCATAAGAGGTTGGGAGAAGTACAAAGAGTTAGTTGAGTGGTTCTCGGAAAGC
GGCTTTGAAGATAGAATCCAAGATAGAGGACTTCTCATCAAAGGATGGTCCCCTCA
AATGCTTATCCTTTCACATCCATCAGTTGGAGGGTTCCTAACACACTGTGGTTGGAA
CTCGACTCTTGAGGGGATAACTGCTGGTCTACCGCTACTTACATGGCCGCTATTCG
CAGACCAATTCTGCAATGAGAAATTGGTCGTTGAGGTACTAAAAGCCGGTGTAAGA
TCCGGGGTTGAACAGCCTATGAAATGGGGAGAAGAGGAGAAAATAGGAGTGTTGG
TGGATAAAGAAGGAGTGAAGAAGGCAGTGGAAGAATTAATGGGTGAGAGTGATGA
TGCAAAAGAGAGAAGAAGAAGAGCCAAAGAGCTTGGAGATTCAGCTCACAAGGCT
GTGGAAGAAGGAGGCTCTTCTCATTCTAACATCTCTTTCTTGCTACAAGACATAATG
GAACTGGCAGAACCCAATAATTGA
SEQ ID NO: 32 >UGT73C6
ATGGCTTTCGAAAAAAACAACGAACCTTTTCCTCTTCACTTTGTTCTCTTCCCTTTCA
TGGCTCAAGGCCACATGATTCCCATGGTTGATATTGCAAGGCTCTTGGCTCAGCGA
GGTGTGCTTATAACAATTGTCACGACGCCTCACAATGCAGCAAGGTTCAAGAATGT
CCTAAACCGTGCCATTGAGTCTGGTTTGCCCATCAACCTAGTGCAAGTCAAGTTTC
CATATCAAGAAGCTGGTCTGCAAGAAGGACAAGAAAATATGGATTTGCTTACCACG
ATGGAGCAGATAACATCTTTCTTTAAAGCGGTTAACTTACTCAAAGAACCAGTCCAG
AACCTTATTGAAGAGATGAGCCCGCGACCAAGCTGTCTAATCTCTGATATGTGTTTG
TCGTATACAAGCGAAATCGCCAAGAAGTTCAAAATACCAAAGATCCTCTTCCATGGC
ATGGGTTGCTTTTGTCTTCTGTGTGTTAACGTTCTGCGCAAGAACCGTGAGATCTTG
GACAATTTAAAGTCTGATAAGGAGTACTTCATTGTTCCTTATTTTCCTGATAGAGTTG
AATTCACAAGACCTCAAGTTCCGGTGGAAACATATGTTCCTGCAGGCTGGAAAGAG
ATCTTGGAGGATATGGTAGAAGCGGATAAGACATCTTATGGTGTTATAGTCAACTCA
TTTCAAGAGCTCGAACCTGCGTATGCCAAAGACTTCAAGGAGGCAAGGTCTGGTAA
AGCATGGACCATTGGACCTGTTTCCTTGTGCAACAAGGTAGGAGTAGACAAAGCAG
AGAGGGGAAACAAATCAGATATTGATCAAGATGAGTGCCTTGAATGGCTCGATTCT
AAGGAACCGGGATCTGTGCTCTACGTTTGCCTTGGAAGTATTTGTAATCTTCCTCTG
TCTCAGCTCCTTGAGCTGGGACTAGGCCTAGAGGAATCCCAAAGACCTTTCATCTG
GGTCATAAGAGGTTGGGAGAAATACAAAGAGTTAGTTGAGTGGTTCTCGGAAAGCG
GCTTTGAAGATAGAATCCAAGATAGAGGACTTCTCATCAAAGGATGGTCCCCTCAA
ATGCTTATCCTTTCACATCCTTCTGTTGGAGGGTTCTTAACGCACTGCGGATGGAAC
TCGACTCTTGAGGGGATAACTGCTGGTCTACCAATGCTTACATGGCCACTATTTGC
AGACCAATTCTGCAACGAGAAACTGGTCGTACAAATACTAAAAGTCGGTGTAAGTG
CCGAGGTTAAAGAGGTCATGAAATGGGGAGAAGAAGAGAAGATAGGAGTGTTGGT
GGATAAAGAAGGAGTGAAGAAGGCAGTGGAAGAACTAATGGGTGAGAGTGATGAT
GCAAAAGAGAGAAGAAGAAGAGCCAAAGAGCTTGGAGAATCAGCTCACAAGGCTG
TGGAAGAAGGAGGCTCCTCTCATTCTAATATCACTTTCTTGCTACAAGACATAATGC
AACTAGCACAGTCCAATAATTGA
SEQ ID NO: 33 >UGT73C7
ATGTGTTCTCATGATCCTCTTCACTTCGTCGTAATACCCTTTATGGCCCAAGGCCAT
ATGATCCCATTGGTCGACATCTCTAGGCTCTTGTCCCAGCGCCAAGGCGTGACTGT
CTGCATCATCACAACTACTCAAAATGTAGCCAAGATCAAGACTTCACTCTCATTTTC
CTCTTTGTTTGCGACTATCAACATCGTTGAAGTTAAGTTTCTGTCTCAACAAACGGG
TTTGCCAGAAGGGTGCGAGAGTTTAGATATGTTGGCTTCAATGGGCGATATGGTGA
AGTTCTTTGATGCTGCCAACTCACTTGAGGAGCAAGTTGAGAAAGCTATGGAAGAG
ATGGTTCAGCCGCGGCCAAGCTGCATCATTGGAGACATGAGCCTTCCTTTCACTTC
AAGACTTGCCAAGAAATTCAAGATCCCCAAACTTATCTTCCATGGGTTTTCTTGTTT
CAGCCTCATGTCTATACAAGTGGTTCGAGAAAGCGGGATCTTGAAAATGATAGAAT
CAAACGACGAGTATTTTGATTTGCCCGGCTTGCCTGACAAAGTTGAGTTCACGAAA
CCTCAGGTCTCTGTGTTGCAACCTGTTGAAGGAAATATGAAAGAGAGTACGGCCAA
GATTATTGAAGCTGATAATGACTCTTATGGTGTTATTGTGAACACTTTTGAAGAGTTA
GAGGTTGATTATGCAAGAGAATATAGGAAAGCAAGGGCTGGAAAAGTTTGGTGCGT
TGGACCTGTTTCCTTGTGCAATAGGTTAGGGTTAGACAAAGCTAAAAGAGGAGATA
AGGCTTCTATTGGTCAAGACCAATGTCTTCAATGGCTTGACTCTCAAGAAACTGGTT
CAGTGCTCTACGTTTGCCTTGGAAGTCTATGTAATCTTCCCTTGGCTCAGCTCAAAG
AGCTGGGACTAGGCCTTGAGGCATCTAATAAACCTTTCATATGGGTTATAAGAGAAT
GGGGAAAATATGGAGATTTAGCAAATTGGATGCAACAAAGCGGATTTGAAGAGCGG
ATCAAAGATAGAGGACTGGTGATCAAAGGTTGGGCGCCGCAAGTTTTCATCCTCTC
ACACGCATCCATTGGAGGGTTTTTGACTCACTGTGGATGGAACTCGACACTAGAAG
GAATTACTGCAGGAGTTCCATTATTGACATGGCCTTTGTTTGCTGAACAATTCTTGA
ATGAGAAGTTAGTTGTGCAGATACTAAAAGCAGGGTTAAAGATAGGAGTAGAGAAA
TTGATGAAATATGGAAAAGAAGAGGAGATAGGAGCGATGGTGAGCAGAGAATGTGT
GAGAAAAGCTGTGGATGAGCTAATGGGTGATAGTGAAGAAGCAGAAGAGAGAAGA
AGAAAAGTTACAGAACTTAGTGACTTGGCAAATAAGGCTTTGGAAAAAGGAGGATC
TTCAGATTCTAATATCACATTGCTCATTCAAGATATTATGGAGCAATCACAAAATCAA
TTTTAA
SEQ ID NO: 34 >UGT73D1
ATGGAATCAAAAATAGTTTCAAAAGCCAAAAGACTTCACTTTGTTTTGATCCCTCTCA
TGGCTCAAGGGCATCTGATCCCCATGGTCGACATCTCCAAGATTCTTGCACGACAA
GGCAACATCGTTACCATAGTTACAACCCCTCAAAATGCTTCTAGGTTTGCGAAGACA
GTTGACCGAGCAAGATTAGAGTCGGGTCTCGAAATCAATGTCGTTAAATTTCCAATT
CCTTACAAAGAATTCGGTCTTCCCAAAGATTGTGAGACTCTGGACACTTTGCCCTCC
AAAGACCTCCTACGAAGATTCTATGACGCTGTGGATAAACTCCAAGAGCCCATGGA
ACGGTTTCTTGAGCAACAAGATATCCCTCCAAGTTGCATAATCTCCGATAAATGCCT
TTTTTGGACGTCAAGAACCGCAAAGAGGTTCAAAATCCCGAGGATCGTGTTCCATG
GAATGTGTTGCTTCTCTCTTTTGAGTTCGCACAATATCCATCTTCATAGCCCGCACC
TCTCGGTTTCTTCGGCCGTAGAGCCATTCCCTATACCAGGAATGCCACATAGGATT
GAGATAGCTAGAGCTCAGTTACCTGGTGCTTTTGAGAAGTTAGCAAATATGGATGA
CGTTCGCGAGAAGATGCGTGAATCTGAATCAGAAGCCTTTGGGGTTATTGTTAATA
GCTTCCAGGAATTGGAGCCTGGCTATGCAGAGGCCTACGCTGAGGCCATCAATAA
GAAGGTATGGTTCGTTGGACCCGTTTCTTTATGCAACGACCGTATGGCTGACCTAT
TCGATAGAGGAAGTAATGGTAACATCGCAATAAGCGAGACCGAATGCTTGCAGTTT
CTTGACTCGATGAGACCAAGGTCAGTCTTATATGTTTCTCTTGGTAGCCTCTGTCGA
CTAATACCTAATCAATTGATAGAACTAGGTTTAGGGTTAGAAGAATCGGGAAAACCC
TTTATTTGGGTGATAAAGACCGAGGAAAAACACATGATTGAGCTAGACGAATGGCT
AAAACGCGAAAATTTTGAAGAGCGAGTTAGAGGAAGAGGGATAGTAATAAAGGGTT
GGAGTCCTCAGGCTATGATACTCTCACATGGTTCAACCGGCGGGTTCTTGACTCAT
TGCGGTTGGAATTCTACAATAGAAGCGATATGTTTTGGTGTACCAATGATCACATGG
CCGTTGTTCGCTGAACAATTTCTCAATGAGAAACTCATCGTGGAGGTTTTGAACATC
GGGGTTAGGGTTGGGGTGGAGATTCCGGTGAGATGGGGAGACGAGGAGAGACTT
GGAGTGTTGGTCAAGAAACCGAGTGTTGTGAAAGCTATAAAGCTTTTGATGGACCA
AGATTGTCAACGTGTAGACGAAAATGATGATGATAATGAATTCGTGAGACGAAGGA
GACGTATTCAAGAACTTGCAGTAATGGCGAAAAAGGCTGTGGAAGAAAAGGGATCT
TCGAGTATTAACGTTTCAATTTTAATCCAAGATGTTTTGGAGCAATTGAGTCTCGTG
TAG
SEQ ID NO: 35 >UGT74B1
ATGGCGGAAACAACTCCCAAAGTGAAAGGCCACGTCGTAATCTTACCATACCCAGT
TCAAGGCCACCTAAACCCAATGGTTCAATTCGCTAAACGTCTAGTCTCCAAAAACGT
CAAAGTCACAATCGCCACCACTACCTACACCGCCTCCTCAATCACAACACCATCACT
CTCCGTCGAACCAATCTCCGATGGATTCGATTTCATCCCCATAGGTATCCCCGGTTT
CAGCGTCGATACTTACTCAGAATCCTTCAAGCTCAACGGATCCGAAACCCTAACTCT
CCTAATCGAGAAATTCAAATCCACAGATTCACCAATCGATTGCTTAATCTACGATTC
GTTTCTTCCTTGGGGACTTGAAGTTGCTAGATCTATGGAACTTTCAGCTGCTTCTTT
CTTCACTAATAATCTCACTGTTTGTTCTGTGTTGCGTAAATTCTCTAACGGTGACTTT
CCTCTTCCCGCTGATCCTAATTCGGCGCCGTTTCGTATCCGTGGCTTACCGTCTTT
GAGCTACGATGAGTTACCTTCGTTTGTGGGACGTCATTGGTTGACTCATCCTGAGC
ATGGCAGAGTTCTTCTGAATCAGTTTCCTAACCATGAAAATGCTGATTGGTTATTCG
TTAATGGCTTTGAAGGGTTAGAAGAAACACAAGATTGTGAAAATGGTGAGTCTGAT
GCAATGAAGGCGACGTTGATCGGACCGATGATTCCATCGGCTTATCTTGATGATCG
GATGGAAGATGATAAAGACTATGGTGCGAGTCTGTTGAAACCGATATCGAAGGAGT
GTATGGAGTGGCTTGAGACTAAGCAGGCTCAGTCAGTAGCATTTGTTTCGTTTGGT
TCGTTTGGGATTCTCTTTGAGAAGCAACTTGCAGAGGTAGCTATTGCGCTACAAGA
ATCGGATTTGAACTTCTTGTGGGTGATTAAAGAAGCTCATATAGCGAAATTGCCTGA
AGGGTTTGTGGAATCGACTAAAGATAGAGCCTTGTTGGTTTCTTGGTGTAACCAGC
TTGAGGTTTTAGCTCATGAATCGATAGGTTGCTTTTTGACTCATTGTGGTTGGAACT
CTACGTTGGAAGGGTTGAGTTTGGGAGTTCCGATGGTTGGTGTGCCTCAGTGGAG
TGATCAGATGAATGATGCTAAGTTTGTGGAGGAAGTTTGGAAAGTTGGGTATAGAG
CGAAAGAGGAAGCTGGGGAAGTAATCGTGAAGAGTGAAGAATTGGTGAGGTGTTT
GAAAGGAGTGATGGAAGGAGAGAGTAGTGTGAAGATTAGAGAGAGTTCGAAGAAG
TGGAAAGATTTGGCTGTGAAGGCAATGAGTGAAGGAGGAAGCTCTGATCGAAGCA
TTAACGAGTTTATAGAGAGTTTAGGGAAGTAA
SEQ ID NO: 36 >UGT74C1
ATGAGTGAAGCAAAGAAGGGTCACGTACTGTTTTTTCCATATCCATTACAAGGCCAC
ATTAACCCAATGATCCAACTCGCTAAACGCTTATCCAAAAAGGGCATCACCAGCACA
CTCATCATCGCCTCCAAAGACCACCGTGAACCTTACACCTCCGACGACTACTCCAT
CACCGTCCACACCATCCACGACGGTTTCTTTCCACATGAACACCCTCACGCCAAGT
TCGTAGATCTTGACCGTTTCCACAACTCTACTTCTCGAAGCCTGACCGATTTCATCT
CTAGTGCGAAGTTGTCGGACAATCCTCCAAAAGCTCTGATCTATGATCCATTTATGC
CCTTTGCATTGGACATAGCCAAGGACTTGGATCTATACGTAGTGGCATATTTCACTC
AACCATGGTTGGCTAGTCTTGTTTACTACCATATCAACGAAGGCACCTACGATGTTC
CCGTTGATAGACACGAGAACCCAACACTTGCATCGTTTCCTGGTTTCCCATTGTTAA
GCCAAGATGATCTGCCTTCGTTCGCCTGCGAAAAAGGGTCGTACCCTCTTCTACAC
GAGTTTGTGGTTAGGCAATTCTCTAATTTATTGCAAGCTGATTGCATTCTCTGCAAC
ACTTTTGATCAACTTGAACCAAAGGTAGTGAAATGGATGAATGATCAATGGCCGGT
GAAGAACATTGGACCGGTGGTTCCATCGAAGTTCTTGGATAACCGGTTGCCAGAAG
ACAAAGATTACGAACTCGAGAACTCCAAGACAGAGCCAGACGAGTCTGTTTTGAAG
TGGTTGGGAAACAGGCCGGCGAAGTCGGTGGTTTACGTGGCGTTTGGGACATTGG
TGGCTTTGAGCGAAAAACAGATGAAGGAAATTGCAATGGCGATTAGCCAAACCGGA
TATCACTTCTTGTGGTCTGTTAGAGAATCCGAGAGAAGCAAACTACCCTCTGGTTTT
ATCGAAGAGGCAGAGGAGAAAGACTCTGGACTTGTGGCTAAGTGGGTTCCTCAGC
TAGAGGTTTTAGCACATGAATCAATCGGGTGTTTCGTGTCACACTGTGGATGGAAC
TCGACATTGGAGGCACTATGCTTAGGGGTTCCAATGGTGGGCGTGCCTCAGTGGA
CTGATCAGCCCACAAATGCTAAGTTTATAGAGGATGTGTGGAAGATTGGGGTTAGA
GTGAGGACCGATGGAGAAGGGCTTTCGAGTAAAGAAGAGATTGCGAGATGCATTG
TTGAGGTCATGGAAGGAGAGAGAGGGAAAGAGATAAGGAAGAATGTTGAGAAGCT
TAAGGTGTTGGCTCGCGAAGCTATCTCTGAAGGAGGTAGTTCCGACAAGAAGATTG
ATGAGTTTGTTGCTCTTTTGACTTAA
SEQ ID NO: 37 >UGT74D1
ATGGGAGAGAAAGCGAAAGCAAATGTGTTAGTCTTCTCATTTCCGATACAAGGTCA
CATAAACCCTCTCCTCCAATTCTCAAAACGCCTACTCTCTAAAAACGTCAACGTCAC
ATTCCTCACCACTTCCTCCACCCACAACTCCATCCTCCGCCGTGCCATCACCGGCG
GAGCCACTGCTCTTCCTCTCTCTTTTGTCCCCATTGACGATGGATTCGAGGAAGAT
CACCCATCTACGGACACATCTCCCGACTACTTCGCAAAGTTCCAAGAAAACGTATCT
CGAAGCCTCTCAGAGCTTATCTCCTCGATGGACCCAAAACCAAACGCCGTCGTTTA
CGACTCGTGCCTGCCTTATGTCCTCGACGTTTGCCGGAAACATCCTGGCGTTGCTG
CGGCGTCGTTTTTCACTCAGTCCTCCACCGTGAACGCGACCTATATTCATTTCTTGC
GTGGAGAGTTTAAGGAGTTTCAAAATGATGTCGTTTTGCCTGCAATGCCTCCGCTG
AAGGGTAATGACTTACCGGTGTTTCTGTACGATAACAATCTCTGCCGGCCGTTGTTT
GAGCTCATTAGTAGCCAGTTCGTGAATGTTGACGACATTGACTTCTTCTTGGTTAAC
TCTTTCGACGAACTCGAAGTCGAGGTGCTACAATGGATGAAAAACCAATGGCCGGT
CAAGAACATAGGACCGATGATTCCATCAATGTACTTAGACAAACGATTAGCAGGTG
ACAAAGACTACGGAATCAACCTCTTCAATGCCCAAGTCAACGAATGCCTTGATTGG
CTTGACTCAAAACCGCCCGGTTCAGTGATCTACGTGTCTTTTGGAAGCTTGGCCGT
CTTAAAAGACGATCAAATGATAGAAGTCGCGGCTGGTCTAAAACAAACTGGCCATA
ACTTCTTATGGGTTGTTAGAGAAACTGAAACAAAGAAGCTTCCAAGCAATTACATAG
AGGACATTTGTGACAAGGGATTGATAGTGAATTGGAGTCCTCAATTACAAGTTCTTG
CACATAAATCAATCGGTTGTTTCATGACTCATTGCGGGTGGAATTCGACTTTAGAGG
CATTGAGCTTAGGAGTTGCTTTGATAGGAATGCCGGCTTATAGCGACCAGCCGACT
AATGCTAAGTTTATTGAAGATGTGTGGAAGGTTGGGGTTAGGGTTAAGGCAGATCA
AAATGGGTTTGTTCCGAAGGAAGAGATTGTGAGATGTGTTGGAGAAGTTATGGAAG
ATATGTCGGAGAAAGGGAAGGAGATTAGAAAAAATGCTCGGAGGTTGATGGAGTTT
GCAAGGGAAGCTTTGTCTGATGGAGGAAATTCTGATAAGAATATTGATGAGTTTGTT
GCTAAAATTGTGAGGTAA
SEQ ID NO: 38 >UGT74E1
ATGAGAGAAGGATCTCATGTTATTGTTTTGCCTTTCCCAGCACAAGGCCACATAACT
CCAATGTCCCAATTCTGTAAACGCTTAGCCTCAAAAAGTCTTAAGATCACTCTTGTC
CTCGTCTCCGACAAGCCCTCTCCGCCGTACAAAACAGAGCACGACACAATCACTGT
CGTCCCCATCTCCAATGGTTTCCAAGAAGGCCAGGAACGATCAGAAGACCTAGATG
AGTACATGGAAAGAGTAGAATCCAGCATCAAAAACCGCTTACCGAAGTTGATAGAA
GACATGAAACTATCGGGAAATCCTCCTAGGGCTCTTGTGTACGACTCCACCATGCC
GTGGCTTCTGGATGTAGCTCATAGTTATGGTTTGAGCGGTGCCGTGTTTTTCACGC
AGCCTTGGCTTGTCTCAGCTATTTACTATCATGTATTCAAGGGCTCGTTCTCTGTAC
CGTCTACAAAGTATGGTCACTCGACGTTAGCATCTTTCCCTTCGTTACCGATTCTGA
ATGCGAATGATTTGCCGTCTTTCCTCTGTGAATCTTCCTCTTACCCATATATTCTAAG
GACTGTGATCGATCAGCTCTCAAACATTGATCGAGTTGATATAGTTTTGTGCAACAC
TTTCGATAAATTGGAAGAAAAGTTGCTGAAATGGATTAAAAGCGTGTGGCCTGTCCT
GAACATAGGACCAACTGTTCCATCAATGTATTTAGATAAGCGACTGGCTGAAGACAA
AAACTACGGATTCAGCCTCTTCGGTGCGAAAATCGCTGAATGCATGGAGTGGCTCA
ACTCAAAGCAGCCTAGTTCAGTTGTTTATGTATCATTTGGGAGCTTGGTGGTTCTAA
AAAAAGATCAACTGATAGAACTAGCGGCGGGTCTGAAACAGAGCGGACATTTCTTT
TTGTGGGTTGTGAGAGAGACGGAGAGAAGAAAACTTCCAGAAAACTATATAGAGGA
AATTGGTGAGAAAGGACTGACCGTGAGCTGGAGTCCACAACTTGAAGTTCTTACAC
ATAAATCGATCGGTTGTTTCGTGACACATTGTGGATGGAACTCGACGTTAGAGGGA
TTGAGTTTGGGAGTTCCAATGATTGGTATGCCTCATTGGGCAGATCAGCCTACAAA
TGCTAAGTTCATGGAGGATGTGTGGAAAGTTGGAGTTAGGGTTAAAGCAGACAGTG
ATGGGTTCGTGAGAAGAGAAGAGTTTGTGAGACGTGTGGAAGAAGTTATGGAGGC
AGAGCAAGGTAAAGAGATTAGAAAGAATGCTGAGAAATGGAAAGTGTTGGCTCAAG
AGGCTGTTTCTGAAGGAGGTAGTTCTGATAAGAACATCAATGAGTTTGTTTCTATGT
TTTGTTGA
SEQ ID NO: 39 >UGT74E2
ATGAGAGAAGGATCTCATCTTATCGTCTTGCCTTTCCCAGGACAAGGCCACATAACT
CCAATGTCCCAGTTCTGCAAACGCTTAGCCTCAAAAGGTCTTAAGCTCACTCTGGT
CCTCGTCTCCGACAAACCCTCTCCTCCATACAAAACAGAGCACGACTCAATCACTGT
CTTCCCCATCTCCAACGGCTTCCAAGAAGGCGAGGAACCATTACAAGACCTCGATG
ATTACATGGAAAGAGTAGAAACCAGCATCAAAAACACCTTACCGAAGTTGGTTGAAG
ACATGAAACTGTCGGGAAATCCACCTAGGGCTATCGTGTACGACTCCACCATGCCA
TGGCTTCTTGATGTAGCTCATAGTTATGGATTGAGCGGTGCCGTGTTTTTCACGCA
ACCTTGGCTTGTCACAGCTATTTACTACCATGTTTTCAAGGGTTCGTTCTCTGTACC
GTCTACAAAGTACGGTCACTCGACATTAGCATCTTTCCCTTCGTTCCCGATGCTGAC
TGCAAATGATTTGCCGTCTTTCCTCTGCGAATCGTCCTCATACCCGAATATACTGAG
GATTGTGGTGGATCAGCTCTCAAACATTGATCGAGTCGACATAGTGTTGTGCAACA
CTTTCGATAAATTGGAGGAAAAGTTGTTGAAATGGGTCCAAAGCTTGTGGCCAGTC
TTGAATATTGGACCAACGGTTCCATCGATGTATTTAGACAAACGACTGTCTGAAGAC
AAGAACTACGGTTTTAGCCTCTTCAATGCGAAAGTCGCTGAATGCATGGAGTGGCT
AAACTCAAAGGAGCCTAATTCTGTTGTCTATTTATCATTCGGAAGTTTGGTGATTCT
AAAAGAAGATCAAATGTTGGAACTCGCTGCGGGTCTGAAACAGAGCGGACGTTTCT
TTCTGTGGGTTGTGAGAGAGACAGAGACACACAAACTTCCAAGAAACTATGTCGAG
GAAATCGGTGAAAAAGGACTTATTGTAAGCTGGAGTCCTCAGCTTGACGTACTTGC
ACATAAATCAATCGGTTGTTTCTTGACACACTGTGGATGGAACTCGACGTTAGAGG
GATTGAGTTTGGGAGTTCCAATGATTGGTATGCCACACTGGACTGATCAGCCCACG
AATGCTAAGTTCATGCAGGATGTGTGGAAGGTTGGGGTAAGGGTTAAGGCAGAAG
GTGATGGGTTTGTGAGAAGAGAAGAGATTATGAGAAGTGTGGAAGAAGTTATGGAG
GGAGAGAAAGGGAAAGAGATTAGAAAGAATGCTGAGAAATGGAAAGTGTTGGCTCA
AGAGGCAGTTTCTGAAGGAGGTAGCTCTGATAAGAGCATCAATGAGTTTGTTTCTA
TGTTTTGTTGA
SEQ ID NO: 40 >UGT74F1
ATGGAGAAGATGAGAGGACATGTATTAGCAGTGCCATTTCCAAGCCAAGGACACAT
CACCCCGATTCGCCAATTCTGCAAACGACTTCACTCCAAAGGTTTCAAAACCACTCA
CACTCTCACCACTTTTATCTTCAACACAATCCACCTCGACCCATCTAGTCCTATCTC
CATAGCCACAATCTCCGATGGCTATGACCAGGGAGGGTTCTCATCAGCCGGTTCTG
TCCCGGAGTACCTACAAAACTTCAAAACCTTCGGCTCCAAAACCGTCGCTGATATCA
TCCGCAAACACCAGAGTACTGATAACCCTATTACTTGTATCGTCTATGATTCTTTCAT
GCCTTGGGCGCTTGACCTTGCAATGGATTTTGGTCTAGCTGCGGCTCCTTTCTTCA
CGCAGTCTTGCGCCGTTAACTATATCAATTATCTTTCTTACATAAACAATGGTAGCTT
GACACTTCCCATCAAGGATTTGCCTCTTCTTGAGCTCCAAGATTTGCCTACTTTCGT
CACTCCTACTGGTTCACACCTTGCTTACTTTGAGATGGTGCTTCAACAGTTCACCAA
CTTCGACAAAGCTGATTTCGTACTCGTTAATTCCTTCCATGACCTCGACCTTCATGA
AGAGGAGTTGTTGTCGAAAGTATGTCCTGTGTTGACAATTGGTCCAACTGTTCCAT
CAATGTACTTAGACCAACAGATCAAATCAGACAACGACTATGATCTGAACCTCTTTG
ACTTAAAAGAAGCTGCCTTATGCACTGACTGGCTAGACAAGAGGCCAGAAGGATCG
GTAGTATATATAGCTTTTGGGAGCATGGCTAAACTGAGTAGTGAGCAGATGGAAGA
GATTGCTTCGGCGATAAGCAACTTCAGCTACCTCTGGGTTGTCAGAGCTTCAGAGG
AGTCAAAGCTCCCACCAGGGTTTCTTGAAACAGTGGATAAAGACAAGAGCTTGGTC
TTGAAGTGGAGTCCTCAGCTTCAAGTTCTGTCAAACAAAGCCATCGGTTGTTTCATG
ACTCACTGTGGCTGGAACTCAACCATGGAGGGTTTGAGTTTAGGGGTTCCCATGGT
GGCTATGCCTCAATGGACTGATCAACCAATGAATGCAAAGTATATACAAGATGTATG
GAAGGTTGGGGTTCGTGTGAAAGCAGAGAAAGAAAGTGGCATTTGCAAAAGAGAG
GAGATTGAGTTTAGCATCAAGGAAGTGATGGAAGGAGAGAAGAGCAAAGAGATGAA
AGAGAATGCGGGAAAATGGAGAGACTTGGCTGTGAAGTCACTCAGTGAAGGAGGT
TCTACAGATATCAACATTAACGAATTTGTATCAAAAATTCAAATCAAATAA
SEQ ID NO: 41 >UGT74F2
ATGGAGCATAAGAGAGGACATGTATTAGCAGTGCCGTACCCAACGCAAGGACACAT
CACACCATTCCGCCAATTCTGCAAACGACTTCACTTCAAAGGTCTCAAAACCACTCT
CGCTCTCACCACTTTCGTCTTCAACTCCATCAATCCTGACCTATCCGGTCCAATCTC
CATAGCCACCATCTCCGATGGCTATGACCATGGGGGTTTCGAGACAGCTGACTCCA
TCGACGACTACCTCAAAGACTTTAAAACTTCCGGCTCGAAAACCATTGCAGACATCA
TCCAAAAACACCAGACTAGTGATAACCCCATCACTTGTATCGTCTATGATGCTTTCC
TGCCTTGGGCACTTGACGTTGCTAGAGAGTTTGGTTTAGTTGCGACTCCTTTCTTTA
CGCAGCCTTGTGCTGTTAACTATGTTTATTATCTTTCTTACATAAACAATGGAAGCTT
GCAACTTCCCATTGAGGAATTGCCTTTTCTTGAGCTCCAAGATTTGCCTTCTTTCTT
CTCTGTTTCTGGCTCTTATCCTGCTTACTTTGAGATGGTGCTTCAACAGTTCATAAA
TTTCGAAAAAGCTGATTTCGTTCTCGTTAATAGCTTCCAAGAGTTGGAACTGCATGA
GAATGAATTGTGGTCGAAAGCTTGTCCTGTGTTGACAATTGGTCCAACTATTCCATC
AATTTACTTAGACCAACGTATCAAATCAGACACCGGCTATGATCTTAATCTCTTTGAA
TCGAAAGATGATTCCTTCTGCATTAACTGGCTCGACACAAGGCCACAAGGGTCGGT
GGTGTACGTAGCATTCGGAAGCATGGCTCAGCTGACTAATGTGCAGATGGAGGAG
CTTGCTTCAGCAGTAAGCAACTTCAGCTTCCTGTGGGTGGTCAGATCTTCAGAGGA
GGAAAAACTCCCATCAGGGTTTCTTGAGACAGTGAATAAAGAAAAGAGCTTGGTCT
TGAAATGGAGTCCTCAGCTTCAAGTTCTGTCAAACAAAGCCATCGGTTGTTTCTTGA
CTCACTGTGGCTGGAACTCAACCATGGAGGCTTTGACCTTCGGGGTTCCCATGGT
GGCAATGCCCCAATGGACTGATCAACCGATGAACGCAAAGTACATACAAGATGTGT
GGAAGGCTGGAGTTCGTGTGAAGACAGAGAAGGAGAGTGGGATTGCCAAGAGAGA
GGAGATTGAGTTTAGCATTAAGGAAGTGATGGAAGGAGAGAGGAGCAAAGAGATG
AAGAAGAACGTGAAGAAATGGAGAGACTTGGCTGTCAAGTCACTCAATGAAGGAGG
TTCTACGGATACTAACATTGATACATTTGTATCAAGGGTTCAGAGCAAATAG
SEQ ID NO: 42 >UGT75B1
ATGGCGCCACCGCATTTTCTACTGGTAACGTTTCCGGCGCAAGGTCACGTGAACCC
ATCTCTCCGTTTTGCTCGTCGGCTCATCAAAAGAACCGGCGCACGTGTCACTTTCG
TCACTTGTGTCTCCGTCTTCCACAACTCCATGATCGCAAACCACAACAAAGTCGAAA
ATCTCTCTTTCCTTACTTTCTCCGACGGTTTCGACGATGGAGGCATTTCCACCTACG
AAGACCGTCAGAAAAGGTCGGTGAATCTCAAGGTTAACGGCGATAAGGCACTATCG
GATTTCATCGAAGCTACTAAGAATGGTGACTCTCCCGTGACTTGCTTGATCTACACG
ATTCTTCTCAATTGGGCTCCAAAAGTAGCACGTAGATTTCAACTTCCCTCCGCTCTT
CTCTGGATCCAACCGGCTTTGGTTTTCAACATCTATTACACTCATTTCATGGGAAAC
AAGTCCGTTTTCGAGTTACCTAATCTGTCTTCTCTGGAAATCAGAGATCTTCCATCT
TTCCTCACACCTTCCAACACAAACAAAGGCGCATACGATGCGTTTCAAGAAATGATG
GAGTTTCTCATAAAAGAAACCAAACCGAAAATTCTCATCAACACTTTCGATTCGCTG
GAACCAGAGGCCTTAACGGCTTTCCCGAATATCGATATGGTGGCGGTTGGTCCTTT
ACTTCCCACGGAGATTTTCTCAGGAAGCACCAACAAATCAGTTAAAGATCAAAGTAG
TAGTTATACACTTTGGCTAGACTCGAAAACAGAGTCCTCTGTTATTTACGTTTCCTTT
GGAACAATGGTTGAGTTGTCCAAGAAACAGATAGAGGAACTAGCGAGAGCACTCAT
AGAAGGGAAACGACCGTTTTTGTGGGTTATAACTGATAAATCCAACAGAGAAACGA
AAACAGAAGGAGAAGAAGAGACAGAGATTGAGAAGATAGCTGGATTCAGACACGA
GCTTGAAGAGGTTGGGATGATTGTGTCGTGGTGTTCGCAGATAGAGGTTTTAAGTC
ACCGAGCCGTAGGTTGTTTTGTGACTCATTGTGGGTGGAGCTCGACGCTGGAGAG
TTTGGTTCTTGGCGTTCCGGTTGTGGCGTTTCCGATGTGGTCGGATCAACCGACGA
ACGCGAAGCTACTGGAAGAAAGTTGGAAGACTGGTGTGAGGGTAAGAGAGAACAA
GGATGGTTTGGTGGAGAGAGGAGAGATCAGGAGGTGTTTGGAAGCCGTGATGGA
GGAGAAGTCGGTGGAGTTGAGGGAAAACGCAAAGAAATGGAAGCGTTTAGCGATG
GAAGCGGGTAGAGAAGGAGGATCTTCGGATAAGAACATGGAGGCTTTTGTGGAGG
ATATTTGTGGAGAATCTCTTATTCAAAACTTGTGTGAAGCAGAGGAGGTAAAAGTAA
AGTAA
SEQ ID NO: 43 >UGT75B2
ATGGCGCAACCGCATTTTCTACTGGTAACGTTTCCGGCGCAAGGTCACGTGAACCC
ATCTCTCCGTTTTGCTCGTCGGCTCATCAAAACAACTGGCGCACGTGTAACTTTCG
CCACGTGTCTCTCTGTCATTCACCGCTCTATGATCCCAAACCACAACAACGTCGAAA
ATCTCTCTTTCCTTACTTTCTCCGACGGATTCGACGACGGAGTCATCTCCAACACCG
ACGACGTCCAAAACCGGTTGGTACACTTCGAACGTAATGGCGATAAAGCTCTATCG
GATTTCATCGAAGCTAATCAGAATGGTGACTCTCCCGTAAGTTGCTTGATCTACACG
ATTCTTCCCAACTGGGTTCCAAAAGTGGCGCGTAGATTTCATCTTCCCTCTGTTCAT
CTCTGGATCCAACCAGCCTTCGCTTTCGACATTTATTACAATTACTCTACAGGAAAC
AACTCCGTTTTCGAGTTCCCGAATCTACCTTCTCTCGAAATCCGCGATCTGCCTTCT
TTCCTCTCACCTTCCAACACGAACAAAGCCGCACAAGCAGTATATCAAGAACTGATG
GATTTTCTCAAAGAAGAATCTAACCCGAAAATTCTCGTCAACACATTCGATTCGCTG
GAGCCAGAGTTCTTAACAGCTATTCCGAATATAGAAATGGTGGCAGTTGGTCCTTTA
CTTCCTGCGGAGATTTTCACTGGAAGCGAATCAGGTAAAGATTTATCAAGAGATCAT
CAAAGTAGTAGTTATACACTTTGGTTAGACTCGAAAACAGAGTCCTCTGTTATTTAT
GTTTCTTTTGGAACAATGGTTGAGTTGTCGAAGAAACAGATAGAGGAACTAGCGAG
AGCACTCATAGAAGGGGGAAGACCGTTCTTGTGGGTTATAACTGATAAACTCAACA
GAGAAGCGAAAATAGAAGGAGAAGAAGAGACAGAGATTGAGAAGATAGCTGGTTTT
AGACACGAGCTTGAAGAGGTTGGGATGATTGTCTCGTGGTGTTCGCAGATAGAGG
TTTTGAGACACCGAGCCATAGGTTGTTTTTTGACTCATTGTGGGTGGAGCTCATCA
CTGGAGAGTTTGGTTCTCGGCGTTCCAGTGGTGGCGTTTCCGATGTGGTCGGATC
AGCCAGCAAATGCGAAGCTTTTGGAAGAAATATGGAAGACAGGTGTGAGGGTGAG
AGAGAACTCGGAAGGTTTAGTAGAGAGAGGAGAGATAATGCGGTGTTTGGAAGCA
GTGATGGAGGCGAAATCGGTGGAGCTGAGGGAAAACGCAGAGAAATGGAAGCGTT
TAGCGACTGAAGCGGGTAGAGAAGGAGGATCTTCGGACAAGAATGTGGAAGCTTT
TGTGAAGAGTCTGTTTTGA
SEQ ID NO: 44 >UGT75C1
ATGGCCACTTCCGTCAATGGTTCCCATCGTCGTCCACATTACTTGCTTGTAACATTC
CCAGCGCAAGGTCACATCAACCCGGCGCTTCAACTAGCCAACCGCCTCATCCACCA
CGGTGCAACCGTCACATACTCCACCGCAGTCTCTGCTCACCGACGTATGGGCGAG
CCACCTTCCACAAAAGGTCTATCCTTCGCTTGGTTCACCGATGGATTCGACGACGG
TCTCAAGTCATTCGAAGACCAGAAAATCTACATGTCCGAACTCAAACGATGTGGTTC
AAACGCCCTGAGAGACATCATCAAAGCCAATCTTGACGCCACCACCGAAACAGAGC
CTATCACCGGGGTAATCTACTCTGTTCTCGTCCCGTGGGTTTCTACGGTAGCGCGT
GAGTTTCACCTCCCAACTACACTTCTCTGGATTGAACCAGCTACTGTACTAGACATC
TACTACTACTACTTCAACACCTCTTACAAACATCTCTTCGACGTTGAACCGATTAAAT
TACCGAAACTGCCACTGATCACCACCGGTGACCTCCCGTCGTTTCTTCAACCTTCG
AAGGCATTACCGTCAGCTCTTGTGACTCTAAGAGAACATATCGAAGCTCTCGAAAC
GGAATCAAACCCTAAGATTCTTGTTAACACATTCTCTGCTTTGGAACACGATGCTTT
AACCTCTGTTGAGAAACTCAAGATGATCCCAATCGGACCGTTGGTTTCTTCCTCCGA
GGGTAAAACCGATCTTTTCAAATCTTCCGACGAGGATTACACGAAATGGTTAGACTC
GAAGCTCGAGAGATCAGTGATTTACATTTCCTTAGGCACACACGCCGATGATTTAC
CAGAGAAACACATGGAAGCGCTTACTCACGGCGTGTTAGCTACAAACAGACCGTTT
TTATGGATCGTGAGGGAGAAAAATCCAGAAGAGAAGAAGAAGAATCGGTTTCTTGA
ATTGATCAGAGGAAGTGATCGAGGATTGGTGGTGGGATGGTGTTCTCAGACAGCT
GTTTTGGCGCATTGTGCTGTGGGATGTTTTGTGACTCATTGTGGTTGGAATTCGAC
GTTGGAGAGTTTAGAGAGTGGTGTTCCGGTGGTTGCGTTTCCGCAGTTTGCTGATC
AGTGTACAACGGCGAAGCTTGTGGAGGATACGTGGAGGATTGGAGTGAAGGTGAA
GGTTGGGGAGGAAGGAGATGTGGATGGGGAGGAGATTAGAAGGTGTTTGGAGAA
GGTGATGAGTGGTGGAGAAGAGGCGGAGGAGATGAGAGAGAATGCAGAGAAGTG
GAAGGCGATGGCTGTTGATGCGGCAGCGGAAGGTGGACCGTCGGATTTGAATCTT
AAAGGTTTTGTGGACGAGGATGAGTAG
SEQ ID NO: 45 >UGT75D1
ATGGCCAACAACAATTCCAACTCTCCCACCGGTCCACACTTTCTATTCGTAACATTT
CCAGCCCAAGGTCACATCAACCCATCTCTCGAGCTAGCCAAACGCCTCGCCGGAA
CAATCTCTGGTGCTCGAGTCACCTTCGCCGCCTCAATCTCTGCCTACAACCGCCGC
ATGTTCTCTACAGAAAACGTCCCCGAAACCCTAATCTTCGCTACCTACTCCGATGGC
CACGACGACGGTTTCAAATCCTCTGCTTACTCCGACAAATCTCGTCAAGACGCCAC
TGGAAACTTCATGTCTGAGATGAGACGACGTGGCAAAGAGACACTAACCGAACTAA
TCGAAGATAACCGGAAACAAAACAGGCCTTTTACTTGCGTGGTTTACACGATTCTCC
TCACTTGGGTCGCTGAGCTAGCGCGTGAGTTTCATCTTCCTTCTGCTCTTCTTTGG
GTCCAACCAGTAACAGTCTTCTCCATTTTTTACCATTACTTCAATGGCTACGAAGAT
GCAATCTCAGAGATGGCTAATACCCCCTCTAGTTCTATTAAATTACCTTCTCTGCCA
CTGCTTACTGTCCGTGATATTCCTTCTTTCATTGTCTCTTCCAATGTCTACGCGTTTC
TTCTACCCGCGTTTCGAGAACAGATTGATTCACTGAAGGAAGAAATAAACCCTAAGA
TCCTCATCAACACTTTCCAAGAGCTTGAGCCAGAAGCCATGAGCTCGGTTCCAGAT
AATTTCAAGATTGTCCCTGTCGGTCCGTTACTAACGTTGAGAACGGATTTTTCGAGT
CGCGGTGAATACATAGAGTGGTTGGATACTAAAGCGGATTCGTCTGTGCTTTATGT
TTCGTTCGGGACGCTTGCCGTGTTGAGCAAGAAACAGCTTGTGGAGCTTTGTAAAG
CGTTGATACAAAGTCGGAGACCATTCTTGTGGGTGATTACGGATAAGTCGTACAGA
AATAAAGAAGATGAGCAAGAGAAGGAAGAAGATTGCATAAGTAGTTTCAGAGAAGA
GCTCGATGAGATAGGAATGGTGGTTTCATGGTGTGATCAGTTTAGGGTTTTGAATC
ATAGATCGATAGGTTGTTTCGTGACGCATTGCGGGTGGAACTCTACGCTGGAGAGC
TTGGTTTCAGGAGTTCCGGTGGTGGCGTTTCCGCAATGGAATGATCAGATGATGAA
CGCGAAGCTTTTAGAAGATTGTTGGAAAACAGGTGTAAGAGTGATGGAGAAGAAGG
AAGAAGAAGGAGTTGTGGTGGTGGATAGTGAGGAGATACGGCGGTGCATTGAGGA
AGTTATGGAAGACAAGGCGGAGGAGTTTAGAGGAAATGCCACGAGGTGGAAGGAT
TTAGCGGCGGAGGCTGTGAGAGAAGGAGGCTCTTCCTTTAATCATCTCAAAGCTTT
TGTCGATGAGCACATGTGA
SEQ ID NO: 46 >UGT76B1
ATGGAGACTAGAGAAACAAAACCAGTGATCTTTCTCTTCCCTTTCCCTTTACAAGGT
CACTTAAACCCAATGTTTCAGCTCGCCAACATCTTCTTCAACAGAGGCTTCTCCATC
ACTGTGATCCACACTGAGTTCAACTCTCCAAACTCTTCCAATTTCCCTCATTTCACTT
TCGTATCCATCCCCGATAGCTTGTCTGAACCTGAATCCTATCCCGATGTCATCGAGA
TTCTCCATGACCTCAATTCCAAGTGTGTTGCTCCTTTTGGTGATTGCTTAAAGAAGC
TTATATCTGAAGAACCAACAGCAGCTTGTGTGATTGTTGACGCTCTTTGGTACTTCA
CTCACGATTTAACCGAGAAATTCAATTTCCCGAGGATTGTTCTCCGAACCGTTAACC
TCTCAGCTTTCGTCGCTTTCTCAAAGTTTCATGTTTTACGAGAGAAAGGGTATCTTT
CTTTACAAGAGACTAAGGCAGACTCACCGGTTCCGGAGCTTCCGTATCTTAGAATG
AAGGATCTTCCATGGTTCCAGACAGAAGATCCAAGATCAGGGGATAAGTTACAGAT
AGGTGTGATGAAGTCACTAAAGTCTTCCTCAGGAATCATATTCAACGCCATTGAAGA
TCTTGAAACAGATCAGCTTGATGAAGCCCGCATAGAATTCCCAGTTCCACTCTTCTG
TATTGGACCCTTTCACAGGTACGTTTCAGCTTCATCCAGTAGCTTACTTGCACACGA
CATGACTTGTCTCTCCTGGTTAGACAAGCAAGCAACAAATTCCGTAATCTACGCAAG
TCTTGGAAGCATTGCTTCGATCGATGAATCTGAATTCTTGGAGATTGCTTGGGGTCT
AAGAAACAGCAACCAACCTTTTCTATGGGTGGTTAGACCCGGTTTAATCCACGGGA
AAGAATGGATCGAGATTCTGCCTAAAGGGTTCATCGAAAATCTCGAGGGCCGGGG
TAAAATAGTGAAATGGGCACCTCAGCCTGAAGTTTTAGCTCACCGTGCAACAGGCG
GATTCTTAACACATTGTGGATGGAACTCAACACTTGAGGGCATATGTGAAGCTATAC
CAATGATATGCAGACCATCTTTTGGGGACCAGAGGGTGAATGCTAGATACATTAAC
GATGTTTGGAAGATCGGATTGCATTTGGAAAACAAGGTAGAGAGACTAGTGATCGA
AAACGCGGTTAGAACACTAATGACGAGCTCGGAAGGGGAAGAGATCCGCAAGAGG
ATTATGCCCATGAAGGAAACTGTTGAACAATGCCTTAAGCTTGGAGGTTCATCATTT
CGGAATCTCGAAAACTTAATTGCTTATATATTGTCTTTCTAA
SEQ ID NO: 47 >UGT76C1
ATGGAGAAGAGAAACGAGAGACAAGTGATTCTTTTTCCTCTACCATTACAAGGTTGC
ATAAACCCTATGCTTCAGCTAGCAAAGATCCTTTACTCAAGAGGTTTTTCGATCACC
ATCATCCACACGCGCTTCAACGCGCCCAAATCTTCAGACCATCCTCTCTTCACTTTC
TTACAAATCCGCGACGGCTTGTCTGAATCTCAGACTCAATCTCGTGATCTTTTGCTT
CAACTCACGCTTCTCAACAACAATTGTCAGATCCCATTTCGAGAGTGTTTGGCTAAA
CTCATTAAACCTAGTTCAGATTCAGGAACAGAGGATAGGAAAATTAGCTGTGTGATC
GATGATTCCGGTTGGGTTTTCACACAATCCGTGGCGGAGAGTTTTAATCTTCCTCG
ATTTGTCCTCTGTGCTTATAAGTTCTCTTTCTTTCTCGGACATTTTCTTGTTCCTCAG
ATTCGTCGTGAAGGGTTTCTTCCAGTACCAGATTCGGAGGCAGATGATCTAGTTCC
TGAGTTTCCACCGCTTCGAAAGAAAGATCTTTCGAGAATTATGGGAACCAGCGCTC
AGAGTAAGCCTCTAGATGCTTACTTGCTTAAGATACTCGACGCGACGAAGCCAGCT
TCAGGGATTATAGTTATGTCCTGCAAAGAGCTTGACCATGATTCACTTGCTGAGTCC
AACAAAGTTTTCAGCATTCCGATATTTCCCATTGGCCCTTTTCACATTCATGACGTC
CCAGCCTCGTCTAGCAGCTTGTTAGAACCGGACCAGAGTTGCATTCCATGGTTAGA
TATGCGTGAAACGAGATCAGTAGTCTACGTGAGCTTAGGGAGCATTGCGAGTCTTA
ACGAGTCTGACTTCTTGGAGATTGCTTGTGGACTAAGAAACACCAACCAATCCTTCT
TGTGGGTTGTCCGGCCTGGTTCAGTCCATGGCAGAGATTGGATCGAATCATTACCT
TCAGGGTTCATGGAAAGTCTCGATGGTAAAGGAAAGATAGTGAGATGGGCACCGC
AGCTAGACGTTCTTGCGCATAGAGCCACGGGAGGGTTTTTGACTCATAATGGATGG
AACTCGACATTAGAGAGTATATGCGAAGGAGTACCTATGATCTGCTTGCCTTGTAA
GTGGGACCAATTTGTAAACGCGAGATTCATAAGCGAAGTTTGGAGGGTTGGGATTC
ACTTGGAAGGTCGGATAGAGCGAAGAGAAATCGAGAGAGCTGTTATAAGACTAATG
GTTGAGTCGAAAGGAGAAGAGATTCGAGGTAGAATCAAAGTCTTGCGAGACGAAGT
AAGAAGGTCAGTTAAACAAGGAGGTTCGTCATATCGATCTTTAGATGAGTTGGTTGA
TCGTATATCAATCATCATCGAGCCACTAGTGCCTACGTGA
SEQ ID NO: 48 >UGT76C2
ATGGAGGAGAAGAGAAATGGTCTGCGTGTGATTCTCTTCCCTCTTCCATTACAAGG
TTGCATCAACCCTATGCTTCAGCTCGCCAACATCCTTCACGTAAGAGGCTTCTCCAT
TACCGTGATCCACACGCGCTTCAACGCGCCAAAAGCTTCAAGCCATCCTCTCTTCA
CTTTCTTACAGATTCCTGATGGTTTGTCTGAAACGGAGATTCAAGATGGTGTTATGT
CTTTGCTCGCGCAAATCAACCTTAACGCTGAGTCTCCGTTTCGTGATTGCTTGCGTA
AAGTGTTGCTGGAATCAAAAGAGTCAGAGAGGGTTACTTGTTTGATCGATGACTGT
GGATGGCTCTTCACACAATCTGTTTCAGAGAGTTTGAAGCTTCCGAGGCTCGTTCT
CTGTACTTTTAAAGCCACTTTCTTCAATGCTTATCCGAGTCTTCCACTTATCCGAACC
AAGGGATATCTTCCAGTTTCAGAATCGGAAGCAGAGGACTCTGTTCCTGAGTTCCC
GCCGCTTCAAAAGAGAGATCTTTCAAAGGTTTTCGGGGAGTTCGGAGAGAAACTCG
ATCCGTTCTTACATGCTGTAGTCGAAACGACAATAAGATCTTCAGGGTTAATATACA
TGTCCTGCGAAGAGCTTGAGAAAGATTCGTTGACTCTTTCTAACGAAATTTTTAAAG
TTCCGGTTTTTGCAATTGGTCCGTTTCACAGCTACTTCTCTGCTTCGTCAAGCAGCT
TGTTCACACAAGACGAGACTTGCATTCTGTGGTTAGATGATCAAGAAGATAAATCTG
TGATCTACGTTAGTCTAGGAAGCGTTGTGAACATAACGGAAACAGAGTTCTTGGAG
ATTGCGTGTGGTTTAAGCAATAGCAAACAGCCTTTCTTGTGGGTAGTACGACCCGG
TTCAGTACTCGGCGCGAAATGGATCGAACCGCTCTCTGAAGGGCTGGTTAGTAGC
CTTGAAGAGAAAGGAAAGATTGTGAAATGGGCACCACAACAGGAGGTTCTTGCGCA
TCGTGCCACAGGAGGGTTTTTGACACACAATGGTTGGAACTCAACGCTAGAGAGTA
TATGCGAAGGGGTTCCTATGATCTGCCTACCAGGAGGTTGGGATCAAATGCTGAAT
TCAAGATTTGTTAGCGATATTTGGAAGATTGGAATTCACTTGGAAGGTCGGATTGAA
AAAAAGGAGATTGAGAAAGCTGTGAGGGTGTTAATGGAGGAAAGTGAAGGAAATAA
GATTCGTGAGAGAATGAAAGTTCTGAAAGATGAGGTCGAGAAATCGGTCAAACAAG
GAGGCTCATCTTTTCAATCTATTGAGACTCTAGCTAATCATATACTATTGTTGTAA
SEQ ID NO: 49 >UGT76C3
ATGGATAAGAGTAATGGCCTACGAGTGATTCTGTTTCCACTTCCATTACAAGGATGC
ATCAACCCCATGATTCAGCTAGCGAAGATCCTCCACTCAAGAGGTTTCTCCATCACT
GTGATCCACACGCGCTTCAATGCGCCAAAAGCTTCAAACCACCCTCTGTTCACCTT
CTTACAGATCCCAGATGGCTTGTCTGAAACAGAGACAAGAACTCACGATATCACACT
TCTCCTAACGCTTCTCAACCGAAGCTGTGAGTCTCCATTTCGTGAATGTTTGACTAA
ACTTTTGCAGTCTGCAGATTCAGAAACAGGGGAAGAGAAACAGAGGATTAGCTGTT
TGATCGATGATTCTGGATGGATATTCACACAGCCCGTTGCTCAGAGTTTCAATCTCC
CGAGATTGGTCCTTAACACCTACAAAGTCTCCTTCTTTCGGGACCATTTTGTTCTTC
CTCAACTCCGTCGTGAAATGTATCTTCCATTACAAGATTCAGAACAAGGTGATGATC
CAGTTGAGGAGTTTCCACCCCTTCGAAAGAAAGATCTTTTACAAATTCTTGATCAAG
AATCGGAGCAACTAGACTCGTACTCCAATATGATTTTGGAAACAACAAAAGCGTCTT
CAGGTCTTATATTTGTATCCACATGTGAAGAGTTGGACCAAGACTCACTGAGTCAAG
CACGTGAAGATTATCAAGTCCCAATCTTTACGATAGGACCTTCTCATAGCTACTTCC
CAGGCTCATCTAGTAGCTTGTTCACAGTGGACGAGACTTGCATTCCATGGTTAGAC
AAGCAAGAAGACAAATCCGTGATTTACGTGAGTTTTGGGAGCATCTCGACCATTGG
CGAAGCAGAATTCATGGAGATTGCTTGGGCTCTAAGAAACAGCGACCAACCGTTCT
TGTGGGTCGTACGGGGTGGTTCGGTAGTCCATGGTGCAGAATGGATCGAACAGCT
TCATGAGAAAGGAAAGATAGTGAATTGGGCCCCACAACAAGAGGTTCTAAAGCATC
AAGCCATTGGAGGATTCTTGACACACAATGGTTGGAACTCGACGGTTGAGAGTGTT
TTTGAAGGCGTCCCTATGATATGTATGCCTTTTGTATGGGACCAATTGCTTAATGCA
AGATTTGTTAGTGATGTATGGATGGTTGGGCTGCATCTAGAGGGTCGGATTGAGAG
GAATGTGATTGAGGGAATGATAAGAAGATTATTTTCGGAAACTGAAGGAAAAGCGA
TCCGAGAGAGGATGGAAATTCTTAAGGAGAATGTAGGAAGATCCGTTAAACCAAAA
GGTTCGGCGTATCGATCGTTACAACATTTGATTGATTATATAACATATTTCTAG
SEQ ID NO: 50 >UGT76C4
ATGGAGAAGAGTAATGGCCTGCGAGTGATTCTGTTTCCACTTCCATTACAAGGCTG
CATCAACCCTATGATTCAGCTCGCCAAGATCCTCCACTCAAGAGGTTTTTCAATCAC
TGTGATCCACACTTGCTTCAACGCGCCAAAAGCTTCAAGCCATCCACTCTTCACCTT
CATACAGATCCAAGATGGCTTGTCTGAAACAGAGACAAGAACTCGCGACGTCAAAC
TTCTCATAACACTTCTCAACCAAAATTGCGAGTCTCCGGTTCGTGAATGTTTGCGTA
AACTGTTGCAATCTGCCAAGGAAGAGAAACAGAGGATTAGCTGTTTGATCAATGATT
CTGGTTGGATCTTCACTCAACACTTAGCCAAGAGTTTGAATCTCATGAGATTGGCCT
TTAATACCTATAAGATCTCCTTCTTTCGAAGCCATTTTGTTCTTCCTCAGCTCCGGC
GTGAAATGTTTCTTCCATTACAAGATTCAGAACAAGATGATCCAGTTGAGAAGTTTC
CACCGCTTAGAAAGAAAGATCTTTTACGGATTCTTGAAGCAGATTCGGTGCAGGGA
GACTCGTACTCGGATATGATTTTGGAAAAGACAAAGGCGTCTTCAGGTCTTATATTC
ATGTCCTGTGAAGAGTTGGACCAAGACTCACTGAGTCAATCACGTGAAGATTTTAA
GGTTCCGATATTTGCGATAGGACCTTCTCATAGCCATTTTCCTGCTTCTTCTAGTAG
CTTGTTCACACCGGACGAGACTTGCATCCCATGGTTAGACAGACAAGAAGACAAAT
CCGTAATATACGTGAGTATTGGGAGCCTCGTGACCATCAACGAAACAGAGCTAATG
GAGATTGCTTGGGGTCTAAGTAACAGCGACCAACCATTTTTATGGGTCGTCCGGGT
TGGTTCAGTCAATGGCACGGAATGGATTGAAGCAATCCCGGAATATTTCATCAAAA
GGCTTAATGAGAAGGGAAAGATAGTGAAATGGGCTCCACAACAAGAGGTTCTAAAG
CATCGAGCTATTGGAGGTTTCTTGACACATAATGGTTGGAACTCGACGGTTGAGAG
TGTTTGTGAAGGCGTCCCTATGATCTGTTTGCCTTTTCGTTGGGACCAATTGTTAAA
TGCAAGATTTGTTAGTGATGTATGGATGGTTGGGATACATCTCGAGGGTCGGATTG
AGAGGGATGAGATCGAGAGAGCGATAAGGAGATTATTGTTGGAAACTGAAGGAGA
AGCCATCCGAGAGAGGATACAACTTCTTAAGGAAAAAGTAGGAAGATCAGTTAAAC
AAAACGGTTCGGCATATCAATCTCTACAAAATTTGATTAATTATATATCATCTTTCTAG
SEQ ID NO: 51 >UGT76C5
ATGGAGAAGAGTAATGGCCTTCGAGTGATTCTGTTTCCACTTCCATTACAAGGCTG
CATCAACCCCATGATTCAGCTCGCCAAGATCCTCCACTCAAGAGGTTTCTCCATCAC
TGTGATCCACACGTGCTTCAACGCGCCAAAAGCTTCAAGCCATCCTCTCTTCACCTT
CTTAGAGATCCCAGATGGCTTGTCCGAAACAGAGAAAAGAACTAACAATACCAAACT
TCTCCTAACGCTTCTCAACCGGAACTGTGAGTCTCCGTTTCGTGAATGTTTGAGTAA
ACTGTTGCAGTCTGCAGATTCAGAAACAGGGGAAGAGAAACAGAGGATTAGCTGTT
TGATCGCTGATTCTGGATGGATGTTCACACAACCCATTGCTCAGAGTTTGAAACTCC
CAATATTGGTCCTCAGTGTGTTTACAGTCTCCTTCTTTCGCTGCCAATTTGTTCTTC
CTAAGCTTCGGCGTGAAGTGTATCTTCCACTTCAAGATTCAGAACAGGAGGATCTA
GTTCAAGAGTTTCCGCCGCTTCGAAAGAAGGATATTGTACGTATTCTTGATGTAGAA
ACAGATATACTAGATCCATTCTTGGACAAAGTTCTACAAATGACAAAGGCGTCTTCA
GGTCTTATATTCATGTCATGTGAAGAGTTGGACCACGACTCAGTGAGTCAGGCACG
TGAAGATTTCAAAATTCCTATCTTTGGGATTGGACCATCTCACAGCCACTTTCCAGC
TACCTCTAGTAGCTTGTCCACACCCGACGAGACTTGCATTCCATGGTTAGACAAAC
AAGAAGACAAATCCGTGATTTACGTCAGTTACGGGAGCATCGTGACCATCAGCGAA
TCAGATTTAATAGAGATTGCTTGGGGTCTAAGAAACAGCGACCAACCCTTCTTGTTG
GTCGTACGGGTTGGTTCAGTCCGTGGCAGAGAATGGATCGAGACAATCCCGGAAG
AGATCATGGAAAAGCTTAATGAGAAGGGAAAGATAGTGAAATGGGCTCCGCAACAA
GACGTTCTAAAGCATCGAGCCATTGGGGGATTCCTGACACATAATGGTTGGAGCTC
GACTGTTGAGAGTGTTTGTGAAGCAGTCCCTATGATCTGTTTGCCTTTTCGTTGGG
ACCAAATGCTAAATGCAAGATTTGTTAGCGATGTATGGATGGTCGGGATAAACCTA
GAGGATCGGGTTGAAAGGAATGAGATCGAGGGAGCGATAAGGAGATTATTGGTGG
AACCTGAAGGAGAAGCCATCCGAGAGAGGATAGAACATCTTAAGGAGAAAGTAGGA
CGATCGTTTCAACAAAACGGTTCCGCATATCAATCGTTACAAAATTTGATTGATTATA
TATCATCTTTTTAG
SEQ ID NO: 52 >UGT76D1
ATGGCAGAGATTCGCCAGAGAAGAGTGTTGATGGTCCCAGCACCGTTCCAAGGCC
ATTTACCTTCGATGATGAATCTAGCGTCCTACCTTTCTTCCCAAGGCTTTTCAATCA
CAATCGTTAGAAACGAATTCAATTTCAAAGATATCTCCCATAATTTCCCTGGTATAAA
ATTCTTCACCATCAAGGACGGCTTGTCAGAATCTGACGTGAAGTCTCTGGGTCTCC
TTGAATTTGTCCTGGAGCTTAACTCTGTCTGTGAACCCCTATTGAAAGAGTTTCTAA
CCAACCATGATGATGTTGTTGACTTTATCATTTATGATGAATTTGTTTACTTCCCTCG
ACGTGTTGCGGAAGATATGAATCTGCCAAAGATGGTCTTTAGCCCTTCTTCCGCCG
CTACCTCGATCAGCCGGTGTGTGCTTATGGAGAACCAATCAAATGGGTTACTTCCT
CCACAAGACGCAAGATCTCAACTAGAAGAAACGGTGCCAGAGTTTCATCCCTTTCG
TTTCAAAGATCTGCCTTTTACAGCTTATGGATCTATGGAGAGATTAATGATACTTTAC
GAGAATGTAAGCAATAGAGCCTCATCTTCTGGCATAATACACAACTCTTCGGATTGC
TTAGAGAACTCATTCATAACAACTGCACAAGAGAAATGGGGAGTTCCGGTATACCC
GGTTGGTCCACTCCATATGACCAATTCCGCAATGTCATGTCCAAGTTTATTTGAAGA
AGAAAGAAACTGTCTTGAATGGCTTGAGAAGCAAGAAACAAGCTCAGTGATCTACA
TAAGCATGGGGAGCTTGGCGATGACACAAGATATAGAGGCTGTGGAGATGGCCAT
GGGATTTGTCCAGAGTAATCAACCCTTCTTGTGGGTGATCCGACCAGGCTCTATAA
ACGGACAAGAATCTTTAGACTTCTTACCGGAACAGTTCAACCAAACGGTGACCGAT
GGAAGAGGTTTTGTTGTGAAATGGGCCCCACAAAAAGAGGTATTAAGGCATAGAGC
AGTGGGAGGGTTTTGGAACCATGGTGGATGGAACTCGTGCTTGGAGAGCATAAGC
AGTGGTGTACCAATGATTTGTAGGCCGTATTCTGGTGATCAGAGGGTGAATACTCG
ACTTATGTCACATGTTTGGCAAACCGCGTATGAGATCGAAGGTGAATTGGAAAGAG
GAGCTGTTGAGATGGCCGTGAGGAGGCTCATTGTGGATCAAGAAGGTCAGGAGAT
GAGAATGAGAGCCACCATATTGAAGGAAGAGGTTGAAGCCTCTGTCACAACCGAAG
GCTCTTCTCACAATTCTTTAAACAATTTGGTCCATGCAATAATGATGCAAATTGACGA
ACAATGA
SEQ ID NO: 53 >UGT76E1
ATGGAAGAACTAGGAGTGAAGAGAAGGATAGTATTGGTTCCAGTTCCAGCACAAGG
TCATGTAACTCCGATTATGCAACTCGGGAAGGCTCTTTACTCCAAGGGCTTCTCCAT
CACTGTTGTTCTCACACAGTATAATCGAGTTAGCTCATCCAAGGACTTCTCTGATTT
TCATTTCCTCACCATCCCAGGCAGCTTGACCGAGTCTGATCTCAAAAACCTTGGAC
CATTCAAGTTTCTCTTCAAGCTCAATCAAATTTGCGAGGCAAGCTTCAAGCAATGTA
TTGGTCAACTATTGCAGGAGCAAGGTAATGATATCGCTTGTGTCGTCTACGATGAG
TACATGTACTTCTCCCAAGCTGCAGTTAAAGAGTTTCAACTTCCTAGCGTCCTCTTC
AGCACGACAAGTGCTACTGCCTTTGTCTGTCGCTCTGTTTTGTCTAGAGTCAACGC
AGAGTCATTCTTGCTTGACATGAAAGATCCCAAAGTGTCAGACAAGGAATTTCCAG
GGTTGCATCCGCTAAGGTACAAGGACCTGCCAACTTCAGCATTTGGGCCATTAGAG
AGTATACTCAAGGTTTACAGTGAGACTGTCAACATTCGAACAGCTTCGGCAGTTATC
ATCAACTCAACAAGCTGTCTAGAGAGCTCATCTTTGGCATGGTTACAAAAACAACTG
CAAGTTCCAGTGTATCCTATAGGCCCACTTCACATTGCAGCTTCAGCGCCTTCTAGT
TTACTTGAAGAGGACAGGAGTTGCCTTGAGTGGTTGAACAAGCAAAAAATAGGCTC
AGTGATTTACATAAGTTTGGGAAGCTTGGCTCTAATGGAAACTAAAGACATGTTGGA
GATGGCTTGGGGTTTACGTAATAGCAACCAACCTTTCTTATGGGTGATCCGACCGG
GTTCTATTCCCGGCTCGGAATGGACAGAGTCTTTACCGGAGGAATTCAGTAGGTTG
GTTTCAGAAAGAGGTTACATTGTGAAATGGGCACCACAGATAGAAGTTCTCAGACA
TCCTGCAGTGGGAGGGTTTTGGAGTCACTGCGGATGGAACTCGACCCTAGAGAGC
ATCGGGGAAGGAGTTCCGATGATCTGTAGGCCTTTTACGGGAGATCAGAAAGTCAA
TGCGAGGTACTTAGAGAGAGTTTGGAGAATTGGGGTTCAATTGGAAGGAGAGCTG
GATAAAGGAACAGTGGAGAGAGCTGTAGAGAGATTGATTATGGATGAAGAAGGAG
CAGAAATGAGGAAGAGAGTTATCAACTTGAAAGAGAAGCTTCAAGCCTCTGTCAAG
AGTAGAGGTTCCTCATTCAGCTCATTAGACAACTTTGTCAATTCCTTAAAAATGATG
AATTTCATGTAG
SEQ ID NO: 54 >UGT76E11
ATGGAGGAAAAGCCGGCGGGCAGAAGAGTAGTGTTGGTTGCAGTTCCAGCTCAAG
GACATATCTCTCCAATAATGCAACTTGCAAAAACACTTCACTTGAAGGGTTTCTCAA
TCACAATCGCTCAGACAAAGTTCAATTACTTTAGCCCTTCAGATGACTTCACTGATTT
TCAGTTTGTCACCATTCCAGAAAGCTTACCAGAGTCTGATTTTGAGGATCTCGGGC
CAATAGAGTTTCTGCATAAGCTCAACAAAGAGTGTCAGGTGAGCTTCAAAGACTGTT
TGGGTCAGTTGTTGCTGCAACAAGGTAATGAGATAGCCTGTGTTGTCTACGACGAG
TTCATGTACTTTGCTGAAGCTGCAGCCAAAGAGTTTAAGCTTCCAAACGTCATTTTC
AGCACCACAAGTGCCACGGCTTTTGTTTGCCGCTCTGCATTCGACAAACTTTATGC
AAACAGTATCCTGACTCCCTTGAAAGAACCCAAAGGACAACAAAACGAGCTAGTGC
CAGAGTTTCATCCCCTGAGATGCAAAGACTTTCCGGTTTCACATTGGGCATCATTAG
AAAGCATGATGGAGCTGTATAGGAATACAGTTGACAAACGGACAGCTTCCTCGGTG
ATAATCAACACAGCGAGCTGTCTAGAGAGCTCATCTCTGTCTCGTCTGCAGCAACA
GCTACAAATTCCAGTTTATCCTATAGGCCCTCTTCACCTGGTGGCATCAGCTTCTAC
GAGTCTTCTTGAAGAGAACAAGAGCTGTATTGAATGGTTGAACAAACAAAAGAAAAA
CTCTGTGATATTCGTAAGCTTGGGAAGCTTAGCTTTGATGGAAATCAATGAGGTGAT
AGAAACTGCTTTGGGATTGGATAGTAGCAAGCAACAGTTCTTGTGGGTCATTCGGC
CAGGGTCAGTACGTGGTTCGGAATGGATAGAGAACTTGCCTAAGGAGTTTAGTAAG
ATAATTTCGGGTCGAGGTTACATTGTGAAATGGGCTCCACAGAAGGAAGTACTTTC
TCATCCTGCAGTAGGAGGATTTTGGAGCCATTGCGGATGGAACTCGACACTAGAGA
GCATCGGGGAAGGAGTTCCAATGATTTGCAAGCCGTTTTCCAGTGATCAAATGGTG
AATGCGAGATACTTGGAGTGTGTATGGAAAATTGGGATTCAAGTTGAGGGTGATCT
AGACAGAGGAGCGGTCGAGAGAGCTGTGAGGAGGTTAATGGTGGAGGAAGAAGG
GGAGGGGATGAGGAAGAGAGCTATCAGTTTGAAAGAGCAACTTAGAGCCTCTGTTA
TAAGTGGAGGTTCTTCACACAACTCGCTAGAGGAGTTTGTACACTACATGAGGACT
CTATGA
SEQ ID NO: 55 >UGT76E12
ATGGAGGAAAAGCCTGCAAGGAGAAGCGTAGTGTTGGTTCCATTTCCAGCACAAG
GACATATATCTCCAATGATGCAACTTGCCAAAACCCTTCACTTAAAGGGTTTCTCGA
TCACAGTTGTTCAGACTAAGTTCAATTACTTTAGCCCTTCAGATGACTTCACTCATG
ATTTTCAGTTCGTCACCATTCCAGAAAGCTTACCAGAGTCTGATTTCAAGAATCTCG
GACCAATACAGTTTCTGTTTAAGCTCAACAAAGAGTGTAAGGTGAGCTTCAAGGACT
GTTTGGGTCAGTTGGTGCTGCAACAAAGTAATGAGATCTCATGTGTCATCTACGAT
GAGTTCATGTACTTTGCTGAAGCTGCAGCCAAAGAGTGTAAGCTTCCAAACATCATT
TTCAGCACAACAAGTGCCACGGCTTTCGCTTGCCGCTCTGTATTTGACAAACTATAT
GCAAACAATGTCCAAGCTCCCTTGAAAGAAACTAAAGGACAACAAGAAGAGCTAGT
TCCGGAGTTTTATCCCTTGAGATATAAAGACTTTCCAGTTTCACGGTTTGCATCATT
AGAGAGCATAATGGAGGTGTATAGGAATACAGTTGACAAACGGACAGCTTCCTCGG
TGATAATCAACACTGCGAGCTGTCTAGAGAGCTCATCTCTGTCTTTTCTGCAACAAC
AACAGCTACAAATTCCAGTGTATCCTATAGGCCCTCTTCACATGGTGGCCTCAGCT
CCTACAAGTCTGCTTGAAGAGAACAAGAGCTGCATCGAATGGTTGAACAAACAAAA
GGTAAACTCGGTGATATACATAAGCATGGGAAGCATAGCTTTAATGGAAATCAACG
AGATAATGGAAGTCGCGTCAGGATTGGCTGCTAGCAACCAACACTTCTTATGGGTG
ATCCGACCAGGGTCAATACCTGGTTCCGAGTGGATAGAGTCCATGCCTGAAGAGTT
TAGTAAGATGGTTTTGGACCGAGGTTACATTGTGAAATGGGCTCCACAGAAGGAAG
TACTTTCTCATCCTGCAGTAGGAGGGTTTTGGAGCCATTGTGGATGGAACTCGACA
CTAGAAAGCATCGGCCAAGGAGTTCCAATGATCTGCAGGCCATTTTCGGGTGATCA
AAAGGTGAACGCTAGATACTTGGAGTGTGTATGGAAAATTGGGATTCAAGTGGAGG
GTGAGCTAGACAGAGGAGTGGTCGAGAGAGCTGTGAAGAGGTTAATGGTTGACGA
AGAAGGAGAGGAGATGAGGAAGAGAGCTTTCAGTTTAAAAGAGCAACTTAGAGCCT
CTGTTAAAAGTGGAGGCTCTTCACACAACTCGCTAGAAGAGTTTGTACACTTCATAA
GGACTCTATGA
SEQ ID NO: 56 >UGT76E2
ATGGAGGAAAAGCAAGTGAAGGAGACAAGGATAGTGTTGGTTCCAGTTCCAGCTCA
AGGTCATGTAACTCCGATGATGCAACTAGGAAAAGCTCTTCACTCAAAGGGTTTCTC
CATCACTGTTGTTCTGACACAGTCTAATCGAGTTAGCTCTTCCAAAGACTTCTCTGA
TTTCCATTTCCTCACCATCCCAGGCAGCTTAACTGAGTCTGATCTCCAAAACCTAGG
ACCACAAAAGTTTGTGCTCAAGCTCAATCAAATTTGTGAGGCAAGCTTCAAGCAGTG
TATAGGTCAACTATTGCATGAACAATGTAATAATGATATTGCTTGTGTCGTCTACGAT
GAGTACATGTACTTCTCTCATGCTGCAGTAAAAGAGTTTCAACTTCCTAGTGTCGTC
TTTAGCACGACAAGTGCTACTGCTTTTGTCTGTCGCTCTGTTTTGTCTAGAGTCAAC
GCAGAGTCGTTCTTGATCGACATGAAAGATCCTGAAACACAAGACAAAGTATTTCCA
GGGTTGCATCCTCTGAGGTACAAGGATCTACCAACTTCAGTATTTGGGCCAATAGA
GAGTACGCTCAAGGTTTACAGTGAGACTGTGAACACTCGAACAGCTTCCGCTGTTA
TCATCAACTCAGCAAGCTGTTTAGAGAGCTCATCTTTGGCAAGGTTGCAACAACAAC
TGCAAGTTCCGGTGTATCCTATAGGCCCACTTCATATTACAGCTTCAGCGCCTTCTA
GTTTACTAGAAGAAGACAGGAGTTGCGTTGAGTGGTTGAACAAGCAAAAATCAAAT
TCAGTTATTTACATAAGCTTGGGAAGCTTGGCTCTAATGGACACCAAAGACATGTTG
GAGATGGCTTGGGGATTAAGTAATAGCAACCAACCTTTCTTATGGGTGGTCAGACC
GGGCTCTATTCCGGGGTCAGAATGGACAGAGTCCTTACCAGAGGAATTCAATAGGT
TGGTTTCAGAAAGAGGTTACATTGTGAAATGGGCTCCGCAGATGGAAGTTCTCAGA
CATCCTGCAGTAGGAGGGTTTTGGAGTCACTGTGGATGGAACTCAACAGTAGAGA
GCATCGGGGAAGGAGTTCCGATGATATGTAGGCCTTTCACCGGGGATCAGAAAGT
CAATGCGAGGTACTTAGAGAGAGTTTGGAGAATTGGGGTTCAATTGGAGGGAGAT
CTGGATAAAGAAACTGTGGAGAGAGCTGTAGAGTGGTTGCTTGTGGATGAAGAAG
GAGCAGAAATGAGGAAGAGAGCCATTGACTTGAAAGAAAAGATTGAAACCTCTGTT
AGAAGTGGAGGTTCCTCATGCAGCTCACTAGACGACTTTGTTAATTCCATGTGA
SEQ ID NO: 57 >UGT76E3
ATGGAGAAAAGAGTAGAGAAGAGAAGGATAGTGTTGGTTCCACTTCCATTACTAGG
ACATTTCACTCCGATGATGCAACTCGGCCAAGCCCTTATCTTGAAGGGATTCTCAAT
TATAGTTCCTCAGGGAGAATTCAATCGAGTAAACTCTTCGCAGAAGTTCCCTGGTTT
TCAATTTATCACCATACCAGATTCTGAACTCGAGGCAAATGGACCAGTCGGGTCTCT
AACACAGCTCAACAAAATTATGGAGGCAAGCTTCAAGGACTGTATAAGGCAGTTGT
TGAAACAACAAGGCAATGATATTGCATGTATCATCTACGACGAGTTCATGTATTTTT
GTGGAGCCGTAGCTGAGGAGTTGAAGCTTCCCAATTTCATCTTCAGTACTCAAACT
GCTACACATAAAGTTTGCTGCAATGTTTTAAGCAAACTTAATGCCAAGAAGTACTTG
ATCGACATGGAAGAGCATGACGTGCAAAACAAGGTAGTGGAAAATATGCATCCATT
AAGATACAAAGACTTACCAACTGCAACATTTGGAGAACTAGAACCTTTTTTGGAGCT
CTGTAGAGATGTAGTCAACAAAAGAACAGCCTCTGCTGTTATCATCAACACCGTGA
CCTGTCTAGAGAGCTCGTCTCTCACAAGGCTGCAACAAGAACTCCAAATTCCGGTG
TATCCATTAGGCCCTCTTCACATTACAGATTCATCGACAGGATTTACTGTGCTGCAA
GAGGATAGGAGCTGCGTTGAATGGCTGAACAAGCAGAAACCAAGGTCTGTCATATA
CATAAGTTTAGGAAGCATGGTTCTCATGGAAACCAAGGAGATGTTAGAGATGGCTT
GGGGAATGTTGAATAGCAACCAACCTTTCTTATGGGTCATCCGACCTGGATCTGTC
TCAGGCTCCGAGGGGATAGAGTCATTGCCAGAGGAAGTCAGTAAGATGGTTTTAGA
GAAAGGATACATTGTGAAATGGGCACCACAAATAGAAGTACTAGGACATCCCTCAG
TGGGAGGCTTTTGGAGCCACTGTGGATGGAACTCAACACTCGAGAGCATTGTGGA
AGGAGTTCCAATGATTTGCAGGCCTTATCAAGGCGAGCAGATGTTAAATGCAATAT
ATCTAGAGAGTGTATGGAGAATAGGGATTCAGGTAGGAGGTGAACTGGAAAGAGG
AGCCGTCGAGAGAGCTGTGAAGAGGTTGATTGTGGATAAAGAAGGTGCAAGCATG
AGGGAGAGAACCCTTGTTTTAAAAGAGAAGCTCAAAGCCTCTATTAGAGGTGGAGG
CTCCTCATGCAATGCATTAGATGAGCTTGTCAAGCACTTGAAGACAGAGTGA
SEQ ID NO: 58 >UGT76E4
ATGGAGAAAAGGGTAGAGAAGAGAAGGATTGTGTTAGTTCCGGTTGCTGCACAAG
GACATGTAACCCCAATGATGCAGCTTGGGAAAGCCCTTCAATCAAAGGGCTTCTTA
ATTACTGTTGCTCAGAGACAGTTCAATCAAATAGGCTCATCATTGCAACACTTTCCT
GGTTTTGACTTTGTCACCATACCAGAAAGCTTACCTCAGTCTGAATCTAAGAAACTA
GGACCAGCTGAGTATCTTATGAATCTCAACAAAACAAGCGAGGCAAGCTTCAAGGA
GTGTATAAGTCAGTTATCGATGCAACAAGGCAATGATATAGCATGTATCATCTATGA
CAAGCTTATGTACTTCTGTGAAGCAGCAGCTAAGGAGTTTAAGATTCCTAGTGTTAT
CTTCAGCACTAGCAGTGCTACAATTCAAGTTTGCTACTGTGTTTTAAGTGAACTCAG
TGCCGAGAAGTTCTTGATCGACATGAAAGATCCTGAAAAGCAAGATAAGGTGTTGG
AAGGTTTGCATCCTTTAAGGTACAAAGACCTACCAACTTCAGGATTTGGACCATTAG
AGCCACTTTTGGAGATGTGTAGGGAAGTAGTTAACAAAAGAACAGCTTCCGCTGTT
ATCATCAACACGGCGAGCTGTCTAGAGAGCTTGTCTCTGTCATGGCTGCAACAAGA
ACTTGGAATTCCAGTGTATCCATTAGGCCCTCTTCACATTACAGCTTCATCGCCGGG
ACCTAGTTTACTGCAAGAGGACATGAGCTGCATTGAATGGCTGAACAAGCAGAAAC
CAAGGTCAGTCATATACATAAGCTTGGGAACCAAAGCTCACATGGAGACCAAGGAG
ATGTTAGAGATGGCCTGGGGATTGTTGAATAGCAACCAACCTTTCTTATGGGTCAT
CCGACCTGGCTCTGTTGCAGGCTTCGAGTGGATAGAGTTATTACCAGAGGAAGTCA
TTAAGATGGTAACAGAAAGAGGATACATAGCGAAATGGGCACCGCAGATAGAAGTA
CTTGGACATCCTGCAGTGGGAGGATTCTGGAGCCACTGTGGATGGAACTCAACAC
TCGAGAGTATTGTGGAAGGAGTCCCAATGATTTGCAGGCCTTTACAAGGCGAACAA
AAGTTAAATGCGATGTATATAGAAAGTGTTTGGAAAATAGGGATTCAACTTGAAGGT
GAAGTGGAAAGGGAAGGTGTAGAGAGAGCTGTGAAGAGGTTGATCATAGATGAAG
AAGGTGCAGCCATGAGGGAGAGGGCTCTTGATTTAAAAGAGAAGCTCAATGCCTC
GGTAAGAAGTGGAGGCTCCTCATACAACGCACTGGATGAGCTTGTCAAGTTCTTGA
ATACAGAGTGA
SEQ ID NO: 59 >UGT76E5
ATGGAGAAAAATGCAGAGAAGAAAAGAATAGTGTTGGTTCCATTTCCATTACAAGGA
CATATCACTCCAATGATGCAACTTGGTCAAGCACTTAACCTGAAAGGCTTCTCGATT
ACCGTTGCTCTTGGAGATTCCAATCGAGTAAGTTCTACGCAACACTTCCCTGGTTTT
CAATTTGTCACAATACCTGAAACCATACCACTATCTCAACACGAGGCACTCGGAGTT
GTCGAGTTTGTGGTTACGCTCAACAAAACAAGCGAGACAAGTTTCAAGGACTGTAT
AGCTCATTTGTTGCTGCAACATGGAAATGATATTGCTTGTATCATTTACGACGAGCT
CATGTACTTCTCTGAAGCTACAGCTAAGGATTTAAGGATTCCTAGTGTCATATTCAC
CACTGGTAGTGCTACAAATCATGTTTGTTCTTGTATTTTAAGCAAACTCAACGCCGA
GAAGTTCTTGATCGACATGAAAGATCCTGAAGTGCAAAACATGGTGGTGGAAAATT
TACATCCACTAAAATACAAAGACTTACCAACTTCAGGAATGGGGCCGCTAGAGCGA
TTTTTGGAGATTTGTGCCGAAGTTGTCAACAAAAGAACAGCTTCCGCTGTTATAATC
AATACGTCAAGTTGTCTAGAGAGCTCGTCTCTGTCATGGCTGAAACAAGAACTCAG
TATTCCAGTGTATCCATTAGGCCCTCTTCACATTACAACTTCAGCAAATTTTAGTTTA
CTTGAAGAGGACAGGAGCTGCATTGAATGGCTGAACAAGCAGAAACTGAGGTCAG
TTATATACATAAGCGTAGGAAGCATAGCTCACATGGAAACCAAGGAAGTATTGGAG
ATGGCTTGGGGATTGTATAATAGCAACCAACCTTTTCTATGGGTAATCCGACCCGG
TACAGAGTCAATGCCAGTGGAAGTCAGTAAGATTGTCTCGGAAAGAGGATGCATTG
TGAAATGGGCGCCACAGAATGAAGTACTTGTGCATCCTGCAGTGGGAGGTTTCTG
GAGCCACTGTGGATGGAACTCAACACTCGAGAGTATTGTGGAAGGAGTTCCAATGA
TTTGCAGACCGTTTAACGGTGAGCAGAAGTTAAACGCGATGTATATAGAAAGTGTTT
GGAGAGTAGGGGTTCTGCTTCAAGGAGAAGTGGAGAGAGGATGTGTAGAGAGAGC
TGTGAAGAGGTTGATTGTGGATGATGAAGGTGTAGGAATGAGGGAGAGAGCCCTT
GTTTTAAAAGAGAAGCTCAATGCCTCTGTAAGAAGTGGAGGCTCTTCATACAATGCA
TTGGATGAGCTCGTCCATTACTTGGAGGCAGAGTATAGAAATACTTGA
SEQ ID NO: 60 >UGT76E6
ATGGAGAAAATGGAAGAGAAGAAAAGGATAGTGTTAGTTCCGGTTCCAGCACAAAG
ACATGTAACTCCAATGATGCAGCTTGGCACAGCCCTAAACATGAAGGGCTTCTCTA
TTACTGTTGTTGAAGGACAGTTCAATAAAGTAAGCTCATCTCAAAACTTTCCTGGTTT
TCAATTTGTAACCATACCAGATACAGAGAGCTTGCCAGAGTCTGTGCTCGAGAGAC
TCGGACCGGTCGAGTTTTTATTCGAGATCAACAAAACCAGTGAGGCAAGCTTCAAG
GACTGTATAAGGCAGTCGTTGCTGCAACAAGGCAATGATATAGCATGTATCATCTAC
GACGAGTATATGTACTTCTGTGGAGCTGCAGCTAAGGAGTTCAACCTTCCTAGTGT
AATATTCAGCACACAAAGTGCTACTAATCAAGTTTCCCGTTGCGTTTTAAGAAAACT
CAGTGCCGAGAAGTTCTTGGTGGACATGGAAGGTATCCTGAAGTGCAGGAAACGT
TGGTGGAAAATTTGCATCCATTAAGATACAAAGACCTACCAACTTCAGGAGTTGGG
CCACTAGATCGATTATTTGAGCTCTGTAGGGAAATAGTCAACAAAAGAACAGCTTCC
GCTGTTATCATCAACACAGTGAGATGTCTAGAGAGCTCGTCTCTGAAACGTCTGCA
ACATGAACTCGGGATTCCGGTGTACGCATTAGGCCCTCTTCACATTACAGTTTCAG
CAGCTTCTAGTTTACTGGAAGAGGACAGGAGCTGCGTTGAATGGTTGAACAAGCAA
AAACCGAGGTCAGTCGTTTACATAAGCTTGGGGAGCGTAGTTCAAATGGAAACCAA
AGAAGTGTTAGAGATGGCTCGGGGTTTATTTAATAGCAACCAGCCTTTCTTATGGG
TCATTCGGCCTGGCTCTATCGCAGGCTCCGAATGGATAGAGTCACTGCCAGAGGA
AGTCATTAAGATGGTCTCCGAAAGAGGGTATATTGTGAAATGGGCACCACAGATAG
AAGTACTTGGACATCCTGCAGTGGGAGGATTCTGGAGCCACTGTGGATGGAACTC
AACGCTTGAAAGCATTGTGGAAGGAGTTCCAATGATATGCAGGCCCTTTCATGGCG
AGCAAAAGTTAAACGCACTGTGTTTAGAGAGTATTTGGAGAATAGGGTTTCAGGTG
CAAGGTAAGGTAGAGAGGGGAGGGGTCGAGAGAGCTGTGAAGAGGTTGATAGTG
GATGAAGAAGGTGCAGACATGAGAGAGAGAGCCCTTGTTTTAAAAGAGAATCTCAA
AGCCTCTGTAAGAAATGGAGGCTCCTCATACAACGCATTGGAGGAGATCGTTAACC
TCATGTAG
SEQ ID NO: 61 >UGT76E7
ATGGAGGAGAAGCTCTCGAGGAGAAGAAGAGTAGTGTTGGTTCCAGTTCCAGCTC
AAGGACATATAACTCCAATGATACAACTTGCAAAAGCACTTCACTCAAAAGGCTTCT
CTATTACAGTTGTTCAAACCAAGTTCAACTACTTAAACCCTTCAAATGATTTGTCTGA
TTTTCAGTTTGTAACCATCCCAGAGAACTTACCAGTGTCTGATCTTAAGAATCTAGG
ACCAGGACGGTTTCTGATTAAGCTAGCTAATGAGTGTTATGTTAGCTTTAAGGATTT
GTTAGGTCAGTTGTTGGTTAATGAAGAAGAAGAGATCGCTTGTGTTATCTACGACG
AGTTCATGTACTTTGTTGAAGTAGCAGTTAAAGAGTTTAAGCTTCGTAATGTTATTTT
AAGTACTACAAGTGCAACGGCTTTTGTTTGTCGCTTTGTTATGTGTGAACTCTATGC
TAAAGATGGTTTGGCTCAACTTAAAGAAGGCGGTGAGCGAGAAGTGGAGTTAGTAC
CGGAGTTGTATCCTATACGGTACAAAGATTTACCAAGTTCGGTATTTGCATCTGTAG
AATCTTCAGTGGAGTTGTTTAAGAATACATGTTATAAAGGGACAGCTTCCTCTGTGA
TAATCAACACAGTGAGGTGTCTAGAGATGTCATCTTTGGAGTGGCTTCAACAAGAA
CTTGAAATCCCGGTGTATTCTATAGGCCCGCTTCATATGGTGGTGTCAGCTCCTCC
TACGAGTCTTTTAGAAGAGAACGAGAGCTGTATAGAATGGTTGAACAAACAAAAGC
CGAGCTCGGTGATATACATAAGCTTGGGAAGTTTTACTTTGATGGAAACTAAAGAAA
TGTTGGAGATGGCTTATGGGTTTGTTAGTAGTAACCAACACTTCTTGTGGGTGATTC
GACCGGGATCTATATGTGGTTCTGAAATCTCTGAGGAAGAGTTGTTGAAGAAGATG
GTAATTACGGATCGAGGTTACATTGTGAAATGGGCGCCGCAAAAACAAGTGCTTGC
ACATTCTGCGGTTGGAGCGTTCTGGAGTCATTGTGGATGGAACTCGACTTTAGAAA
GTCTTGGTGAAGGAGTTCCATTGATATGTAGGCCTTTTACTACTGATCAAAAGGGG
AATGCAAGGTACTTGGAGTGTGTGTGGAAAGTAGGAATTCAAGTGGAGGGTGAGC
TAGAGAGAGGCGCAATCGAGAGAGCTGTGAAGAGGTTAATGGTGGATGAAGAAGG
AGAAGAGATGAAGAGAAGAGCTCTAAGTTTAAAAGAGAAACTCAAAGCCTCTGTTTT
AGCTCAAGGTTCTTCACATAAATCACTAGATGACTTCATCAAGACTCTGTGA
SEQ ID NO: 62 >UGT76E9
ATGGAGGAAAAGCAAGAGAGGAGGAGAAGGATCGTGTTGATTCCCGCTCCAGCAC
AAGGACACATATCTCCGATGATGCAACTTGCAAGAGCCCTTCACTTAAAGGGCTTC
TCCATTACAGTTGCTCAAACCAAGTTCAATTACTTGAAGCCTTCAAAAGACTTAGCT
GATTTTCAGTTTATCACCATCCCAGAGAGCTTACCAGCCTCGGATCTTAAGAATCTA
GGACCAGTTTGGTTTCTTCTTAAACTCAATAAAGAGTGTGAGTTTAGCTTCAAGGAG
TGTTTAGGTCAATTGTTGCTGCAAAAACAACTTATACCGGAAGAAGAGATCGCTTGT
GTCATCTACGACGAGTTCATGTACTTTGCTGAAGCTGCAGCCAAAGAGTTTAACCTT
CCCAAAGTTATTTTCAGTACCGAAAATGCGACGGCTTTTGCTTGTCGCTCTGCCATG
TGCAAACTCTATGCAAAAGATGGTTTGGCTCCCCTTAAAGAAGGATGTGGGCGAGA
AGAGGAGCTAGTGCCAAAGTTGCATCCCCTTAGATACAAAGACCTACCAACTTCAG
CATTTGCACCAGTAGAAGCCTCAGTGGAAGTGTTTAAAAGTTCATGTGATAAAGGG
ACAGCTTCCGCTATGATAATCAACACAGTGAGGTGTCTAGAGATATCATCCTTGGA
GTGGCTTCAACAAGAACTTAAGATTCCGATATATCCTATAGGCCCTCTTCACATGGT
TTCTTCAGCTCCTCCTACGAGTCTACTAGACGAGAATGAGAGTTGCATTGATTGGCT
GAACAAACAAAAGCCGAGCTCGGTGATTTACATAAGTTTGGGAAGCTTTACTTTGTT
GGAAACTAAAGAAGTGTTGGAAATGGCTTCGGGCTTGGTTAGTAGTAACCAACACT
TCTTGTGGGTGATTCGACCCGGGTCCATACTTGGTTCTGAATTGACTAATGAGGAA
TTATTGAGTATGATGGAAATACCGGATCGAGGCTACATTGTGAAATGGGCTCCACA
AAAGCAAGTGCTTGCACATTCTGCGGTTGGAGCATTTTGGAGTCATTGTGGATGGA
ACTCGACTCTAGAGAGCATGGGTGAAGGAGTTCCGATGATTTGTAGGCCTTTTACT
ACTGATCAAAAGGTAAATGCGCGGTATGTGGAGTGTGTCTGGAGAGTTGGGGTTC
AAGTGGAGGGTGAACTAAAGAGAGGAGTAGTCGAGAGAGCTGTGAAGAGGTTACT
GGTGGATGAAGAAGGAGAAGAGATGAAGTTGAGAGCTCTCAGTTTGAAAGAGAAA
CTCAAAGTTTCTGTTCTACCGGGAGGTTCTTCACACAGTTCACTAGATGACTTAATC
AAGACTCTATGA
SEQ ID NO: 63 >UGT76F1
ATGGAAGAGAGAAAAGTGAAGAGAATTATCATGTTCCCTCTACCGTTTACAGGACA
CTTCAACCCTATGATCGAGCTTGCTGGAATATTCCACAACCGTGGCTTCTCCGTCA
CGATACTCCACACTTCTTTCAACTTCCCGGATCCTTCTCGCCATCCACAGTTTACTT
TTCGAACTATCACTCACAAAAACGAAGGAGAAGAAGACCCTCTCTCTCAATCAGAAA
CTTCTTCGGGTAAGGACCTCGTCGTCCTTATTAGTCTGCTGAAACAATACTACACCG
AGCCGTCTCTTGCAGAGGAAGTAGGCGAAGGAGGGACGGTGTGTTGTTTGGTCTC
CGACGCTCTATGGGGGAGGAACACGGAGATTGTAGCGAAAGAGATTGGAGTGTGT
ACAATGGTGATGAGGACTAGTGGTGCGGCAACGTTTTGTGCTTATACAGCTTTCCC
TCTCCTTATAGATAAGGGTTACCTTCCTATACAAGGTTCTAGATTAGATGAGCTAGT
GACAGAGCTTCCACCTTTGAAAGTGAAGGATCTTCCTGTAATAAAAACGAAAGAGC
CTGAGGGACTAAACCGAATACTTAACGACATGGTGGAAGGAGCCAAGTTATCTTCC
GGAGTCGTATGGAACACATTTGAAGATCTTGAAAGACATTCACTCATGGATTGTCG
CAGCAAGTTACAAGTTCCGTTGTTCCCAATCGGACCGTTTCACAAACATAGAACCGA
TCTTCCACCGAAGCCAAAGAACAAGGACAAGGACGATGATGAAATATTAACCGATT
GGCTTAACAAGCAAGCTCCGCAGTCTGTGGTCTATGTGAGTTTTGGAAGCCTTGCA
GCTATAGAAGAGAATGAGTTTTTCGAAATTGCTTGGGGTCTAAGAAACAGCGAACT
ACCATTCTTGTGGGTGGTTAGGCCCGGGATGGTCCGGGGAACCGAGTGGCTTGAG
TCATTGCCTTGTGGGTTTTTGGAAAATATTGGTCATCAGGGAAAAATTGTGAAATGG
GTGAATCAACTAGAGACATTGGCCCATCCTGCGGTTGGAGCGTTTTGGACGCACTG
TGGATGGAACTCAACAATAGAGAGCATATGTGAAGGTGTTCCAATGATATGTACGC
CGTGTTTCTCGGACCAGCATGTGAACGCGAGGTACATCGTTGATGTATGGCGAGTC
GGGATGATGTTAGAGAGATGTAAGATGGAAAGGACGGAGATTGAGAAGGTAGTAA
CAAGTGTAATGATGGAGAATGGAGCTGGATTGACAGAGATGTGTTTGGAGTTGAAA
GAGAAAGCTAATGTTTGCTTAAGTGAAGATGGGTCTTCTTCCAAGTATCTAGACAAA
CTTGTCAGTCATGTCCTGTCTTTTGATTCCTCGGCTTTTGCAAGTTAA
SEQ ID NO: 64 >UGT76F2
ATGGAAGAGAGAAAAGGGAGGAGAATAATCATGTTCCCTCTTCCATTTCCAGGGCA
CTTCAACCCCATGATCGAGCTCGCTGGAATATTCCACCACCGTGGCTTCTCCGTGA
CGATCCTCCACACTTCCTACAACTTCCCCGATCCTTCTCGCCACCCACACTTCACTT
TTCGAACCATCTCTCACAACAAAGAAGGAGAAGAAGATCCTCTGTCTCAGTCAGAAA
CTTCGAGTATGGACCTAATCGTTCTCGTTCGTCGGCTGAAACAACGCTACGCCGAA
CCGTTTCGTAAGTCTGTGGCGGCGGAAGTAGGTGGAGGAGAGACGGTGTGTTGTT
TGGTCTCCGACGCTATATGGGGGAAGAACACGGAGGTTGTAGCGGAAGAGATTGG
AGTTCGTAGGGTGGTGTTGAGGACAGGTGGTGCGTCGTCGTTTTGTGCTTTTGCC
GCTTTCCCTCTCCTTAGGGATAAGGGTTACCTCCCTATACAAGATTCTAGATTAGAT
GAGCCAGTGACAGAGCTTCCACCTTTGAAAGTGAAGGATCTTCCGGTAATGGAAAC
GAATGAGCCGGAGGAACTTTACCGGGTAGTTAACGACATGGTGGAAGGAGCCAAG
TCTTCTTCAGGAGTCATATGGAACACATTTGAAGATCTTGAAAGACTATCACTTATG
AATTGTAGCAGCAAATTACAAGTTCCATTTTTCCCGATCGGACCGTTTCACAAATAT
AGCGAAGATCCTACACCGAAGACAGAGAACAAGGAAGATACCGATTGGCTCGACAA
GCAAGACCCACAGTCGGTGGTCTATGCGAGTTTCGGAAGCCTTGCAGCTATAGAA
GAGAAGGAGTTTCTCGAGATTGCTTGGGGTCTAAGAAACAGTGAACGACCGTTTTT
GTGGGTGGTTAGGCCGGGGTCTGTCAGGGGGACCGAGTGGCTCGAGTCATTGCC
TTTAGGGTTTATGGAAAACATTGGAGATAAGGGAAAAATCGTGAAATGGGCGAATC
AGTTAGAGGTATTGGCGCATCCTGCCATTGGAGCGTTTTGGACACATTGTGGATGG
AACTCGACACTAGAGAGCATATGTGAAGGTGTTCCTATGATATGTACGTCATGTTTC
ACGGACCAGCATGTGAACGCGAGATACATCGTTGATGTATGGCGAGTCGGGATGT
TGTTAGAGAGAAGTAAGATGGAAAAGAAGGAGATTGAAAAGGTGCTAAGAAGTGTA
ATGATGGAGAAGGGAGATGGATTGAGGGAAAGGAGTTTGAAGTTGAAAGAGAGAG
CTGATTTTTGCTTAAGTAAAGATGGGTCTTCTTCCAAGTATTTAGACAAACTTGTGA
GTCATGTCCTGTCTTTTGATTCTTATGCTTTTGCAAGTTAA
SEQ ID NO: 65 >UGT78D1
ATGACCAAATTCTCCGAGCCAATCAGAGACTCCCACGTGGCAGTTCTCGCGTTTTT
CCCCGTTGGCGCTCATGCCGGTCCTCTCTTAGCCGTCACTCGCCGTCTCGCCGCC
GCTTCTCCCTCCACCATCTTTTCTTTCTTCAACACCGCAAGATCAAACGCGTCGTTG
TTCTCCTCTGATCATCCCGAGAACATCAAGGTCCACGACGTCTCTGACGGTGTTCC
GGAGGGAACCATGCTCGGGAATCCACTGGAGATGGTCGAGCTGTTTCTCGAAGCG
GCTCCACGTATTTTCCGGAGCGAAATCGCGGCGGCAGAGATAGAAGTTGGAAAGA
AAGTGACATGCATGCTAACAGATGCCTTCTTCTGGTTCGCAGCGGACATAGCGGCT
GAGCTGAACGCGACTTGGGTTGCCTTCTGGGCCGGCGGAGCAAACTCACTCTGTG
CTCATCTCTACACTGATCTCATCAGAGAAACCATCGGTCTCAAAGATGTGAGTATGG
AAGAGACATTAGGGTTTATACCAGGAATGGAGAATTACAGAGTTAAAGATATACCAG
AGGAAGTTGTATTTGAAGATTTGGACTCTGTTTTCCCAAAGGCTTTATACCAAATGA
GTCTTGCTTTACCTCGTGCCTCTGCTGTTTTCATCAGTTCCTTTGAAGAGTTAGAAC
CTACATTGAACTATAACCTAAGATCCAAACTTAAACGTTTCTTGAACATCGCCCCTCT
CACGTTATTATCTTCTACATCGGAGAAAGAGATGCGTGATCCTCATGGCTGCTTTGC
TTGGATGGGGAAGAGATCAGCTGCTTCTGTAGCGTACATTAGCTTCGGCACCGTCA
TGGAACCTCCTCCTGAAGAGCTTGTGGCGATAGCACAAGGGTTGGAATCAAGCAAA
GTGCCGTTTGTTTGGTCGCTGAAGGAGAAGAACATGGTTCATCTACCAAAAGGGTT
TTTGGATCGGACAAGAGAGCAAGGGATAGTGGTTCCTTGGGCTCCACAAGTGGAA
CTGCTGAAACACGAGGCAATGGGTGTGAATGTGACACATTGTGGATGGAACTCAGT
GTTGGAGAGTGTGTCGGCAGGTGTACCGATGATCGGCAGACCGATTTTGGCGGAT
AATAGGCTCAACGGAAGAGCAGTGGAGGTTGTGTGGAAGGTTGGAGTGATGATGG
ATAATGGAGTCTTCACGAAAGAAGGATTTGAGAAGTGTTTGAATGATGTTTTTGTTC
ATGATGATGGTAAGACGATGAAGGCTAATGCCAAGAAGCTTAAAGAAAAACTCCAA
GAAGATTTCTCCATGAAAGGAAGCTCTTTAGAGAATTTCAAAATATTGTTGGACGAA
ATTGTGAAAGTTTAG
SEQ ID NO: 66 >UGT78D2
ATGACCAAACCCTCCGACCCAACCAGAGACTCCCACGTGGCAGTTCTCGCTTTTCC
TTTCGGCACTCATGCAGCTCCTCTCCTCACCGTCACGCGCCGCCTCGCCTCCGCCT
CTCCTTCCACCGTCTTCTCTTTCTTCAACACCGCACAATCCAACTCTTCGTTATTTTC
CTCCGGTGACGAAGCAGATCGTCCGGCGAACATCAGAGTATACGATATTGCCGAC
GGTGTTCCGGAGGGATACGTGTTTAGCGGGAGACCACAGGAGGCGATCGAGCTGT
TTCTTCAAGCTGCGCCGGAGAATTTCCGGAGAGAAATCGCGAAGGCGGAGACGGA
GGTTGGTACGGAAGTGAAATGTTTGATGACTGATGCGTTCTTCTGGTTCGCGGCTG
ATATGGCGACGGAGATAAATGCGTCGTGGATTGCGTTTTGGACCGCCGGAGCAAA
CTCACTCTCTGCTCATCTCTACACAGATCTCATCAGAGAAACCATCGGTGTCAAAGA
AGTAGGTGAGCGTATGGAGGAGACAATAGGGGTTATCTCAGGAATGGAGAAGATC
AGAGTCAAAGATACACCAGAAGGAGTTGTGTTTGGGAATTTAGACTCTGTTTTCTCA
AAGATGCTTCATCAAATGGGTCTTGCTTTGCCTCGTGCCACTGCTGTTTTCATCAAT
TCTTTTGAAGATTTGGATCCTACATTGACGAATAACCTCAGATCGAGATTTAAACGA
TATCTGAACATCGGTCCTCTCGGGTTATTATCTTCTACATTGCAACAACTAGTGCAA
GATCCTCACGGTTGTTTGGCTTGGATGGAGAAGAGATCTTCTGGTTCTGTGGCGTA
CATTAGCTTTGGTACGGTCATGACACCGCCTCCTGGAGAGCTTGCGGCGATAGCA
GAAGGGTTGGAATCGAGTAAAGTGCCGTTTGTTTGGTCGCTTAAGGAGAAGAGCTT
GGTTCAGTTACCAAAAGGGTTTTTGGATAGGACAAGAGAGCAAGGGATAGTGGTTC
CATGGGCACCGCAAGTGGAACTGCTGAAACACGAAGCAACGGGTGTGTTTGTGAC
GCATTGTGGATGGAACTCGGTGTTGGAGAGTGTATCGGGTGGTGTACCGATGATT
TGCAGGCCATTTTTTGGGGATCAGAGATTGAACGGAAGAGCGGTGGAGGTTGTGT
GGGAGATTGGAATGACGATTATCAATGGAGTCTTCACGAAAGATGGGTTTGAGAAG
TGTTTGGATAAAGTTTTAGTTCAAGATGATGGTAAGAAGATGAAATGTAATGCTAAG
AAACTTAAAGAACTAGCTTACGAAGCTGTCTCTTCTAAAGGAAGGTCCTCTGAGAAT
TTCAGAGGATTGTTGGATGCAGTTGTAAACATTATTTGA
SEQ ID NO: 67 >UGT78D3
ATGGCCAAACCCTCGCAGCCAACGCGAGACTCCCACGTGGCAGTTCTCGTTTTCCC
CTTCGGCACTCATGCAGCTCCTCTCCTCGCCGTCACGTGCCGTCTCGCCACCGCT
GCTCCCTCCACCGTCTTCTCCTTCTTCAGCACCGCACGATCCAACTCGTCGTTACT
CTCCTCCGATATCCCCACAAACATTCGTGTCCACAACGTCGATGACGGTGTTCCTG
AGGGATTCGTGTTGACGGGGAATCCACAGCACGCTGTTGAGCTGTTTCTTGAAGC
GGCGCCAGAGATTTTCCGAAGAGAAATCAAGGCGGCCGAGACCGAAGTTGGTAGG
AAGTTCAAGTGCATCCTTACGGATGCGTTCCTCTGGTTAGCAGCGGAGACGGCGG
CTGCGGAGATGAAAGCGTCGTGGGTTGCGTACTATGGAGGCGGAGCAACCTCGCT
CACTGCTCATCTCTACACAGATGCCATCAGAGAAAACGTCGGTGTCAAAAGTAGGT
GAGCGTATGGAGGAGACAATAGGGTTTATCTCAGGAATGGAGAAGATCAGAGTCAA
AGACACACAAGAAGGCGTTGTGTTTGGGAACTTAGACTCTGTTTTCTCTAAAACGTT
GCACCAAATGGGTCTTGCTTTACCTCGTGCCACTGCTGTTTTCATCAATTCCTTTGA
AGAATTGGATCCTACGTTTACAAATGATTTCAGATCGGAATTCAAACGTTACCTAAA
CATCGGTCCTCTCGCTTTATTATCTTCTCCATCGCAAACATCAACGCTAGTGCACGA
TCCTCACGGTTGCTTGGCTTGGATCGAGAAGCGGTCCACTGCTTCTGTAGCGTACA
TTGCCTTTGGTAGAGTCGCGACACCGCCTCCTGTAGAGCTTGTGGCGATAGCACAA
GGATTGGAATCGAGTAAAGTGCCTTTTGTTTGGTCGCTACAAGAGATGAAAATGAC
TCATTTACCAGAAGGCTTTTTGGATCGGACCAGAGAGCAAGGGATGGTGGTTCCAT
GGGCACCACAAGTGGAGCTGCTAAACCATGAAGCAATGGGTGTGTTTGTTTCGCAT
GGTGGGTGGAACTCAGTGTTGGAGAGTGTGTCGGCAGGTGTACCGATGATTTGTA
GACCGATTTTCGGGGATCATGCAATCAATGCAAGATCTGTGGAAGCTGTGTGGGAG
ATCGGAGTGACGATTAGTAGTGGAGTCTTCACGAAGGATGGATTTGAGGAGAGTTT
GGATCGGGTTTTGGTTCAAGATGATGGCAAGAAGATGAAGGTTAATGCTAAAAAGC
TTGAAGAACTAGCACAAGAAGCTGTCTCTACCAAAGGAAGCTCCTTTGAGAATTTTG
GAGGATTGTTGGACGAAGTTGTGAACTTTGGATAA
SEQ ID NO: 68 >UGT79B1
ATGGGTGTTTTTGGATCGAATGAATCGTCAAGCATGAGTATTGTGATGTATCCGTG
GTTAGCCTTTGGTCACATGACTCCTTTTCTTCACCTATCCAACAAGCTCGCAGAGAA
AGGTCACAAGATTGTTTTCTTGCTTCCCAAGAAAGCACTAAACCAGCTTGAACCTCT
TAATCTCTACCCAAATCTCATCACTTTCCACACCATCTCTATCCCTCAGGTCAAAGG
GCTCCCTCCGGGTGCGGAGACAAACTCCGACGTCCCTTTCTTCTTGACACATTTGC
TTGCAGTTGCAATGGACCAAACCCGGCCAGAGGTCGAGACCATTTTCCGTACAATC
AAACCGGACTTGGTTTTCTATGATTCTGCCCATTGGATACCGGAAATTGCTAAACCG
ATCGGTGCTAAAACCGTTTGCTTCAACATCGTTAGCGCTGCGTCAATCGCACTGTC
TCTTGTCCCTTCTGCGGAGAGAGAGGTCATTGATGGCAAGGAAATGTCAGGGGAG
GAGTTAGCTAAGACGCCTCTAGGTTACCCATCTTCGAAAGTAGTCTTACGTCCGCA
CGAAGCAAAATCCCTGAGTTTCGTGTGGAGGAAGCACGAGGCGATTGGCTCTTTCT
TTGATGGGAAAGTTACCGCGATGAGAAACTGCGACGCAATCGCTATAAGGACTTGC
CGTGAGACAGAAGGCAAATTCTGCGATTACATAAGTAGGCAGTACAGTAAACCGGT
TTACCTAACAGGACCGGTTCTCCCTGGATCCCAACCTAATCAGCCCTCCTTAGATC
CTCAATGGGCGGAGTGGCTAGCCAAATTCAACCACGGTTCGGTTGTGTTCTGCGCT
TTCGGTAGCCAACCCGTTGTAAACAAGATAGATCAGTTTCAAGAACTCTGTTTAGGT
CTAGAATCAACTGGTTTTCCGTTTCTGGTTGCCATTAAGCCTCCTTCGGGTGTATCA
ACCGTCGAGGAAGCCTTACCGGAAGGATTCAAAGAGAGGGTTCAAGGACGTGGCG
TTGTGTTTGGAGGTTGGATTCAGCAACCGTTGGTGTTGAACCATCCTTCAGTGGGT
TGTTTTGTTAGCCATTGCGGGTTTGGGTCGATGTGGGAGTCGTTGATGAGTGATTG
TCAGATCGTTTTGGTTCCGCAGCACGGAGAACAGATTTTGAACGCAAGGCTGATGA
CGGAGGAGATGGAGGTGGCGGTTGAAGTGGAGAGGGAAAAGAAAGGGTGGTTCT
CGCGGCAAAGCTTGGAGAATGCTGTGAAGAGTGTGATGGAGGAAGGTAGTGAGAT
CGGTGAGAAAGTGAGGAAGAATCATGACAAGTGGAGATGTGTTTTGACTGACTCTG
GTTTTTCAGATGGTTATATTGATAAGTTTGAACAAAATTTAATTGAACTTGTGAAGTC
ATGA
SEQ ID NO: 69 >UGT79B10
ATGGGCCAAACGTTTCACGCCTTTATGTTCCCATGGTTCGCTTTTGGTCATATGACT
CCATACTTGCATTTAGCCAACAAGTTAGCTGAGAGAGGTCACAGAATCACTTTCTTG
ATCCCCAAGAAAGCTCAGAAGCAGCTTGAACATCTCAATCTGTTTCCAGACAGCATC
GTCTTTCACTCTCTTACTATTCCTCATGTTGATGGTCTCCCCGCTGGAGCCGAGACT
TTCTCGGATATCCCTATGCCATTGTGGAAGTTCTTGCCCCCAGCTATAGATCTCACA
CGCGATCAAGTTGAAGCAGCGGTTAGTGCCTTGAGTCCGGACCTGATCTTGTTCGA
TATTGCTTCATGGGTTCCAGAAGTGGCTAAAGAGTATAGAGTCAAGAGTATGTTGTA
CAACATCATATCAGCTACTTCTATAGCTCATGACTTTGTCCCAGGTGGTGAACTTGG
AGTTCCTCCACCTGGTTATCCTTCCTCAAAGTTGTTGTACCGCAAACACGATGCTCA
CGCCTTGTTGTCCTTCTCCGTCTACTACAAGAGGTTTTCTCATCGGCTCATCACAGG
TCTTATGAATTGTGATTTCATTTCGATAAGGACATGCAAAGAAATCGAGGGTAAATT
CTGCGAGTATCTTGAGCGTCAATACCATAAAAAGGTTTTCTTGACGGGTCCAATGCT
TCCTGAGCCAAACAAAGGTAAACCACTGGAAGATCGATGGAGTCATTGGCTGAACG
GGTTTGAACAAGGCTCTGTAGTGTTCTGTGCATTGGGAAGTCAAGTCACTCTAGAG
AAGGACCAGTTCCAAGAACTTTGTTTAGGAATAGAGCTTACAGGTTTACCGTTTTTT
GTAGCTGTAACACCACCAAAAGGCGCAAAGACGATTCAAGATGCGTTACCAGAAGG
GTTCGAGGAGAGGGTGAAAGATCGTGGAGTGGTTTTGGGAGAATGGGTGCAACAA
CCGTTATTATTGGCTCATCCATCAGTAGGCTGCTTCTTGAGTCATTGCGGATTCGG
GTCAATGTGGGAATCTATAATGAGTGATTGCCAAATAGTTTTGCTTCCATTTTTGGC
TGATCAAGTTCTCAACACAAGATTGATGACCGAAGAACTCAAGGTTTCGGTTGAAGT
GCAAAGAGAAGAAACAGGATGGTTCTCGAAGGAGAGCTTGAGTGTTGCTATCACAT
CTGTGATGGACCAAGCTAGTGAGATCGGGAATCTGGTGAGAAGGAACCATTCCAAA
TTGAAGGAGGTTTTGGTTAGTGATGGATTATTAACCGGTTACACCGATAAATTTGTT
GACACTTTGGAGAATCTTGTCAGCGAGACAAAGCGTGAATGA
SEQ ID NO: 70 >UGT79B11
ATGGGCCAAAAGATTCACGCTTTTATGTTCCCCTGGTTTGCTTTTGGTCATATGACT
CCGTACTTGCATCTAGGCAACAAGTTAGCCGAGAAAGGTCATAGGGTTACTTTCTT
GCTACCTAAGAAAGCTCAGAAACAATTGGAACATCAGAATCTATTTCCACACGGTAT
CGTCTTTCATCCTCTTGTTATTCCTCATGTTGATGGCCTCCCTGCTGGTGCCGAGAC
AGCCTCGGATATCCCCATCTCGTTGGTGAAGTTCTTGTCTATAGCCATGGATCTTAC
ACGCGATCAGATCGAAGCCGCGATTGGTGCCTTGAGACCGGACCTAATCTTGTTCG
ATTTAGCTCACTGGGTTCCGGAAATGGCTAAAGCGCTTAAAGTCAAGAGTATGTTG
TATAACGTGATGTCAGCTACCTCTATAGCTCACGACCTTGTCCCAGGTGGTGAACT
TGGAGTTGCTCCACCTGGTTATCCTTCATCAAAGGCGTTGTACCGCGAACACGATG
CTCACGCCTTGTTAACCTTCTCCGGCTTCTACAAGAGGTTTTATCACCGGTTCACCA
CAGGTCTTATGAATTGCGATTTCATTTCGATTCGGACATGTGAAGAAATCGAAGGTA
AATTTTGTGACTATATTGAGAGTCAATACAAGAAGAAGGTTCTTTTAACCGGTCCAA
TGCTTCCCGAGCCTGACAAGAGTAAACCACTTGAAGATCAATGGAGTCATTGGCTG
AGTGGGTTTGGACAAGGCTCTGTAGTGTTCTGTGCATTGGGAAGTCAAACCATTCT
AGAGAAAAACCAATTCCAAGAACTCTGTTTAGGAATAGAGCTTACGGGTTTACCATT
TCTTGTCGCGGTTAAGCCACCAAAAGGCGCAAACACAATTCATGAAGCGTTACCAG
AAGGGTTCGAGGAAAGGGTGAAGGGTCGTGGAATAGTTTGGGGAGAATGGGTGCA
GCAACCATCCTGGCAACCATTGATATTGGCTCATCCATCAGTAGGTTGCTTTGTGA
GCCATTGCGGATTCGGGTCAATGTGGGAATCTTTAATGAGTGATTGTCAAATAGTC
TTTATTCCAGTTTTGAATGATCAAGTTCTCACCACGAGAGTAATGACGGAGGAACTC
GAGGTCTCCGTTGAGGTACAGAGAGAAGAAACAGGATGGTTCTCAAAAGAAAACTT
GAGTGGTGCAATCATGTCTTTGATGGACCAAGACAGCGAGATAGGGAACCAAGTGA
GGAGGAACCATTCTAAATTGAAGGAGACTTTGGCTAGTCCTGGATTATTAACCGGT
TACACCGATAAATTTGTTGACACTTTGGAGAATCTAGTCAACGAACAAGGATACATA
TCTTGA
SEQ ID NO: 71 >UGT79B2
ATGGGTGGTTTGAAGTTTCATGTACTTATGTATCCATGGTTCGCAACAGGCCATATG
ACCCCGTTCCTTTTTCTTGCCAACAAATTGGCTGAGAAAGGTCATACGGTCACTTTT
TTGATTCCCAAGAAAGCTCTGAAACAGTTGGAAAATCTCAATCTGTTTCCACACAAC
ATTGTCTTTCGCTCTGTCACCGTCCCTCATGTGGATGGTCTCCCCGTTGGCACAGA
GACAGTCTCTGAGATCCCCGTGACATCAGCTGATCTCTTGATGTCTGCTATGGATC
TCACACGTGATCAAGTTGAAGGTGTGGTCCGAGCCGTGGAACCGGACCTGATCTT
CTTTGACTTCGCTCATTGGATTCCAGAGGTAGCTAGAGACTTTGGCCTTAAGACTGT
AAAGTACGTCGTGGTATCTGCATCGACTATAGCTAGTATGCTTGTTCCAGGTGGTG
AGTTAGGTGTTCCTCCGCCGGGATATCCTTCATCGAAGGTGCTGCTTCGTAAACAA
GATGCTTACACCATGAAGAATCTGGAGTCTACAAATACAATCAATGTCGGACCAAAC
TTATTGGAAAGAGTCACTACAAGTCTTATGAACTCTGATGTCATTGCGATAAGGACA
GCCAGAGAAATCGAAGGAAACTTTTGCGACTATATCGAAAAACATTGCAGGAAAAA
GGTTCTCTTGACAGGTCCGGTGTTCCCTGAGCCAGACAAGACTAGAGAGCTAGAG
GAACGATGGGTTAAGTGGCTAAGTGGGTATGAACCAGACTCAGTGGTGTTTTGTGC
GTTGGGCTCACAAGTCATTTTAGAGAAAGATCAATTCCAAGAACTCTGCTTAGGAAT
GGAGCTAACAGGTTCACCGTTTCTTGTAGCGGTTAAGCCACCTAGAGGCTCATCAA
CGATTCAAGAAGCACTTCCTGAAGGATTCGAGGAGAGGGTTAAAGGAAGAGGAGT
TGTTTGGGGAGAATGGGTTCAACAACCATTGCTATTGTCTCATCCATCAGTCGGGT
GCTTTGTGAGCCATTGTGGGTTTGGATCAATGTGGGAGTCTTTGCTGAGTGATTGT
CAGATAGTCTTGGTACCACAGTTGGGTGATCAGGTCCTCAACACAAGATTGCTGAG
TGACGAACTCAAGGTTTCGGTTGAAGTGGCAAGAGAGGAAACAGGATGGTTCTCG
AAAGAGAGCTTGTTCGATGCTATCAATAGTGTGATGAAAAGGGACAGTGAGATCGG
GAATCTGGTGAAGAAGAATCACACCAAGTGGAGGGAGACACTAACTAGTCCTGGAC
TTGTGACCGGTTATGTCGATAATTTCATAGAGTCATTGCAGGATCTTGTCTCTGGGA
CCAACCATGTTTCGAAGTAG
SEQ ID NO: 72 >UGT79B3
ATGGGTGGTTTGAAGTTTCATGTACTTATGTATCCATGGTTCGCAACAGGCCATATG
ACCCCGTTCCTTTTTCTTGCCAACAAATTGGCTGAGAAAGGTCATACGGTCACTTTC
TTGCTTCCCAAGAAATCTCTGAAACAGTTGGAACATTTCAATCTGTTTCCACACAAC
ATTGTCTTTCGCTCTGTCACCGTCCCTCATGTGGATGGTCTCCCCGTTGGCACAGA
GACAGCCTCTGAGATCCCTGTGACATCAACTGATCTCTTGATGTCTGCTATGGATCT
CACACGTGATCAAGTTGAAGCTGTGGTCCGAGCCGTTGAACCGGACCTGATCTTCT
TTGACTTTGCTCATTGGATTCCAGAAGTAGCTAGGGACTTCGGCCTTAAGACTGTAA
AGTACGTCGTGGTGTCTGCATCGACTATAGCTAGTATGCTTGTCCCAGGTGGTGAG
TTAGGTGTTCCTCCACCGGGATATCCATCATCAAAGGTGCTGCTTCGTAAACAAGAT
GCTTACACTATGAAGAAACTGGAGCCTACAAATACAATCGATGTCGGACCAAACCT
CTTGGAACGAGTCACTACAAGTCTTATGAACTCTGATGTCATTGCGATAAGGACAG
CCAGAGAAATCGAAGGAAACTTTTGCGACTATATAGAAAAACATTGCAGGAAAAAG
GTTCTCTTGACAGGTCCGGTGTTCCCTGAGCCAGACAAGACTAGAGAGCTAGAGG
AACGATGGGTTAAGTGGCTAAGTGGGTATGAACCAGACTCAGTGGTGTTTTGTGCA
CTGGGCTCACAAGTCATTTTAGAGAAAGATCAATTCCAAGAACTCTGCTTAGGAATG
GAGCTAACAGGTTCACCGTTTCTTGTAGCGGTTAAGCCCCCTAGAGGCTCATCAAC
GATTCAAGAAGCACTTCCTGAAGGATTCGAAGAGCGGGTTAAAGGAAGAGGCCTTG
TTTGGGGAGGATGGGTTCAACAACCATTGATATTGTCTCATCCATCAGTCGGGTGC
TTTGTGAGCCATTGTGGGTTTGGATCAATGTGGGAGTCTTTGCTGAGTGATTGTCA
GATAGTCTTAGTACCACAGTTGGGTGATCAAGTCCTGAACACAAGATTGCTGAGTG
ACGAACTCAAGGTTTCGGTTGAAGTGGCAAGAGAGGAAACAGGATGGTTCTCGAAA
GAGAGCTTGTGCGATGCTGTCAATAGTGTGATGAAAAGGGACAGCGAGCTCGGGA
ACCTGGTGAGGAAGAATCACACCAAGTGGAGGGAGACAGTAGCTAGTCCTGGACT
AATGACTGGTTATGTCGATGCTTTCGTAGAGTCATTGCAGGATCTTGTCTCTGGGA
CCACCCATGACTGA
SEQ ID NO: 73 >UGT79B4
ATGGGGTCAAAGTTTCATGCTTTTCTTTATCCATGGTTTGGTTTTGGTCATATGATTC
CGTATCTTCATCTAGCTAACAAATTAGCTGAAAAAGGTCATAGGGTTACTTTCTTGG
CTCCCAAGAAAGCTCAGAAACAACTCGAACCTCTCAACTTGTTCCCAAACAGCATTC
ACTTCGAGAATGTTACTCTTCCTCATGTTGATGGTCTCCCTGTTGGCGCAGAGACA
ACCGCGGATCTCCCGAACTCATCTAAGAGAGTCCTCGCTGATGCCATGGATCTTCT
ACGCGAACAGATTGAAGTTAAGATTCGTTCTTTGAAACCTGACCTAATTTTCTTCGA
TTTTGTTGATTGGATTCCACAAATGGCAAAAGAATTAGGAATCAAAAGTGTAAGTTA
CCAGATCATATCGGCAGCTTTTATAGCTATGTTTTTCGCTCCTCGTGCTGAATTAGG
TTCTCCTCCACCTGGGTTTCCTTCATCAAAAGTAGCATTACGTGGACATGACGCTAA
CATCTATTCACTCTTCGCAAACACCCGCAAATTTCTCTTTGATCGAGTCACCACAGG
CCTTAAGAACTGCGACGTCATTGCCATAAGGACATGTGCAGAAATCGAAGGTAACT
TATGTGATTTCATCGAAAGACAATGTCAGAGAAAAGTTCTCTTAACCGGTCCAATGT
TCCTTGATCCACAAGGGAAGAGTGGTAAGCCGCTAGAAGATCGATGGAATAATTGG
TTAAACGGATTTGAACCAAGCTCGGTAGTGTACTGTGCGTTTGGCACCCATTTCTTT
TTCGAGATAGATCAATTTCAAGAACTCTGTTTAGGAATGGAGCTCACGGGTCTACCT
TTTTTGGTAGCGGTTATGCCACCGAGAGGGTCTTCAACGATTCAAGAAGCATTACC
AGAAGGGTTCGAAGAACGGATTAAAGGGCGTGGAATTGTTTGGGGAGGATGGGTG
GAACAACCTTTGATATTGTCTCATCCATCAATAGGTTGCTTTGTGAACCATTGCGGG
TTCGGTTCAATGTGGGAGTCTTTGGTTAGTGATTGCCAGATTGTGTTTATTCCACAA
TTGGTTGATCAAGTTCTCACAACGAGATTGTTGACCGAAGAACTCGAGGTCTCCGT
GAAAGTAAAGAGAGATGAAATTACTGGTTGGTTTTCGAAGGAGAGCTTGAGGGATA
CGGTCAAATCTGTGATGGATAAAAATAGTGAGATTGGGAATCTAGTGAGGAGGAAT
CATAAGAAACTGAAGGAAACTTTGGTTAGTCCTGGATTGTTGAGTAGTTATGCTGAT
AAGTTTGTTGACGAATTAGAGAATCATATCCACAGTAAGAATTGA
SEQ ID NO: 74 >UGT79B5
ATGGGATCAAAATTTCATGCTTTTATGTATCCATGGTTTGGTTTTGGTCATATGATTC
CATATCTTCATTTAGCCAACAAACTAGCTGAGAAAGGTCATAGGGTCACTTTCTTCC
TCCCCAAGAAAGCTCATAAGCAGCTCCAACCTCTCAATCTGTTCCCAGACAGCATT
GTCTTTGAGCCTCTTACTCTCCCTCCTGTCGATGGTCTCCCTTTTGGCGCCGAGAC
AGCCTCGGATCTCCCAAACTCAACTAAGAAACCCATATTCGTTGCCATGGATCTCTT
ACGCGATCAGATCGAAGCAAAGGTCCGTGCTTTGAAACCAGATCTAATCTTTTTCGA
TTTTGTTCATTGGGTTCCAGAAATGGCAGAAGAGTTTGGAATAAAGAGTGTCAATTA
CCAGATCATATCGGCAGCTTGTGTAGCTATGGTTCTTGCACCTAGGGCTGAATTAG
GGTTTCCTCCGCCGGATTATCCTTTATCCAAAGTGGCGTTACGTGGACATGAAGCT
AACGTCTGTTCTCTCTTTGCGAATTCCCATGAGCTTTTCGGTCTGATCACCAAAGGC
CTTAAGAACTGTGACGTCGTTTCCATAAGGACCTGCGTGGAACTTGAAGGTAAGCT
ATGCGGTTTCATCGAAAAAGAATGTCAAAAGAAACTTCTCTTAACCGGTCCAATGCT
CCCTGAACCGCAAAATAAGAGTGGTAAATTTCTAGAAGACCGATGGAATCACTGGT
TAAACGGATTTGAACCAGGGTCGGTAGTGTTTTGTGCGTTTGGCACTCAATTCTTTT
TCGAGAAGGATCAATTTCAAGAATTCTGTTTAGGAATGGAGCTAATGGGTCTACCGT
TTTTAATATCGGTTATGCCGCCAAAAGGCTCACCAACGGTTCAAGAAGCGTTACCAA
AAGGATTCGAAGAACGGGTTAAAAAGCATGGAATCGTTTGGGAAGGATGGTTGGAA
CAACCTTTGATATTGTCTCATCCATCAGTAGGTTGCTTTGTGAACCATTGTGGCTTT
GGTTCAATGTGGGAGTCTTTGGTTAGTGATTGTCAGATTGTGTTTATTCCACAATTG
GCAGATCAAGTTCTCATCACAAGATTGTTGACTGAAGAACTCGAAGTCTCTGTGAAA
GTGCAGAGAGAAGATTCCGGATGGTTCTCGAAAGAGGACTTGAGAGATACTGTTAA
ATCTGTGATGGATATAGATAGTGAGATTGGGAACTTAGTGAAGAGGAATCATAAGA
AATTGAAAGAGACTTTAGTTAGTCCTGGATTGTTAAGTGGTTATGCTGATAAGTTTG
TAGAAGCATTGGAGATTGAAGTCAACAACACCAAATTTTCTTGA
SEQ ID NO: 75 >UGT79B6
ATGGGGTCAAAGTTTCATGCTTTTATGTTCCCATGGTTTGGTTTTGGTCACATGACT
GCATTTTTGCATCTGGCTAACAAACTAGCGGAGAAAGACCACAAAATAACTTTCTTG
CTCCCCAAGAAAGCTCGAAAGCAACTTGAATCTCTCAATCTCTTCCCAGACTGCATT
GTCTTTCAGACTCTTACCATCCCATCTGTAGATGGCCTCCCTGATGGTGCTGAGAC
AACCTCGGATATCCCGATCTCGTTAGGCAGTTTTCTCGCCTCGGCTATGGATCGGA
CACGCATTCAGGTCAAAGAAGCAGTTTCTGTTGGTAAACCGGATCTGATTTTCTTCG
ATTTTGCTCACTGGATTCCGGAAATAGCTAGAGAGTATGGAGTCAAGAGTGTCAATT
TCATAACGATTTCTGCAGCATGTGTAGCTATTTCGTTCGTCCCTGGTCGTAGTCAAG
ATGACTTGGGTAGTACTCCACCGGGATACCCTTCCTCCAAGGTGTTGCTTCGGGGA
CACGAAACCAACAGTTTGTCGTTCCTCTCCTATCCGTTTGGAGATGGAACTAGTTTT
TACGAACGGATCATGATAGGACTTAAGAACTGCGATGTCATTTCGATAAGGACATG
CCAAGAAATGGAAGGAAAGTTCTGCGATTTCATCGAAAACCAATTTCAAAGAAAAGT
TCTCTTGACAGGTCCAATGCTTCCTGAGCCGGACAATAGCAAACCGCTAGAAGATC
AATGGCGTCAGTGGCTTAGCAAGTTCGATCCGGGATCAGTAATATATTGTGCATTG
GGCAGCCAAATCATTCTTGAAAAGGATCAATTCCAAGAACTCTGTTTAGGAATGGAG
CTGACAGGTTTACCATTTCTTGTAGCGGTAAAGCCACCAAAAGGTTCATCGACAATC
CAAGAAGCCTTACCAAAAGGGTTTGAAGAGAGGGTTAAAGCACGTGGAGTGGTTTG
GGGAGGATGGGTGCAGCAACCATTGATATTAGCTCATCCATCAATAGGCTGCTTTG
TGAGCCATTGTGGTTTCGGGTCAATGTGGGAGGCTCTAGTGAATGACTGCCAAATA
GTGTTTATTCCACATTTGGGTGAGCAAATATTGAACACAAGACTGATGAGCGAGGA
ACTCAAGGTCTCGGTAGAGGTGAAAAGAGAGGAAACGGGATGGTTTTCGAAGGAG
AGCTTGAGCGGTGCGGTCAGGTCTGTGATGGACAGAGATAGCGAGCTCGGGAATT
GGGCGAGGAGGAACCACGTAAAGTGGAAGGAGTCTCTGCTTCGTCATGGACTAAT
GAGTGGTTATCTTAATAAGTTCGTAGAAGCATTGGAGAAACTAGTCCAAAATATAAA
TCTTGAATGA
SEQ ID NO: 76 >UGT79B7
ATGGAGCCAAAGTTTCATGCTTTTATGTTTCCATGGTTTGCTTTTGGTCATATGATTC
CATTTCTACATCTTGCAAACAAACTAGCTGAAAAAGGTCACCGAGTTACTTTCTTGC
TACCTAAGAAAGCACAAAAACAGTTGGAACATCACAACTTGTTCCCAGACAGTATTG
TCTTTCACCCTCTCACAGTTCCTCCTGTCAATGGCCTCCCTGCTGGTGCCGAGACA
ACCTCGGATATCCCCATCTCGTTGGACAACCTCTTGTCCAAAGCCTTGGATCTCACT
CGCGATCAGGTTGAAGCTGCGGTTCGTGCTTTGAGACCTGACTTGATCTTTTTCGA
TTTTGCTCAATGGATTCCAGATATGGCTAAAGAACATATGATCAAGAGTGTGAGTTA
CATCATTGTATCTGCGACAACAATAGCTCATACACATGTCCCTGGAGGTAAATTAGG
TGTTCGCCCACCGGGTTATCCGTCATCAAAGGTGATGTTCCGTGAAAACGATGTTC
ATGCCTTAGCAACCTTATCGATATTTTACAAGAGACTGTATCATCAGATCACTACAG
GTCTTAAGAGCTGTGATGTCATTGCATTGAGGACTTGCAAAGAAGTCGAAGGTATG
TTCTGCGACTTTATATCGCGTCAATACCATAAGAAGGTTCTCTTGACTGGTCCAATG
TTCCCTGAGCCAGACACAAGTAAACCACTAGAAGAACGCTGGAATCATTTTCTAAGC
GGGTTCGCGCCGAAGTCAGTAGTGTTTTGTTCACCTGGCAGCCAAGTAATTCTTGA
GAAAGATCAATTCCAAGAACTCTGTTTAGGGATGGAGCTAACAGGTTTACCATTTCT
TTTAGCGGTAAAGCCACCAAGAGGATCATCAACGGTCCAAGAAGGGTTACCAGAAG
GGTTCGAGGAGCGGGTGAAAGATCGTGGTGTTGTTTGGGGAGGATGGGTGCAACA
ACCTTTGATATTGGCTCATCCATCAATAGGTTGCTTTGTGAACCATTGTGGTCCCGG
AACAATATGGGAGTCTTTGGTGAGTGATTGCCAAATGGTTTTGATTCCATTTTTAAG
TGATCAAGTTCTCTTCACAAGATTGATGACCGAGGAATTCGAGGTCTCTGTAGAAGT
GCCGAGGGAAAAAACAGGATGGTTTTCAAAGGAGAGCTTGAGCAATGCTATCAAAT
CTGTGATGGATAAAGACAGTGACATTGGGAAGTTAGTGAGGAGTAACCACACCAAA
TTGAAGGAGATTTTAGTTAGTCCTGGATTATTGACTGGTTACGTTGATCACTTTGTA
GAGGGATTGCAAGAGAATTTGATTTGA
SEQ ID NO: 77 >UGT79B8
ATGGAGCCAACGTTCCATGCTTTTATGTTTCCCTGGTTTGCTTTTGGTCATATGATT
CCTTTTCTACATCTTGCAAACAAACTAGCTGAGAAAGGTCATCAAATCACTTTCTTG
CTACCTAAGAAAGCCCAAAAACAGTTGGAACATCACAATCTGTTCCCAGACAGTATT
GTCTTTCACCCTCTCACAATCCCTCATGTCAATGGCCTCCCTGCTGGTGCTGAGAC
AACCTCGGATATCTCAATCTCGATGGACAACTTACTGTCGGAAGCCTTGGATCTCA
CTCGCGATCAGGTTGAAGCTGCGGTTCGTGCTCTGAGACCGGACTTGATCTTTTTT
GATTTTGCTCATTGGATTCCAGAAATTGCCAAAGAGCATATGATCAAGAGTGTGAGT
TACATGATAGTATCTGCAACAACAATAGCTTATACATTTGCCCCTGGTGGTGTATTA
GGTGTTCCCCCACCAGGTTATCCTTCATCAAAGGTGTTGTACCGTGAAAACGATGC
TCATGCCTTAGCAACCTTATCTATCTTCTACAAGAGACTTTATCATCAGATCACTACA
GGTTTTAAGAGCTGTGACATCATTGCATTGAGGACATGTAATGAAATCGAAGGTAAA
TTCTGCGACTATATATCAAGTCAATACCATAAGAAGGTTCTCTTGACTGGTCCAATG
CTCCCTGAGCAAGACACAAGTAAACCACTAGAAGAACAGTTGAGTCATTTTCTGAG
CAGGTTCCCACCGAGGTCAGTGGTGTTTTGTGCACTTGGTAGCCAGATCGTTCTTG
AAAAGGATCAATTCCAAGAACTCTGCTTAGGGATGGAGCTGACAGGTTTACCGTTT
CTTATAGCGGTAAAGCCACCGAGAGGATCATCGACGGTCGAAGAAGGGTTACCAG
AAGGGTTCCAGGAGCGGGTGAAAGGGCGTGGTGTGGTTTGGGGAGGATGGGTGC
AACAACCATTGATATTGGATCATCCGTCAATAGGCTGCTTTGTGAACCATTGTGGTC
CGGGAACAATATGGGAGTGTCTTATGACTGATTGTCAAATGGTTTTGCTTCCATTTT
TAGGTGATCAAGTTCTCTTCACAAGATTGATGACCGAGGAATTCAAGGTGTCTGTA
GAAGTGTCGAGAGAAAAAACAGGATGGTTTTCAAAGGAGAGCTTGAGCGATGCGAT
CAAGTCTGTGATGGATAAAGATAGCGACCTCGGAAAGCTAGTGAGGAGTAACCACG
CCAAATTGAAGGAGACTCTTGGTAGTCATGGATTATTAACTGGTTACGTGGATAAAT
TTGTAGAGGAATTGCAAGAGTATTTGATTTGA
SEQ ID NO: 78 >UGT79B9
ATGGGCCAAAATTTTCACGCTTTTATGTTCCCATGGTTCGCTTTTGGTCATATGACT
CCATACTTGCATCTAGCCAACAAGCTAGCTGCTAAAGGTCATAGGGTTACTTTCTTG
CTGCCTAAGAAAGCTCAAAAACAGTTGGAACATCACAATCTGTTTCCAGACAGGATC
ATCTTTCATTCTCTTACTATTCCCCATGTTGATGGCCTACCTGCTGGCGCGGAGACC
GCCTCGGACATCCCCATCTCGTTGGGGAAGTTTCTTACCGCAGCCATGGATCTCAC
TCGCGATCAGGTCGAAGCCGCGGTTCGTGCTTTGAGACCAGACCTGATCTTTTTCG
ATACTGCTTATTGGGTTCCGGAAATGGCGAAAGAACACAGAGTCAAGAGTGTGATA
TACTTTGTGATATCAGCTAACTCCATAGCTCATGAACTTGTACCAGGTGGTGAATTA
GGAGTTCCTCCACCTGGCTATCCTTCGTCAAAAGTGTTGTACCGTGGACACGATGC
TCACGCTTTGTTGACTTTTTCCATCTTCTACGAGAGGCTTCATTACCGGATAACAAC
AGGTCTAAAGAATTGTGATTTTATCTCAATTAGGACTTGTAAAGAAATCGAAGGTAA
ATTCTGCGACTATATAGAGCGTCAATACCAGAGGAAGGTTCTTTTGACAGGTCCAAT
GCTTCCAGAGCCAGATAACAGTAGACCACTCGAAGATCGATGGAATCACTGGCTGA
ATCAGTTCAAACCCGGCTCGGTAATATATTGTGCATTGGGAAGTCAAATCACTCTAG
AGAAGGATCAATTCCAAGAACTCTGTTTAGGAATGGAGCTCACTGGTTTACCGTTTC
TCGTAGCGGTAAAACCACCAAAAGGCGCAAAGACGATCCAAGAAGCGTTGCCAGA
AGGGTTTGAGGAGAGGGTGAAGAATCATGGAGTAGTTTGGGGAGAATGGGTGCAG
CAACCATTGATATTGGCTCATCCATCAGTAGGCTGCTTTGTGACCCATTGTGGGTTT
GGATCAATGTGGGAGTCTCTAGTGAGTGATTGTCAAATAGTCTTGCTTCCATATTTG
TGTGATCAAATTCTCAACACTAGATTGATGAGTGAGGAACTCGAGGTTTCGGTGGA
AGTGAAAAGAGAAGAAACAGGATGGTTCTCGAAAGAGAGCTTAAGTGTTGCGATCA
CCTCGGTGATGGACAAAGATAGTGAGTTAGGGAATCTGGTGAGGAGGAACCACGC
TAAATTAAAGGAGGTTTTGGTTAGTCCTGGATTATTAACCGGTTACACCGATGAATT
TGTTGAAACTTTGCAGAATATAGTCAACGATACAAATCTTGAATGA
SEQ ID NO: 79 >UGT82A1
ATGAAAGTAACACAAAAGCCAAAGATAATATTCATCCCTTATCCGGCGCAAGGCCAC
GTCACTCCGATGCTTCACCTTGCATCGGCTTTCCTCAGCCGTGGATTCTCCCCTGT
CGTTATGACTCCCGAGTCTATCCACCGTAGGATCTCGGCTACTAACGAGGATCTTG
GGATCACGTTCTTGGCCTTATCTGACGGTCAAGATCGTCCGGACGCACCTCCCTCG
GACTTCTTCTCGATAGAGAACTCAATGGAGAACATCATGCCACCACAGCTCGAACG
GCTCCTACTAGAAGAAGACTTGGATGTGGCTTGTGTTGTGGTTGATTTGCTGGCTT
CGTGGGCTATAGGAGTGGCTGATCGGTGTGGAGTTCCGGTCGCCGGATTCTGGCC
GGTGATGTTCGCTGCTTACCGTTTGATCCAAGCAATACCGGAGCTAGTCCGAACAG
GCTTAGTTTCCCAAAAAGGTTGTCCTCGTCAACTAGAAAAAACAATAGTCCAGCCAG
AGCAACCGCTCCTATCCGCAGAAGATCTACCGTGGCTGATCGGAACTCCCAAAGCT
CAGAAAAAACGATTCAAGTTCTGGCAAAGAACTCTAGAACGAACAAAAAGTCTCCGT
TGGATCTTGACAAGCTCCTTTAAAGATGAATATGAAGATGTCGACAACCACAAAGCA
TCCTACAAAAAATCTAACGATTTAAACAAAGAAAACAATGGTCAAAACCCTCAAATCC
TTCATTTAGGTCCATTGCATAACCAAGAAGCAACAAATAATATAACTATAACCAAGAC
TAGTTTTTGGGAAGAAGACATGTCTTGTCTAGGTTGGCTTCAAGAACAAAACCCGAA
CTCAGTCATTTATATCTCATTTGGAAGTTGGGTTTCTCCTATAGGAGAATCAAATATT
CAAACGTTGGCATTGGCGTTGGAAGCGTCAGGGAGACCTTTCCTTTGGGCGTTAAA
CCGAGTGTGGCAAGAGGGACTACCACCAGGTTTTGTGCATAGAGTCACAATTACCA
AAAACCAAGGAAGGATCGTCTCATGGGCTCCGCAACTTGAAGTTCTTAGAAACGAT
TCTGTGGGATGTTACGTGACTCATTGTGGCTGGAACTCGACTATGGAGGCAGTGG
CAAGTTCCCGGAGGCTACTATGTTATCCGGTGGCCGGAGACCAGTTTGTTAACTGT
AAATACATCGTGGACGTTTGGAAGATTGGAGTGAGATTGAGCGGGTTTGGAGAGAA
GGAGGTTGAAGATGGACTAAGGAAAGTAATGGAGGATCAAGATATGGGTGAGAGA
TTGAGGAAGTTAAGAGACAGAGCAATGGGGAATGAAGCTCGTTTGAGTTCGGAAAT
GAATTTTACATTTTTAAAAAACGAGCTTAATTAG
SEQ ID NO: 80 >UGT83A1
ATGGATAATAACTCAAATAAAAGAATGGGAAGGCCACATGTTGTGGTCATACCTTAC
CCTGCACAAGGTCATGTTCTTCCTCTAATAAGTTTCTCACGTTACCTTGCGAAACAA
GGAATCCAAATTACATTCATAAACACCGAGTTTAACCATAACCGCATCATCAGTTCC
TTACCCAATTCACCTCATGAAGATTATGTTGGGGATCAGATCAATCTTGTTTCAATC
CCTGACGGTTTAGAAGATTCACCAGAAGAGAGGAACATTCCAGGGAAGTTGTCGGA
GTCTGTTTTGCGTTTTATGCCTAAAAAAGTAGAGGAATTGATCGAGAGGATGATGG
CAGAAACTAGCGGTGGTACGATCATTAGCTGCGTTGTAGCGGATCAGAGCTTGGG
ATGGGCAATTGAAGTTGCAGCTAAGTTTGGGATCAGACGCACCGCGTTTTGTCCTG
CTGCAGCTGCGTCTATGGTTCTTGGATTTAGTATTCAAAAACTTATCGATGATGGTC
TCATAGATTCTGATGGGACTGTGAGAGTAAATAAGACAATTCAACTATCTCCCGGGA
TGCCAAAGATGGAAACAGACAAGTTTGTGTGGGTTTGTCTGAAGAACAAAGAATCT
CAGAAAAACATATTCCAACTTATGCTTCAAAACAATAACTCGATCGAGTCAACGGAT
TGGTTGTTGTGTAACTCTGTCCATGAACTTGAAACTGCAGCATTTGGATTGGGCCC
GAATATAGTACCAATTGGGCCCATTGGTTGGGCTCATAGTCTTGAAGAGGGATCCA
CGTCACTAGGAAGCTTTTTACCTCATGACCGGGATTGTCTAGATTGGTTGGACCGG
CAGATTCCCGGTTCGGTTATATATGTTGCCTTTGGGAGTTTTGGGGTCATGGGCAA
CCCTCAGTTAGAAGAGCTAGCAATTGGTCTAGAGCTTACCAAGAGGCCAGTTTTGT
GGGTCACTGGTGATCAACAACCAATCAAACTTGGGTCGGATCGAGTCAAAGTGGTG
AGATGGGCTCCACAACGGGAGGTCCTTTCTTCTGGAGCCATTGGGTGTTTTGTGAG
CCATTGTGGATGGAATTCAACTCTGGAAGGAGCCCAAAATGGCATACCATTTCTAT
GCATCCCTTATTTTGCAGACCAATTTATCAACAAAGCATATATATGCGATGTGTGGA
AGATTGGATTAGGACTTGAAAGAGACGCACGAGGAGTGGTTCCGAGGTTAGAGGT
TAAGAAGAAGATCGATGAGATCATGAGAGACGGTGGAGAGTATGAAGAACGAGCTA
TGAAGGTTAAAGAGATTGTGATGAAAAGTGTTGCAAAAGATGGAATATCTTGTGAGA
ATCTTAATAAATTTGTCAACTGGATCAAATCACAAGTGAATTGA
SEQ ID NO: 81 >UGT84A1
ATGGTGTTCGAAACTTGTCCATCTCCAAACCCAATTCATGTAATGCTCGTCTCGTTT
CAAGGACAAGGCCACGTCAACCCTCTTCTTCGTCTCGGCAAGTTAATTGCTTCAAA
GGGTTTACTCGTTACCTTCGTTACAACGGAGCTTTGGGGCAAGAAAATGAGACAAG
CCAACAAAATCGTTGACGGTGAACTTAAACCGGTTGGTTCCGGTTCAATCCGGTTT
GAGTTCTTTGATGAAGAATGGGCAGAGGATGATGACCGGAGAGCTGATTTCTCTTT
GTACATTGCTCACCTAGAGAGCGTTGGGATACGAGAAGTGTCTAAGCTTGTGAGAA
GATACGAGGAAGCGAACGAGCCTGTCTCGTGTCTTATCAATAACCCGTTTATCCCA
TGGGTCTGCCACGTGGCGGAAGAGTTCAACATTCCTTGTGCGGTTCTCTGGGTTCA
GTCTTGTGCTTGTTTCTCTGCTTATTACCATTACCAAGATGGCTCTGTTTCATTCCCT
ACGGAAACAGAGCCTGAGCTCGATGTGAAGCTTCCTTGTGTTCCTGTCTTGAAGAA
CGACGAGATTCCTAGCTTTCTCCATCCTTCTTCTAGGTTCACGGGTTTTCGACAAGC
GATTCTTGGGCAATTCAAGAATCTGAGCAAGTCCTTCTGTGTTCTAATCGATTCTTT
TGACTCATTGGAACAAGAAGTTATCGATTACATGTCAAGTCTTTGTCCGGTTAAAAC
CGTTGGACCGCTTTTCAAAGTTGCTAGGACAGTTACTTCTGACGTAAGCGGTGACA
TTTGCAAATCAACAGATAAATGCCTCGAGTGGTTAGACTCGAGGCCTAAATCGTCA
GTTGTCTACATTTCGTTCGGGACAGTTGCATATTTGAAGCAAGAACAGATCGAAGA
GATCGCTCACGGAGTTTTGAAGTCGGGTTTATCGTTCTTGTGGGTGATTAGACCTC
CACCACACGATCTGAAGGTCGAGACACATGTCTTGCCTCAAGAACTTAAAGAGAGT
AGTGCTAAAGGTAAAGGGATGATTGTGGATTGGTGCCCACAAGAGCAAGTCTTGTC
TCATCCTTCAGTGGCATGCTTCGTGACTCATTGTGGATGGAACTCGACAATGGAAT
CTTTGTCTTCAGGTGTTCCGGTGGTTTGTTGTCCGCAATGGGGAGATCAAGTGACT
GATGCAGTGTATTTGATCGATGTTTTCAAGACCGGGGTTAGACTAGGCCGTGGAGC
GACCGAGGAGAGGGTAGTGCCAAGGGAGGAAGTGGCGGAGAAGCTTTTGGAAGC
GACAGTTGGGGAGAAGGCAGAGGAGTTGAGAAAGAACGCTTTGAAATGGAAGGCG
GAGGCGGAAGCAGCGGTGGCTCCAGGAGGTTCGTCGGATAAGAATTTTAGGGAGT
TTGTGGAGAAGTTAGGTGCGGGAGTAACGAAGACTAAAGATAATGGATACTAG
SEQ ID NO: 82 >UGT84A2
ATGGAGCTAGAATCTTCTCCTCCTCTACCTCCTCATGTGATGCTCGTATCTTTTCCA
GGGCAAGGCCACGTTAATCCACTTCTTCGTCTTGGTAAGCTCTTAGCTTCAAAGGG
TTTGCTCATAACCTTCGTCACCACTGAGTCATGGGGCAAAAAGATGCGAATCTCCA
ACAAAATCCAAGACCGTGTCCTCAAACCGGTTGGTAAAGGCTATCTCCGGTATGAT
TTCTTCGACGACGGGCTTCCTGAAGACGACGAAGCTAGCAGAACCAACTTAACCAT
CCTCCGACCACATCTAGAGCTGGTCGGCAAAAGAGAGATCAAGAACCTTGTGAAAC
GTTACAAGGAAGTAACGAAACAGCCCGTGACATGTCTTATCAACAACCCTTTCGTCT
CTTGGGTCTGTGACGTGGCAGAAGATCTTCAAATCCCTTGTGCTGTTCTTTGGGTT
CAATCTTGTGCCTGCTTAGCTGCTTATTACTATTACCACCACAACCTAGTTGACTTC
CCGACCAAAACAGAACCCGAGATCGATGTCCAAATCTCTGGCATGCCTCTCTTGAA
ACATGACGAGATCCCTTCTTTCATTCACCCTTCAAGTCCTCACTCCGCTTTGCGAGA
AGTGATCATAGATCAGATTAAACGGCTTCACAAGACTTTCTCCATTTTCATCGACAC
TTTCAACTCATTGGAGAAAGACATCATTGACCACATGTCGACGCTCTCTCTCCCCG
GTGTTATCAGACCGCTAGGACCACTCTACAAAATGGCTAAAACCGTAGCTTATGAT
GTCGTTAAAGTAAACATCTCTGAGCCAACGGATCCTTGCATGGAGTGGTTAGACTC
GCAGCCAGTTTCCTCCGTTGTTTACATCTCATTCGGGACCGTTGCTTACTTGAAACA
AGAACAAATAGACGAGATCGCTTACGGTGTGTTAAACGCCGACGTTACGTTCTTGT
GGGTGATTAGACAACAAGAGTTAGGTTTCAACAAAGAGAAACATGTTTTGCCGGAA
GAAGTTAAAGGGAAAGGGAAGATCGTTGAATGGTGTTCACAAGAGAAAGTATTATC
TCATCCTTCAGTGGCATGTTTCGTGACTCACTGTGGATGGAACTCAACGATGGAAG
CTGTGTCTTCCGGAGTCCCGACGGTTTGTTTTCCTCAATGGGGAGATCAAGTCACG
GACGCCGTTTACATGATCGATGTTTGGAAGACGGGAGTGAGGCTAAGCCGTGGAG
AGGCGGAGGAGAGGTTAGTGCCGAGGGAGGAAGTTGCGGAGAGGTTGAGAGAGG
TTACTAAAGGAGAGAAAGCGATCGAGTTGAAAAAGAATGCTTTGAAGTGGAAGGAA
GAGGCGGAGGCGGCGGTTGCTCGCGGTGGTTCGTCGGATAGGAATCTTGAAAAG
TTTGTGGAGAAGTTGGGTGCCAAACCTGTGGGGAAAGTACAAAACGGGAGTCATAA
TCATGTCTTGGCTGGATCAATCAAAAGCTTTTAA
SEQ ID NO: 83 >UGT84A3
ATGGACCCGTCTCGTCATACTCATGTGATGCTCGTATCTTTCCCCGGCCAAGGTCA
CGTAAACCCTCTACTTCGTCTCGGAAAGCTCATAGCCTCTAAAGGCTTACTCGTCAC
CTTTGTCACCACAGAGAAGCCATGGGGCAAGAAGATGCGTCAAGCCAACAAGATTC
AAGACGGTGTGCTCAAACCGGTCGGTCTAGGTTTCATCCGGTTTGAGTTCTTCTCT
GACGGCTTCGCCGACGACGATGAAAAAAGATTCGACTTCGATGCCTTCCGACCACA
CCTTGAAGCTGTCGGAAAACAAGAGATCAAGAATCTCGTTAAGAGATATAACAAGG
AGCCGGTGACGTGTCTCATAAACAACGCTTTTGTCCCATGGGTATGTGATGTCGCC
GAGGAGCTTCACATCCCTTCGGCTGTTCTATGGGTCCAGTCTTGTGCTTGTCTCAC
GGCTTATTACTATTACCACCACCGGTTAGTTAAGTTCCCGACCAAAACCGAGCCGG
ACATCAGCGTTGAAATCCCTTGCTTGCCATTGTTAAAGCATGACGAGATCCCAAGCT
TTCTTCACCCTTCGTCTCCGTATACAGCTTTTGGAGATATCATTTTAGACCAGTTAAA
GAGATTCGAAAACCACAAGTCTTTCTATCTTTTCATCGACACTTTTCGCGAACTAGA
AAAAGACATCATGGACCACATGTCACAACTTTGTCCTCAAGCCATCATCAGTCCTGT
CGGTCCGCTCTTCAAGATGGCTCAAACCTTGAGTTCTGACGTTAAGGGAGATATAT
CCGAGCCAGCGAGTGACTGCATGGAATGGCTTGACTCAAGAGAACCATCCTCAGT
CGTTTACATCTCCTTTGGGACTATAGCCAACTTGAAGCAAGAGCAGATGGAGGAGA
TCGCTCATGGCGTTTTGAGCTCTGGCTTGTCGGTCTTATGGGTGGTTCGGCCTCCC
ATGGAAGGGACATTTGTAGAACCACATGTTTTGCCTCGAGAGCTCGAAGAAAAGGG
TAAAATCGTGGAATGGTGTCCCCAAGAGAGAGTCTTGGCTCATCCTGCGATTGCTT
GTTTCTTAAGTCACTGCGGATGGAACTCGACAATGGAGGCTTTAACTGCCGGAGTC
CCCGTTGTTTGTTTTCCGCAATGGGGAGATCAAGTGACTGATGCGGTGTACTTGGC
TGATGTTTTCAAGACAGGAGTGAGACTAGGCCGCGGAGCCGCTGAGGAGATGATT
GTTTCGAGGGAGGTTGTAGCAGAGAAGCTGCTTGAGGCCACAGTTGGGGAAAAGG
CGGTGGAGCTGAGAGAAAACGCTCGGAGGTGGAAGGCGGAGGCCGAGGCCGCC
GTGGCGGACGGTGGATCATCTGATATGAACTTTAAAGAGTTTGTGGACAAGTTGGT
TACGAAACATGTGACGAGAGAAGACAACGGAGAACACTAG
SEQ ID NO: 84 >UGT84A4
ATGGAGATGGAATCGTCGTTACCTCATGTGATGCTCGTATCATTCCCAGGGCAAGG
TCACATAAGCCCTCTTCTTCGTCTCGGAAAGATCATTGCCTCTAAAGGCTTAATCGT
CACCTTTGTAACCACAGAGGAACCATTGGGCAAGAAGATGCGTCAAGCCAACAATA
TTCAAGACGGTGTGCTCAAACCGGTCGGGCTAGGTTTTCTCCGGTTCGAGTTCTTC
GAGGATGGATTTGTCTACAAAGAAGACTTTGATTTGTTACAAAAATCACTTGAAGTT
TCCGGAAAACGAGAGATCAAGAATCTTGTCAAGAAATATGAGAAGCAACCAGTGAG
ATGTCTCATAAATAATGCCTTTGTTCCATGGGTTTGTGACATAGCCGAGGAGCTTCA
AATCCCATCAGCTGTTCTTTGGGTCCAGTCTTGTGCTTGCCTCGCCGCTTATTACTA
TTACCACCACCAGTTAGTTAAGTTTCCGACCGAAACCGAGCCGGAAATAACCGTTG
ACGTCCCTTTCAAGCCATTAACATTGAAGCATGACGAGATCCCTAGCTTTCTTCACC
CTTCCTCTCCGCTGTCCTCTATAGGAGGTACCATTTTAGAGCAGATCAAGCGACTTC
ACAAGCCTTTCTCTGTTCTCATCGAAACTTTTCAAGAACTTGAAAAAGATACCATTGA
CCACATGTCCCAGCTCTGCCCTCAAGTCAACTTCAACCCCATCGGTCCGCTTTTTAC
TATGGCTAAAACCATAAGGTCTGACATCAAGGGAGACATCTCCAAGCCAGATAGTG
ACTGCATAGAGTGGCTTGACTCGAGAGAACCATCCTCCGTTGTTTACATCTCTTTTG
GGACTTTGGCTTTCTTGAAGCAAAACCAGATCGACGAGATTGCTCACGGCATTCTC
AACTCCGGGTTGTCCTGCTTATGGGTTTTGCGGCCTCCCTTAGAAGGCTTAGCCAT
AGAACCGCATGTCTTGCCTCTAGAGCTTGAAGAGAAAGGGAAGATTGTGGAATGGT
GTCAACAAGAGAAAGTTTTGGCTCATCCTGCGGTTGCTTGCTTCTTAAGTCACTGTG
GATGGAACTCAACCATGGAGGCTTTAACTTCAGGAGTTCCCGTTATTTGTTTCCCG
CAGTGGGGAGATCAGGTGACAAATGCGGTGTACATGATTGATGTTTTCAAGACAGG
ATTGAGACTCAGCCGTGGAGCTTCCGATGAGAGGATTGTTCCAAGGGAGGAGGTT
GCTGAGCGACTGCTTGAGGCCACCGTTGGAGAGAAGGCGGTGGAGCTGAGAGAA
AACGCTCGGAGGTGGAAGGAGGAGGCGGAGTCTGCCGTGGCTTACGGTGGAACA
TCGGAAAGGAATTTTCAAGAGTTTGTTGACAAGTTGGTTGATGTCAAGACAATGACA
AACATTAATAATGTCGTGTAA
SEQ ID NO: 85 >UGT84B1
ATGGGCAGTAGTGAGGGTCAAGAAACACATGTCCTAATGGTAACACTACCATTCCA
AGGTCACATCAATCCAATGCTCAAACTCGCAAAACATCTCTCGTTATCATCAAAGAA
CCTACACATCAATCTCGCCACTATTGAGTCAGCCCGTGATCTCCTCTCCACCGTAG
AAAAACCTCGTTATCCGGTGGACCTCGTGTTCTTCTCCGATGGTCTACCTAAAGAA
GATCCAAAGGCCCCTGAAACTCTTTTGAAGTCATTGAATAAAGTCGGAGCCATGAA
CTTGTCTAAAATCATCGAAGAAAAGAGATACTCTTGTATCATCTCTTCGCCTTTTACT
CCATGGGTTCCAGCTGTTGCAGCCTCTCATAACATCTCTTGTGCAATACTTTGGATC
CAAGCTTGTGGAGCTTACTCGGTTTATTACCGTTACTACATGAAGACAAACTCTTTC
CCTGATCTTGAAGATCTGAATCAAACGGTGGAGTTACCAGCTTTACCATTGTTGGAA
GTTCGAGATCTTCCATCGTTTATGTTACCTTCTGGTGGTGCTCACTTCTATAATCTA
ATGGCGGAATTTGCAGATTGTTTGAGGTATGTGAAATGGGTTTTGGTTAATTCATTC
TATGAACTCGAATCAGAGATAATCGAATCGATGGCTGATTTAAAACCTGTAATTCCA
ATTGGTCCTCTGGTTTCTCCATTTCTGTTGGGCGATGGTGAGGAGGAAACCCTAGA
CGGTAAAAACCTAGATTTTTGTAAATCTGATGATTGTTGTATGGAGTGGCTTGACAA
GCAAGCTAGGTCTTCTGTTGTGTACATATCTTTCGGAAGTATGCTCGAAACATTGGA
GAATCAGGTCGAGACCATAGCGAAGGCGCTGAAGAACAGAGGACTTCCATTTCTTT
GGGTGATAAGGCCAAAGGAGAAAGCCCAAAACGTTGCTGTTTTGCAGGAGATGGT
GAAAGAAGGACAAGGGGTTGTTCTCGAGTGGAGTCCACAAGAGAAGATTTTGAGC
CACGAGGCAATCTCTTGTTTTGTCACGCATTGCGGCTGGAACTCGACTATGGAGAC
GGTGGTGGCTGGTGTTCCTGTGGTAGCGTACCCTAGCTGGACGGATCAGCCCATT
GACGCGCGGTTGCTTGTTGATGTGTTTGGAATCGGAGTAAGGATGAGGAATGACA
GTGTCGATGGCGAGCTTAAGGTCGAAGAAGTAGAAAGATGCATTGAGGCCGTGAC
GGAGGGACCCGCTGCCGTGGATATAAGAAGGAGAGCGGCGGAGCTAAAGCGCGT
GGCGAGATTGGCGTTGGCACCTGGTGGATCTTCGACACGGAATTTAGACTTGTTCA
TTAGTGATATCACAATCGCCTAA
SEQ ID NO: 86 >UGT84B2
ATGGGAAGTAATGAGGGTCAAGAAACACATGTCCTAATGGTAGCATTAGCATTCCA
AGGTCATCTCAATCCAATGCTCAAATTCGCAAAACATCTCGCACGAACCAATCTACA
CTTCACTCTCGCCACCACTGAGCAAGCCCGTGACCTCCTCTCTTCCACCGCTGACG
AACCTCATAGACCGGTGGACCTCGCTTTCTTCTCAGACGGTCTACCTAAAGACGAT
CCAAGAGATCCCGACACTCTCGCAAAGTCATTGAAAAAAGATGGAGCCAAGAACTT
GTCAAAAATCATCGAAGAAAAGAGATTTGATTGCATCATCTCTGTGCCTTTTACTCC
CTGGGTTCCAGCTGTTGCAGCTGCACATAACATTCCTTGTGCAATCCTCTGGATCC
AAGCTTGTGGAGCTTTTTCTGTTTATTACCGTTATTACATGAAGACAAATCCTTTCCC
CGACCTTGAAGATCTGAATCAAACAGTGGAGTTACCAGCTTTACCATTGTTGGAAGT
CCGAGATCTCCCGTCATTGATGTTACCTTCTCAAGGAGCTAATGTCAATACCCTAAT
GGCGGAATTTGCAGATTGTTTGAAAGATGTGAAATGGGTTTTGGTTAACTCGTTTTA
CGAACTCGAATCAGAGATCATCGAGTCTATGTCTGATTTAAAACCTATAATCCCAAT
TGGTCCTCTTGTTTCTCCATTCCTGTTGGGAAATGATGAAGAAAAAACCCTAGATAT
GTGGAAAGTTGATGATTATTGTATGGAGTGGCTTGACAAGCAAGCTAGGTCTTCAG
TTGTTTACATATCTTTCGGAAGCATACTCAAATCATTGGAGAATCAAGTTGAGACCA
TAGCAACGGCATTAAAAAACAGAGGAGTTCCATTTCTTTGGGTGATACGGCCGAAG
GAGAAAGGCGAAAACGTCCAGGTTTTGCAGGAGATGGTTAAAGAAGGTAAAGGGG
TTGTAACTGAATGGGGTCAACAAGAAAAGATATTGAGCCACATGGCGATTTCTTGCT
TCATCACGCATTGTGGATGGAACTCGACGATCGAGACGGTGGTGACTGGTGTTCC
CGTGGTGGCGTATCCGACTTGGATAGATCAGCCGCTTGATGCGAGACTGCTTGTG
GATGTGTTTGGAATCGGAGTAAGGATGAAGAACGACGCTATCGATGGAGAGCTTAA
GGTTGCAGAGGTGGAGAGATGCATTGAGGCCGTGACAGAGGGACCTGCCGCCGC
GGATATGAGGAGGAGAGCGACGGAGCTGAAGCACGCCGCAAGATCGGCGATGTC
ACCTGGTGGATCTTCCGCTCAGAATTTAGACTCGTTCATTAGTGATATCCCAATCAC
TTGA
SEQ ID NO: 87 >UGT85A1
ATGGGATCTCAGATCATTCATAACTCACAAAAACCACATGTAGTTTGTGTTCCATAT
CCGGCTCAAGGCCACATCAACCCTATGATGAGAGTGGCTAAACTCCTCCACGCCAG
AGGCTTCTACGTCACCTTCGTCAACACCGTCTACAACCACAATCGTTTCCTTCGTTC
TCGTGGGTCCAATGCCCTAGATGGACTTCCTTCGTTCCGATTTGAGTCCATTGCTG
ACGGTCTACCAGAGACAGACATGGATGCCACGCAGGACATCACAGCTCTTTGCGA
GTCCACCATGAAGAACTGTCTCGCTCCGTTCAGAGAGCTTCTCCAGCGGATCAACG
CTGGAGATAATGTTCCTCCGGTAAGCTGTATTGTATCTGACGGTTGTATGAGCTTTA
CTCTTGATGTTGCGGAGGAGCTTGGAGTCCCGGAGGTTCTTTTTTGGACAACCAGT
GGCTGTGCGTTCCTGGCTTATCTACACTTTTATCTCTTCATCGAGAAGGGCTTATGT
CCGCTAAAAGATGAGAGTTACTTGACGAAGGAGTACTTAGAAGACACGGTTATAGA
TTTTATACCAACCATGAAGAATGTGAAACTAAAGGATATTCCTAGCTTCATACGTAC
CACTAATCCTGATGATGTTATGATTAGTTTCGCCCTCCGCGAGACCGAGCGAGCCA
AACGTGCTTCTGCTATCATTCTAAACACATTTGATGACCTTGAGCATGATGTTGTTC
ATGCTATGCAATCTATCTTACCTCCGGTTTATTCAGTTGGACCGCTTCATCTCTTAG
CAAACCGGGAGATTGAAGAAGGTAGTGAGATTGGAATGATGAGTTCGAATTTATGG
AAAGAGGAGATGGAGTGTTTGGATTGGCTTGATACTAAGACTCAAAATAGTGTCATT
TATATCAACTTTGGGAGCATAACGGTTTTGAGTGTGAAGCAGCTTGTGGAGTTTGC
TTGGGGTTTGGCGGGAAGTGGGAAAGAGTTTTTATGGGTGATCCGGCCAGATTTA
GTAGCGGGAGAGGAGGCTATGGTTCCGCCGGACTTTTTAATGGAGACTAAAGACC
GCAGTATGCTAGCGAGTTGGTGTCCTCAAGAGAAAGTACTTTCTCATCCTGCTATT
GGAGGGTTTTTGACGCATTGCGGGTGGAACTCGATATTGGAAAGTCTTTCGTGTGG
AGTTCCGATGGTGTGTTGGCCATTTTTTGCTGACCAGCAAATGAATTGTAAGTTTTG
TTGTGACGAGTGGGATGTTGGGATTGAGATAGGTGGAGATGTGAAGAGAGAGGAA
GTTGAGGCGGTGGTTAGAGAGCTCATGGATGGAGAGAAGGGAAAGAAAATGAGAG
AAAAGGCGGTAGAGTGGCAGCGCTTAGCCGAGAAAGCGACGGAACATAAACTTGG
TTCTTCCGTTATGAATTTTGAGACGGTTGTTAGCAAGTTTCTTTTGGGACAAAAATC
ACAGGATTAA
SEQ ID NO: 88 >UGT85A2
ATGGGATCTCATGTCGCACAAAAACAACACGTAGTTTGCGTTCCTTATCCGGCTCAA
GGCCACATCAACCCAATGATGAAAGTGGCTAAACTCCTTTACGCCAAAGGCTTCCA
TATTACCTTCGTCAACACCGTCTACAACCACAACCGTCTCCTCCGGTCCCGTGGGC
CTAACGCCGTTGACGGGCTTCCTTCTTTCCGGTTTGAGTCCATCCCTGACGGTCTA
CCCGAGACTGACGTGGACGTCACTCAGGACATCCCTACTCTTTGCGAGTCCACAAT
GAAGCACTGTCTCGCTCCATTCAAGGAGCTTCTCCGGCAGATCAACGCAAGGGAT
GATGTTCCTCCTGTGAGCTGTATCGTATCCGACGGTTGTATGAGCTTCACACTTGA
TGCTGCGGAGGAGCTCGGTGTCCCGGAGGTTCTTTTTTGGACAACTAGTGCTTGT
GGCTTCTTGGCTTACCTTTACTACTATCGCTTCATCGAGAAGGGATTATCACCAATA
AAAGATGAGAGTTACTTAACCAAGGAACACTTGGACACAAAAATAGACTGGATACCA
TCGATGAAGAACCTAAGACTAAAAGACATCCCTAGCTTCATCCGAACGACTAATCCT
GACGACATCATGCTCAACTTTATCATCCGTGAGGCTGACCGAGCCAAACGCGCTTC
AGCTATCATTCTCAACACGTTTGATGATCTCGAACACGACGTTATCCAATCTATGAA
ATCCATTGTACCTCCGGTTTATTCTATTGGACCGTTACATTTACTAGAGAAACAAGA
GAGCGGCGAGTATAGTGAAATCGGACGGACAGGATCGAATCTTTGGAGAGAGGAG
ACTGAGTGTCTGGACTGGCTAAACACGAAAGCTAGAAACAGTGTTGTGTACGTTAA
CTTCGGGAGTATAACTGTTTTGAGCGCAAAACAGCTTGTGGAGTTTGCATGGGGTT
TGGCTGCAACGGGGAAAGAGTTTTTGTGGGTGATCCGGCCGGATTTAGTAGCCGG
GGATGAGGCAATGGTTCCACCGGAGTTTTTAACGGCTACGGCGGACCGGAGGATG
TTGGCAAGTTGGTGTCCTCAAGAGAAAGTCCTTTCTCATCCGGCCATTGGAGGGTT
CTTGACGCATTGCGGGTGGAACTCGACGTTGGAAAGTCTATGCGGTGGAGTTCCA
ATGGTGTGTTGGCCGTTTTTTGCAGAGCAACAAACTAATTGTAAGTTTTCTCGTGAC
GAATGGGAGGTTGGGATTGAGATTGGTGGAGATGTGAAGAGAGAAGAGGTTGAGG
CGGTGGTTAGGGAGTTGATGGATGAAGAGAAGGGAAAGAATATGAGAGAGAAGGC
GGAAGAGTGGCGGCGCTTGGCGAATGAAGCGACGGAGCATAAGCATGGTTCTTCT
AAATTGAACTTTGAGATGCTCGTTAATAAGGTTCTTTTAGGGGAGTAG
SEQ ID NO: 89 >UGT85A3
ATGGGATCCCGTTTTGTTTCTAACGAACAAAAACCACACGTAGTTTGCGTGCCTTAC
CCAGCTCAAGGCCACATTAACCCTATGATGAAAGTGGCTAAACTCCTCCACGTCAA
AGGCTTCCACGTCACCTTCGTCAACACCGTCTACAACCACAACCGTCTACTCCGAT
CCCGTGGGGCCAACGCACTCGATGGACTTCCTTCCTTCCAGTTCGAGTCAATACCT
GACGGTCTTCCGGAGACTGGCGTGGACGCCACGCAGGACATCCCTGCCCTTTCCG
AGTCCACAACGAAAAACTGTCTCGTTCCGTTCAAGAAGCTTCTCCAGCGGATTGTC
ACGAGAGAGGATGTCCCTCCGGTGAGCTGTATTGTATCAGATGGTTCGATGAGCTT
TACTCTTGACGTAGCGGAAGAGCTTGGTGTTCCGGAGATTCATTTTTGGACCACTA
GTGCTTGTGGCTTCATGGCTTATCTACACTTTTATCTCTTCATCGAGAAGGGTTTAT
GTCCAGTAAAAGATGCGAGTTGCTTGACGAAGGAATACTTGGACACAGTTATAGAT
TGGATACCGTCAATGAACAATGTAAAACTAAAAGACATTCCTAGTTTTATACGTACC
ACTAATCCTAACGACATAATGCTCAACTTCGTTGTCCGTGAGGCATGTCGAACCAAA
CGTGCCTCTGCTATCATTCTGAACACGTTTGATGACCTTGAACATGACATAATCCAG
TCTATGCAATCCATTTTACCACCGGTTTATCCAATCGGACCGCTTCATCTCTTAGTA
AACAGGGAGATTGAAGAAGATAGTGAGATTGGAAGGATGGGATCAAATCTATGGAA
AGAGGAGACTGAGTGCTTGGGATGGCTTAATACTAAGTCTCGAAATAGCGTTGTTT
ATGTTAACTTTGGGAGCATAACAATAATGACCACGGCACAGCTTTTGGAGTTTGCTT
GGGGTTTGGCGGCAACGGGAAAGGAGTTTCTATGGGTGATGCGGCCGGATTCAGT
AGCCGGAGAGGAGGCAGTGATTCCAAAAGAGTTTTTAGCGGAGACAGCTGATCGA
AGAATGCTGACAAGTTGGTGTCCTCAGGAGAAAGTTCTTTCTCATCCGGCGGTCGG
AGGGTTCTTGACCCATTGCGGGTGGAATTCGACGTTAGAAAGTCTTTCATGCGGAG
TTCCAATGGTATGTTGGCCATTTTTTGCTGAGCAACAAACAAATTGTAAGTTTTCTTG
TGATGAATGGGAGGTTGGTATTGAGATCGGTGGAGATGTCAAGAGGGGAGAGGTT
GAGGCGGTGGTTAGAGAGCTCATGGATGGAGAGAAAGGAAAGAAAATGAGAGAGA
AGGCTGTAGAGTGGCGGCGCTTGGCCGAGAAAGCTACAAAGCTTCCGTGTGGTTC
GTCGGTGATAAATTTTGAGACGATTGTCAACAAGGTTCTCTTGGGAAAGATCCCTAA
CACGTAA
SEQ ID NO: 90 >UGT85A4
ATGGAACAACATGGCGGTTCTAGCTCACAGAAACCTCACGCAATGTGCATACCTTA
TCCAGCACAAGGCCACATCAACCCAATGCTGAAACTAGCCAAGCTCCTCCACGCTA
GAGGCTTCCACGTCACTTTCGTCAACACCGACTACAACCACCGCCGTATCCTCCAA
TCACGTGGCCCTCACGCTCTCAACGGTCTCCCCTCGTTTCGCTTCGAGACTATCCC
CGACGGTCTTCCTTGGACAGACGTCGACGCTAAGCAAGACATGCTCAAGCTTATTG
ACTCCACAATAAACAACTGTTTAGCTCCATTCAAAGACCTCATCCTCCGGTTAAACT
CCGGTTCTGATATACCACCGGTTAGCTGTATCATCTCCGACGCTTCAATGAGCTTCA
CAATTGACGCAGCGGAGGAGCTTAAAATTCCGGTAGTTCTCCTCTGGACCAACAGT
GCTACTGCTTTAATCTTGTATCTCCATTACCAAAAACTCATCGAGAAAGAGATAATTC
CCCTCAAAGATTCGAGTGACTTGAAGAAGCATTTAGAGACGGAGATTGATTGGATA
CCGTCGATGAAGAAGATTAAGCTTAAGGATTTTCCAGATTTCGTCACCACGACGAAT
CCTCAAGATCCGATGATTAGTTTCATCCTTCATGTAACCGGAAGAATCAAAAGAGCT
TCTGCGATCTTCATCAACACTTTCGAAAAACTCGAGCATAACGTTCTCTTATCTCTG
CGATCTCTTCTCCCTCAGATCTACTCCGTTGGACCGTTCCAGATTCTGGAGAATCG
CGAAATCGATAAGAACAGCGAAATCAGAAAGCTAGGATTGAATCTCTGGGAAGAAG
AGACGGAGTCTTTGGATTGGCTAGATACTAAAGCTGAGAAAGCTGTGATTTACGTC
AACTTCGGGAGTCTAACGGTTTTGACTAGTGAGCAGATCTTAGAGTTCGCTTGGGG
TTTAGCGAGGAGCGGGAAAGAGTTTCTCTGGGTGGTGAGATCTGGTATGGTCGAC
GGAGATGATTCGATTCTTCCGGCGGAGTTTTTATCGGAGACGAAGAATCGAGGAAT
GTTAATTAAAGGATGGTGTTCTCAGGAGAAGGTACTTTCGCATCCGGCGATTGGAG
GATTTTTGACTCACTGTGGATGGAATTCGACGTTGGAGAGTTTGTACGCCGGTGTT
CCGATGATCTGTTGGCCATTTTTTGCTGATCAGTTGACGAATCGAAAGTTCTGTTGC
GAGGATTGGGGGATTGGGATGGAGATCGGCGAGGAGGTGAAGAGGGAGAGAGTG
GAGACGGTGGTTAAAGAGCTCATGGACGGAGAGAAGGGAAAGAGGTTAAGAGAGA
AGGTGGTGGAGTGGCGGCGCTTGGCGGAAGAAGCTTCGGCGCCACCGTTGGGAT
CATCGTACGTGAATTTTGAAACGGTGGTTAATAAAGTCCTTACATGTCACACGATTA
GATCGACCTAA
SEQ ID NO: 91 >UGT85A5
ATGGCGTCTCATGCTGTTACAAGCGGACAAAAACCACACGTAGTTTGCATACCTTTC
CCGGCTCAAGGCCACATCAATCCGATGCTCAAAGTGGCTAAACTCCTCTATGCCAG
AGGCTTCCATGTTACCTTCGTCAACACTAACTACAACCATAACCGTCTCATCCGGTC
ACGTGGTCCCAACTCCCTTGATGGGCTTCCTTCTTTTCGGTTCGAGTCCATCCCTG
ACGGTCTACCGGAGGAAAACAAGGACGTCATGCAGGATGTCCCTACCCTTTGTGA
GTCCACCATGAAAAACTGTCTAGCTCCTTTCAAGGAGCTTCTCCGGCGGATCAACA
CCACAAAGGATGTTCCTCCGGTAAGCTGTATTGTATCCGACGGTGTGATGAGCTTT
ACTCTTGATGCTGCAGAGGAGCTTGGAGTCCCGGATGTTCTTTTTTGGACACCAAG
TGCTTGTGGCTTCTTGGCTTATCTACACTTCTATCGCTTCATCGAGAAGGGGTTATC
ACCAATAAAAGATGAAAGTTCTTTGGACACAAAAATAAATTGGATACCATCGATGAA
AAACCTAGGACTTAAAGACATCCCAAGCTTTATCCGTGCAACTAATACTGAAGACAT
AATGCTTAACTTTTTTGTCCATGAGGCTGACCGAGCCAAACGCGCTTCCGCTATCAT
TCTCAACACATTCGATAGTCTTGAGCATGATGTCGTCCGTTCTATTCAATCTATCATA
CCTCAAGTGTACACTATTGGACCGCTTCATCTATTTGTGAATCGGGATATCGACGA
GGAAAGTGACATCGGACAGATAGGAACGAATATGTGGAGAGAGGAGATGGAGTGT
TTGGATTGGCTTGATACTAAGTCTCCAAACAGTGTCGTTTATGTTAATTTCGGTAGC
ATAACAGTGATGAGTGCGAAACAACTCGTGGAGTTTGCTTGGGGTTTAGCAGCGAC
CAAAAAAGATTTTTTGTGGGTGATTAGGCCGGATTTAGTAGCCGGTGATGTGCCAA
TGCTTCCGCCGGACTTTCTAATAGAGACGGCTAACCGAAGGATGCTAGCGAGTTG
GTGTCCTCAAGAAAAAGTTCTTTCTCATCCGGCAGTTGGAGGGTTCTTAACGCATA
GTGGATGGAATTCGACTTTGGAGAGTCTCTCCGGTGGAGTTCCAATGGTGTGTTGG
CCGTTCTTTGCGGAACAGCAAACAAATTGTAAATATTGTTGTGATGAATGGGAAGTG
GGGATGGAGATCGGTGGAGATGTGAGGAGGGAGGAGGTTGAGGAGTTGGTTAGA
GAACTCATGGACGGAGACAAAGGAAAGAAAATGAGGCAAAAGGCCGAAGAGTGGC
AGCGCTTGGCTGAGGAAGCGACGAAGCCTATTTATGGTTCGTCGGAACTAAATTTT
CAGATGGTCGTTGACAAGGTTCTTTTAGGGGAGTAG
SEQ ID NO: 92 >UGT85A7
ATGGAATCTCATGTTGTTCATAACGCACAAAAGCCACACGTAGTTTGCGTGCCTTAC
CCGGCTCAAGGCCACATCAATCCGATGCTGAAAGTGGCTAAACTCCTCTACGCTAA
AGGCTTTCACGTCACCTTCGTTAACACTCTCTACAACCACAACCGTCTCCTCCGGTC
CCGTGGTCCCAACGCGCTCGACGGGTTTCCTTCATTCCGGTTCGAGTCCATCCCTG
ACGGTCTACCGGAGACTGATGGCGATAGGACGCAGCATACTCCTACCGTTTGCAT
GTCCATTGAGAAAAACTGTCTCGCTCCATTCAAAGAGATTCTGCGCCGGATCAACG
ATAAAGATGATGTTCCTCCAGTGAGTTGTATTGTATCGGACGGTGTGATGAGTTTTA
CTCTTGACGCAGCCGAGGAACTAGGTGTCCCAGAGGTTATTTTTTGGACCAATAGT
GCTTGTGGTTTCATGACTATTCTACACTTTTATCTTTTCATCGAGAAGGGTCTATCTC
CTTTTAAAGACGAAAGTTACATGTCAAAGGAGCATCTAGACACAGTTATAGATTGGA
TACCATCAATGAAGAATCTTAGGTTAAAGGACATCCCTAGCTATATACGTACCACAA
ATCCTGACAACATAATGCTTAATTTCCTCATTCGAGAAGTTGAGCGATCTAAACGCG
CTAGTGCTATCATTCTCAACACGTTTGATGAACTCGAGCATGATGTTATCCAGTCTA
TGCAATCTATTTTACCTCCGGTTTATTCTATTGGGCCACTCCATCTCCTTGTGAAGG
AAGAAATAAACGAGGCTAGTGAAATAGGACAGATGGGATTAAATTTGTGGAGAGAG
GAGATGGAATGTTTGGATTGGCTCGATACAAAAACTCCAAACAGTGTTCTTTTTGTT
AACTTTGGATGCATAACGGTGATGAGTGCAAAACAGCTTGAAGAATTTGCTTGGGG
TTTGGCGGCAAGTAGGAAAGAGTTTTTATGGGTGATCCGTCCTAATTTAGTGGTGG
GAGAGGCGATGGTGGTTCTTCCACAAGAGTTTTTAGCGGAGACGATAGACCGGAG
AATGTTAGCTAGTTGGTGTCCTCAGGAGAAAGTTCTTTCTCATCCCGCGATAGGAG
GGTTCTTGACGCATTGCGGGTGGAACTCAACATTGGAGAGTCTCGCTGGTGGTGT
TCCGATGATATGTTGGCCATGTTTTTCGGAGCAACCGACGAATTGTAAGTTTTGTTG
TGATGAGTGGGGAGTGGGTATAGAGATTGGTAAAGATGTGAAGAGAGAGGAGGTC
GAGACGGTGGTTAGAGAACTTATGGATGGAGAAAAGGGGAAAAAGCTGAGAGAAA
AGGCGGAAGAGTGGCGGCGGTTGGCCGAGGAAGCGACGAGGTATAAACATGGTT
CGTCGGTCATGAATCTTGAGACGCTTATACATAAAGTTTTCTTAGAAAATCTTAGAT
GA
SEQ ID NO: 93 >UGT86A1
ATGGAGAGAGCAAAGTCGAGGAAGCCTCATATCATGATGATACCATACCCACTTCA
AGGTCACGTTATCCCTTTTGTCCACTTAGCCATCAAACTTGCTTCTCATGGCTTCAC
CATCACTTTCGTCAACACCGACTCCATCCACCACCACATCTCCACCGCTCACCAAG
ATGACGCCGGTGACATCTTCTCCGCCGCTCGCAGCTCCGGCCAGCACGACATACG
TTACACCACCGTGAGCGACGGCTTCCCTTTAGACTTTGACCGGTCACTGAACCATG
ACCAGTTTTTCGAAGGCATTCTCCACGTCTTCTCTGCCCACGTGGATGATCTCATC
GCCAAACTCTCCCGCCGTGATGATCCTCCCGTGACTTGCTTGATCGCCGACACGTT
TTATGTTTGGTCATCTATGATTTGCGACAAGCACAACCTTGTAAATGTCTCGTTTTG
GACCGAACCTGCCTTGGTCCTCAATCTCTATTATCACATGGATCTCCTCATATCTAA
CGGTCATTTCAAATCTCTTGATAATCGTAAAGACGTGATCGATTACGTACCAGGGGT
TAAAGCAATAGAACCAAAGGACTTGATGTCATATCTTCAAGTAAGCGACAAAGACGT
AGACACAAATACAGTAGTATACAGAATATTATTCAAGGCCTTTAAAGACGTCAAGAG
AGCCGACTTCGTCGTATGCAACACGGTGCAAGAGCTCGAACCAGACTCTCTCTCG
GCTCTACAAGCCAAACAACCGGTTTACGCTATCGGTCCGGTTTTCTCAACTGATTC
GGTAGTTCCCACAAGCTTATGGGCCGAGTCAGACTGTACCGAGTGGCTTAAGGGC
CGGCCCACTGGGTCAGTTCTCTACGTCTCGTTTGGTAGCTATGCACATGTTGGTAA
GAAGGAGATTGTTGAGATAGCTCATGGGCTTTTGCTTAGTGGGATTAGTTTCATTTG
GGTTTTACGTCCGGATATAGTTGGATCCAACGTACCAGATTTTCTTCCAGCCGGGT
TTGTGGACCAAGCCCAAGATCGAGGTCTTGTGGTCCAATGGTGCTGCCAGATGGA
AGTTATTTCAAATCCGGCCGTGGGAGGGTTTTTCACACATTGTGGATGGAATTCAAT
TCTAGAGAGCGTTTGGTGTGGTTTGCCTTTGTTGTGTTATCCACTTTTGACAGATCA
GTTCACGAATAGGAAGCTTGTGGTCGATGATTGGTGCATTGGGATTAATCTTTGTG
AGAAGAAGACAATCACAAGGGACCAAGTCTCAGCGAATGTTAAAAGATTGATGAAT
GGAGAAACTTCAAGTGAGCTAAGAAACAATGTTGAAAAGGTTAAACGTCATCTCAAA
GATGCGGTTACAACCGTTGGATCTTCGGAGACGAATTTTAACTTGTTTGTTAGTGAG
GTCCGAAATAGAATAGAAACTAAATTGTGTAATGTAAATGGACTAGAAATAAGTCCA
TCAAACTAA
SEQ ID NO: 94 >UGT86A2
ATGGCGGACGTTAGAAACCCTACAAAAAATCATCATGGTCATCATCATCTTCATGCT
CTCTTGATCCCATATCCATTTCAAGGGCATGTAAACCCATTTGTACACTTAGCCATC
AAGCTCGCGTCACAGGGGATCACCGTCACTTTCGTCAACACTCATTACATCCACCA
CCAGATCACAAACGGCTCCGATGGAGATATTTTCGCTGGAGTTAGGTCAGAGTCTG
GCCTTGACATAAGGTACGCGACGGTTTCCGATGGTTTACCGGTCGGATTTGACCG
GTCGTTGAACCATGACACGTACCAATCGTCGCTGTTGCACGTGTTCTATGCGCATG
TGGAAGAGCTTGTGGCGAGTCTTGTTGGAGGAGACGGCGGTGTGAATGTGATGAT
CGCCGACACATTCTTTGTTTGGCCGTCTGTGGTGGCTAGGAAGTTTGGTTTGGTTT
GTGTCTCGTTTTGGACCGAAGCTGCTTTAGTATTTTCACTTTATTACCATATGGATCT
GCTTCGGATTCATGGCCATTTTGGTGCTCAAGAAACCCGCAGCGATCTAATCGACT
ACATTCCCGGAGTCGCCGCAATTAACCCAAAAGACACGGCGTCGTATCTTCAAGAA
ACCGACACGTCATCAGTAGTTCATCAAATCATCTTCAAAGCATTCGAAGACGTGAAA
AAAGTCGATTTTGTACTCTGCAACACAATTCAGCAATTCGAAGACAAAACAATCAAA
GCCCTAAACACAAAAATCCCATTTTACGCAATCGGACCAATCATACCATTCAATAAC
CAAACCGGTTCAGTCACAACCTCACTCTGGTCTGAATCAGATTGTACACAATGGCT
CAACACTAAACCAAAAAGCTCCGTACTTTATATCTCCTTTGGTAGTTACGCTCATGT
CACAAAGAAGGATCTTGTTGAGATAGCTCACGGGATTTTGTTGAGTAAAGTTAATTT
CGTTTGGGTGGTGAGACCAGACATTGTTAGTTCAGACGAAACCAATCCATTACCAG
AAGGGTTTGAAACAGAAGCTGGAGATCGTGGGATTGTAATACCATGGTGTTGTCAA
ATGACGGTTTTGTCACATGAGAGTGTTGGTGGGTTTTTGACACATTGTGGTTGGAA
CTCGATATTGGAGACGATTTGGTGTGAGGTTCCTGTGTTGTGTTTTCCATTGTTGAC
TGATCAGGTTACGAATAGGAAGCTTGTGGTTGATGATTGGGAGATTGGGATTAATC
TTTGTGAAGATAAGAGTGATTTTGGTAGAGATGAAGTTGGGAGGAATATTAACCGTT
TGATGTGTGGTGTTTCGAAAGAGAAGATCGGACGGGTTAAAATGAGTTTGGAAGGT
GCGGTGAGAAACAGTGGATCTTCTTCGGAGATGAATTTAGGTTTGTTTATTGATGG
ACTTTTGTCTAAGGTTGGTTTATCTAATGGGAAAGCTTAA
SEQ ID NO: 95 >UGT87A1
ATGAATCCAATCAAACCTCAGCCACTCGGAGTCCGCCACGTGGTGGCCATGCCTTG
GCCAGGAAGAGGCCACATCAACCCAATGTTAAACCTCTGCAAAAGCCTCGTCCGGC
GAGACCCAAACCTCACCGTCACATTCGTCGTCACCGAAGAATGGCTCGGGTTCATC
GGGTCCGACCCGAAACCTAACCGGATCCATTTCGCCACTCTCCCCAACATCATTCC
CTCCGAGCTCGTCCGAGCCAACGACTTCATCGCCTTCATCGACGCCGTCCTCACCA
GATTAGAAGAGCCGTTCGAACAGCTACTTGACCGTCTAAACTCTCCTCCCACCGCA
ATCATCGCCGATACTTACATCATTTGGGCAGTACGTGTAGGCACAAAAAGGAATATT
CCGGTGGCTTCTTTCTGGACTACGTCAGCCACGATTCTCTCCCTCTTCATTAACTCC
GATCTTCTCGCAAGTCACGGCCATTTTCCGATCGAACCATCAGAATCAAAACTAGAC
GAGATTGTTGATTACATCCCCGGTTTATCTCCGACAAGACTCAGTGACTTACAGATC
TTACACGGCTATAGTCATCAAGTCTTCAATATATTCAAAAAGTCTTTCGGTGAGCTTT
ATAAAGCTAAGTATCTTCTCTTCCCTTCTGCTTATGAGCTCGAACCAAAAGCCATTG
ACTTTTTCACTTCCAAGTTTGATTTCCCGGTTTACTCCACTGGTCCGTTAATACCCTT
GGAAGAACTATCCGTTGGAAATGAGAATAGAGAACTTGATTACTTTAAGTGGCTTGA
TGAGCAACCTGAAAGCTCTGTTCTTTACATATCTCAAGGGAGTTTTCTTTCAGTCTC
CGAAGCTCAGATGGAGGAGATTGTTGTAGGAGTTAGAGAGGCTGGAGTTAAGTTCT
TTTGGGTGGCTCGTGGGGGTGAGTTAAAGCTTAAGGAGGCTCTTGAAGGTAGCTT
GGGTGTTGTGGTGAGCTGGTGTGATCAGCTACGTGTTTTGTGTCATGCGGCTATAG
GCGGGTTTTGGACGCATTGCGGGTATAACTCGACATTGGAAGGGATATGTTCGGG
AGTACCGTTGCTTACATTTCCTGTTTTTTGGGATCAGTTTCTGAATGCTAAGATGATT
GTTGAGGAGTGGAGAGTTGGAATGGGGATCGAGAGGAAGAAGCAGATGGAGTTGT
TGATAGTGAGTGATGAGATCAAGGAATTGGTAAAAAGGTTTATGGATGGAGAGAGT
GAAGAAGGGAAAGAGATGAGAAGAAGGACTTGTGATCTCAGTGAGATATGTCGTG
GAGCGGTTGCGAAAGGTGGTTCTTCTGATGCTAACATCGATGCTTTCATTAAAGATA
TTACTAAGATCGTGTGA
SEQ ID NO: 96 >UGT87A2
ATGGATCCAAATGAATCTCCACCAAACCAATTTCGCCACGTGGTGGCCATGCCTTA
TCCAGGTCGAGGACACATCAACCCTATGATGAACCTCTGCAAACGCCTTGTCCGTC
GATACCCTAACCTTCACGTCACCTTCGTCGTCACAGAAGAATGGCTCGGGTTTATT
GGACCCGACCCGAAACCCGACCGGATCCATTTCTCCACTCTCCCTAATCTCATCCC
TTCCGAGCTTGTCAGGGCCAAAGACTTCATAGGCTTCATTGATGCCGTCTACACAA
GATTGGAAGAACCATTCGAGAAGCTTCTTGACAGCCTCAATTCACCACCTCCGAGT
GTAATATTCGCCGACACTTACGTCATTTGGGCTGTGCGAGTCGGCAGAAAAAGGAA
TATTCCGGTGGTTTCTCTCTGGACCATGTCAGCCACGATTCTCTCCTTCTTCCTCCA
CTCTGATCTACTCATAAGTCATGGCCATGCTCTGTTCGAACCATCAGAAGAAGAGG
TTGTTGATTACGTCCCCGGTTTATCTCCGACGAAACTCCGAGATTTGCCGCCGATA
TTTGACGGTTACAGCGACCGAGTCTTCAAGACAGCTAAGTTGTGTTTCGATGAACT
ACCAGGAGCTAGGTCTTTACTCTTCACCACCGCCTATGAGCTTGAACACAAAGCTA
TTGACGCTTTCACCTCCAAGCTCGATATCCCGGTCTACGCTATTGGTCCTTTAATAC
CTTTTGAAGAACTTTCTGTTCAAAATGATAACAAGGAACCTAATTACATCCAGTGGC
TTGAGGAACAACCGGAAGGCTCTGTTCTTTACATATCTCAGGGAAGTTTTCTTTCGG
TCTCGGAAGCTCAGATGGAGGAAATAGTGAAAGGACTGAGAGAAAGTGGAGTCCG
GTTTCTTTGGGTGGCTCGTGGGGGCGAGTTAAAGCTTAAGGAGGCTCTTGAAGGT
AGCTTAGGTGTAGTGGTGAGCTGGTGTGATCAGCTTCGGGTGCTGTGTCACAAAG
CTGTAGGCGGGTTTTGGACTCATTGCGGGTTTAACTCGACATTGGAAGGGATATAT
TCAGGAGTACCAATGCTAGCGTTTCCGTTGTTTTGGGATCAGATTCTGAACGCTAA
GATGATTGTTGAGGACTGGAGAGTCGGAATGAGGATCGAGAGGACGAAAAAGAAT
GAGTTGTTGATAGGGAGAGAGGAGATCAAGGAAGTAGTGAAGAGGTTTATGGATA
GAGAGAGTGAAGAAGGGAAAGAGATGAGAAGAAGGGCTTGTGACCTTAGTGAAAT
CAGTCGAGGAGCTGTTGCGAAAAGCGGTTCGTCTAATGTAAACATCGATGAGTTCG
TTCGGCATATTACCAATACAAATTAA
SEQ ID NO: 97 >UGT88A1
ATGGGTGAAGAAGCTATAGTTCTGTATCCTGCACCACCAATAGGTCACTTAGTGTC
CATGGTTGAGTTAGGTAAAACCATCCTCTCCAAAAACCCATCTCTCTCCATCCACAT
TATCTTAGTTCCACCGCCTTATCAGCCGGAATCAACCGCCACTTACATCTCCTCCGT
CTCCTCCTCCTTCCCTTCAATAACCTTCCACCATCTTCCCGCCGTCACACCGTACTC
CTCCTCCTCCACCTCTCGCCACCACCACGAATCTCTCCTCCTAGAGATCCTCTGTTT
TAGCAACCCAAGTGTCCACCGAACTCTTTTCTCACTCTCTCGGAATTTCAATGTCCG
AGCAATGATCATCGATTTCTTCTGCACCGCCGTTTTAGACATCACCGCTGACTTCAC
GTTCCCGGTTTACTTCTTCTACACCTCTGGAGCCGCATGTCTCGCCTTTTCCTTCTA
TCTCCCGACCATCGACGAAACAACCCCCGGAAAAAACCTCAAAGACATTCCTACAG
TTCATATCCCCGGCGTTCCTCCGATGAAGGGCTCCGATATGCCTAAGGCGGTGCTC
GAACGAGACGATGAGGTCTACGATGTTTTTATAATGTTCGGTAAACAGCTCTCGAA
GTCGTCAGGGATTATTATCAATACGTTTGATGCTTTAGAAAACAGAGCCATCAAGGC
CATAACAGAGGAGCTCTGTTTTCGCAATATTTATCCAATTGGACCGCTCATTGTAAA
CGGAAGAATCGAAGATAGAAACGACAACAAGGCAGTTTCTTGTCTCAATTGGCTGG
ATTCGCAGCCGGAAAAGAGTGTTGTGTTTCTCTGTTTTGGAAGCTTAGGTTTGTTCT
CAAAAGAACAGGTGATAGAGATTGCTGTTGGTTTAGAGAAAAGTGGGCAGAGATTC
TTGTGGGTGGTCCGTAATCCACCCGAGTTAGAAAAGACAGAACTGGATTTGAAATC
ACTCTTACCAGAAGGATTCTTAAGCCGAACCGAAGACAAAGGGATGGTCGTGAAAT
CATGGGCTCCGCAAGTTCCGGTTCTGAATCATAAAGCAGTCGGGGGATTCGTCACT
CATTGCGGTTGGAATTCAATTCTTGAAGCTGTTTGTGCTGGTGTGCCGATGGTGGC
TTGGCCGTTGTACGCTGAGCAGAGGTTTAATAGAGTGATGATTGTGGATGAGATCA
AGATTGCGATTTCGATGAATGAATCAGAGACGGGTTTCGTGAGCTCTACAGAGGTG
GAGAAACGAGTCCAAGAGATAATTGGGGAGTGTCCGGTTAGGGAGCGAACCATGG
CTATGAAGAACGCAGCCGAATTAGCCTTGACAGAAACTGGTTCGTCTCATACCGCA
TTAACTACTTTACTCCAGTCGTGGAGCCCAAAGTGA
SEQ ID NO: 98 >UGT89A2
ATGACGGAAGTGTTATTGTTGCCGGGAACTAAATCGGAGAATTCAAAACCACCGCA
CATAGTGGTGTTTCCATTCCCAGCACAAGGCCACTTACTTCCTCTACTTGACTTAAC
TCACCAACTCTGCCTCCGTGGATTCAACGTCTCCGTCATCGTTACTCCCGGTAACC
TTACTTACCTCTCTCCTCTTCTCTCCGCTCATCCCTCCTCCGTCACCTCCGTCGTTT
TCCCTTTCCCTCCTCATCCTTCACTCTCTCCCGGCGTCGAAAACGTTAAAGACGTC
GGAAATTCAGGAAATCTCCCGATCATGGCTTCTCTTCGTCAGCTACGAGAACCAAT
CATCAACTGGTTCCAATCTCATCCGAATCCGCCTATCGCTCTCATCTCCGATTTCTT
CCTCGGATGGACTCACGATCTCTGCAATCAAATCGGTATCCCCAGATTCGCTTTCTT
CTCCATCAGCTTCTTCTTAGTTTCCGTTCTTCAATTTTGCTTCGAGAACATCGATCTA
ATCAAATCAACGGATCCGATTCATCTCCTTGATCTTCCTCGCGCTCCGATTTTCAAA
GAAGAGCATCTTCCGTCTATAGTCCGACGAAGTCTCCAAACTCCGTCACCGGATCT
CGAATCAATCAAAGATTTCTCCATGAATTTGTTGAGCTACGGATCTGTTTTCAATTCT
TCTGAGATTCTGGAAGATGATTATCTTCAGTACGTGAAACAGAGGATGGGTCATGA
TCGGGTTTATGTTATTGGCCCGCTTTGTTCAATCGGGTCGGGTCTTAAATCGAATTC
GGGTTCTGTAGACCCGAGTTTGCTGAGTTGGTTAGACGGATCCCCAAACGGGTCA
GTTCTATACGTTTGTTTCGGAAGTCAAAAGGCGTTGACTAAAGACCAGTGTGATGCT
TTGGCTCTAGGCTTAGAGAAAAGCATGACCCGGTTTGTTTGGGTGGTTAAGAAAGA
TCCGATACCCGACGGGTTTGAGGATCGGGTTTCCGGAAGGGGATTGGTGGTAAGA
GGATGGGTCTCCCAGCTGGCGGTGTTGCGACACGTGGCGGTTGGTGGATTTTTGA
GCCATTGTGGATGGAACTCAGTGCTTGAAGGGATAACGAGTGGGGCTGTGATCTT
GGGCTGGCCCATGGAGGCGGACCAGTTTGTGAACGCGAGGTTGCTTGTGGAGCAT
TTGGGTGTTGCGGTTAGGGTTTGCGAAGGTGGTGAAACTGTGCCTGACTCGGATG
AGTTGGGTCGGGTCATAGCGGAAACGATGGGTGAGGGAGGACGCGAGGTGGCTG
CTCGGGCTGAGGAGATACGGCGGAAGACCGAGGCTGCCGTGACGGAGGCAAATG
GAAGCTCCGTTGAAAATGTACAAAGACTTGTCAAAGAATTTGAAAAAGTCTAA
SEQ ID NO: 99 >UGT89B1
ATGAAAGTGAACGAGGAAAACAACAAGCCGACAAAGACCCATGTCTTAATCTTCCC
ATTTCCGGCGCAAGGTCACATGATTCCCCTCCTCGACTTCACCCACCGCCTTGCTC
TCCGCGGCGGCGCCGCCTTAAAAATAACCGTCCTAGTCACTCCAAAAAACCTTCCT
TTTCTCTCTCCGCTTCTCTCCGCCGTAGTTAACATCGAACCACTTATCCTCCCTTTT
CCCTCCCACCCTTCAATCCCCTCCGGCGTCGAAAACGTCCAAGACTTACCTCCTTC
AGGCTTCCCTTTAATGATCCACGCGCTTGGTAATCTCCACGCGCCGCTTATCTCTT
GGATTACTTCTCACCCTTCTCCTCCAGTAGCCATCGTATCTGATTTCTTCCTTGGTT
GGACCAAAAACCTCGGAATCCCTCGTTTCGATTTCTCTCCCTCCGCTGCTATCACTT
GCTGCATACTCAATACTCTCTGGATCGAAATGCCCACCAAGATCAACGAAGATGAC
GATAACGAGATCCTCCACTTTCCCAAGATCCCGAATTGTCCAAAATACCGTTTTGAT
CAGATCTCCTCTCTTTACAGAAGTTACGTTCACGGAGATCCAGCTTGGGAGTTCATA
AGAGACTCCTTTAGAGATAACGTGGCGAGTTGGGGACTCGTCGTGAACTCGTTCAC
CGCCATGGAAGGTGTTTATCTCGAACATCTTAAGCGAGAGATGGGCCATGATCGTG
TATGGGCTGTAGGCCCAATTATTCCGTTATCTGGGGATAACCGTGGTGGCCCGACT
TCTGTTTCTGTTGATCACGTGATGTCGTGGCTTGACGCACGTGAGGATAACCACGT
GGTGTACGTGTGCTTTGGAAGTCAAGTAGTTTTGACTAAAGAGCAGACTCTTGCAC
TCGCCTCTGGGCTTGAGAAAAGCGGCGTCCATTTCATATGGGCCGTAAAGGAGCC
CGTTGAGAAAGACTCAACACGTGGCAACATCCTGGACGGTTTCGACGATCGCGTG
GCTGGGAGAGGTCTGGTGATCAGAGGATGGGCTCCACAAGTAGCTGTGCTACGTC
ACCGAGCCGTTGGCGCGTTTTTAACGCACTGTGGTTGGAACTCTGTGGTGGAGGC
GGTTGTCGCCGGCGTTTTGATGCTGACGTGGCCGATGAGAGCTGACCAGTACACT
GACGCGTCTCTGGTGGTTGATGAGTTGAAAGTAGGTGTGCGTGCTTGCGAAGGAC
CTGACACGGTGCCTGACCCGGACGAGTTAGCTCGAGTTTTCGCTGATTCCGTGAC
CGGAAATCAAACGGAGAGGATCAAAGCCGTGGAGCTGAGGAAAGCAGCGTTGGAT
GCGATTCAAGAACGTGGGAGCTCAGTGAATGATTTAGATGGATTTATCCAACATGT
CGTTAGTTTAGGACTAAACAAATGA
SEQ ID NO: 100 >UGT89C1
ATGACAACAACAACAACGAAGAAGCCGCACGTTCTGGTGATACCGTTTCCACAATC
CGGTCACATGGTTCCACATCTTGACCTCACGCATCAGATTCTTCTCCGTGGAGCCA
CCGTCACTGTCCTCGTCACACCCAAAAACTCTTCCTATCTCGATGCTCTCCGTTCTC
TTCACTCCCCGGAACACTTCAAAACCCTAATCCTTCCTTTTCCTTCTCACCCTTGTAT
ACCTTCCGGTGTCGAATCTCTCCAGCAACTTCCTCTCGAAGCTATAGTTCACATGTT
TGATGCTCTCTCTCGTCTCCACGACCCTCTCGTTGACTTTCTCAGCCGTCAACCAC
CGTCGGATCTCCCCGACGCCATCCTAGGAAGCTCATTTCTCAGCCCTTGGATTAAC
AAAGTAGCTGATGCTTTCTCTATTAAGTCCATTAGTTTCTTACCCATCAATGCTCATT
CGATCTCCGTCATGTGGGCTCAAGAAGATAGAAGCTTCTTCAACGATCTCGAGACT
GCCACAACGGAAAGCTACGGGCTCGTCATCAACAGTTTCTACGACCTCGAGCCTGA
GTTTGTAGAAACTGTTAAAACACGTTTCCTGAATCACCACCGTATATGGACCGTCGG
ACCGTTGCTCCCCTTTAAAGCTGGCGTTGACCGTGGCGGACAAAGCTCAATCCCG
CCGGCGAAAGTCTCGGCTTGGTTAGATTCGTGCCCCGAGGATAACTCCGTCGTATA
CGTCGGTTTTGGAAGCCAGATCCGGCTCACGGCGGAGCAAACAGCTGCTTTAGCG
GCGGCGTTGGAGAAAAGCAGTGTGCGTTTCATATGGGCGGTGAGAGACGCAGCTA
AGAAGGTGAACTCCAGCGATAACTCCGTTGAGGAAGATGTGATCCCGGCGGGATT
TGAAGAGAGAGTGAAGGAGAAAGGACTCGTGATAAGAGGATGGGCCCCACAAACT
ATGATTCTTGAGCATCGAGCCGTTGGATCTTACCTAACTCATTTGGGTTGGGGTTC
GGTTCTGGAAGGAATGGTCGGAGGAGTTATGTTGCTAGCGTGGCCGATGCAAGCA
GACCATTTCTTTAACACGACGCTCATCGTTGATAAACTAAGAGCCGCAGTGCGAGT
TGGAGAGAACAGAGACTCGGTTCCTGACTCGGACAAGCTCGCTAGGATTTTGGCT
GAGTCGGCGAGAGAGGACTTGCCGGAGAGAGTTACGTTGATGAAGCTGAGGGAG
AAAGCTATGGAGGCCATTAAAGAAGGTGGGAGCTCTTACAAGAACTTGGATGAGCT
CGTTGCAGAGATGTGTTTGTAA
SEQ ID NO: 101 >UGT90A1
ATGTCCGTTTCAACACATCACCACCACGTGGTCCTCTTCCCTTTCATGTCAAAAGGC
CACATCATCCCTCTCCTCCAATTCGGTCGTCTCCTCCTCCGTCACCACCGCAAAGA
ACCAACCATCACCGTCACCGTTTTCACCACTCCCAAGAACCAACCTTTCATCTCAGA
CTTCCTCTCGGATACGCCGGAGATCAAAGTCATCTCTCTCCCTTTCCCGGAAAACA
TCACCGGAATCCCTCCCGGCGTCGAGAACACCGAAAAGCTCCCATCCATGTCACTT
TTCGTCCCCTTCACACGCGCCACGAAGCTTCTCCAACCTTTCTTCGAAGAAACACTC
AAGACTCTTCCAAAAGTTTCGTTCATGGTCTCTGATGGATTCCTCTGGTGGACATCG
GAGTCTGCAGCTAAGTTCAACATTCCAAGATTTGTCTCCTACGGCATGAACTCTTAC
TCCGCCGCTGTCTCCATCTCTGTTTTCAAACACGAACTCTTTACCGAACCGGAAAGT
AAATCTGATACCGAACCGGTCACTGTACCAGACTTTCCATGGATCAAGGTCAAGAA
GTGTGATTTCGACCATGGCACTACCGAGCCGGAAGAATCAGGTGCAGCCCTCGAA
CTATCTATGGACCAAATCAAGTCGACCACCACAAGCCATGGGTTTTTAGTCAATAGC
TTCTACGAGCTCGAGTCAGCATTTGTTGATTACAACAACAACTCTGGTGATAAACCA
AAGTCGTGGTGTGTTGGGCCACTGTGTTTGACAGATCCTCCTAAACAGGGGAGTG
CTAAACCGGCTTGGATTCATTGGTTGGATCAGAAGCGAGAGGAAGGGCGTCCGGT
TTTGTACGTGGCGTTTGGAACGCAGGCAGAGATATCGAACAAGCAGCTTATGGAAC
TAGCTTTCGGCTTGGAAGATTCAAAGGTGAACTTTCTGTGGGTCACAAGAAAAGAT
GTGGAGGAGATTATTGGAGAAGGATTCAACGATAGAATAAGAGAGAGTGGGATGAT
AGTGAGAGATTGGGTGGACCAATGGGAGATATTGTCACATGAAAGTGTCAAAGGAT
TTTTGAGCCATTGTGGGTGGAACTCAGCACAAGAGAGCATATGTGTCGGGGTCCCA
TTGTTGGCTTGGCCGATGATGGCCGAGCAACCGCTCAATGCGAAGATGGTTGTGG
AGGAGATAAAGGTGGGAGTAAGAGTTGAAACGGAAGATGGGAGTGTAAAAGGTTTT
GTGACAAGAGAAGAACTAAGTGGAAAGATTAAAGAACTGATGGAAGGAGAAACGG
GGAAAACCGCAAGAAAGAATGTAAAAGAATATTCGAAAATGGCGAAAGCGGCTTTG
GTCGAAGGGACTGGTTCGTCATGGAAGAATTTAGATATGATTCTTAAGGAGTTATGT
AAGAGTAGAGATTCAAACGGTGCTAGTGAGTAG
SEQ ID NO: 102 >UGT90A2
ATGGAGTTAGAAAAAGTTCACGTGGTTTTGTTCCCATACTTGTCCAAAGGGCACATG
ATTCCTATGCTCCAATTAGCTCGTCTCCTCTTATCCCACTCCTTCGCCGGAGACATC
TCCGTCACCGTCTTCACCACTCCTTTGAACCGTCCTTTCATCGTTGACTCACTCTCC
GGCACCAAAGCGACCATCGTCGACGTACCTTTCCCTGATAACGTCCCGGAGATCCC
ACCCGGCGTCGAGTGCACTGACAAACTCCCTGCTTTGTCGTCCTCCCTCTTCGTTC
CTTTCACAAGAGCCACCAAGTCAATGCAGGCAGACTTTGAGCGAGAGCTCATGTCA
CTGCCACGTGTCAGTTTCATGGTCTCAGACGGTTTCTTGTGGTGGACGCAAGAGTC
AGCTCGAAAGCTAGGGTTTCCTCGGCTTGTTTTCTTTGGTATGAATTGCGCTTCCAC
CGTTATATGTGACAGTGTTTTTCAAAACCAGCTTCTATCTAATGTTAAGTCCGAGAC
GGAGCCAGTTTCTGTACCGGAGTTTCCGTGGATTAAGGTTAGGAAATGTGATTTCG
TTAAAGATATGTTTGATCCAAAAACCACCACAGATCCTGGATTCAAGCTTATCCTAG
ATCAAGTCACGTCTATGAATCAAAGCCAAGGTATCATATTCAATACATTTGACGACC
TTGAACCCGTGTTTATTGATTTCTACAAGCGTAAACGCAAACTCAAGCTTTGGGCAG
TTGGACCGCTTTGTTACGTAAATAACTTGGCTTGGATGATGAAGTAGAAGAGAAGG
TCAAACCTAGTTGGATGAAATGGCTAGATGAAAAGCGAGACAAGGGATGCAATGTT
CTGTATGTGGCTTTCGGGTCACAAGCCGAGATCTCGAGAGAACAACTAGAGGAGAT
TGCGTTAGGGTTGGAAGAATCGAAGGTGAACTTCTTGTGGGTGGTCAAAGGAAATG
AAATAGGAAAAGGGTTTGAAGAGAGAGTGGGAGAAAGAGGAATGATGGTGAGAGA
TGAATGGGTTGATCAGAGGAAGATATTAGAGCACGAGAGTGTTAGAGGGTTCTTGA
GCCATTGTGGGTGGAATTCTCTGACGGAGAGCATTTGCTCGGAGGTTCCAATCTTG
GCGTTTCCTTTAGCAGCGGAGCAACCTCTGAATGCGATTTTGGTGGTGGAAGAGCT
GAGAGTGGCGGAGAGAGTGGTGGCGGCGAGTGAAGGGGTTGTGAGAAGAGAAGA
GATTGCAGAGAAAGTGAAGGAGTTGATGGAGGGAGAGAAAGGGAAAGAGCTGAGG
AGGAATGTCGAGGCATATGGTAAGATGGCGAAGAAGGCTTTGGAGGAAGGTATTG
GTTCGTCTAGGAAGAATTTAGACAACCTTATCAACGAGTTTTGTAACAATGGAACAT
GA
SEQ ID NO: 103 >UGT90A4
ATGGCCGTTTCATCGTCGCATCATGCGGTTCTCTTCCCTTACATGTCAAAAGGCCA
CACGATTCCTCTCCTCCAATTCGCCCGTCTCCTCCTCCGTCACCGCCGTATCGTCT
CCGTAGACGACGAAGAACCAACCATTTCCGTCACCGTCTTCACCACCCCAAAAAAC
CAACCATTCGTCTCAAACTTCCTCTCTGACGTCGCATCATCTATCAAAGTAATCTCC
CTCCCTTTCCCTGAAAACATCGCCGGAATCCCTCCCGGCGTCGAGAGCACCGACAT
GCTCCCTTCCATATCACTTTACGTGCCCTTCACGCGCGCAACCAAATCTCTCCAGC
CTTTCTTCGAAGCAGAACTCAAGAATCTTGAGAAAGTTTCTTTCATGGTCTCCGATG
GATTCTTATGGTGGACATCGGAATCCGCCGCTAAATTTGAGATCCCGAGACTTGCC
TTCTACGGCATGAACTCCTACGCATCGGCTATGTGCTCCGCCATTTCGGTACACGA
GCTCTTTACCAAACCGGAAAGTGTTAAATCTGATACTGAACCGGTTACTGTACCGGA
TTTTCCATGGATATGTGTTAAGAAGTGTGAGTTCGATCCGGTTTTGACCGAACCGG
ATCAATCGGATCCAGCGTTCGAGCTACTCATTGACCATCTTATGTCCACCAAGAAAA
GCCGTGGAGTTATAGTGAACAGCTTTTACGAGCTCGAGTCAACGTTCGTTGACTAC
CGGCTCCGTGATAACGATGAACCAAAACCGTGGTGTGTTGGGCCTTTGTGTTTGGT
AAATCCTCCAAAACCGGAGAGTGATAAACCGGATTGGATTCATTGGTTGGACCGGA
AACTAGAGGAAAGATGTCCGGTTATGTATGTGGCGTTTGGAACGCAGGCTGAGATA
TCGAACGAGCAGCTCAAGGAAATAGCATTAGGGTTGGAAGATTCCAAGGTCAATTT
CTTGTGGGTCACGAGAAAGGACTTGGAAGAAGTAACTGGAGGATTAGGGTTCGAA
AAGAGAGTGAAAGAGCATGGGATGATTGTGAGAGATTGGGTAGACCAATGGGAGA
TATTGTCACATAAAAGTGTCAAAGGGTTTTTGAGTCATTGTGGATGGAACTCGGCG
CAAGAGAGTATTTGCGCTGGGGTTCCACTACTCGCTTGGCCAATGATGGCAGAGC
AGCCACTCAATGCGAAGTTGGTAGTGGAGGAGCTAAAGATCGGAGTAAGAATCGAA
ACAGAAGATGTAAGTGTGAAAGGATTCGTGACAAGAGAAGAACTTAGTCGAAAGGT
TAAACAATTGATGGAGGGAGAGATGGGGAAGACAACGATGAAGAATGTAAAAGAGT
ATGCGAAAATGGCGAAAAAAGCTATGGCTCAAGGGACTGGTTCGTCTTGGAAGAGT
TTGGATTCGCTTCTGGAAGAGCTTTGTAAGAGTAGAGAGCCAGACGGTGTTAATAA
GTTGTCAAGTTCTGATGCTTAG
SEQ ID NO: 104 >UGT91A1
ATGACAAACTTCAAAGACAACGATGGAGATGGAACCAAACTCCACGTGGTAATGTT
TCCATGGTTAGCCTTTGGTCACATGGTTCCATACTTGGAGCTCTCTAAACTCATAGC
TCAAAAGGGTCACAAAGTCTCTTTCATTTCCACTCCACGTAACATCGACCGTCTCCT
CCCATGGTTACCGGAAAATCTCTCCTCCGTCATTAACTTCGTCAAGCTATCACTTCC
CGTCGGCGACAACAAACTCCCGGAAGACGGTGAAGCTACCACAGACGTCCCTTTC
GAACTCATACCTTACTTAAAAATCGCTTACGACGGGTTAAAAGTTCCGGTGACGGA
GTTTCTTGAATCTTCGAAACCCGATTGGGTTCTTCAAGATTTCGCGGGGTTTTGGCT
TCCTCCAATCTCTCGTCGTCTCGGAATCAAAACCGGATTCTTTAGCGCTTTCAACGG
CGCGACGCTCGGTATTCTTAAACCGCCGGGGTTCGAAGAGTACCGTACTTCGCCG
GCGGATTTTATGAAGCCGCCTAAGTGGGTTCCGTTTGAAACTTCGGTAGCTTTCAA
GTTATTTGAATGCAGGTTCATTTTCAAAGGATTTATGGCGGAAACCACCGAAGGGA
ATGTTCCCGACATCCACCGTGTCGGCGGCGTAATTGACGGCTGTGACGTCATCTTC
GTACGGAGCTGTTACGAGTATGAAGCGGAGTGGTTAGGACTTACACAAGAACTTCA
CCGGAAACCGGTTATACCGGTCGGAGTTTTGCCTCCAAAACCGGACGAAAAGTTTG
AAGATACCGACACGTGGCTGTCTGTTAAAAAATGGTTGGACTCACGGAAAAGTAAG
TCCATTGTCTACGTAGCTTTTGGTTCAGAAGCTAAACCGAGTCAAACGGAGCTAAAT
GAGATCGCTCTCGGTTTAGAGCTTTCTGGTTTACCTTTCTTTTGGGTGTTAAAGACT
CGTCGTGGTCCGTGGGATACCGAACCGGTCGAGCTTCCGGAAGGATTCGAAGAGC
GTACAGCGGATAGAGGGATGGTGTGGAGAGGTTGGGTTGAGCAATTGCGTACATT
GAGCCATGACTCGATCGGTTTGGTTCTGACTCATCCCGGTTGGGGAACGATAATTG
AAGCTATCCGGTTTGCTAAACCGATGGCAATGCTGGTTTTTGTGTATGACCAAGGA
TTGAATGCGAGAGTCATTGAAGAGAAGAAAATTGGGTATATGATCCCTCGAGACGA
GACAGAAGGTTTCTTTACTAAAGAAAGTGTTGCGAATTCGCTAAGATTGGTAATGGT
GGAAGAAGAAGGAAAGGTTTATAGAGAGAATGTGAAGGAGATGAAAGGAGTGTTTG
GAGATATGGATAGACAAGATCGTTATGTGGATTCATTCTTGGAATATCTTGTTACTA
ATCGTTAA
SEQ ID NO: 105 >UGT91B1
ATGGCCGAGCCAAAACCGAAGCTTCATGTTGCAGTGTTCCCATGGTTAGCTTTAGG
TCACATGATTCCTTACTTGCAACTCTCAAAGCTCATAGCAAGGAAAGGCCATACTGT
GTCCTTCATCTCCACAGCTCGTAACATTTCACGTCTTCCCAATATATCCTCCGACCT
TTCCGTGAATTTCGTTTCTTTGCCGTTAAGTCAAACCGTCGACCATCTCCCAGAGAA
CGCTGAGGCCACCACTGATGTCCCGGAGACTCACATAGCTTATCTGAAGAAAGCAT
TTGATGGGCTTTCTGAAGCTTTCACAGAGTTTTTAGAAGCTTCCAAACCAAACTGGA
TAGTGTATGATATCTTGCACCATTGGGTCCCGCCTATCGCTGAGAAGCTCGGCGTG
AGACGAGCCATCTTCTGCACGTTCAACGCAGCTTCCATCATCATCATCGGTGGGCC
AGCATCAGTCATGATTCAAGGTCATGACCCTCGAAAGACTGCTGAAGATCTTATCGT
GCCTCCACCATGGGTCCCGTTTGAGACCAACATAGTTTACCGTCTCTTTGAAGCTA
AGAGGATCATGGAGTATCCCACGGCAGGTGTAACTGGAGTTGAATTGAACGACAAC
TGTAGATTGGGTTTGGCTTACGTTGGCTCTGAGGTTATTGTGATTAGATCATGTATG
GAACTCGAACCTGAGTGGATTCAATTGCTCAGTAAACTCCAAGGAAAGCCTGTGAT
TCCAATTGGTTTACTCCCGGCTACACCAATGGATGATGCAGATGACGAGGGAACAT
GGTTAGACATCAGAGAATGGCTAGACAGACATCAAGCAAAGTCTGTGGTTTATGTA
GCCTTAGGAACTGAAGTGACAATTAGTAACGAAGAGATTCAAGGTTTAGCTCATGG
GTTGGAGCTTTGCAGGTTACCTTTCTTTTGGACGCTAAGGAAGAGGACTAGAGCTT
CTATGCTACTACCTGATGGGTTCAAAGAGAGAGTCAAAGAGCGTGGAGTCATTTGG
ACCGAGTGGGTACCTCAGACCAAGATACTGAGCCATGGTTCAGTTGGTGGGTTTGT
TACTCATTGTGGTTGGGGATCAGCTGTGGAAGGGCTTAGCTTTGGTGTCCCTTTGA
TCATGTTTCCATGTAACCTAGACCAGCCGCTAGTGGCTAGGTTGCTCAGTGGGATG
AATATAGGCTTGGAGATTCCAAGGAATGAGCGAGACGGGCTGTTCACGAGTGCTTC
TGTTGCAGAGACAATCAGACATGTTGTTGTGGAAGAAGAAGGAAAGATCTACAGGA
ACAATGCTGCATCTCAGCAAAAGAAAATATTCGGGAACAAGAGATTGCAAGATCAGT
ATGCGGATGGTTTTATCGAGTTTCTGGAGAATCCTATAGCAGGAGTGTAG
SEQ ID NO: 106 >UGT91C1
ATGGTCGACAAGAGAGAAGAAGTTATGCACGTAGCCATGTTTCCATGGCTAGCTAT
GGGTCATCTCCTTCCTTTTCTTCGTCTCTCCAAGTTACTAGCTCAAAAGGGTCACAA
GATCTCTTTCATATCAACACCAAGAAACATCGAAAGACTTCCTAAATTACAATCAAAC
CTCGCCTCCTCCATCACCTTCGTCTCTTTCCCTCTCCCTCCCATCTCAGGCTTGCCT
CCTTCTTCAGAATCATCCATGGACGTTCCTTACAACAAGCAACAGTCTCTTAAAGCC
GCTTTTGATCTTCTTCAGCCACCGTTGAAAGAGTTTCTCCGACGGTCTTCTCCGGAT
TGGATCATATACGACTATGCTTCTCACTGGCTTCCTTCTATTGCGGCCGAGCTTGG
AATCTCTAAGGCTTTCTTTAGTCTCTTTAACGCAGCTACTCTCTGTTTCATGGGACC
GTCTTCGTCTTTGATTGAAGAAATTAGATCAACGCCGGAAGATTTCACGGTGGTGC
CACCGTGGGTCCCGTTCAAGTCAAACATCGTGTTTCGTTATCATGAAGTTACTAGAT
ACGTTGAGAAGACAGAGGAAGATGTAACCGGAGTCTCTGACTCAGTTCGGTTTGGT
TACTCGATTGACGAAAGCGATGCGGTTTTTGTCCGTAGCTGTCCGGAGTTTGAACC
GGAATGGTTTGGTTTACTAAAAGACCTGTACCGTAAACCGGTATTTCCAATCGGGTT
TTTGCCTCCGGTTATTGAAGACGACGATGCCGTTGATACTACATGGGTTCGTATAAA
GAAGTGGCTCGACAAGCAACGGCTTAATTCAGTTGTTTACGTGTCACTTGGCACCG
AAGCGAGTCTTCGTCATGAGGAAGTAACTGAGCTAGCTCTTGGGTTAGAGAAGTCA
GAGACACCGTTCTTTTGGGTCCTAAGGAACGAGCCAAAGATTCCAGATGGGTTCAA
AACACGAGTCAAGGGACGTGGAATGGTTCATGTTGGTTGGGTTCCACAAGTGAAAA
TACTTAGTCACGAGTCAGTAGGAGGGTTCTTGACACATTGTGGTTGGAACTCAGTG
GTGGAAGGGTTAGGGTTTGGTAAAGTTCCAATCTTTTTTCCGGTGTTGAATGAGCA
AGGACTTAATACGAGGTTGTTGCATGGGAAAGGACTTGGTGTTGAGGTTTCAAGAG
ATGAGAGAGATGGGTCGTTTGATTCTGACTCGGTCGCTGACTCGATTAGGTTGGTG
ATGATTGATGATGCTGGCGAGGAGATAAGGGCTAAGGCTAAAGTGATGAAGGATTT
GTTTGGGAACATGGATGAGAATATTCGTTATGTTGACGAACTTGTTAGGTTTATGAG
AAGTAAAGGATCATCATCATCATCATGA
SEQ ID NO: 107 >UGT92A1
ATGGCGGAAGCTAAACCCAGAAATCTGAGAATCGTGATGTTCCCTTTCATGGGACA
AGGCCATATCATCCCGTTTGTAGCTTTAGCCCTTCGTTTAGAGAAGATTATGATTAT
GAACAGAGCCAACAAAACCACCATCTCTATGATCAATACTCCTTCGAACATCCCCAA
AATACGCTCCAATCTTCCACCTGAATCCTCCATAAGTCTCATAGAGTTACCTTTCAA
CAGCTCTGATCATGGCCTTCCTCACGACGGCGAGAATTTCGATTCTCTTCCTTACTC
TCTCGTCATCAGCCTTCTTGAAGCTTCTAGGTCGCTTCGTGAGCCCTTTCGAGACTT
CATGACGAAGATCTTGAAGGAAGAAGGGCAGAGCTCGGTTATAGTGATCGGTGATT
TCTTCTTGGGTTGGATCGGTAAGGTTTGCAAAGAGGTTGGTGTTTATTCAGTGATCT
TTAGTGCTTCTGGTGCTTTTGGTTTAGGTTGTTATAGATCCATATGGTTAAACTTGC
CACATAAAGAAACCAAACAAGATCAGTTTCTCTTAGATGATTTCCCTGAAGCAGGGG
AGATTGAGAAAACTCAGTTGAATTCTTTCATGTTAGAAGCTGATGGAACCGATGATT
GGTCTGTTTTCATGAAGAAGATTATACCTGGATGGTCTGACTTCGATGGATTCTTGT
TCAACACGGTTGCTGAAATCGATCAGATGGGATTATCCTACTTCCGTAGAATAACCG
GTGTTCCGGTTTGGCCAGTTGGGCCGGTTTTGAAGTCTCCGGATAAGAAGGTGGG
ATCGAGGTCGACAGAGGAAGCAGTGAAGTCATGGCTTGACTCAAAACCGGACCATT
CGGTTGTGTACGTATGTTTCGGTTCAATGAACTCGATTTTGCAAACGCATATGTTAG
AATTGGCTATGGCATTAGAGAGTAGCGAGAAGAACTTCATATGGGTGGTGAGGCC
GCCCATAGGTGTGGAGGTGAAGAGTGAGTTTGATGTGAAAGGGTATCTACCGGAA
GGATTTGAGGAAAGAATAACAAGATCGGAAAGAGGGTTACTTGTGAAGAAATGGGC
ACCACAAGTTGATATATTGTCACACAAGGCAACATGTGTGTTTTTGAGTCATTGCGG
ATGGAACTCGATACTCGAATCACTTAGCCACGGTGTGCCACTGCTCGGATGGCCCA
TGGCAGCCGAGCAGTTCTTCAATTCCATATTGATGGAGAAACATATTGGGGTATCG
GTTGAGGTGGCGCGTGGGAAGAGATGTGAGATCAAATGTGATGACATTGTTTCTAA
GATCAAACTGGTGATGGAGGAGACTGAAGTAGGGAAAGAGATTAGGAAGAAGGCT
AGAGAGGTGAAGGAGTTAGTGAGGAGAGCAATGGTAGATGGAGTTAAAGGTTCCT
CCGTCATTGGTTTGGAAGAGTTTCTTGACCAAGCAATGGTCAAGAAAGTGGAGAAT
TGA

TABLE 2

71C1
Nucleotide sequence (SEQ ID NO: 7)
ATGGGGAAGCAAGAAGATGCAGAGCTCGTCATCATACCTTTCCCTTTCTCCGGACA
CATTCTCGCAACAATCGAACTCGCCAAACGTCTCATAAGTCAAGACAATCCTCGGAT
CCACACCATCACCATCCTCTATTGGGGATTACCTTTTATTCCTCAAGCTGACACAAT
CGCTTTCCTCCGATCCCTAGTCAAAAATGAGCCTCGTATCCGTCTCGTTACGTTGC
CCGAAGTCCAAGACCCTCCACCAATGGAACTCTTTGTGGAATTTGCCGAATCTTAC
ATTCTTGAATACGTCAAGAAAATGGTTCCCATCATCAGAGAAGCTCTCTCCACTCTC
TTGTCTTCCCGCGATGAATCGGGTTCAGTTCGTGTGGCTGGATTGGTTCTTGACTT
CTTCTGCGTCCCTATGATCGATGTAGGAAACGAGTTTAATCTCCCTTCTTACATTTT
CTTGACGTGTAGCGCAGGGTTCTTGGGTATGATGAAGTATCTTCCAGAGAGACACC
GCGAAATCAAATCGGAATTCAACCGGAGCTTCAACGAGGAGTTGAATCTCATTCCT
GGTTATGTCAACTCTGTTCCTACTAAGGTTTTGCCGTCAGGTCTATTCATGAAAGAG
ACCTACGAGCCTTGGGTCGAACTAGCAGAGAGGTTTCCTGAAGCTAAGGGTATTTT
GGTTAATTCATACACAGCTCTCGAGCCAAACGGTTTTAAATATTTCGATCGTTGTCC
GGATAACTACCCAACCATTTACCCAATCGGGCCGATATTATGCTCCAACGACCGTC
CGAATTTGGACTCATCGGAACGAGATCGGATCATAACTTGGCTAGATGACCAACCC
GAGTCATCGGTCGTGTTCCTCTGTTTCGGGAGCTTGAAGAATCTCAGCGCTACTCA
GATCAACGAGATAGCTCAAGCCTTAGAGATCGTTGACTGCAAATTCATCTGGTCGT
TTCGAACCAACCCGAAGGAGTACGCGAGCCCTTACGAGGCTCTACCACACGGGTT
CATGGACCGGGTCATGGATCAAGGCATTGTTTGTGGTTGGGCTCCTCAAGTTGAAA
TCCTAGCCCATAAAGCTGTGGGAGGATTCGTATCTCATTGTGGTTGGAACTCGATA
TTGGAGAGTTTGGGTTTCGGCGTTCCAATCGCCACGTGGCCGATGTACGCGGAAC
AACAACTAAACGCGTTCACGATGGTGAAGGAGCTTGGTTTAGCCTTGGAGATGCGG
TTGGATTACGTGTCGGAAGATGGAGATATAGTGAAAGCTGATGAGATCGCAGGAAC
CGTTAGATCTTTAATGGACGGTGTGGATGTGCCGAAGAGTAAAGTGAAGGAGATTG
CTGAGGCGGGAAAAGAAGCTGTGGACGGTGGATCTTCGTTTCTTGCGGTTAAAAG
ATTCATCGGTGACTTGATCGACGGCGTTTCTATAAGTAAGTAG
Amino acid sequence (SEQ ID NO: 108)
MGKQEDAELVIIPFPFSGHILATIELAKRLISQDNPRIHTITILYWGLPFIPQADTIAFLRSLVKNE
PRIRLVTLPEVQDPPPMELFVEFAESYILEYVKKMVPIIREALSTLLSSRDESGSVRVAGLVLD
FFCVPMIDVGNEFNLPSYIFLTCSAGFLGMMKYLPERHREIKSEFNRSFNEELNLIPGYVNSV
PTKVLPSGLFMKETYEPWVELAERFPEAKGILVNSYTALEPNGFKYFDRCPDNYPTIYPIGPI
LCSNDRPNLDSSERDRIITWLDDQPESSVVFLCFGSLKNLSATQINEIAQALEIVDCKFIWSFR
TNPKEYASPYEALPHGFMDRVMDQGIVCGWAPQVEILAHKAVGGFVSHCGWNSILESLGF
GVPIATWPMYAEQQLNAFTMVKELGLALEMRLDYVSEDGDIVKADEIAGTVRSLMDGVDVP
KSKVKEIAEAGKEAVDGGSSFLAVKRFIGDLIDGVSISK
71C2
Nucleotide sequence (SEQ ID NO: 8)
ATGGCGAAGCAGCAAGAAGCAGAGCTCATCTTCATCCCATTTCCAATCCCCGGACA
CATTCTCGCCACAATCGAACTCGCGAAACGTCTCATCAGTCACCAACCTAGTCGGA
TCCACACCATCACCATCCTCCATTGGAGCTTACCTTTTCTTCCTCAATCTGACACTA
TCGCCTTCCTCAAATCCCTAATCGAAACAGAGTCTCGTATCCGTCTCATTACCTTAC
CCGATGTCCAAAACCCTCCACCAATGGAGCTATTTGTGAAAGCTTCCGAATCTTACA
TTCTTGAATACGTCAAGAAAATGGTTCCTTTGGTCAGAAACGCTCTCTCCACTCTCT
TGTCTTCTCGTGATGAATCGGATTCAGTTCATGTCGCCGGATTAGTTCTTGATTTCT
TCTGTGTCCCTTTGATCGATGTCGGAAACGAGTTTAATCTCCCTTCTTACATCTTCT
TGACGTGTAGCGCAAGTTTCTTGGGTATGATGAAGTATCTTCTGGAGAGAAACCGC
GAAACCAAACCGGAACTTAACCGGAGCTCTGACGAGGAAACAATATCAGTTCCTGG
TTTTGTTAACTCCGTTCCGGTTAAAGTTTTGCCACCGGGTTTGTTCACGACTGAGTC
TTACGAAGCTTGGGTCGAAATGGCGGAAAGGTTCCCTGAAGCCAAGGGTATTTTGG
TCAATTCATTTGAATCTCTAGAACGTAACGCTTTTGATTATTTCGATCGTCGTCCGG
ATAATTACCCACCCGTTTACCCAATCGGGCCAATTCTATGCTCCAACGATCGTCCGA
ATTTGGATTTATCGGAACGAGACCGGATCTTGAAATGGCTCGATGACCAACCCGAG
TCATCTGTTGTGTTTCTCTGCTTCGGGAGCTTGAAGAGTCTCGCTGCGTCTCAGAT
TAAAGAGATCGCTCAAGCCTTAGAGCTCGTCGGAATCAGATTCCTCTGGTCGATTC
GAACGGACCCGAAGGAGTACGCGAGCCCGAACGAGATTTTACCGGACGGGTTTAT
GAACCGAGTCATGGGTTTGGGCCTTGTTTGTGGTTGGGCTCCTCAAGTTGAAATTC
TGGCCCATAAAGCAATTGGAGGGTTCGTGTCACACTGCGGTTGGAACTCGATATTG
GAGAGTTTGCGTTTCGGAGTTCCAATTGCCACGTGGCCAATGTACGCGGAACAACA
ACTAAACGCGTTCACGATTGTGAAGGAGCTTGGTTTGGCGTTGGAGATGCGGTTG
GATTACGTGTCGGAATATGGAGAAATCGTGAAAGCTGATGAAATCGCAGGAGCCGT
ACGATCTTTGATGGACGGTGAGGATGTGCCGAGGAGGAAACTGAAGGAGATTGCG
GAGGCGGGAAAAGAGGCTGTGATGGACGGTGGATCTTCGTTTGTTGCGGTTAAAA
GATTCATAGATGGGCTTTGA
Amino acid sequence (SEQ ID NO: 109)
MAKQQEAELIFIPFPIPGHILATIELAKRLISHQPSRIHTITILHWSLPFLPQSDTIAFLKSLIE
TESRIRLITLPDVQNPPPMELFVKASESYILEYVKKMVPLVRNALSTLLSSRDESDSVHVA
GLVLDFFCVPLIDVGNEFNLPSYIFLTCSASFLGMMKYLLERNRETKPELNRSSDEETISV
PGFVNSVPVKVLPPGLFTTESYEAWVEMAERFPEAKGILVNSFESLERNAFDYFDRRPD
NYPPVYPIGPILCSNDRPNLDLSERDRILKWLDDQPESSVVFLCFGSLKSLAASQIKEIAQ
ALELVGIRFLWSIRTDPKEYASPNEILPDGFMNRVMGLGLVCGWAPQVEILAHKAIGGF
VSHCGWNSILESLRFGVPIATWPMYAEQQLNAFTIVKELGLALEMRLDYVSEYGEIVKA
DEIAGAVRSLMDGEDVPRRKLKEIAEAGKEAVMDGGSSFVAVKRFIDGL
71C4
Nucleotide sequence (SEQ ID NO: 10)
ATGGTGAAGGAAACAGAGCTAATCTTCATTCCAGTTCCATCCACAGGTCATATTCTC
GTCCATATTGAATTCGCCAAGCGTCTCATCAATCTCGACCATCGGATCCACACCATC
ACTATTCTCAACTTATCCTCACCCTCTTCTCCTCACGCCTCCGTCTTCGCCAGATCT
CTCATCGCTTCCCAGCCCAAAATCCGTCTCCACGACCTTCCCCCTATCCAAGATCCT
CCTCCATTCGATCTTTACCAAAGAGCTCCCGAAGCTTACATAGTAAAACTCATCAAG
AAAAATACTCCTCTGATAAAAGACGCCGTCTCCAGCATCGTCGCGTCGCGTCGTGG
AGGCTCAGATTCGGTTCAAGTCGCCGGTTTGGTTCTCGATTTATTCTGCAATTCATT
GGTAAAAGATGTTGGCAACGAGCTTAATCTTCCTTCTTACATATACCTTACGTGTAA
CGCTAGATACTTGGGGATGATGAAATATATTCCGGATCGGCATCGGAAAATCGCAT
CTGAGTTCGATTTGAGCTCCGGCGATGAAGAATTGCCGGTTCCGGGATTCATAAAC
GCTATTCCGACGAAATTTATGCCGCCTGGATTGTTCAATAAGGAAGCTTACGAGGC
TTACGTAGAGCTAGCGCCGAGATTCGCAGATGCGAAGGGTATTTTGGTTAATTCCT
TCACGGAGCTTGAGCCGCACCCGTTTGACTATTTCTCTCACCTGGAGAAATTCCCT
CCGGTTTACCCGGTCGGACCGATTCTCAGCTTGAAAGATCGAGCGAGTCCGAACG
AAGAAGCAGTCGATCGGGATCAGATCGTTGGGTGGCTCGATGATCAGCCGGAGTC
ATCGGTGGTGTTCCTCTGTTTCGGGAGCAGAGGAAGCGTTGATGAGCCGCAAGTG
AAGGAGATAGCTCGAGCTTTGGAACTCGTCGGCTGCAGATTTCTTTGGTCAATTAG
AACAAGCGGCGACGTCGAGACGAATCCTAACGATGTGTTGCCGGAGGGGTTCATG
GGCCGAGTAGCAGGCCGAGGTTTGGTATGTGGTTGGGCTCCACAAGTGGAAGTGT
TGGCCCATAAAGCAATAGGAGGATTTGTGTCTCACTGTGGTTGGAACTCCACGCTT
GAAAGCTTATGGTTCGGGGTTCCTGTCGCAACGTGGCCGATGTACGCAGAGCAAC
AGCTTAACGCCTTCACGCTGGTGAAAGAGCTTGGGCTTGCGGTGGACCTGCGGAT
GGATTACGTGTCGAGTCGTGGGGGTTTGGTGACTTGTGATGAGATAGCCAGAGCC
GTACGATCTTTGATGGACGGTGGAGATGAGAAGAGAAAAAAGGTTAAGGAGATGG
CTGATGCGGCAAGGAAGGCTTTGATGGATGGAGGATCGTCTTCTTTGGCAACTGCT
CGATTCATCGCAGAATTGTTTGAAGATGGTTCGTCGTGCTAA
Amino acid sequence (SEQ ID NO: 110)
MVKETELIFIPVPSTGHILVHIEFAKRLINLDHRIHTITILNLSSPSSPHASVFARSLIASQPKI
RLHDLPPIQDPPPFDLYQRAPEAYIVKLIKKNTPLIKDAVSSIVASRRGGSDSVQVAGLVL
DLFCNSLVKDVGNELNLPSYIYLTCNARYLGMMKYIPDRHRKIASEFDLSSGDEELPVPG
FINAIPTKFMPPGLFNKEAYEAYVELAPRFADAKGILVNSFTELEPHPFDYFSHLEKFPPV
YPVGPILSLKDRASPNEEAVDRDQIVGWLDDQPESSVVFLCFGSRGSVDEPQVKEIARA
LELVGCRFLWSIRTSGDVETNPNDVLPEGFMGRVAGRGLVCGWAPQVEVLAHKAIGG
FVSHCGWNSTLESLWFGVPVATWPMYAEQQLNAFTLVKELGLAVDLRMDYVSSRGGL
VTCDEIARAVRSLMDGGDEKRKKVKEMADAARKALMDGGSSSLATARFIAELFEDGSSC
71D1
Nucleotide sequence (SEQ ID NO: 12)
ATGCGGAATGTAGAGCTCATCTTCATCCCCACACCAACCGTTGGTCATCTTGTTCC
GTTTCTTGAATTTGCTAGGCGTCTCATTGAGCAAGATGATAGGATCCGTATCACAAT
CCTCTTGATGAAACTACAAGGTCAGTCTCATCTAGACACTTATGTTAAATCAATTGC
CTCCTCTCAACCGTTTGTTAGATTCATTGATGTCCCTGAGTTAGAGGAGAAACCTAC
ACTTGGTAGTACACAATCTGTGGAAGCTTATGTGTATGATGTTATTGAGAGAAATAT
CCCTCTTGTGAGGAATATAGTCATGGATATTTTAACTTCTCTTGCATTGGATGGAGT
TAAGGTCAAGGGATTAGTTGTTGACTTTTTCTGTCTCCCTATGATTGACGTTGCTAA
AGATATAAGTCTCCCTTTCTATGTGTTCTTGACTACAAATTCCGGGTTCTTAGCTAT
GATGCAGTATCTAGCAGATCGACATAGTAGAGATACATCGGTTTTTGTAAGAAACTC
GGAAGAAATGTTGTCGATACCTGGATTTGTAAACCCTGTCCCAGCCAATGTTCTGC
CGTCAGCTCTGTTTGTTGAAGATGGTTATGATGCTTACGTTAAGCTGGCCATATTGT
TTACAAAGGCCAATGGAATCCTAGTGAATAGCTCCTTTGATATTGAGCCTTACTCTG
TGAATCATTTTCTTCAAGAACAGAATTATCCTTCTGTTTATGCTGTTGGCCCCATATT
TGACTTGAAAGCCCAGCCTCATCCAGAGCAGGACCTAACCCGTCGTGACGAGTTGA
TGAAATGGCTTGATGATCAACCCGAGGCATCGGTTGTATTCCTTTGTTTTGGGAGT
ATGGCAAGGTTAAGAGGTTCTCTAGTGAAGGAAATAGCTCATGGACTTGAGCTATG
TCAATATAGATTCCTCTGGTCACTCCGTAAAGAAGAGGTGACAAAGGATGATTTGCC
AGAGGGGTTCCTTGACCGTGTCGATGGACGTGGAATGATATGTGGTTGGTCTCCT
CAGGTAGAAATACTGGCCCATAAGGCAGTGGGAGGCTTTGTTTCTCACTGTGGATG
GAACTCAATAGTAGAGAGTTTGTGGTTTGGCGTGCCAATTGTGACATGGCCAATGT
ATGCAGAGCAACAACTCAATGCGTTTCTGATGGTGAAGGAACTGAAGCTAGCTGTG
GAGCTGAAGCTTGATTACAGGGTACATAGTGATGAGATAGTAAACGCAAACGAGAT
AGAGACCGCTATTCGTTATGTAATGGACACGGATAATAATGTTGTGAGGAAACGAG
TGATGGATATCTCGCAGATGATCCAGAGAGCTACGAAGAATGGTGGATCTTCGTTT
GCCGCAATTGAGAAATTCATATATGACGTGATAGGAATTAAGCCCTAG
Amino acid sequence (SEQ ID NO: 111)
MRNVELIFIPTPTVGHLVPFLEFARRLIEQDDRIRITILLMKLQGQSHLDTYVKSIASSQPF
VRFIDVPELEEKPTLGSTQSVEAYVYDVIERNIPLVRNIVMDILTSLALDGVKVKGLVVDF
FCLPMIDVAKDISLPFYVFLTTNSGFLAMMQYLADRHSRDTSVFVRNSEEMLSIPGFVNP
VPANVLPSALFVEDGYDAYVKLAILFTKANGILVNSSFDIEPYSVNHFLQEQNYPSVYAV
GPIFDLKAQPHPEQDLTRRDELMKWLDDQPEASVVFLCFGSMARLRGSLVKEIAHGLEL
CQYRFLWSLRKEEVTKDDLPEGFLDRVDGRGMICGWSPQVEILAHKAVGGFVSHCGW
NSIVESLWFGVPIVTWPMYAEQQLNAFLMVKELKLAVELKLDYRVHSDEIVNANEIETAI
RYVMDTDNNVVRKRVMDISQMIQRATKNGGSSFAAIEKFIYDVIGIKP
72B1
Nucleotide sequence (SEQ ID NO: 14)
ATGGAGGAATCCAAAACACCTCACGTTGCGATCATACCAAGTCCGGGAATGGGTCA
TCTCATACCACTCGTCGAGTTTGCTAAACGACTCGTCCATCTTCACGGCCTCACCG
TTACCTTCGTCATCGCCGGCGAAGGTCCACCATCAAAAGCTCAGAGAACCGTCCTC
GACTCTCTCCCTTCTTCAATCTCCTCCGTCTTTCTCCCTCCTGTTGATCTCACCGAT
CTCTCTTCGTCCACTCGCATCGAATCTCGGATCTCCCTCACCGTGACTCGTTCAAA
CCCGGAGCTCCGGAAAGTCTTCGACTCGTTCGTGGAGGGAGGTCGTTTGCCAACG
GCGCTCGTCGTCGATCTCTTCGGTACGGACGCTTTCGACGTGGCCGTAGAATTTCA
CGTGCCACCGTATATTTTCTACCCAACAACGGCCAACGTCTTGTCGTTTTTTCTCCA
TTTGCCTAAACTAGACGAAACGGTGTCGTGTGAGTTCAGGGAATTAACCGAACCGC
TTATGCTTCCTGGATGTGTACCGGTTGCCGGGAAAGATTTCCTTGACCCGGCCCAA
GACCGGAAAGACGATGCATACAAATGGCTTCTCCATAACACCAAGAGGTACAAAGA
AGCCGAAGGTATTCTTGTGAATACCTTCTTTGAGCTAGAGCCAAATGCTATAAAGGC
CTTGCAAGAACCGGGTCTTGATAAACCACCGGTTTATCCGGTTGGACCGTTGGTTA
ACATTGGTAAGCAAGAGGCTAAGCAAACCGAAGAGTCTGAATGTTTAAAGTGGTTG
GATAACCAGCCGCTCGGTTCGGTTTTATATGTGTCCTTTGGTAGTGGCGGTACCCT
CACATGTGAGCAGCTCAATGAGCTTGCTCTTGGTCTTGCAGATAGTGAGCAACGGT
TTCTTTGGGTCATACGAAGTCCTAGTGGGATCGCTAATTCGTCGTATTTTGATTCAC
ATAGCCAAACAGATCCATTGACATTTTTACCACCGGGATTTTTAGAGCGGACTAAAA
AAAGAGGTTTTGTGATCCCTTTTTGGGCTCCACAAGCCCAAGTCTTGGCGCATCCA
TCCACGGGAGGATTTTTAACTCATTGTGGATGGAATTCGACTCTAGAGAGTGTAGT
AAGCGGTATTCCACTTATAGCATGGCCATTATACGCAGAACAGAAGATGAATGCGG
TTTTGTTGAGTGAAGATATTCGTGCGGCACTTAGGCCGCGTGCCGGGGACGATGG
GTTAGTTAGAAGAGAAGAGGTGGCTAGAGTGGTAAAAGGATTGATGGAAGGTGAA
GAAGGCAAAGGAGTGAGGAACAAGATGAAGGAGTTGAAGGAAGCAGCTTGTAGGG
TGTTGAAGGATGATGGGACTTCGACAAAAGCACTTAGTCTTGTGGCCTTAAAGTGG
AAAGCCCACAAAAAAGAGTTAGAGCAAAATGGCAACCACTAA
Amino acid sequence (SEQ ID NO: 112)
MEESKTPHVAIIPSPGMGHLIPLVEFAKRLVHLHGLTVTFVIAGEGPPSKAQRTVLDSLPS
SISSVFLPPVDLTDLSSSTRIESRISLTVTRSNPELRKVFDSFVEGGRLPTALVVDLFGTD
AFDVAVEFHVPPYIFYPTTANVLSFFLHLPKLDETVSCEFRELTEPLMLPGCVPVAGKDF
LDPAQDRKDDAYKWLLHNTKRYKEAEGILVNTFFELEPNAIKALQEPGLDKPPVYPVGP
LVNIGKQEAKQTEESECLKWLDNQPLGSVLYVSFGSGGTLTCEQLNELALGLADSEQR
FLWVIRSPSGIANSSYFDSHSQTDPLTFLPPGFLERTKKRGFVIPFWAPQAQVLAHPST
GGFLTHCGWNSTLESVVSGIPLIAWPLYAEQKMNAVLLSEDIRAALRPRAGDDGLVRRE
EVARVVKGLMEGFEGKGVRNKMKELKEAACRVLKDDGTSTKALSLVALKWKAHKKELE
QNGNH
72D1
Nucleotide sequence (SEQ ID NO: 18)
ATGGACCAGCCTCACGCGCTTCTAGTGGCTAGCCCTGGCTTGGGTCACCTCATCC
CTATCCTGGAGCTCGGCAACCGTCTCTCCTCCGTCCTAAACATCCACGTCACCATT
CTCGCGGTCACCTCCGGCTCCTCTTCACCGACAGAAACCGAAGCCATACATGCAG
CCGCGGCTAGAACAATCTGTCAAATTACGGAAATTCCCTCGGTGGATGTAGACAAC
CTCGTGGAGCCAGATGCTACAATTTTCACTAAGATGGTGGTGAAGATGCGAGCCAT
GAAGCCCGCGGTACGAGATGCCGTGAAATTAATGAAACGAAAACCAACGGTCATGA
TTGTTGACTTTTTGGGTACGGAACTGATGTCCGTAGCCGATGACGTAGGCATGACG
GCTAAATACGTTTACGTTCCAACTCATGCGTGGTTCTTGGCAGTCATGGTGTACTTG
CCGGTGTTAGATACGGTAGTGGAAGGTGAGTATGTTGATATTAAGGAGCCTTTGAA
GATACCGGGTTGTAAACCGGTCGGACCGAAGGAGCTGATGGAAACGATGTTAGAC
CGGTCGGGCCAGCAATATAAAGAGTGTGTACGAGCTGGCTTAGAGGTACCTATGA
GCGATGGTGTTTTGGTAAATACTTGGGAGGAGTTACAAGGAAACACTCTCGCTGCG
CTTAGAGAGGACGAAGAATTGAGCCGGGTCATGAAAGTACCGGTTTATCCTATTGG
GCCAATTGTTAGGACTAACCAGCATGTAGACAAACCCAATAGTATATTCGAGTGGCT
AGACGAGCAACGGGAAAGGTCAGTGGTGTTTGTGTGTTTAGGGAGCGGTGGAACG
TTGACGTTTGAGCAAACAGTGGAACTCGCTTTGGGTTTAGAGTTAAGTGGTCAAAG
GTTCGTTTGGGTTCTACGTAGGCCCGCTTCATATCTCGGGGCGATCTCCAGCGATG
ATGAACAGGTAAGTGCCAGTCTACCTGAAGGTTTCTTGGACCGCACGCGTGGTGT
GGGGATTGTGGTTACGCAATGGGCACCACAAGTTGAGATCTTGAGCCATAGATCGA
TCGGTGGGTTCTTGTCTCACTGCGGTTGGAGTTCGGCTTTGGAAAGTTTGACTAAA
GGAGTTCCGATCATCGCTTGGCCTCTTTATGCGGAGCAGTGGATGAATGCCACGTT
ATTGACTGAGGAGATCGGTGTGGCCGTTCGTACATCGGAGTTACCGTCGGAGAGA
GTCATCGGAAGGGAAGAAGTGGCATCTCTGGTGAGAAAGATTATGGCGGAAGAGG
ATGAAGAAGGACAGAAAATTAGGGCTAAAGCTGAGGAGGTGAGGGTTAGCTCCGA
ACGAGCTTGGAGTAAAGACGGGTCATCTTATAATTCTCTATTCGAATGGGCAAAAC
GATGTTATCTTGTACCGTGA
Amino acid sequence (SEQ ID NO: 113)
MDQPHALLVASPGLGHLIPILELGNRLSSVLNIHVTILAVTSGSSSPTETEAIHAAAARTIC
QITEIPSVDVDNLVEPDATIFTKMVVKMRAMKPAVRDAVKLMKRKPTVMIVDFLGTELMS
VADDVGMTAKYVYVPTHAWFLAVMVYLPVLDTVVEGEYVDIKEPLKIPGCKPVGPKELM
ETMLDRSGQQYKECVRAGLEVPMSDGVLVNTWEELQGNTLAALREDEELSRVMKVPV
YPIGPIVRTNQHVDKPNSIFEWLDEQRERSVVFVCLGSGGTLTFEQTVELALGLELSGQ
RFVWVLRRPASYLGAISSDDEQVSASLPEGFLDRTRGVGIVVTQWAPQVEILSHRSIGG
FLSHCGWSSALESLTKGVPIIAWPLYAEQWMNATLLTEEIGVAVRTSELPSERVIGREE
VASLVRKIMAEEDEEGQKIRAKAEEVRVSSERAWSKDGSSYNSLFEWAKRCYLVP
73B1
Nucleotide sequence (SEQ ID NO: 22)
ATGGGAACTCCTGTCGAAGTCTCTAAGCTCCATTTCTTGCTCTTCCCTTTCATGGCT
CATGGCCATATGATACCAACTCTAGACATGGCTAAGCTCTTTGCCACCAAAGGAGC
TAAATCCACTATCCTCACTACACCTCTCAATGCCAAGCTCTTCTTCGAGAAACCCAT
CAAATCATTCAACCAAGACAACCCGGGACTCGAAGACATCACCATCCAGATCCTTAA
TTTCCCTTGCACAGAGCTTGGTTTGCCTGATGGCTGTGAGAATACTGATTTCATCTT
CTCCACACCTGACCTAAACGTAGGTGACTTGAGTCAAAAGTTTTTACTCGCAATGAA
ATATTTCGAAGAGCCACTAGAGGAGCTCCTCGTGACAATGAGACCAGACTGTCTTG
TCGGTAACATGTTCTTCCCTTGGTCCACTAAAGTTGCTGAGAAGTTCGGAGTACCG
AGACTTGTGTTCCACGGCACAGGCTACTTCTCTTTATGTGCTTCTCATTGCATAAGG
CTCCCTAAGAATGTGGCAACAAGTTCTGAGCCCTTTGTGATTCCTGATCTCCCGGG
AGACATTTTGATTACAGAGGAACAGGTCATGGAGACAGAAGAAGAGTCTGTAATGG
GGAGGTTTATGAAGGCAATAAGAGACTCAGAGAGAGATAGCTTTGGCGTGTTGGT
GAACAGCTTCTACGAGCTTGAACAGGCTTACTCAGATTATTTCAAGAGCTTTGTGGC
GAAAAGAGCGTGGCATATCGGTCCGCTTTCCTTAGGAAATAGAAAGTTCGAGGAGA
AAGCAGAAAGAGGCAAAAAGGCAAGCATTGATGAGCATGAATGTTTGAAATGGCTC
GACTCCAAGAAATGTGATTCAGTGATTTACATGGCCTTTGGAACCATGTCTAGCTTT
AAAAACGAGCAGCTGATAGAGATTGCAGCTGGTTTAGATATGTCAGGACATGATTTT
GTCTGGGTGGTTAACAGAAAAGGCAGCCAAGGTACCATAGACATCACTCTCTTTGC
AGCAAAATCCTCTGTTTTTGTTTTAGAGAAAAACCAATGATCTAATTAGGATTCTACT
GTTTCAAACTCTAACTTTTGCGTTTGCATTACATATAAATAGTTGAGAAGGAAGATTG
GTTACCAGAGGGGTTTGAAGAGAAGACCAAGGGAAAAGGATTGATAATCCGAGGG
TGGGCGCCACAAGTGCTGATACTTGAGCACAAAGCAATTGGCGGATTTTTGACGCA
TTGTGGATGGAACTCGTTATTAGAAGGGGTGGCAGCGGGCCTGCCAATGGTGACA
TGGCCCGTGGGAGCCGAGCAGTTCTACAACGAGAAATTGGTGACACAAGTGTTGA
AAACAGGAGTGAGTGTGGGAGTGAAGAAGATGATGCAAGTAGTTGGAGACTTCATT
AGCAGAGAGAAAGTGGAGGGAGCGGTGAGGGAAGTGATGGTTGGAGAAGAGAGG
AGGAAACGGGCCAAGGAGTTAGCAGAAATGGCGAAAAATGCGGTGAAAGAAGGAG
GATCTTCAGATCTAGAGGTAGATAGGTTGATGGAAGAGCTTACGTTAGTTAAACTG
CAAAAAGAGAAGGTATAA
Amino acid sequence (SEQ ID NO: 114)
MGTPVEVSKLHFLLFPFMAHGHMIPTLDMAKLFATKGAKSTILTTPLNAKLFFEKPIKSFN
QDNPGLEDITIQILNFPCTELGLPDGCENTDFIFSTPDLNVGDLSQKFLLAMKYFEEPLEE
LLVTMRPDCLVGNMFFPWSTKVAEKFGVPRLVFHGTGYFSLCASHCIRLPKNVATSSE
PFVIPDLPGDILITEEQVMETEEESVMGRFMKAIRDSERDSFGVLVNSFYELEQAYSDYF
KSFVAKRAWHIGPLSLGNRKFEEKAERGKKASIDEHECLKWLDSKKCDSVIYMAFGTM
SSFKNEQLIEIAAGLDMSGHDFVWVVNRKGSQEEKEDWLPEGFEEKTKGKGLIIRGWA
PQVLILEHKAIGGFLTHCGWNSLLEGVAAGLPMVTWPVGAEQFYNEKLVTQVLKTGVS
VGVKKMMQVVGDFISREKVEGAVREVMVGEERRKRAKELAEMAKNAVKEGGSSDLEV
DRLMEELTLVKLQKEKV
73B2
Nucleotide sequence (SEQ ID NO: 23)
ATGGGTAGTGATCATCATCATCGAAAGCTCCACGTTATGTTCTTCCCTTTCATGGCT
TATGGTCACATGATACCAACTCTAGACATGGCTAAGCTTTTCTCTAGCAGAGGAGC
CAAATCCACAATCCTCACCACATCTCTCAACTCCAAGATCCTCCAAAAACCCATCGA
CACATTCAAGAATCTGAATCCGGGTCTCGAAATCGACATCCAGATCTTCAATTTCCC
TTGCGTGGAGCTGGGGTTACCAGAAGGATGTGAAAACGTTGATTTCTTCACTTCAA
ACAACAATGATGATAAAAACGAGATGATCGTGAAATTCTTTTTCTCGACAAGGTTTTT
CAAAGACCAGCTTGAGAAACTCCTCGGGACAACGAGACCAGACTGTCTTATCGCCG
ACATGTTCTTCCCCTGGGCTACTGAAGCTGCTGGGAAGTTCAATGTGCCAAGACTT
GTGTTCCACGGCACTGGCTACTTCTCTTTATGCGCTGGTTATTGCATCGGAGTGCA
TAAACCACAGAAGAGAGTGGCTTCAAGCTCTGAGCCATTTGTGATTCCCGAGCTCC
CTGGGAACATTGTGATAACTGAAGAACAGATCATAGATGGCGATGGAGAATCCGAC
ATGGGAAAGTTTATGACTGAAGTTAGGGAATCGGAAGTGAAGAGCTCAGGAGTTGT
TTTGAATAGTTTCTACGAGCTAGAACATGATTACGCCGATTTTTACAAAAGTTGTGTA
CAAAAGAGAGCGTGGCATATCGGTCCGCTATCGGTTTACAACAGGGGATTTGAGG
AGAAGGCTGAGAGAGGAAAGAAAGCGAACATTGATGAGGCTGAATGCCTCAAATG
GCTTGACTCCAAGAAACCAAATTCAGTCATTTATGTTTCCTTTGGGAGCGTGGCTTT
CTTCAAGAATGAACAGTTATTCGAGATCGCTGCAGGGTTAGAAGCTTCCGGTACAA
GTTTCATTTGGGTTGTTAGGAAAACCAAAGGTATTGAAATTGACGTTTGAAGCCTAT
ATTATATAGCTGTAATTTGGGTAGCTTTGATTTTAATCTGACACAAGATTTGGTGTGA
ACAGATGATAGAGAAGAATGGTTACCAGAAGGGTTCGAAGAGAGGGTGAAAGGGA
AAGGTATGATAATAAGAGGATGGGCACCACAGGTGCTGATACTTGACCACCAAGCA
ACCGGTGGGTTTGTGACCCATTGCGGCTGGAACTCGCTTCTTGAAGGAGTGGCTG
CAGGGCTACCAATGGTGACATGGCCTGTAGGAGCGGAGCAATTCTACAATGAGAA
ATTGGTTACGCAAGTGCTCAGAACAGGAGTGAGCGTGGGAGCGAGCAAGCATATG
AAAGTTATGATGGGAGATTTCATTAGCAGAGAGAAAGTGGATAAAGCGGTGAGGGA
GGTTTTGGCTGGGGAAGCAGCAGAGGAGAGGCGGAGACGGGCAAAGAAGCTAGC
GGCGATGGCTAAAGCTGCCGTGGAAGAAGGAGGGTCTTCCTTCAACGATCTAAAC
AGCTTCATGGAAGAGTTTAGTTCATAA
Amino acid sequence (SEQ ID NO: 115)
MGSDHHHRKLHVMFFPFMAYGHMIPTLDMAKLFSSRGAKSTILTTSLNSKILQKPIDTFK
NLNPGLEIDIQIFNFPCVELGLPEGCENVDFFTSNNNDDKNEMIVKFFFSTRFFKDQLEK
LLGTTRPDCLIADMFFPWATEAAGKFNVPRLVFHGTGYFSLCAGYCIGVHKPQKRVASS
SEPFVIPELPGNIVITEEQIIDGDGESDMGKFMTEVRESEVKSSGVVLNSFYELEHDYAD
FYKSCVQKRAWHIGPLSVYNRGFEEKAERGKKANIDEAECLKWLDSKKPNSVIYVSFG
SVAFFKNEQLFEIAAGLEASGTSFIWVVRKTKDDREEWLPEGFEERVKGKGMIIRGWAP
QVLILDHQATGGFVTHCGWNSLLEGVAAGLPMVTWPVGAEQFYNEKLVTQVLRTGVS
VGASKHMKVMMGDFISREKVDKAVREVLAGEAAEERRRRAKKLAAMAKAAVEEGGSS
FNDLNSFMEEFSS
73B3
Nucleotide sequence (SEQ ID NO: 24)
ATGAGTAGTGATCCTCATCGTAAGCTCCATGTTGTGTTCTTCCCTTTCATGGCTTAT
GGTCACATGATACCAACTCTAGACATGGCTAAGCTTTTCTCTAGCAGAGGAGCCAA
ATCTACAATCCTCACCACACCTCTCAACTCCAAGATCTTCCAAAAACCCATCGAAAG
ATTCAAGAACCTGAATCCGAGTTTCGAAATCGACATCCAGATCTTCGATTTCCCTTG
CGTGGATCTCGGGTTACCAGAAGGATGCGAAAACGTCGATTTCTTCACCTCAAACA
ACAATGATGATAGACAGTATCTGACCTTGAAGTTCTTTAAGTCGACAAGGTTTTTCA
AAGATCAGCTTGAGAAGCTCCTCGAGACAACGAGACCAGACTGTCTTATCGCCGAC
ATGTTCTTCCCCTGGGCTACGGAAGCTGCTGAGAAGTTCAATGTGCCAAGACTTGT
GTTCCACGGTACTGGCTACTTTTCTTTATGCTCTGAATATTGCATCAGAGTGCATAA
CCCACAAAACATAGTAGCTTCAAGGTACGAGCCATTTGTGATTCCTGATCTCCCGG
GGAACATAGTGATAACTCAAGAACAGATAGCAGACCGTGACGAAGAAAGCGAGATG
GGGAAGTTTATGATTGAGGTCAAAGAATCTGATGTGAAGAGCTCAGGTGTTATTGT
AAACAGCTTCTACGAGCTTGAACCTGATTACGCCGACTTTTACAAGAGTGTTGTACT
GAAGAGAGCGTGGCATATCGGTCCGCTTTCGGTTTACAACAGAGGATTTGAGGAG
AAGGCTGAGAGAGGAAAGAAAGCAAGCATTAATGAGGTTGAATGCCTCAAATGGCT
TGACTCCAAGAAACCAGATTCAGTCATTTACATTTCTTTTGGGAGCGTGGCTTGCTT
CAAGAACGAGCAGCTATTCGAGATCGCTGCAGGATTAGAAACTTCTGGAGCAAATT
TCATCTGGGTTGTTAGGAAAAACATAGGTATTGAAAAAGAAGAATGGTTACCAGAAG
GGTTCGAAGAGAGGGTGAAAGGAAAAGGGATGATTATAAGAGGATGGGCACCACA
GGTGCTCATACTTGATCATCAAGCAACTTGTGGGTTTGTGACCCATTGCGGCTGGA
ACTCGCTTCTGGAAGGAGTGGCTGCAGGGCTACCAATGGTGACATGGCCTGTAGC
AGCGGAGCAATTCTACAATGAGAAATTGGTTACGCAAGTGCTCAGAACAGGAGTGA
GCGTGGGAGCGAAAAAGAATGTAAGAACTACGGGAGATTTCATTAGCAGAGAGAAA
GTGGTTAAAGCGGTGAGGGAGGTGTTGGTTGGGGAAGAGGCGGATGAGAGGCGG
GAGAGGGCAAAGAAGTTGGCAGAGATGGCTAAAGCTGCCGTGGAAGGAGGGTCTT
CTTTCAACGATCTAAACAGCTTCATAGAAGAGTTTACCTCGTAA
Amino acid sequence (SEQ ID NO: 116)
MSSDPHRKLHVVFFPFMAYGHMIPTLDMAKLFSSRGAKSTILTTPLNSKIFQKPIERFKNL
NPSFEIDIQIFDFPCVDLGLPEGCENVDFFTSNNNDDRQYLTLKFFKSTRFFKDQLEKLL
ETTRPDCLIADMFFPWATEAAEKFNVPRLVFHGTGYFSLCSEYCIRVHNPQNIVASRYE
PFVIPDLPGNIVITQEQIADRDEESEMGKFMIEVKESDVKSSGVIVNSFYELEPDYADFYK
SVVLKRAWHIGPLSVYNRGFEEKAERGKKASINEVECLKWLDSKKPDSVIYISFGSVAC
FKNEQLFEIAAGLETSGANFIWVVRKNIGIEKEEWLPEGFEERVKGKGMIIRGWAPQVLI
LDHQATCGFVTHCGWNSLLEGVAAGLPMVTWPVAAEQFYNEKLVTQVLRTGVSVGAK
KNVRTTGDFISREKVVKAVREVLVGEEADERRERAKKLAEMAKAAVEGGSSFNDLNSFI
EEFTS
73B4
Nucleotide sequence (SEQ ID NO: 25)
ATGAACAGAGAGCAAATTCATATTTTGTTCTTCCCCTTCATGGCTCATGGCCACATG
ATTCCACTCTTAGACATGGCCAAGCTTTTCGCTAGAAGAGGAGCCAAATCAACTCTC
CTCACAACCCCAATAAATGCTAAGATCTTGGAGAAACCCATTGAAGCATTCAAAGTT
CAAAATCCTGATCTCGAAATCGGAATCAAGATCCTCAATTTCCCTTGTGTAGAGCTT
GGATTGCCAGAAGGATGCGAGAACCGTGACTTCATTAACTCATACCAAAAATCTGA
CTCATTTGACTTGTTCTTGAAGTTTCTTTTCTCTACCAAGTATATGAAACAGCAGTTG
GAGAGTTTCATTGAAACAACCAAACCGAGTGCTCTTGTAGCCGATATGTTCTTCCCT
TGGGCAACAGAATCCGCGGAGAAGATCGGTGTTCCAAGACTTGTGTTCCACGGCA
CATCATCCTTTGCCTTGTGTTGTTCGTATAACATGAGGATTCATAAGCCACACAAGA
AAGTCGCTTCGAGTTCTACTCCATTTGTAATCCCTGGTCTCCCTGGAGACATAGTTA
TTACAGAAGACCAAGCCAATGTCACCAACGAAGAAACTCCATTCGGAAAGTTTTGG
AAAGAAGTCAGGGAATCAGAGACCAGTAGCTTTGGTGTTTTGGTGAATAGCTTCTA
CGAGCTGGAATCATCTTATGCTGATTTTTACCGTAGTTTTGTGGCGAAAAAAGCGTG
GCATATAGGTCCACTTTCACTATCCAACAGAGGGATTGCAGAGAAAGCCGGAAGAG
GGAAAAAGGCAAACATTGATGAGCAAGAATGCCTCAAATGGCTTGACTCTAAGACA
CCTGGCTCAGTAGTTTACTTGTCCTTTGGTAGCGGAACCGGCTTACCCAACGAACA
GCTGTTAGAGATTGCTTTCGGCCTTGAAGGCTCTGGACAAAATTTCATTTGGGTGG
TTAGCAAAAATGAAAACCAAGGTAATTTTTTTCCTCCTTAACCATTATTAATCAATGT
AGTCTTTATTAGTATATTTCCAAAAATATTAACATTTGTGTATACATTTTCCTATTGCC
AAATATGCTATGATGCCATAGCAATGAGTAGATTGGTTTGTGTACTTTATATATTACT
TTGTAGAACTTCTAACAATTATGACTTGGTGTTGGTGTAGTTGGGACAGGTGAAAAT
GAAGATTGGTTGCCTAAAGGGTTTGAAGAGAGGAATAAAGGAAAAGGGCTGATAAT
ACGCGGATGGGCCCCGCAAGTGCTGATACTTGACCACAAAGCAATCGGAGGATTT
GTGACGCATTGCGGATGGAACTCGACTTTGGAGGGCATTGCCGCAGGGCTGCCTA
TGGTGACTTGGCCGATGGGGGCAGAACAGTTCTACAACGAGAAGTTATTGACAAAA
GTGTTGAGAATAGGAGTGAACGTTGGAGCTACCGAGTTGGTGAAAAAAGGAAAGTT
GATTAGTAGAGCACAAGTGGAGAAGGCAGTAAGGGAAGTGATTGGTGGTGAGAAG
GCAGAGGAAAGGCGGCTAAGGGCTAAGGAGCTGGGCGAGATGGCTAAAGCCGCT
GTGGAAGAAGGAGGGTCTTCTTATAATGATGTGAACAAGTTTATGGAAGAGCTGAA
TGGTAGAAAGTAG
Amino acid sequence (SEQ ID NO: 117)
MNREQIHILFFPFMAHGHMIPLLDMAKLFARRGAKSTLLTTPINAKILEKPIEAFKVQNPDL
EIGIKILNFPCVELGLPEGCENRDFINSYQKSDSFDLFLKFLFSTKYMKQQLESFIETTKPS
ALVADMFFPWATESAEKIGVPRLVFHGTSSFALCCSYNMRIHKPHKKVASSSTPFVIPGL
PGDIVITEDQANVTNEETPFGKFWKEVRESETSSFGVLVNSFYELESSYADFYRSFVAK
KAWHIGPLSLSNRGIAEKAGRGKKANIDEQECLKWLDSKTPGSVVYLSFGSGTGLPNE
QLLEIAFGLEGSGQNFIWVVSKNENQGENEDWLPKGFEERNKGKGLIIRGWAPQVLILD
HKAIGGFVTHCGWNSTLEGIAAGLPMVTWPMGAEQFYNEKLLTKVLRIGVNVGATELV
KKGKLISRAQVEKAVREVIGGEKAEERRLRAKELGEMAKAAVEEGGSSYNDVNKFMEE
LNGRK
73B5
Nucleotide sequence (SEQ ID NO: 26)
ATGAACAGAGAAGTCTCTGAGAGAATTCATATTTTGTTCTTCCCCTTCATGGCTCAA
GGCCACATGATTCCAATTTTGGACATGGCCAAGCTTTTCTCGAGGAGAGGAGCCAA
GTCAACCCTTCTCACAACCCCAATCAACGCTAAGATCTTCGAGAAACCTATTGAAGC
ATTCAAAAATCAAAACCCTGATCTCGAAATCGGAATCAAGATCTTCAATTTCCCTTGT
GTAGAGCTTGGATTGCCTGAAGGATGCGAGAACGCTGACTTTATCAACTCATACCA
AAAATCTGACTCAGGTGACTTGTTCTTGAAGTTTCTTTTCTCTACCAAGTATATGAAA
CAACAGTTGGAGAGTTTCATTGAAACAACCAAACCAAGTGCTCTTGTTGCCGATATG
TTCTTCCCTTGGGCGACAGAATCTGCTGAGAAGCTCGGTGTACCAAGACTTGTGTT
CCACGGTACATCTTTCTTTTCTTTGTGTTGTTCGTATAACATGAGGATTCATAAGCC
ACACAAGAAAGTCGCTACGAGTTCTACTCCTTTTGTAATCCCTGGTCTCCCAGGAG
ACATAGTTATTACAGAAGACCAAGCCAATGTTGCCAAAGAAGAAACGCCAATGGGA
AAGTTTATGAAAGAGGTTAGGGAATCAGAGACCAATAGCTTTGGTGTATTGGTTAAT
AGCTTCTACGAGCTGGAATCAGCTTATGCTGATTTTTATCGTAGTTTTGTGGCGAAA
AGAGCTTGGCATATCGGTCCGCTTTCGCTATCTAACAGAGAGTTAGGAGAGAAAGC
CAGAAGAGGGAAAAAGGCTAACATTGATGAGCAAGAATGCCTAAAATGGCTGGACT
CTAAGACACCTGGTTCAGTAGTTTACTTGTCCTTTGGGAGCGGAACTAATTTCACCA
ACGACCAGCTGTTAGAGATCGCTTTTGGTCTTGAAGGTTCTGGACAAAGTTTCATCT
GGGTGGTTAGGAAAAATGAAAACCAAGGTAAATTGTTTCTCCCCAGCCATTATTAAC
CAACATAGTAATGTTAATATTTGTGTATATATTCGTATTGCCAAATATGCTCTGATAC
CATGGCAAGTAATAGATTGGCTCATGTATTTTATTTGTGATCATGTAGAATTTTCTTA
ACAGTTATGACTTGGTGTTGGTATGGTTGGGACAGGTGACAATGAAGAGTGGTTGC
CTGAAGGGTTTAAAGAGAGGACAACAGGGAAAGGGCTAATAATACCTGGATGGGC
GCCGCAAGTGCTGATACTTGACCATAAAGCAATTGGAGGATTTGTGACTCATTGCG
GATGGAACTCGGCTATAGAGGGCATTGCCGCGGGGCTGCCTATGGTAACATGGCC
AATGGGGGCAGAACAGTTCTACAATGAGAAGCTATTGACAAAAGTGTTGAGAATAG
GAGTGAACGTTGGAGCTACCGAGTTGGTGAAAAAAGGAAAGTTGATTAGTAGAGCA
CAAGTGGAGAAGGCAGTAAGGGAAGTGATTGGTGGTGAGAAGGCAGAGGAAAGG
CGGCTATGGGCTAAGAAGCTGGGCGAGATGGCTAAAGCCGCTGTGGAAGAAGGA
GGGTCCTCTTATAATGATGTGAACAAGTTTATGGAAGAGCTGAATGGTAGAAAGTAG
Amino acid sequence (SEQ ID NO: 118)
MNREVSERIHILFFPFMAQGHMIPILDMAKLFSRRGAKSTLLTTPINAKIFEKPIEAFKNQN
PDLEIGIKIFNFPCVELGLPEGCENADFINSYQKSDSGDLFLKFLFSTKYMKQQLESFIET
TKPSALVADMFFPWATESAEKLGVPRLVFHGTSFFSLCCSYNMRIHKPHKKVATSSTPF
VIPGLPGDIVITEDQANVAKEETPMGKFMKEVRESETNSFGVLVNSFYELESAYADFYR
SFVAKRAWHIGPLSLSNRELGEKARRGKKANIDEQECLKWLDSKTPGSVVYLSFGSGT
NFTNDQLLEIAFGLEGSGQSFIWVVRKNENQGDNEEWLPEGFKERTTGKGLIIPGWAP
QVLILDHKAIGGFVTHCGWNSAIEGIAAGLPMVTWPMGAEQFYNEKLLTKVLRIGVNVG
ATELVKKGKLISRAQVEKAVREVIGGEKAEERRLWAKKLGEMAKAAVEEGGSSYNDVN
KFMEELNGRK
73C1
Nucleotide sequence (SEQ ID NO: 27)
ATGGCATCGGAATTTCGTCCTCCTCTTCATTTTGTTCTCTTCCCTTTCATGGCTCAA
GGCCACATGATCCCAATGGTAGATATTGCAAGGCTCCTGGCTCAGCGCGGGGTGA
CTATAACCATTGTCACTACACCTCAAAACGCAGGCCGGTTCAAGAACGTTCTTAGCC
GGGCTATCCAATCCGGCTTGCCCATCAATCTCGTGCAAGTAAAGTTTCCATCTCAA
GAATCGGGTTCACCGGAAGGACAGGAGAATTTGGACTTGCTCGATTCATTGGGGG
CTTCATTAACCTTCTTCAAAGCATTTAGCCTGCTCGAGGAACCAGTCGAGAAGCTCT
TGAAAGAGATTCAACCTAGGCCAAACTGCATAATCGCTGACATGTGTTTGCCTTATA
CAAACAGAATTGCCAAGAATCTTGGTATACCAAAAATCATCTTTCATGGCATGTGTT
GCTTCAATCTTCTTTGTACGCACATAATGCACCAAAACCACGAGTTCTTGGAAACTA
TAGAGTCTGACAAGGAATACTTCCCCATTCCTAATTTCCCTGACAGAGTTGAGTTCA
CAAAATCTCAGCTTCCAATGGTATTAGTTGCTGGAGATTGGAAAGACTTCCTTGACG
GAATGACAGAAGGGGATAACACTTCTTATGGTGTGATTGTTAACACGTTTGAAGAG
CTCGAGCCAGCTTATGTTAGAGACTACAAGAAGGTTAAAGCGGGTAAGATATGGAG
CATCGGACCGGTTTCCTTGTGCAACAAGTTAGGAGAAGACCAAGCTGAGAGGGGA
AACAAGGCGGACATTGATCAAGACGAGTGTATTAAATGGCTTGATTCTAAAGAAGAA
GGGTCGGTGCTATATGTTTGCCTTGGAAGTATATGCAATCTTCCTCTGTCTCAGCTC
AAAGAGCTCGGCTTAGGCCTCGAGGAATCCCAAAGACCTTTCATTTGGGTCATAAG
AGGTTGGGAGAAGTATAACGAGTTACTTGAATGGATCTCAGAGAGCGGTTATAAGG
AAAGAATCAAAGAAAGAGGCCTTCTCATAACAGGATGGTCGCCTCAAATGCTTATCC
TTACACATCCTGCCGTTGGAGGATTCTTGACACATTGTGGATGGAACTCTACTCTTG
AAGGAATCACTTCAGGCGTTCCATTACTCACGTGGCCACTGTTTGGAGACCAATTC
TGCAATGAGAAATTGGCGGTGCAGATACTAAAAGCCGGTGTGAGAGCTGGGGTTG
AAGAGTCCATGAGATGGGGAGAAGAGGAGAAAATAGGAGTACTGGTGGATAAAGA
AGGAGTAAAGAAGGCAGTGGAGGAATTGATGGGTGATAGTAATGATGCTAAGGAG
AGAAGAAAAAGAGTGAAAGAGCTTGGAGAATTAGCTCACAAGGCTGTGGAAGAAG
GAGGCTCTTCTCATTCCAACATCACATTCTTGCTACAAGACATAATGCAATTAGAAC
AACCCAAGAAATGA
Amino acid sequence (SEQ ID NO: 119)
MASEFRPPLHFVLFPFMAQGHMIPMVDIARLLAQRGVTITIVTTPQNAGRFKNVLSRAIQ
SGLPINLVQVKFPSQESGSPEGQENLDLLDSLGASLTFFKAFSLLEEPVEKLLKEIQPRP
NCIIADMCLPYTNRIAKNLGIPKIIFHGMCCFNLLCTHIMHQNHEFLETIESDKEYFPIPNFP
DRVEFTKSQLPMVLVAGDWKDFLDGMTEGDNTSYGVIVNTFEELEPAYVRDYKKVKAG
KIWSIGPVSLCNKLGEDQAERGNKADIDQDECIKWLDSKEEGSVLYVCLGSICNLPLSQ
LKELGLGLEESQRPFIWVIRGWEKYNELLEWISESGYKERIKERGLLITGWSPQMLILTH
PAVGGFLTHCGWNSTLEGITSGVPLLTWPLFGDQFCNEKLAVQILKAGVRAGVEESMR
WGEEEKIGVLVDKEGVKKAVEELMGDSNDAKERRKRVKELGELAHKAVEEGGSSHSNI
TFLLQDIMQLEQPKK
73C3
Nucleotide sequence (SEQ ID NO: 29)
ATGGCTACGGAAAAAACCCACCAATTTCATCCTTCTCTTCACTTTGTCCTCTTCCCTT
TCATGGCTCAAGGCCACATGATTCCCATGATTGATATTGCAAGACTCTTGGCTCAG
CGTGGTGTGACCATAACAATTGTCACGACACCTCACAACGCAGCAAGGTTTAAGAA
TGTCCTAAACCGAGCGATCGAGTCTGGCTTGGCCATCAACATACTGCATGTGAAGT
TTCCATATCAAGAGTTTGGTTTGCCAGAAGGAAAAGAGAATATAGATTCGTTAGACT
CAACGGAGTTGATGGTACCTTTCTTCAAAGCGGTGAACTTGCTTGAAGATCCGGTC
ATGAAGCTCATGGAAGAGATGAAACCTAGACCTAGCTGTCTAATTTCTGATTGGTGT
TTGCCTTATACAAGCATAATCGCCAAGAACTTCAATATACCAAAGATAGTTTTCCAC
GGCATGGGTTGCTTTAATCTTTTGTGTATGCATGTTCTACGCAGAAACTTAGAGATC
CTAGAGAATGTAAAGTCGGATGAAGAGTATTTCTTGGTTCCTAGTTTTCCTGATAGA
GTTGAATTTACAAAGCTTCAACTTCCTGTGAAAGCAAATGCAAGTGGAGATTGGAAA
GAGATAATGGATGAAATGGTAAAAGCAGAATACACATCCTATGGTGTGATCGTCAA
CACATTTCAGGAGTTGGAGCCACCTTATGTCAAAGACTACAAAGAGGCAATGGATG
GAAAAGTATGGTCCATTGGACCCGTTTCCTTGTGTAACAAGGCAGGTGCAGACAAA
GCTGAGAGGGGAAGCAAGGCCGCCATTGATCAAGATGAGTGTCTTCAATGGCTTG
ATTCTAAAGAAGAAGGTTCGGTGCTCTATGTTTGCCTTGGAAGTATATGTAATCTTC
CTTTGTCTCAGCTCAAGGAGCTGGGGCTAGGCCTTGAGGAATCTCGAAGATCTTTT
ATTTGGGTCATAAGAGGTTCGGAAAAGTATAAAGAACTATTTGAGTGGATGTTGGA
GAGCGGTTTTGAAGAAAGAATCAAAGAGAGAGGACTTCTCATTAAAGGGTGGGCAC
CTCAAGTCCTTATCCTTTCACATCCTTCCGTTGGAGGATTCCTGACACACTGTGGAT
GGAACTCGACTCTCGAAGGAATCACCTCAGGCATTCCACTGATCACTTGGCCGCTG
TTTGGAGACCAATTCTGCAACCAAAAACTGGTCGTTCAAGTACTAAAAGCCGGTGTA
AGTGCCGGGGTTGAAGAAGTCATGAAATGGGGAGAAGAAGATAAAATAGGAGTGT
TAGTGGATAAAGAAGGAGTGAAAAAGGCTGTGGAAGAATTGATGGGTGATAGTGAT
GATGCAAAAGAGAGGAGAAGAAGAGTCAAAGAGCTTGGAGAATTAGCTCACAAAGC
TGTGGAAAAAGGAGGCTCTTCTCATTCTAACATCACACTCTTGCTACAAGACATAAT
GCAACTAGCACAATTCAAGAATTGA
Amino acid sequence (SEQ ID NO: 120)
MATEKTHQFHPSLHFVLFPFMAQGHMIPMIDIARLLAQRGVTITIVTTPHNAARFKNVLN
RAIESGLAINILHVKFPYQEFGLPEGKENIDSLDSTELMVPFFKAVNLLEDPVMKLMEEM
KPRPSCLISDWCLPYTSIIAKNFNIPKIVFHGMGCFNLLCMHVLRRNLEILENVKSDEEYF
LVPSFPDRVEFTKLQLPVKANASGDWKEIMDEMVKAEYTSYGVIVNTFQELEPPYVKDY
KEAMDGKVWSIGPVSLCNKAGADKAERGSKAAIDQDECLQWLDSKEEGSVLYVCLGSI
CNLPLSQLKELGLGLEESRRSFIWVIRGSEKYKELFEWMLESGFEERIKERGLLIKGWA
PQVLILSHPSVGGFLTHCGWNSTLEGITSGIPLITWPLFGDQFCNQKLVVQVLKAGVSA
GVEEVMKWGEEDKIGVLVDKEGVKKAVEELMGDSDDAKERRRRVKELGELAHKAVEK
GGSSHSNITLLLQDIMQLAQFKN
73C4
Nucleotide sequence (SEQ ID NO: 30)
ATGGCTTCCGAAAAATCCCACAAAGTTCATCCTCCTCTTCACTTTATTCTTTTCCCTT
TCATGGCTCAGGGCCACATGATTCCCATGATTGATATAGCAAGGCTCTTGGCTCAG
CGCGGTGCGACAGTAACTATTGTCACGACACGTTATAATGCAGGGAGGTTCGAGAA
TGTCTTAAGTCGTGCCATGGAGTCTGGTTTACCCATCAACATAGTGCATGTGAATTT
TCCATATCAAGAATTTGGTTTGCCAGAAGGAAAAGAGAATATAGATTCGTATGACTC
AATGGAGCTGATGGTACCTTTCTTTCAAGCAGTTAACATGCTCGAAGATCCGGTCAT
GAAGCTCATGGAAGAGATGAAACCTAGACCTAGCTGTATTATTTCTGATTTGCTCTT
GCCTTATACAAGCAAAATCGCAAGGAAATTCAGTATACCAAAGATAGTTTTCCACGG
CACGGGTTGCTTTAATCTTTTGTGTATGCATGTTCTACGCAGAAACCTCGAGATCTT
GAAGAACTTAAAGTCGGATAAAGATTATTTCCTGGTTCCTAGTTTTCCTGATAGAGT
TGAATTTACAAAGCCTCAAGTTCCAGTGGAAACAACTGCAAGTGGAGATTGGAAAG
CGTTCTTGGACGAAATGGTAGAAGCAGAATACACATCCTATGGTGTGATCGTCAAC
ACATTTCAGGAGTTGGAGCCTGCTTATGTCAAAGACTACACGAAGGCTAGGGCTGG
AAAAGTATGGTCCATTGGACCTGTTTCCTTGTGCAACAAGGCAGGTGCTGATAAAG
CTGAGAGGGGAAACCAGGCCGCCATTGATCAAGATGAGTGTCTTCAATGGCTTGAT
TCTAAAGAAGATGGTTCGGTGTTATATGTTTGCCTTGGAAGTATCTGTAATCTACCT
TTGTCTCAGCTCAAGGAGCTGGGGCTAGGCCTTGAAAAATCCCAAAGATCTTTTATT
TGGGTCATAAGAGGTTGGGAAAAGTATAATGAACTATATGAGTGGATGATGGAGAG
CGGTTTTGAAGAAAGAATCAAAGAGAGAGGACTTCTTATTAAAGGGTGGTCACCTC
AAGTCCTTATCCTTTCACATCCTTCCGTTGGAGGATTCCTGACACACTGTGGATGGA
ACTCGACTCTCGAAGGAATCACCTCAGGCATTCCACTGATCACTTGGCCGCTGTTT
GGAGACCAATTCTGCAACCAAAAACTGGTCGTTCAAGTACTAAAAGCCGGTGTAAG
TGCCGGGGTTGAAGAAGTCATGAAATGGGGAGAAGAGGAGAAAATAGGAGTGTTA
GTGGATAAAGAAGGAGTAAAGAAGGCAGTGGAAGAGTTAATGGGTGCGAGTGATG
ATGCAAAAGAGAGGAGAAGAAGAGTCAAAGAGCTTGGAGAATCAGCTCACAAGGCT
GTGGAAGAAGGAGGCTCTTCTCATTCTAACATCACATACTTGCTACAAGACATAATG
CAACAAGTGAAATCCAAGAACTGA
Amino acid sequence (SEQ ID NO: 121)
MASEKSHKVHPPLHFILFPFMAQGHMIPMIDIARLLAQRGATVTIVTTRYNAGRFENVLS
RAMESGLPINIVHVNFPYQEFGLPEGKENIDSYDSMELMVPFFQAVNMLEDPVMKLMEE
MKPRPSCIISDLLLPYTSKIARKFSIPKIVFHGTGCFNLLCMHVLRRNLEILKNLKSDKDYF
LVPSFPDRVEFTKPQVPVETTASGDWKAFLDEMVEAEYTSYGVIVNTFQELEPAYVKDY
TKARAGKVWSIGPVSLCNKAGADKAERGNQAAIDQDECLQWLDSKEDGSVLYVCLGSI
CNLPLSQLKELGLGLEKSQRSFIWVIRGWEKYNELYEWMMESGFEERIKERGLLIKGW
SPQVLILSHPSVGGFLTHCGWNSTLEGITSGIPLITWPLFGDQFCNQKLVVQVLKAGVS
AGVEEVMKWGEEEKIGVLVDKEGVKKAVEELMGASDDAKERRRRVKELGESAHKAVE
EGGSSHSNITYLLQDIMQQVKSKN
73C5
Nucleotide sequence (SEQ ID NO: 31)
ATGGTTTCCGAAACAACCAAATCTTCTCCACTTCACTTTGTTCTCTTCCCTTTCATGG
CTCAAGGCCACATGATTCCCATGGTTGATATTGCAAGGCTCTTGGCTCAGCGTGGT
GTGATCATAACAATTGTCACGACGCCTCACAATGCAGCGAGGTTCAAGAATGTCCT
AAACCGTGCCATTGAGTCTGGCTTGCCCATCAACTTAGTGCAAGTCAAGTTTCCATA
TCTAGAAGCTGGTTTGCAAGAAGGACAAGAGAATATCGATTCTCTTGACACAATGG
AGCGGATGATACCTTTCTTTAAAGCGGTTAACTTTCTCGAAGAACCAGTCCAGAAGC
TCATTGAAGAGATGAACCCTCGACCAAGCTGTCTAATTTCTGATTTTTGTTTGCCTT
ATACAAGCAAAATCGCCAAGAAGTTCAATATCCCAAAGATCCTCTTCCATGGCATGG
GTTGCTTTTGTCTTCTGTGTATGCATGTTTTACGCAAGAACCGTGAGATCTTGGACA
ATTTAAAGTCAGATAAGGAGCTTTTCACTGTTCCTGATTTTCCTGATAGAGTTGAATT
CACAAGAACGCAAGTTCCGGTAGAAACATATGTTCCAGCTGGAGACTGGAAAGATA
TCTTTGATGGTATGGTAGAAGCGAATGAGACATCTTATGGTGTGATCGTCAACTCAT
TTCAAGAGCTCGAGCCTGCTTATGCCAAAGACTACAAGGAGGTAAGGTCCGGTAAA
GCATGGACCATTGGACCCGTTTCCTTGTGCAACAAGGTAGGAGCCGACAAAGCAG
AGAGGGGAAACAAATCAGACATTGATCAAGATGAGTGCCTTAAATGGCTCGATTCT
AAGAAACATGGCTCGGTGCTTTACGTTTGTCTTGGAAGTATCTGTAATCTTCCTTTG
TCTCAACTCAAGGAGCTGGGACTAGGCCTAGAGGAATCCCAAAGACCTTTCATTTG
GGTCATAAGAGGTTGGGAGAAGTACAAAGAGTTAGTTGAGTGGTTCTCGGAAAGC
GGCTTTGAAGATAGAATCCAAGATAGAGGACTTCTCATCAAAGGATGGTCCCCTCA
AATGCTTATCCTTTCACATCCATCAGTTGGAGGGTTCCTAACACACTGTGGTTGGAA
CTCGACTCTTGAGGGGATAACTGCTGGTCTACCGCTACTTACATGGCCGCTATTCG
CAGACCAATTCTGCAATGAGAAATTGGTCGTTGAGGTACTAAAAGCCGGTGTAAGA
TCCGGGGTTGAACAGCCTATGAAATGGGGAGAAGAGGAGAAAATAGGAGTGTTGG
TGGATAAAGAAGGAGTGAAGAAGGCAGTGGAAGAATTAATGGGTGAGAGTGATGA
TGCAAAAGAGAGAAGAAGAAGAGCCAAAGAGCTTGGAGATTCAGCTCACAAGGCT
GTGGAAGAAGGAGGCTCTTCTCATTCTAACATCTCTTTCTTGCTACAAGACATAATG
GAACTGGCAGAACCCAATAATTGA
Amino acid sequence (SEQ ID NO: 122)
MVSETTKSSPLHFVLFPFMAQGHMIPMVDIARLLAQRGVIITIVTTPHNAARFKNVLNRAI
ESGLPINLVQVKFPYLEAGLQEGQENIDSLDTMERMIPFFKAVNFLEEPVQKLIEEMNPR
PSCLISDFCLPYTSKIAKKFNIPKILFHGMGCFCLLCMHVLRKNREILDNLKSDKELFTVPD
FPDRVEFTRTQVPVETYVPAGDWKDIFDGMVEANETSYGVIVNSFQELEPAYAKDYKE
VRSGKAWTIGPVSLCNKVGADKAERGNKSDIDQDECLKWLDSKKHGSVLYVCLGSICN
LPLSQLKELGLGLEESQRPFIWVIRGWEKYKELVEWFSESGFEDRIQDRGLLIKGWSPQ
MLILSHPSVGGFLTHCGWNSTLEGITAGLPLLTWPLFADQFCNEKLVVEVLKAGVRSGV
EQPMKWGEEEKIGVLVDKEGVKKAVEELMGESDDAKERRRRAKELGDSAHKAVEEGG
SSHSNISFLLQDIMELAEPNN
73C6
Nucleotide sequence (SEQ ID NO: 32)
ATGGCTTTCGAAAAAAACAACGAACCTTTTCCTCTTCACTTTGTTCTCTTCCCTTTCA
TGGCTCAAGGCCACATGATTCCCATGGTTGATATTGCAAGGCTCTTGGCTCAGCGA
GGTGTGCTTATAACAATTGTCACGACGCCTCACAATGCAGCAAGGTTCAAGAATGT
CCTAAACCGTGCCATTGAGTCTGGTTTGCCCATCAACCTAGTGCAAGTCAAGTTTC
CATATCAAGAAGCTGGTCTGCAAGAAGGACAAGAAAATATGGATTTGCTTACCACG
ATGGAGCAGATAACATCTTTCTTTAAAGCGGTTAACTTACTCAAAGAACCAGTCCAG
AACCTTATTGAAGAGATGAGCCCGCGACCAAGCTGTCTAATCTCTGATATGTGTTTG
TCGTATACAAGCGAAATCGCCAAGAAGTTCAAAATACCAAAGATCCTCTTCCATGGC
ATGGGTTGCTTTTGTCTTCTGTGTGTTAACGTTCTGCGCAAGAACCGTGAGATCTTG
GACAATTTAAAGTCTGATAAGGAGTACTTCATTGTTCCTTATTTTCCTGATAGAGTTG
AATTCACAAGACCTCAAGTTCCGGTGGAAACATATGTTCCTGCAGGCTGGAAAGAG
ATCTTGGAGGATATGGTAGAAGCGGATAAGACATCTTATGGTGTTATAGTCAACTCA
TTTCAAGAGCTCGAACCTGCGTATGCCAAAGACTTCAAGGAGGCAAGGTCTGGTAA
AGCATGGACCATTGGACCTGTTTCCTTGTGCAACAAGGTAGGAGTAGACAAAGCAG
AGAGGGGAAACAAATCAGATATTGATCAAGATGAGTGCCTTGAATGGCTCGATTCT
AAGGAACCGGGATCTGTGCTCTACGTTTGCCTTGGAAGTATTTGTAATCTTCCTCTG
TCTCAGCTCCTTGAGCTGGGACTAGGCCTAGAGGAATCCCAAAGACCTTTCATCTG
GGTCATAAGAGGTTGGGAGAAATACAAAGAGTTAGTTGAGTGGTTCTCGGAAAGCG
GCTTTGAAGATAGAATCCAAGATAGAGGACTTCTCATCAAAGGATGGTCCCCTCAA
ATGCTTATCCTTTCACATCCTTCTGTTGGAGGGTTCTTAACGCACTGCGGATGGAAC
TCGACTCTTGAGGGGATAACTGCTGGTCTACCAATGCTTACATGGCCACTATTTGC
AGACCAATTCTGCAACGAGAAACTGGTCGTACAAATACTAAAAGTCGGTGTAAGTG
CCGAGGTTAAAGAGGTCATGAAATGGGGAGAAGAAGAGAAGATAGGAGTGTTGGT
GGATAAAGAAGGAGTGAAGAAGGCAGTGGAAGAACTAATGGGTGAGAGTGATGAT
GCAAAAGAGAGAAGAAGAAGAGCCAAAGAGCTTGGAGAATCAGCTCACAAGGCTG
TGGAAGAAGGAGGCTCCTCTCATTCTAATATCACTTTCTTGCTACAAGACATAATGC
AACTAGCACAGTCCAATAATTGA
Amino acid sequence (SEQ ID NO: 123)
MAFEKNNEPFPLHFVLFPFMAQGHMIPMVDIARLLAQRGVLITIVTTPHNAARFKNVLNR
AIESGLPINLVQVKFPYQEAGLQEGQENMDLLTTMEQITSFFKAVNLLKEPVQNLIEEMS
PRPSCLISDMCLSYTSEIAKKFKIPKILFHGMGCFCLLCVNVLRKNREILDNLKSDKEYFIV
PYFPDRVEFTRPQVPVETYVPAGWKEILEDMVEADKTSYGVIVNSFQELEPAYAKDFKE
ARSGKAWTIGPVSLCNKVGVDKAERGNKSDIDQDECLEWLDSKEPGSVLYVCLGSICN
LPLSQLLELGLGLEESQRPFIWVIRGWEKYKELVEWFSESGFEDRIQDRGLLIKGWSPQ
MLILSHPSVGGFLTHCGWNSTLEGITAGLPMLTWPLFADQFCNEKLVVQILKVGVSAEV
KEVMKWGEEEKIGVLVDKEGVKKAVEELMGESDDAKERRRRAKELGESAHKAVEEGG
SSHSNITFLLQDIMQLAQSNN
74B1
Nucleotide sequence (SEQ ID NO: 35)
ATGGCGGAAACAACTCCCAAAGTGAAAGGCCACGTCGTAATCTTACCATACCCAGT
TCAAGGCCACCTAAACCCAATGGTTCAATTCGCTAAACGTCTAGTCTCCAAAAACGT
CAAAGTCACAATCGCCACCACTACCTACACCGCCTCCTCAATCACAACACCATCACT
CTCCGTCGAACCAATCTCCGATGGATTCGATTTCATCCCCATAGGTATCCCCGGTTT
CAGCGTCGATACTTACTCAGAATCCTTCAAGCTCAACGGATCCGAAACCCTAACTCT
CCTAATCGAGAAATTCAAATCCACAGATTCACCAATCGATTGCTTAATCTACGATTC
GTTTCTTCCTTGGGGACTTGAAGTTGCTAGATCTATGGAACTTTCAGCTGCTTCTTT
CTTCACTAATAATCTCACTGTTTGTTCTGTGTTGCGTAAATTCTCTAACGGTGACTTT
CCTCTTCCCGCTGATCCTAATTCGGCGCCGTTTCGTATCCGTGGCTTACCGTCTTT
GAGCTACGATGAGTTACCTTCGTTTGTGGGACGTCATTGGTTGACTCATCCTGAGC
ATGGCAGAGTTCTTCTGAATCAGTTTCCTAACCATGAAAATGCTGATTGGTTATTCG
TTAATGGCTTTGAAGGGTTAGAAGAAACACAAGTAAGAGTTTTGATTCTACTATAAA
GTTTGAAACTTTATGTTACATTGTTGAATTGAAATTAGAACTGTTGTTTTGATTAGGA
TTGTGAAAATGGTGAGTCTGATGCAATGAAGGCGACGTTGATCGGACCGATGATTC
CATCGGCTTATCTTGATGATCGGATGGAAGATGATAAAGACTATGGTGCGAGTCTG
TTGAAACCGATATCGAAGGAGTGTATGGAGTGGCTTGAGACTAAGCAGGCTCAGTC
AGTAGCATTTGTTTCGTTTGGTTCGTTTGGGATTCTCTTTGAGAAGCAACTTGCAGA
GGTAGCTATTGCGCTACAAGAATCGGATTTGAACTTCTTGTGGGTGATTAAAGAAG
CTCATATAGCGAAATTGCCTGAAGGGTTTGTGGAATCGACTAAAGATAGAGCCTTG
TTGGTTTCTTGGTGTAACCAGCTTGAGGTTTTAGCTCATGAATCGATAGGTTGCTTT
TTGACTCATTGTGGTTGGAACTCTACGTTGGAAGGGTTGAGTTTGGGAGTTCCGAT
GGTTGGTGTGCCTCAGTGGAGTGATCAGATGAATGATGCTAAGTTTGTGGAGGAA
GTTTGGAAAGTTGGGTATAGAGCGAAAGAGGAAGCTGGGGAAGTAATCGTGAAGA
GTGAAGAATTGGTGAGGTGTTTGAAAGGAGTGATGGAAGGAGAGAGTAGTGTGAA
GATTAGAGAGAGTTCGAAGAAGTGGAAAGATTTGGCTGTGAAGGCAATGAGTGAAG
GAGGAAGCTCTGATCGAAGCATTAACGAGTTTATAGAGAGTTTAGGGAAGTAA
Amino acid sequence (SEQ ID NO: 124)
MAETTPKVKGHVVILPYPVQGHLNPMVQFAKRLVSKNVKVTIATTTYTASSITTPSLSVE
PISDGFDFIPIGIPGFSVDTYSESFKLNGSETLTLLIEKFKSTDSPIDCLIYDSFLPWGLEVA
RSMELSAASFFTNNLTVCSVLRKFSNGDFPLPADPNSAPFRIRGLPSLSYDELPSFVGR
HWLTHPEHGRVLLNQFPNHENADWLFVNGFEGLEETQDCENGESDAMKATLIGPMIP
SAYLDDRMEDDKDYGASLLKPISKECMEWLETKQAQSVAFVSFGSFGILFEKQLAEVAI
ALQESDLNFLWVIKEAHIAKLPEGFVESTKDRALLVSWCNQLEVLAHESIGCFLTHCGW
NSTLEGLSLGVPMVGVPQWSDQMNDAKFVEEVWKVGYRAKEEAGEVIVKSEELVRCL
KGVMEGESSVKIRESSKKWKDLAVKAMSEGGSSDRSINEFIESLGK
74E2
Nucleotide sequence (SEQ ID NO: 39)
ATGAGAGAAGGATCTCATCTTATCGTCTTGCCTTTCCCAGGACAAGGCCACATAACT
CCAATGTCCCAGTTCTGCAAACGCTTAGCCTCAAAAGGTCTTAAGCTCACTCTGGT
CCTCGTCTCCGACAAACCCTCTCCTCCATACAAAACAGAGCACGACTCAATCACTGT
CTTCCCCATCTCCAACGGCTTCCAAGAAGGCGAGGAACCATTACAAGACCTCGATG
ATTACATGGAAAGAGTAGAAACCAGCATCAAAAACACCTTACCGAAGTTGGTTGAAG
ACATGAAACTGTCGGGAAATCCACCTAGGGCTATCGTGTACGACTCCACCATGCCA
TGGCTTCTTGATGTAGCTCATAGTTATGGATTGAGCGGTGCCGTGTTTTTCACGCA
ACCTTGGCTTGTCACAGCTATTTACTACCATGTTTTCAAGGGTTCGTTCTCTGTACC
GTCTACAAAGTACGGTCACTCGACATTAGCATCTTTCCCTTCGTTCCCGATGCTGAC
TGCAAATGATTTGCCGTCTTTCCTCTGCGAATCGTCCTCATACCCGAATATACTGAG
GATTGTGGTGGATCAGCTCTCAAACATTGATCGAGTCGACATAGTGTTGTGCAACA
CTTTCGATAAATTGGAGGAAAAGGTACAGAATATAAATCCATATAGAGGAACATGTC
TCTGTCTTTTGTAGGAAGTGTTTTAAGTTTTATTTTCTCTGCTTGTAGTTGTTGAAAT
GGGTCCAAAGCTTGTGGCCAGTCTTGAATATTGGACCAACGGTTCCATCGATGTAT
TTAGACAAACGACTGTCTGAAGACAAGAACTACGGTTTTAGCCTCTTCAATGCGAAA
GTCGCTGAATGCATGGAGTGGCTAAACTCAAAGGAGCCTAATTCTGTTGTCTATTTA
TCATTCGGAAGTTTGGTGATTCTAAAAGAAGATCAAATGTTGGAACTCGCTGCGGG
TCTGAAACAGAGCGGACGTTTCTTTCTGTGGGTTGTGAGAGAGACAGAGACACACA
AACTTCCAAGAAACTATGTCGAGGAAATCGGTGAAAAAGGACTTATTGTAAGCTGG
AGTCCTCAGCTTGACGTACTTGCACATAAATCAATCGGTTGTTTCTTGACACACTGT
GGATGGAACTCGACGTTAGAGGGATTGAGTTTGGGAGTTCCAATGATTGGTATGCC
ACACTGGACTGATCAGCCCACGAATGCTAAGTTCATGCAGGATGTGTGGAAGGTTG
GGGTAAGGGTTAAGGCAGAAGGTGATGGGTTTGTGAGAAGAGAAGAGATTATGAG
AAGTGTGGAAGAAGTTATGGAGGGAGAGAAAGGGAAAGAGATTAGAAAGAATGCT
GAGAAATGGAAAGTGTTGGCTCAAGAGGCAGTTTCTGAAGGAGGTAGCTCTGATAA
GAGCATCAATGAGTTTGTTTCTATGTTTTGTTGA
Amino acid sequence (SEQ ID NO: 125)
MREGSHLIVLPFPGQGHITPMSQFCKRLASKGLKLTLVLVSDKPSPPYKTEHDSITVFPIS
NGFQEGEEPLQDLDDYMERVETSIKNTLPKLVEDMKLSGNPPRAIVYDSTMPWLLDVA
HSYGLSGAVFFTQPWLVTAIYYHVFKGSFSVPSTKYGHSTLASFPSFPMLTANDLPSFL
CESSSYPNILRIVVDQLSNIDRVDIVLCNTFDKLEEKLLKWVQSLWPVLNIGPTVPSMYLD
KRLSEDKNYGFSLFNAKVAECMEWLNSKEPNSVVYLSFGSLVILKEDQMLELAAGLKQS
GRFFLWVVRETETHKLPRNYVEEIGEKGLIVSWSPQLDVLAHKSIGCFLTHCGWNSTLE
GLSLGVPMIGMPHWTDQPTNAKFMQDVWKVGVRVKAEGDGFVRREEIMRSVEEVME
GEKGKEIRKNAEKWKVLAQEAVSEGGSSDKSINEFVSMFC
74F1
Nucleotide sequence (SEQ ID NO: 40)
ATGGAGAAGATGAGAGGACATGTATTAGCAGTGCCATTTCCAAGCCAAGGACACAT
CACCCCGATTCGCCAATTCTGCAAACGACTTCACTCCAAAGGTTTCAAAACCACTCA
CACTCTCACCACTTTTATCTTCAACACAATCCACCTCGACCCATCTAGTCCTATCTC
CATAGCCACAATCTCCGATGGCTATGACCAGGGAGGGTTCTCATCAGCCGGTTCTG
TCCCGGAGTACCTACAAAACTTCAAAACCTTCGGCTCCAAAACCGTCGCTGATATCA
TCCGCAAACACCAGAGTACTGATAACCCTATTACTTGTATCGTCTATGATTCTTTCAT
GCCTTGGGCGCTTGACCTTGCAATGGATTTTGGTCTAGCTGCGGCTCCTTTCTTCA
CGCAGTCTTGCGCCGTTAACTATATCAATTATCTTTCTTACATAAACAATGGTAGCTT
GACACTTCCCATCAAGGATTTGCCTCTTCTTGAGCTCCAAGATTTGCCTACTTTCGT
CACTCCTACTGGTTCACACCTTGCTTACTTTGAGATGGTGCTTCAACAGTTCACCAA
CTTCGACAAAGCTGATTTCGTACTCGTTAATTCCTTCCATGACCTCGACCTTCATGT
TAGTTCATTTCCTAACTACTCTGTTTTTGCCCTAGTTACTCTGTTCTTTTTGACCTAG
CTACCCTGTTTTTCCCTTAGCTACTCTGTTTTATCACCTAATGACTATTTTTCTGTTC
TCTGATTTCCGTCTACAGGAAGAGGAGTTGTTGTCGAAAGTATGTCCTGTGTTGAC
AATTGGTCCAACTGTTCCATCAATGTACTTAGACCAACAGATCAAATCAGACAACGA
CTATGATCTGAACCTCTTTGACTTAAAAGAAGCTGCCTTATGCACTGACTGGCTAGA
CAAGAGGCCAGAAGGATCGGTAGTATATATAGCTTTTGGGAGCATGGCTAAACTGA
GTAGTGAGCAGATGGAAGAGATTGCTTCGGCGATAAGCAACTTCAGCTACCTCTGG
GTTGTCAGAGCTTCAGAGGAGTCAAAGCTCCCACCAGGGTTTCTTGAAACAGTGGA
TAAAGACAAGAGCTTGGTCTTGAAGTGGAGTCCTCAGCTTCAAGTTCTGTCAAACAA
AGCCATCGGTTGTTTCATGACTCACTGTGGCTGGAACTCAACCATGGAGGGTTTGA
GTTTAGGGGTTCCCATGGTGGCTATGCCTCAATGGACTGATCAACCAATGAATGCA
AAGTATATACAAGATGTATGGAAGGTTGGGGTTCGTGTGAAAGCAGAGAAAGAAAG
TGGCATTTGCAAAAGAGAGGAGATTGAGTTTAGCATCAAGGAAGTGATGGAAGGAG
AGAAGAGCAAAGAGATGAAAGAGAATGCGGGAAAATGGAGAGACTTGGCTGTGAA
GTCACTCAGTGAAGGAGGTTCTACAGATATCAACATTAACGAATTTGTATCAAAAAT
TCAAATCAAATAA
Amino acid sequence (SEQ ID NO: 126)
MEKMRGHVLAVPFPSQGHITPIRQFCKRLHSKGFKTTHTLTTFIFNTIHLDPSSPISIATIS
DGYDQGGFSSAGSVPEYLQNFKTFGSKTVADIIRKHQSTDNPITCIVYDSFMPWALDLA
MDFGLAAAPFFTQSCAVNYINYLSYINNGSLTLPIKDLPLLELQDLPTFVTPTGSHLAYFE
MVLQQFTNFDKADFVLVNSFHDLDLHEEELLSKVCPVLTIGPTVPSMYLDQQIKSDNDY
DLNLFDLKEAALCTDWLDKRPEGSVVYIAFGSMAKLSSEQMEEIASAISNFSYLWVVRA
SEESKLPPGFLETVDKDKSLVLKWSPQLQVLSNKAIGCFMTHCGWNSTMEGLSLGVPM
VAMPQWTDQPMNAKYIQDVWKVGVRVKAEKESGICKREEIEFSIKEVMEGEKSKEMKE
NAGKWRDLAVKSLSEGGSTDININEFVSKIQIK
76E1
Nucleotide sequence (SEQ ID NO: 53)
ATGGAAGAACTAGGAGTGAAGAGAAGGATAGTATTGGTTCCAGTTCCAGCACAAGG
TCATGTAACTCCGATTATGCAACTCGGGAAGGCTCTTTACTCCAAGGGCTTCTCCAT
CACTGTTGTTCTCACACAGTATAATCGAGTTAGCTCATCCAAGGACTTCTCTGATTT
TCATTTCCTCACCATCCCAGGCAGCTTGACCGAGTCTGATCTCAAAAACCTTGGAC
CATTCAAGTTTCTCTTCAAGCTCAATCAAATTTGCGAGGCAAGCTTCAAGCAATGTA
TTGGTCAACTATTGCAGGAGCAAGGTAATGATATCGCTTGTGTCGTCTACGATGAG
TACATGTACTTCTCCCAAGCTGCAGTTAAAGAGTTTCAACTTCCTAGCGTCCTCTTC
AGCACGACAAGTGCTACTGCCTTTGTCTGTCGCTCTGTTTTGTCTAGAGTCAACGC
AGAGTCATTCTTGCTTGACATGAAAGGTACTCAAGATTTTTTAGCTTGTTAACTCAAA
CTTTAAAAGTGCATTTAGGTATATAAACCAATCCAAATGCTGTTGTTTGCTTTGCAGA
TCCCAAAGTGTCAGACAAGGAATTTCCAGGGTTGCATCCGCTAAGGTACAAGGACC
TGCCAACTTCAGCATTTGGGCCATTAGAGAGTATACTCAAGGTTTACAGTGAGACT
GTCAACATTCGAACAGCTTCGGCAGTTATCATCAACTCAACAAGCTGTCTAGAGAG
CTCATCTTTGGCATGGTTACAAAAACAACTGCAAGTTCCAGTGTATCCTATAGGCCC
ACTTCACATTGCAGCTTCAGCGCCTTCTAGTTTACTTGAAGAGGACAGGAGTTGCC
TTGAGTGGTTGAACAAGCAAAAAATAGGCTCAGTGATTTACATAAGTTTGGGAAGCT
TGGCTCTAATGGAAACTAAAGACATGTTGGAGATGGCTTGGGGTTTACGTAATAGC
AACCAACCTTTCTTATGGGTGATCCGACCGGGTTCTATTCCCGGCTCGGAATGGAC
AGAGTCTTTACCGGAGGAATTCAGTAGGTTGGTTTCAGAAAGAGGTTACATTGTGA
AATGGGCACCACAGATAGAAGTTCTCAGACATCCTGCAGTGGGAGGGTTTTGGAGT
CACTGCGGATGGAACTCGACCCTAGAGAGCATCGGGGAAGGAGTTCCGATGATCT
GTAGGCCTTTTACGGGAGATCAGAAAGTCAATGCGAGGTACTTAGAGAGAGTTTGG
AGAATTGGGGTTCAATTGGAAGGAGAGCTGGATAAAGGAACAGTGGAGAGAGCTG
TAGAGAGATTGATTATGGATGAAGAAGGAGCAGAAATGAGGAAGAGAGTTATCAAC
TTGAAAGAGAAGCTTCAAGCCTCTGTCAAGAGTAGAGGTTCCTCATTCAGCTCATTA
GACAACTTTGTCAATTCCTTAAAAATGATGAATTTCATGTAG
Amino acid sequence (SEQ ID NO: 127)
MEELGVKRRIVLVPVPAQGHVTPIMQLGKALYSKGFITVVLTQYNRVSSSKDFSDFHFL
TIPGSLTESDLKNLGPFKFLFKLNQICEASFKQCIGQLLQEQGNDIACVVYDEYMYFSQA
AVKEFQLPSVLFSTTSATAFVCRSVLSRVNAESFLLDMKDPKVSDKEFPGLHPLRYKDL
PTSAFGPLESILKVYSETVNIRTASAVIINSTSCLESSSLAWLQKQLQVPVYPIGPLHIAAS
APSSLLEEDRSCLEWLNKQKIGSVIYISLGSLALMETKDMLEMAWGLRNSNQPFLWVIR
PGSIPGSEWTESLPEEFSRLVSERGYIVKWAPQIEVLRHPAVGGFWSHCGWNSTLESI
GEGVPMICRPFTGDQKVNARYLERVWRIGVQLEGELDKGTVERAVERLIMDEEGAEMR
KRVINLKEKLQASVKSRGSSFSSLDNFVNSLKMMNFM
76E12
Nucleotide sequence (SEQ ID NO: 55)
ATGCAGGTTTTGGGAATGGAGGAAAAGCCTGCAAGGAGAAGCGTAGTGTTGGTTC
CATTTCCAGCACAAGGACATATATCTCCAATGATGCAACTTGCCAAAACCCTTCACT
TAAAGGGTTTCTCGATCACAGTTGTTCAGACTAAGTTCAATTACTTTAGCCCTTCAG
ATGACTTCACTCATGATTTTCAGTTCGTCACCATTCCAGAAAGCTTACCAGAGTCTG
ATTTCAAGAATCTCGGACCAATACAGTTTCTGTTTAAGCTCAACAAAGAGTGTAAGG
TGAGCTTCAAGGACTGTTTGGGTCAGTTGGTGCTGCAACAAAGTAATGAGATCTCA
TGTGTCATCTACGATGAGTTCATGTACTTTGCTGAAGCTGCAGCCAAAGAGTGTAA
GCTTCCAAACATCATTTTCAGCACAACAAGTGCCACGGCTTTCGCTTGCCGCTCTG
TATTTGACAAACTATATGCAAACAATGTCCAAGCTCCCTTGAAAGGTACTCTAAAAC
TCTCTGTTTCGTGGTTTCCGCGAGTGGCTATAAGATTGAAACAGCATTGTTTTTGAC
CTTTTTTGCAGAAACTAAAGGACAACAAGAAGAGCTAGTTCCGGAGTTTTATCCCTT
GAGATATAAAGACTTTCCAGTTTCACGGTTTGCATCATTAGAGAGCATAATGGAGGT
GTATAGGAATACAGTTGACAAACGGACAGCTTCCTCGGTGATAATCAACACTGCGA
GCTGTCTAGAGAGCTCATCTCTGTCTTTTCTGCAACAACAACAGCTACAAATTCCAG
TGTATCCTATAGGCCCTCTTCACATGGTGGCCTCAGCTCCTACAAGTCTGCTTGAA
GAGAACAAGAGCTGCATCGAATGGTTGAACAAACAAAAGGTAAACTCGGTGATATA
CATAAGCATGGGAAGCATAGCTTTAATGGAAATCAACGAGATAATGGAAGTCGCGT
CAGGATTGGCTGCTAGCAACCAACACTTCTTATGGGTGATCCGACCAGGGTCAATA
CCTGGTTCCGAGTGGATAGAGTCCATGCCTGAAGAGTTTAGTAAGATGGTTTTGGA
CCGAGGTTACATTGTGAAATGGGCTCCACAGAAGGAAGTACTTTCTCATCCTGCAG
TAGGAGGGTTTTGGAGCCATTGTGGATGGAACTCGACACTAGAAAGCATCGGCCA
AGGAGTTCCAATGATCTGCAGGCCATTTTCGGGTGATCAAAAGGTGAACGCTAGAT
ACTTGGAGTGTGTATGGAAAATTGGGATTCAAGTGGAGGGTGAGCTAGACAGAGG
AGTGGTCGAGAGAGCTGTGAAGAGGTTAATGGTTGACGAAGAAGGAGAGGAGATG
AGGAAGAGAGCTTTCAGTTTAAAAGAGCAACTTAGAGCCTCTGTTAAAAGTGGAGG
CTCTTCACACAACTCGCTAGAAGAGTTTGTACACTTCATAAGGACTCTATGA
Amino acid sequence (SEQ ID NO: 128)
MEEKPARRSVVLVPFPAQGHISPMMQLAKTLHLKGFSITVVQTKFNYFSPSDDFTHDFQ
FVTIPESLPESDFKNLGPIQFLFKLNKECKVSFKDCLGQLVLQQSNEISCVIYDEFMYFAE
AAAKECKLPNIIFSTTSATAFACRSVFDKLYANNVQAPLKETKGQQEELVPEFYPLRYKD
FPVSRFASLESIMEVYRNTVDKRTASSVIINTASCLESSSLSFLQQQQLQIPVYPIGPLHM
VASAPTSLLEENKSCIEWLNKQKVNSVIYISMGSIALMEINEIMEVASGLAASNQHFLWVI
RPGSIPGSEWIESMPEEFSKMVLDRGYIVKWAPQKEVLSHPAVGGFWSHCGWNSTLE
SIGQGVPMICRPFSGDQKVNARYLECVWKIGIQVEGELDRGVVERAVKRLMVDEEGEE
MRKRAFSLKEQLRASVKSGGSSHNSLEEFVHFIRTL
78D2
Nucleotide sequence (SEQ ID NO: 66)
ATGACCAAACCCTCCGACCCAACCAGAGACTCCCACGTGGCAGTTCTCGCTTTTCC
TTTCGGCACTCATGCAGCTCCTCTCCTCACCGTCACGCGCCGCCTCGCCTCCGCCT
CTCCTTCCACCGTCTTCTCTTTCTTCAACACCGCACAATCCAACTCTTCGTTATTTTC
CTCCGGTGACGAAGCAGATCGTCCGGCGAACATCAGAGTATACGATATTGCCGAC
GGTGTTCCGGAGGGATACGTGTTTAGCGGGAGACCACAGGAGGCGATCGAGCTGT
TTCTTCAAGCTGCGCCGGAGAATTTCCGGAGAGAAATCGCGAAGGCGGAGACGGA
GGTTGGTACGGAAGTGAAATGTTTGATGACTGATGCGTTCTTCTGGTTCGCGGCTG
ATATGGCGACGGAGATAAATGCGTCGTGGATTGCGTTTTGGACCGCCGGAGCAAA
CTCACTCTCTGCTCATCTCTACACAGATCTCATCAGAGAAACCATCGGTGTCAAAGG
TAATATATACAAATTTTTGAATGCTTCCCAATTCCGACTTGTGATTTTGTCTTTTATCT
CATAAATAAATATGCAACTAGAGGAAAATTTAGCTAAAAGAAGAAACAGAGGTTAAG
ATACTATTGATTTGAAGATTTATATGTATTTGTGGTAATGTTTATGATTCCATTCTAAT
TTACAGAAGTAGGTGAGCGTATGGAGGAGACAATAGGGGTTATCTCAGGAATGGA
GAAGATCAGAGTCAAAGATACACCAGAAGGAGTTGTGTTTGGGAATTTAGACTCTG
TTTTCTCAAAGATGCTTCATCAAATGGGTCTTGCTTTGCCTCGTGCCACTGCTGTTT
TCATCAATTCTTTTGAAGATTTGGATCCTACATTGACGAATAACCTCAGATCGAGATT
TAAACGATATCTGAACATCGGTCCTCTCGGGTTATTATCTTCTACATTGCAACAACT
AGTGCAAGATCCTCACGGTTGTTTGGCTTGGATGGAGAAGAGATCTTCTGGTTCTG
TGGCGTACATTAGCTTTGGTACGGTCATGACACCGCCTCCTGGAGAGCTTGCGGC
GATAGCAGAAGGGTTGGAATCGAGTAAAGTGCCGTTTGTTTGGTCGCTTAAGGAGA
AGAGCTTGGTTCAGTTACCAAAAGGGTTTTTGGATAGGACAAGAGAGCAAGGGATA
GTGGTTCCATGGGCACCGCAAGTGGAACTGCTGAAACACGAAGCAACGGGTGTGT
TTGTGACGCATTGTGGATGGAACTCGGTGTTGGAGAGTGTATCGGGTGGTGTACC
GATGATTTGCAGGCCATTTTTTGGGGATCAGAGATTGAACGGAAGAGCGGTGGAG
GTTGTGTGGGAGATTGGAATGACGATTATCAATGGAGTCTTCACGAAAGATGGGTT
TGAGAAGTGTTTGGATAAAGTTTTAGTTCAAGATGATGGTAAGAAGATGAAATGTAA
TGCTAAGAAACTTAAAGAACTAGCTTACGAAGCTGTCTCTTCTAAAGGAAGGTCCTC
TGAGAATTTCAGAGGATTGTTGGATGCAGTTGTAAACATTATTTGA
Amino acid sequence (SEQ ID NO: 129)
MTKPSDPTRDSHVAVLAFPFGTHAAPLLTVTRRLASASPSTVFSFFNTAQSNSSLFSSG
DEADRPANIRVYDIADGVPEGYVFSGRPQEAIELFLQAAPENFRREIAKAETEVGTEVKC
LMTDAFFWFAADMATEINASWIAFWTAGANSLSAHLYTDLIRETIGVKEVGERMEETIG
VISGMEKIRVKDTPEGVVFGNLDSVFSKMLHQMGLALPRATAVFINSFEDLDPTLTNNLR
SRFKRYLNIGPLGLLSSTLQQLVQDPHGCLAWMEKRSSGSVAYISFGTVMTPPPGELA
AIAEGLESSKVPFVWSLKEKSLVQLPKGFLDRTREQGIVVPWAPQVELLKHEATGVFVT
HCGWNSVLESVSGGVPMICRPFFGDQRLNGRAVEVVWEIGMTIINGVFTKDGFEKCLD
KVLVQDDGKKMKCNAKKLKELAYEAVSSKGRSSENFRGLLDAVVNII
84A1
Nucleotide sequence (SEQ ID NO: 81)
ATGGTGTTCGAAACTTGTCCATCTCCAAACCCAATTCATGTAATGCTCGTCTCGTTT
CAAGGACAAGGCCACGTCAACCCTCTTCTTCGTCTCGGCAAGTTAATTGCTTCAAA
GGGTTTACTCGTTACCTTCGTTACAACGGAGCTTTGGGGCAAGAAAATGAGACAAG
CCAACAAAATCGTTGACGGTGAACTTAAACCGGTTGGTTCCGGTTCAATCCGGTTT
GAGTTCTTTGATGAAGAATGGGCAGAGGATGATGACCGGAGAGCTGATTTCTCTTT
GTACATTGCTCACCTAGAGAGCGTTGGGATACGAGAAGTGTCTAAGCTTGTGAGAA
GATACGAGGAAGCGAACGAGCCTGTCTCGTGTCTTATCAATAACCCGTTTATCCCA
TGGGTCTGCCACGTGGCGGAAGAGTTCAACATTCCTTGTGCGGTTCTCTGGGTTCA
GTCTTGTGCTTGTTTCTCTGCTTATTACCATTACCAAGATGGCTCTGTTTCATTCCCT
ACGGAAACAGAGCCTGAGCTCGATGTGAAGCTTCCTTGTGTTCCTGTCTTGAAGAA
CGACGAGATTCCTAGCTTTCTCCATCCTTCTTCTAGGTTCACGGGTTTTCGACAAGC
GATTCTTGGGCAATTCAAGAATCTGAGCAAGTCCTTCTGTGTTCTAATCGATTCTTT
TGACTCATTGGAACAAGAAGTTATCGATTACATGTCAAGTCTTTGTCCGGTTAAAAC
CGTTGGACCGCTTTTCAAAGTTGCTAGGACAGTTACTTCTGACGTAAGCGGTGACA
TTTGCAAATCAACAGATAAATGCCTCGAGTGGTTAGACTCGAGGCCTAAATCGTCA
GTTGTCTACATTTCGTTCGGGACAGTTGCATATTTGAAGCAAGAACAGATCGAAGA
GATCGCTCACGGAGTTTTGAAGTCGGGTTTATCGTTCTTGTGGGTGATTAGACCTC
CACCACACGATCTGAAGGTCGAGACACATGTCTTGCCTCAAGAACTTAAAGAGAGT
AGTGCTAAAGGTAAAGGGATGATTGTGGATTGGTGCCCACAAGAGCAAGTCTTGTC
TCATCCTTCAGTGGCATGCTTCGTGACTCATTGTGGATGGAACTCGACAATGGAAT
CTTTGTCTTCAGGTGTTCCGGTGGTTTGTTGTCCGCAATGGGGAGATCAAGTGACT
GATGCAGTGTATTTGATCGATGTTTTCAAGACCGGGGTTAGACTAGGCCGTGGAGC
GACCGAGGAGAGGGTAGTGCCAAGGGAGGAAGTGGCGGAGAAGCTTTTGGAAGC
GACAGTTGGGGAGAAGGCAGAGGAGTTGAGAAAGAACGCTTTGAAATGGAAGGCG
GAGGCGGAAGCAGCGGTGGCTCCAGGAGGTTCGTCGGATAAGAATTTTAGGGAGT
TTGTGGAGAAGTTAGGTGCGGGAGTAACGAAGACTAAAGATAATGGATACTAG
Amino acid sequence (SEQ ID NO: 130)
MVFETCPSPNPIHVMLVSFQGQGHVNPLLRLGKLIASKGLLVTFVTTELWGKKMRQAN
KIVDGELKPVGSGSIRFEFFDEEWAEDDDRRADFSLYIAHLESVGIREVSKLVRRYEEAN
EPVSCLINNPFIPWVCHVAEEFNIPCAVLWVQSCACFSAYYHYQDGSVSFPTETEPELD
VKLPCVPVLKNDEIPSFLHPSSRFTGFRQAILGQFKNLSKSFCVLIDSFDSLEQEVIDYMS
SLCPVKTVGPLFKVARTVTSDVSGDICKSTDKCLEWLDSRPKSSVVYISFGTVAYLKQE
QIEEIAHGVLKSGLSFLWVIRPPPHDLKVETHVLPQELKESSAKGKGMIVDWCPQEQVL
SHPSVACFVTHCGWNSTMESLSSGVPVVCCPQWGDQVTDAVYLIDVFKTGVRLGRGA
TEERVVPREEVAEKLLEATVGEKAEELRKNALKWKAEAEAAVAPGGSSDKNFREFVEK
LGAGVTKTKDNGY
84B1
Nucleotide sequence (SEQ ID NO: 84)
ATGGGCAGTAGTGAGGGTCAAGAAACACATGTCCTAATGGTAACACTACCATTCCA
AGGTCACATCAATCCAATGCTCAAACTCGCAAAACATCTCTCGTTATCATCAAAGAA
CCTACACATCAATCTCGCCACTATTGAGTCAGCCCGTGATCTCCTCTCCACCGTAG
AAAAACCTCGTTATCCGGTGGACCTCGTGTTCTTCTCCGATGGTCTACCTAAAGAA
GATCCAAAGGCCCCTGAAACTCTTTTGAAGTCATTGAATAAAGTCGGAGCCATGAA
CTTGTCTAAAATCATCGAAGAAAAGAGATACTCTTGTATCATCTCTTCGCCTTTTACT
CCATGGGTTCCAGCTGTTGCAGCCTCTCATAACATCTCTTGTGCAATACTTTGGATC
CAAGCTTGTGGAGCTTACTCGGTTTATTACCGTTACTACATGAAGACAAACTCTTTC
CCTGATCTTGAAGATCTGAATCAAACGGTGGAGTTACCAGCTTTACCATTGTTGGAA
GTTCGAGATCTTCCATCGTTTATGTTACCTTCTGGTGGTGCTCACTTCTATAATCTA
ATGGCGGAATTTGCAGATTGTTTGAGGTATGTGAAATGGGTTTTGGTTAATTCATTC
TATGAACTCGAATCAGAGATAATCGAATCGATGGCTGATTTAAAACCTGTAATTCCA
ATTGGTCCTCTGGTTTCTCCATTTCTGTTGGGCGATGGTGAGGAGGAAACCCTAGA
CGGTAAAAACCTAGATTTTTGTAAATCTGATGATTGTTGTATGGAGTGGCTTGACAA
GCAAGCTAGGTCTTCTGTTGTGTACATATCTTTCGGAAGTATGCTCGAAACATTGGA
GAATCAGGTCGAGACCATAGCGAAGGCGCTGAAGAACAGAGGACTTCCATTTCTTT
GGGTGATAAGGCCAAAGGAGAAAGCCCAAAACGTTGCTGTTTTGCAGGAGATGGT
GAAAGAAGGACAAGGGGTTGTTCTCGAGTGGAGTCCACAAGAGAAGATTTTGAGC
CACGAGGCAATCTCTTGTTTTGTCACGCATTGCGGCTGGAACTCGACTATGGAGAC
GGTGGTGGCTGGTGTTCCTGTGGTAGCGTACCCTAGCTGGACGGATCAGCCCATT
GACGCGCGGTTGCTTGTTGATGTGTTTGGAATCGGAGTAAGGATGAGGAATGACA
GTGTCGATGGCGAGCTTAAGGTCGAAGAAGTAGAAAGATGCATTGAGGCCGTGAC
GGAGGGACCCGCTGCCGTGGATATAAGAAGGAGAGCGGCGGAGCTAAAGCGCGT
GGCGAGATTGGCGTTGGCACCTGGTGGATCTTCGACACGGAATTTAGACTTGTTCA
TTAGTGATATCACAATCGCCTAA
Amino acid sequence (SEQ ID NO: 131)
MGSSEGQETHVLMVTLPFQGHINPMLKLAKHLSLSSKNLHINLATIESARDLLSTVEKPR
YPVDLVFFSDGLPKEDPKAPETLLKSLNKVGAMNLSKIIEEKRYSCIISSPFTPWVPAVAA
SHNISCAILWIQACGAYSVYYRYYMKTNSFPDLEDLNQTVELPALPLLEVRDLPSFMLPS
GGAHFYNLMAEFADCLRYVKWVLVNSFYELESEIIESMADLKPVIPIGPLVSPFLLGDGE
EETLDGKNLDFCKSDDCCMEWLDKQARSSVVYISFGSMLETLENQVETIAKALKNRGLP
FLWVIRPKEKAQNVAVLQEMVKEGQGVVLEWSPQEKILSHEAISCFVTHCGWNSTMET
VVAGVPVVAYPSWTDQPIDARLLVDVFGIGVRMRNDSVDGELKVEEVERCIEAVTEGP
AAVDIRRRAAELKRVARLALAPGGSSTRNLDLFISDITIA
85A5
Nucleotide sequence (SEQ ID NO: 91)
ATGGCGTCTCATGCTGTTACAAGCGGACAAAAACCACACGTAGTTTGCATACCTTTC
CCGGCTCAAGGCCACATCAATCCGATGCTCAAAGTGGCTAAACTCCTCTATGCCAG
AGGCTTCCATGTTACCTTCGTCAACACTAACTACAACCATAACCGTCTCATCCGGTC
ACGTGGTCCCAACTCCCTTGATGGGCTTCCTTCTTTTCGGTTCGAGTCCATCCCTG
ACGGTCTACCGGAGGAAAACAAGGACGTCATGCAGGATGTCCCTACCCTTTGTGA
GTCCACCATGAAAAACTGTCTAGCTCCTTTCAAGGAGCTTCTCCGGCGGATCAACA
CCACAAAGGATGTTCCTCCGGTAAGCTGTATTGTATCCGACGGTGTGATGAGCTTT
ACTCTTGATGCTGCAGAGGAGCTTGGAGTCCCGGATGTTCTTTTTTGGACACCAAG
TGCTTGTGGCTTCTTGGCTTATCTACACTTCTATCGCTTCATCGAGAAGGGGTTATC
ACCAATAAAAGGTAAGTAAAAGGTTATTATTAGTTTAGGTTTTCATCACAAAGTATAT
TATTATTATTATTTCATTAACAATTTACATTATCTATGACACCTAGAACAGAGGTACCT
ATAATACAGATACGTAAGAAGTACCGTCGTCTAGGCCTTTTTCTGTCATTGTTAGGG
CGACCAAGAATAACTCATCCTTACTCTGAAATTAATCTATAGTATTAATTGATCAAAA
TTAAATGCATCAAAAATTTGCATATAATACGGTGCTTGAATGTTTTTATAGTAAATAT
TGAGATATAAAATTATACTTATAAAATGGAAGTGGATTATGGCAGATGAAAGTTCTTT
GGACACAAAAATAAATTGGATACCATCGATGAAAAACCTAGGACTTAAAGACATCCC
AAGCTTTATCCGTGCAACTAATACTGAAGACATAATGCTTAACTTTTTTGTCCATGAG
GCTGACCGAGCCAAACGCGCTTCCGCTATCATTCTCAACACATTCGATAGTCTTGA
GCATGATGTCGTCCGTTCTATTCAATCTATCATACCTCAAGTGTACACTATTGGACC
GCTTCATCTATTTGTGAATCGGGATATCGACGAGGAAAGTGACATCGGACAGATAG
GAACGAATATGTGGAGAGAGGAGATGGAGTGTTTGGATTGGCTTGATACTAAGTCT
CCAAACAGTGTCGTTTATGTTAATTTCGGTAGCATAACAGTGATGAGTGCGAAACAA
CTCGTGGAGTTTGCTTGGGGTTTAGCAGCGACCAAAAAAGATTTTTTGTGGGTGAT
TAGGCCGGATTTAGTAGCCGGTGATGTGCCAATGCTTCCGCCGGACTTTCTAATAG
AGACGGCTAACCGAAGGATGCTAGCGAGTTGGTGTCCTCAAGAAAAAGTTCTTTCT
CATCCGGCAGTTGGAGGGTTCTTAACGCATAGTGGATGGAATTCGACTTTGGAGAG
TCTCTCCGGTGGAGTTCCAATGGTGTGTTGGCCGTTCTTTGCGGAACAGCAAACAA
ATTGTAAATATTGTTGTGATGAATGGGAAGTGGGGATGGAGATCGGTGGAGATGTG
AGGAGGGAGGAGGTTGAGGAGTTGGTTAGAGAACTCATGGACGGAGACAAAGGAA
AGAAAATGAGGCAAAAGGCCGAAGAGTGGCAGCGCTTGGCTGAGGAAGCGACGAA
GCCTATTTATGGTTCGTCGGAACTAAATTTTCAGATGGTCGTTGACAAGGTTCTTTT
AGGGGAGTAG
Amino acid sequence (SEQ ID NO: 132)
MASHAVTSGQKPHVVCIPFPAQGHINPMLKVAKLLYARGFHVTFVNTNYNHNRLIRSRG
PNSLDGLPSFRFESIPDGLPEENKDVMQDVPTLCESTMKNCLAPFKELLRRINTTKDVP
PVSCIVSDGVMSFTLDAAEELGVPDVLFWTPSACGFLAYLHFYRFIEKGLSPIKDESSLD
TKINWIPSMKNLGLKDIPSFIRATNTEDIMLNFFVHEADRAKRASAIILNTFDSLEHDVVRS
IQSIIPQVYTIGPLHLFVNRDIDEESDIGQIGTNMWREEMECLDWLDTKSPNSVVYVNFG
SITVMSAKQLVEFAWGLAATKKDFLWVIRPDLVAGDVPMLPPDFLIETANRRMLASWCP
QEKVLSHPAVGGFLTHSGWNSTLESLSGGVPMVCWPFFAEQQTNCKYCCDEWEVGM
EIGGDVRREEVEELVRELMDGDKGKKMRQKAEEWQRLAEEATKPIYGSSELNFQMVV
DKVLLGE
88A1
Nucleotide sequence (SEQ ID NO: 97)
ATGGGTGAAGAAGCTATAGTTCTGTATCCTGCACCACCAATAGGTCACTTAGTGTC
CATGGTTGAGTTAGGTAAAACCATCCTCTCCAAAAACCCATCTCTCTCCATCCACAT
TATCTTAGTTCCACCGCCTTATCAGCCGGAATCAACCGCCACTTACATCTCCTCCGT
CTCCTCCTCCTTCCCTTCAATAACCTTCCACCATCTTCCCGCCGTCACACCGTACTC
CTCCTCCTCCACCTCTCGCCACCACCACGAATCTCTCCTCCTAGAGATCCTCTGTTT
TAGCAACCCAAGTGTCCACCGAACTCTTTTCTCACTCTCTCGGAATTTCAATGTCCG
AGCAATGATCATCGATTTCTTCTGCACCGCCGTTTTAGACATCACCGCTGACTTCAC
GTTCCCGGTTTACTTCTTCTACACCTCTGGAGCCGCATGTCTCGCCTTTTCCTTCTA
TCTCCCGACCATCGACGAAACAACCCCCGGAAAAAACCTCAAAGACATTCCTACAG
TTCATATCCCCGGCGTTCCTCCGATGAAGGGCTCCGATATGCCTAAGGCGGTGCTC
GAACGAGACGATGAGGTCTACGATGTTTTTATAATGTTCGGTAAACAGCTCTCGAA
GTCGTCAGGGATTATTATCAATACGTTTGATGCTTTAGAAAACAGAGCCATCAAGGC
CATAACAGAGGAGCTCTGTTTTCGCAATATTTATCCAATTGGACCGCTCATTGTAAA
CGGAAGAATCGAAGATAGAAACGACAACAAGGCAGTTTCTTGTCTCAATTGGCTGG
ATTCGCAGCCGGAAAAGAGTGTTGTGTTTCTCTGTTTTGGAAGCTTAGGTTTGTTCT
CAAAAGAACAGGTGATAGAGATTGCTGTTGGTTTAGAGAAAAGTGGGCAGAGATTC
TTGTGGGTGGTCCGTAATCCACCCGAGTTAGAAAAGACAGAACTGGATTTGAAATC
ACTCTTACCAGAAGGATTCTTAAGCCGAACCGAAGACAAAGGGATGGTCGTGAAAT
CATGGGCTCCGCAAGTTCCGGTTCTGAATCATAAAGCAGTCGGGGGATTCGTCACT
CATTGCGGTTGGAATTCAATTCTTGAAGCTGTTTGTGCTGGTAAATAATGTATATAT
ATACACATTTTTCGATTATATATATGCTTAAAATGTTCATTGTGGTTAATTGAATTGGT
TTACTATATAATAGGTGTGCCGATGGTGGCTTGGCCGTTGTACGCTGAGCAGAGGT
TTAATAGAGTGATGATTGTGGATGAGATCAAGATTGCGATTTCGATGAATGAATCAG
AGACGGGTTTCGTGAGCTCTACAGAGGTGGAGAAACGAGTCCAAGAGATAATTGG
GGAGTGTCCGGTTAGGGAGCGAACCATGGCTATGAAGAACGCAGCCGAATTAGCC
TTGACAGAAACTGGTTCGTCTCATACCGCATTAACTACTTTACTCCAGTCGTGGAGC
CCAAAGTGA
Amino acid sequence (SEQ ID NO: 133)
MGEEAIVLYPAPPIGHLVSMVELGKTILSKNPSLSIHIILVPPPYQPESTATYISSVSSSFPS
ITFHHLPAVTPYSSSSTSRHHHESLLLEILCFSNPSVHRTLFSLSRNFNVRAMIIDFFCTA
VLDITADFTFPVYFFYTSGAACLAFSFYLPTIDETTPGKNLKDIPTVHIPGVPPMKGSDMP
KAVLERDDEVYDVFIMFGKQLSKSSGIIINTFDALENRAIKAITEELCFRNIYPIGPLIVNGRI
EDRNDNKAVSCLNWLDSQPEKSVVFLCFGSLGLFSKEQVIEIAVGLEKSGQRFLWVVR
NPPELEKTELDLKSLLPEGFLSRTEDKGMVVKSWAPQVPVLNHKAVGGFVTHCGWNSI
LEAVCAGVPMVAWPLYAEQRFNRVMIVDEIKIAISMNESETGFVSSTEVEKRVQEIIGEC
PVRERTMAMKNAAELALTETGSSHTALTTLLQSWSPK
89B1
Nucleotide sequence (SEQ ID NO: 99)
ATGAAAGTGAACGAGGAAAACAACAAGCCGACAAAGACCCATGTCTTAATCTTCCC
ATTTCCGGCGCAAGGTCACATGATTCCCCTCCTCGACTTCACCCACCGCCTTGCTC
TCCGCGGCGGCGCCGCCTTAAAAATAACCGTCCTAGTCACTCCAAAAAACCTTCCT
TTTCTCTCTCCGCTTCTCTCCGCCGTAGTTAACATCGAACCACTTATCCTCCCTTTT
CCCTCCCACCCTTCAATCCCCTCCGGCGTCGAAAACGTCCAAGACTTACCTCCTTC
AGGCTTCCCTTTAATGATCCACGCGCTTGGTAATCTCCACGCGCCGCTTATCTCTT
GGATTACTTCTCACCCTTCTCCTCCAGTAGCCATCGTATCTGATTTCTTCCTTGGTT
GGACCAAAAACCTCGGAATCCCTCGTTTCGATTTCTCTCCCTCCGCTGCTATCACTT
GCTGCATACTCAATACTCTCTGGATCGAAATGCCCACCAAGATCAACGAAGATGAC
GATAACGAGATCCTCCACTTTCCCAAGATCCCGAATTGTCCAAAATACCGTTTTGAT
CAGATCTCCTCTCTTTACAGAAGTTACGTTCACGGAGATCCAGCTTGGGAGTTCATA
AGAGACTCCTTTAGAGATAACGTGGCGAGTTGGGGACTCGTCGTGAACTCGTTCAC
CGCCATGGAAGGTGTTTATCTCGAACATCTTAAGCGAGAGATGGGCCATGATCGTG
TATGGGCTGTAGGCCCAATTATTCCGTTATCTGGGGATAACCGTGGTGGCCCGACT
TCTGTTTCTGTTGATCACGTGATGTCGTGGCTTGACGCACGTGAGGATAACCACGT
GGTGTACGTGTGCTTTGGAAGTCAAGTAGTTTTGACTAAAGAGCAGACTCTTGCAC
TCGCCTCTGGGCTTGAGAAAAGCGGCGTCCATTTCATATGGGCCGTAAAGGAGCC
CGTTGAGAAAGACTCAACACGTGGCAACATCCTGGACGGTTTCGACGATCGCGTG
GCTGGGAGAGGTCTGGTGATCAGAGGATGGGCTCCACAAGTAGCTGTGCTACGTC
ACCGAGCCGTTGGCGCGTTTTTAACGCACTGTGGTTGGAACTCTGTGGTGGAGGC
GGTTGTCGCCGGCGTTTTGATGCTGACGTGGCCGATGAGAGCTGACCAGTACACT
GACGCGTCTCTGGTGGTTGATGAGTTGAAAGTAGGTGTGCGTGCTTGCGAAGGAC
CTGACACGGTGCCTGACCCGGACGAGTTAGCTCGAGTTTTCGCTGATTCCGTGAC
CGGAAATCAAACGGAGAGGATCAAAGCCGTGGAGCTGAGGAAAGCAGCGTTGGAT
GCGATTCAAGAACGTGGGAGCTCAGTGAATGATTTAGATGGATTTATCCAACATGT
CGTTAGTTTAGGACTAAACAAATGA
Amino acid sequence (SEQ ID NO: 134)
MKVNEENNKPTKTHVLIFPFPAQGHMIPLLDFTHRLALRGGAALKITVLVTPKNLPFLSPL
LSAVVNIEPLILPFPSHPSIPSGVENVQDLPPSGFPLMIHALGNLHAPLISWITSHPSPPVA
IVSDFFLGWTKNLGIPRFDFSPSAAITCCILNTLWIEMPTKINEDDDNEILHFPKIPNCPKY
RFDQISSLYRSYVHGDPAWEFIRDSFRDNVASWGLVVNSFTAMEGVYLEHLKREMGH
DRVWAVGPIIPLSGDNRGGPTSVSVDHVMSWLDAREDNHVVYVCFGSQVVLTKEQTL
ALASGLEKSGVHFIWAVKEPVEKDSTRGNILDGFDDRVAGRGLVIRGWAPQVAVLRHR
AVGAFLTHCGWNSVVEAVVAGVLMLTWPMRADQYTDASLVVDELKVGVRACEGPDTV
PDPDELARVFADSVTGNQTERIKAVELRKAALDAIQERGSSVNDLDGFIQHVVSLGLNK

1. (canceled)

2. The method of claim 27 wherein said glycosyltransferase is encoded by a nucleic acid molecule consisting of a nucleic acid sequence of SEQ ID NO: 7, 8, 10, 12, 14, 18, 22-27, 29-32, 35, 39, 40, 53, 55, 66, 81, 84, 91, 97 or 99.

3. The method of claim 27 wherein said nucleic acid molecule has at least about 80%, 90% or 99% homology to a nucleic acid sequence of SEQ ID NO: 7, 8, 10, 12, 14, 18, 22-27, 29-32, 35, 39, 40, 53, 55, 66, 81, 84, 91, 97 or 99 and regioselectively modifies an aglycone with a sugar moiety.

4. The method of claim 27 wherein said aglycone is an isoflavone.

5. The method of claim 4 wherein said isoflavone is daidzein.

6. The method of claim 27 wherein said aglycone is a stilbene.

7. The method of claim 6 wherein said stilbene is trans-resveratrol.

8-25. (canceled)

26. A modified aglycone formed by the method of claim 27.

27. A method for regioselective modification of an aglycone with a sugar moiety, comprising contacting the aglycone with a glycosyltransferase encoded by a nucleic acid molecule selected from the group consisting of:
i) nucleic acid molecules comprising a nucleic acid sequence of SEQ ID NO: 7, 8, 10, 12, 14, 18, 22-27, 29-32, 35, 39, 40, 53, 55, 66, 81, 84, 91, 97 or 99;
ii) nucleic acid molecules that hybridize under stringent hybridization conditions to a nucleic acid molecule in (i) and that regioselectively modify an aglycone with a sugar moiety; and
iii) nucleic acid molecules that are degenerate as a result of the genetic code to the sequences as defined in (i) and (ii) above.

28. The modified aglycone of claim 26 wherein said aglycone is an isoflavone.

29. The modified aglycone of claim 28 wherein said isoflavone is daidzein.

30. The modified aglycone of claim 26 wherein said aglycone is a stilbene.

31. The modified aglycone of claim 30 wherein said stilbene is trans-resveratrol.

32. Glycosylated resveratrol prepared by the method of claim 27.

33. The glycosylated resveratrol of claim 32, wherein the resveratrol is glycosylated at the 3-OH position.

34. The glycosylated resveratrol of claim 32, wherein the resveratrol is glycosylated at the 4′-OH position.

35. Glycosylated resveratrol, wherein the resveratrol is glycosylated at the 3-OH position.

36. Glycosylated resveratrol, wherein the resveratrol is glycosylated at the 4′-OH position.


SEQUENCE LISTING (134 sequences)

SEQ ID NO: 1 - DNA, 1422 bp, Arabidopsis thaliana
atgaaagtagaacttgtgttcataccatcgccgggcgttggccatatccgagcaacaacggcgttagcaaagcttctcgttgccagcgacaaccgcctctccgtcactctcatcgtcattccttcacgagtctccgacgacgcttcttcctccgtctacacgaactccgaagaccgtctccgctacatcctcctccccgcccgagatcaaactactgatctcgtatcttacatcgacagccagaaaccacaagtaagagccgtcgtgtccaaggtcgctggagatgtttcaacacgttcagactcacggctagctgggattgtcgtagacatgttctgcacgtccatgatagacatcgccgatgagtttaacctctcggcttatatcttctacacgtccaacgcttcttatctcgggctacagttccacgttcaatctctttacgacgagaaagaactcgacgtaagtgagttcaaagatacggagatgaagtttgacgttccaactctgactcagccttttccggcaaaatgtttgccttcagtgatgctaaacaagaaatggtttccttacgttttgggtcgagctagaagttttagagcaacgaagggtattttggtaaattcggtggctgacatggaacctcaggcgttgagtttcttttccggtggaaatgggaatacaaatatccctccggtgtacgcggttgggcccattatggacttagaatctagcggcgatgaagagaagagaaaggagattttacattggctaaaagagcaaccgacgaaatctgtagtgtttctctgttttgggagcatgggaggtttcagtgaggaacaagcaagagaaatagctgtggcgctcgagcgaagcggacacaggtttctctggtcgcttcgccgcgcttctcctgttggaaacaagtctaatcctcctcccggagaattcacgaacttagaggagattcttccaaaagggtttttagatcggacggtggagatagggaagatcataagctgggcaccacaagtagatgtgttgaatagtcctgctataggagcgttcgtgacacattgtggatggaactcaattctcgagagtctttggttcggtgttccgatggcggcgtggcctatctatgctgagcaacagtttaacgcgtttcatatggtggatgagcttggtttagcggcggaggtaaagaaggagtaccgtagagattttctggtggaggagccggagattgtgacggctgatgagatagagagagggatcaagtgtgcgatggagcaggatagcaagatgaggaagagggtgatggagatgaaggataagctccacgtggcgttggtggacggtggatcttcgaactgtgctctaaagaagtttgttcaagacgtggtcgataatgttccataa

SEQ ID NO: 2 - DNA, 1458 bp, Arabidopsis thaliana
atgaaactggagctggtgttcataccatcacctggtgacggacatctccggccattagtggaggtagctaagcttcatgttgaccgtgacgaccatctctccatcaccatcatcatcatccctcagatgcatggatttagtagcagtaactcttcttcttacatcgcttctctctcctctgattctgaagaacgtcttagctacaacgttctctccgtccctgataaaccagactccgatgacaccaaaccacattttttcgactacattgataacttcaagccgcaggtcaaagccacggtggaaaaacttactgacccgggtccaccagattcgccgtcgcgtcttgctggattcgtggtggatatgttttgcatgatgatgattgatgtcgctaatgagtttggtgttcccagttacatgttttacacatccaacgcaacgtttcttggattgcaagttcatgttgaatacctttacgacgttaagaactatgacgttagtgacctcaaggactcggacactactgagctggaagttccttgtttgactcgtcctttaccggttaagtgtttcccctcggttctattaaccaaggagtggttaccggttatgtttagacaaaccagaagattccgagaaactaaaggtattttggtaaatacattcgctgagcttgagcctcaagctatgaagtttttctccggcgtagatagtcctctgcctacggtgtacacagttggaccggttatgaatcttaaaatcaacggtccaaattcatctgacgataagcaatcggagatcctacggtggctagacgagcagccacgtaaatccgttgttttcctctgtttcggaagcatgggaggtttccgtgagggccaagctaaagaaatcgcaatcgcgcttgagcgaagtggtcaccgctttgtctggtctcttcgtcgtgctcaaccaaaaggatcgataggacctcccgaagaatttacgaatcttgaggaaattctcccggaaggattcttggaacggacggcagagataggaaagattgtaggttgggctccacaaagcgccattctagcaaatcctgcgatcggagggttcgtgtcgcattgtggatggaactcgacgctagagagtctatggttcggagttccgatggctacgtggccgctttacgcagagcaacaagttaacgcgttcgagatggttgaggagctagggctagcggtggaggtccgaaatagtttccgaggagatttcatggcggcggatgatgagttgatgacggcagaggagatagagagagggatccggtgtttgatggagcaggatagtgacgtgaggagtagagtgaaggagatgagcgagaagagtcacgtagctttaatggacggtggatcttcgcacgttgctcttctaaagtttattcaagacgtcactaagaatatctcttga

SEQ ID NO: 3 - DNA, 1437 bp, Arabidopsis thaliana
atgaagattgagcttgtgttcatacctttgccggggattggtcatctcaggccaaccgtgaagctagcgaagcaactcataggcagcgaaaaccgtctttcgatcaccataatcatcatcccttcaagatttgacgccggtgatgcatccgcctgtatcgcatctctcaccacgttgtctcaagatgatcgcctccattacgaatccatatccgtcgcaaaacaaccaccaacctccgacccggatcctgttccggctcaagtgtacatagagaaacaaaagacgaaagtgagagatgcagtcgcggcgagaatcgtcgatccaacaagaaagctcgcgggattcgtggtggacatgttctgttcctcgatgatcgatgtagctaacgagtttggagttccgtgttatatggtatacacatcgaacgctacgtttttaggaaccatgcttcacgttcaacaaatgtacgatcaaaagaagtatgacgtcagcgagttagaaaactcggtcaccgagttggagtttccgtctctgactcgtccttatccagtgaagtgtcttcctcatatcctcacttcaaaggagtggttacctctctctctagctcaagctaggtgtttccggaagatgaagggtattttggtaaatacagttgctgagcttgaacctcacgctttgaaaatgttcaatattaatggtgacgatcttcctcaagtttatcctgttggaccagtgttgcatctcgaaaacggcaatgacgatgatgagaagcaatcggaaattttgcggtggctcgacgagcaaccgtctaaatctgttgtgtttctctgctttgggagcttgggaggtttcactgaagaacaaacaagagaaaccgctgtggccctagatagaagcggtcagcggtttctttggtgtcttcgtcacgcatcgccaaatataaaaacagatcgtcccagagattacacgaatcttgaggaggttttaccggaggggttcttggaacggactttggatagagggaaagtgattggatgggcaccacaagtggcggtactagagaagccggcgataggagggtttgtcactcactgcggttggaactctattttagagagcttgtggttcggtgttccaatggtgacgtggccgctatacgcggaacagaaggttaacgcgtttgagatggttgaggagctgggtttggcggtggagatacggaagtacttaaaaggagatttgttcgccggagagatggagacggttaccgcggaggatatagagagagccattaggcgtgtgatggagcaagacagtgacgttaggaacaacgtgaaagagatggcggagaagtgccacttcgcgttaatggacggtggatcttcgaaggcggctttggaaaagtttattcaagacgtgatagagaatatggattaa

SEQ ID NO: 4 - DNA, 1440 bp, Arabidopsis thaliana
atgaaaatagagctagtattcattccctctccggcaattagtcatctcatggcgacggtagagatggcggagcaactagttgataaaaacgacaacctctctatcaccgtaatcatcatatcttttagttctaaaaatacatccatgatcacctctcttacatccaacaaccgcctccggtacgaaataatctccggaggagatcaacaaccaacggagctcaaagcaactgattcccacatccaaagtctaaagccactggtgagagacgcggttgctaaactcgtagattccactctaccagacgcgcctcgtcttgcgggattcgttgttgacatgtactgcacgtcgatgatcgatgtcgctaacgaatttggcgtccctagttacttgttttacacctctaacgctggatttcttggacttttgcttcacattcagttcatgtacgatgcagaggatatctatgacatgagcgaattagaagactctgacgtagagttggtggttccgagtttgactagtccttatccgttgaaatgtcttccttacattttcaaatcaaaagagtggctcactttttttgtaactcaagcgagaagattcagagaaactaagggcattttggtaaacacggttcctgacttggaacctcaagcgttgacgtttctttccaatggtaacattccacgtgcttacccagtaggaccattgttgcatctcaaaaacgtaaattgtgattacgtggacaagaagcaatcggagattttacggtggctagacgagcaaccgccaagatctgtagtgttcctctgtttcgggagcatgggagggttcagtgaggaacaagtgagagaaaccgcattagctctcgatcgaagcggccaccggtttctttggtctctccgtcgtgcatctccgaatatattgagagagcctcccggagaattcacaaacctagaggagattctcccagaagggtttttcgatcggacggctaacagaggaaaggttatcggatgggctgaacaggtggccatattggcgaagccggcgatcggaggttttgtttctcacggcggatggaattcgacgttggagagtttgtggtttggtgttccgatggcgatttggccgctttacgctgaacagaagtttaacgctttcgagatggtggaagagcttggtttggctgtggagatcaagaagcattggcgaggagatcttttgttggggaggtcggagattgtgacggcggaggagattgagaaaggaatcatatgtttgatggagcaagacagtgacgtcaggaagagagtgaatgagatcagcgagaagtgccacgtggctttaatggacggtggatcgtcagaaactgctttgaaaagatttattcaagacgtaacggagaatattgcttggtcggaaactgaaagctag

SEQ ID NO: 5 - DNA, 1488 bp, Arabidopsis thaliana
atgaaatttgagcttgttttcatcccctatcccggaatcggtcatctccgatcaacggtagaaatggcaaagctactagtggaccgtgaaactcgtctctctatctccgttatcatccttcctttcatttccgaaggcgaagtcggtgcttccgattacatcgcagccctctccgcctcatccaacaaccgcctccgctacgaagttatctccgccgtagatcaaccaaccatcgagatgacgacaattgaaatccatatgaagaaccaagaaccaaaggtgagaagcaccgttgcaaaactccttgaagactattcgtctaaaccggactcgccgaagatcgctggctttgttctagacatgttttgcacttcgatggtagatgtagcgaacgagtttggtttcccgagttatatgttttacacctccagtgccgggattctctcagttacatatcatgttcaaatgttgtgcgatgagaacaagtacgatgttagtgaaaatgattatgcagactcggaagctgtgttgaactttccgagtttgagtcgtccttatccggtgaagtgtcttcctcacgctctggcagctaatatgtggctcccggtgtttgtaaaccaagcgagaaagtttagggagatgaaaggtattttggtaaatactgttgctgagcttgaaccttatgtgttaaagtttctttctagtagtgatactcctcctgtttatcctgttggaccattgttgcatcttgagaaccaacgtgatgattctaaggacgagaaacggttggagattatacggtggttggatcagcaaccaccaagttcggttgtgtttctctgctttgggagcatgggaggcttcggtgaggaacaagtaagagagatcgcaatcgcgttagagcgaagtgggcaccggtttctctggtctcttcgtcgcgcatctccgaatatattcaaagaacttccaggagagtttactaatctagaggaagttctcccggaaggattctttgatcgaacgaaagatataggtaaagtgattggatgggctccacaagtagccgttcttgcgaatccggctataggaggtttcgtaactcattgcgggtggaattctacgctagagagtctttggtttggtgttccaacagctgcatggccgttatacgcagagcagaagttcaatgctttcttaatggtggaggagcttggattggcggtggagataaggaagtattggcgaggtgaacatttggcgggattaccgacggctactgtgacagcggaggagatagagaaagcaatcatgtgtctaatggaacaagatagtgacgtgaggaaaagagtgaaggatatgagcgagaaatgccatgtggctttaatggatggtggatcgtcgcgtactgcgttgcaaaagtttattgaagaggttgcgaagaatatagtttcactagataaggaatttgagcatgtagctcttaaatga

SEQ ID NO: 6 - DNA, 1443 bp, Arabidopsis thaliana
atgaacaaatttgcgcttgtcttcgtaccatttcctatacttggtcatctcaaatcaaccgccgagatggctaagctactagtggagcaagaaactcgcctctctatctccattatcatccttcctcttctttccggagacgacgtcagtgcttccgcttatatctcagctctttccgccgcatccaacgaccgccttcactatgaagtgatctcggacggagatcaaccaaccgtcgggttacatgtcgataaccacatcccgatggtgaaacgtaccgttgcaaaactcgttgatgactactcaaggcggccggactcgccgaggctcgctggtttagttgttgacatgttttgtatctcggtgatagacgtggctaatgaggttagtgttccgtgttacttgttttacacgtcaaacgttgggattcttgctcttgggttacatattcagatgttgtttgataagaaggagtacagtgtcagtgaaactgattttgaagactcggaagttgtgttggatgttccgagtttgacttgtccttatccggtgaagtgtcttccttatggtttggcaacgaaagagtggcttcctatgtatctaaatcaaggtagaagattcagagagatgaaaggtattttggtaaatacttttgctgagcttgaaccttatgcgttggagtctcttcactctagtggtgatactcctcgtgcttatccagtgggaccattgttgcatctcgagaaccatgttgacggttctaaagacgagaagggttcggacattttacggtggttagatgaacaaccacctaaatcggtagtgttcctctgctttggaagcataggaggctttaacgaggaacaagcaagagaaatggccattgcacttgagagaagtggtcaccgcttcttgtggtctcttcgccgtgcatctcgagatatagataaggaacttcccggagaattcaagaatcttgaagaaattctcccggaaggattctttgatcggacaaaggataaaggaaaggtgatcggatgggctccacaagtagccgtgctggctaagccagcaatcggaggttttgttactcattgcgggtggaactcgatactcgagagtctttggttcggtgttcctatagcgccatggccgttatacgctgagcagaagtttaatgctttcgtgatggtggaggagcttggtttggcagtgaagataagaaagtattggcgaggcgatcagttggtgggaacggcgacggtcatagtgacggcagaggagatagagagaggaatcagatgtttgatggagcaagatagtgacgtgaggaatagagtgaaggagatgagtaagaaatgtcacatggctttaaaggatggtggctcgtctcaatctgctttgaaattatttattcaagacgttacgaagtatattgcttga

SEQ ID NO: 7 - DNA, 1446 bp, Arabidopsis thaliana
atggggaagcaagaagatgcagagctcgtcatcatacctttccctttctccggacacattctcgcaacaatcgaactcgccaaacgtctcataagtcaagacaatcctcggatccacaccatcaccatcctctattggggattaccttttattcctcaagctgacacaatcgctttcctccgatccctagtcaaaaatgagcctcgtatccgtctcgttacgttgcccgaagtccaagaccctccaccaatggaactctttgtggaatttgccgaatcttacattcttgaatacgtcaagaaaatggttcccatcatcagagaagctctctccactctcttgtcttcccgcgatgaatcgggttcagttcgtgtggctggattggttcttgacttcttctgcgtccctatgatcgatgtaggaaacgagtttaatctcccttcttacattttcttgacgtgtagcgcagggttcttgggtatgatgaagtatcttccagagagacaccgcgaaatcaaatcggaattcaaccggagcttcaacgaggagttgaatctcattcctggttatgtcaactctgttcctactaaggttttgccgtcaggtctattcatgaaagagacctacgagccttgggtcgaactagcagagaggtttcctgaagctaagggtattttggttaattcatacacagctctcgagccaaacggttttaaatatttcgatcgttgtccggataactacccaaccatttacccaatcgggccgatattatgctccaacgaccgtccgaatttggactcatcggaacgagatcggatcataacttggctagatgaccaacccgagtcatcggtcgtgttcctctgtttcgggagcttgaagaatctcagcgctactcagatcaacgagatagctcaagccttagagatcgttgactgcaaattcatctggtcgtttcgaaccaacccgaaggagtacgcgagcccttacgaggctctaccacacgggttcatggaccgggtcatggatcaaggcattgtttgtggttgggctcctcaagttgaaatcctagcccataaagctgtgggaggattcgtatctcattgtggttggaactcgatattggagagtttgggtttcggcgttccaatcgccacgtggccgatgtacgcggaacaacaactaaacgcgttcacgatggtgaaggagcttggtttagccttggagatgcggttggattacgtgtcggaagatggagatatagtgaaagctgatgagatcgcaggaaccgttagatctttaatggacggtgtggatgtgccgaagagtaaagtgaaggagattgctgaggcgggaaaagaagctgtggacggtggatcttcgtttcttgcggttaaaagattcatcggtgacttgatcgacggcgtttctataagtaagtag

SEQ ID NO: 8 - DNA, 1425 bp, Arabidopsis thaliana
atggcgaagcagcaagaagcagagctcatcttcatcccatttccaatccccggacacattctcgccacaatcgaactcgcgaaacgtctcatcagtcaccaacctagtcggatccacaccatcaccatcctccattggagcttaccttttcttcctcaatctgacactatcgccttcctcaaatccctaatcgaaacagagtctcgtatccgtctcattaccttacccgatgtccaaaaccctccaccaatggagctatttgtgaaagcttccgaatcttacattcttgaatacgtcaagaaaatggttcctttggtcagaaacgctctctccactctcttgtcttctcgtgatgaatcggattcagttcatgtcgccggattagttcttgatttcttctgtgtccctttgatcgatgtcggaaacgagtttaatctcccttcttacatcttcttgacgtgtagcgcaagtttcttgggtatgatgaagtatcttctggagagaaaccgcgaaaccaaaccggaacttaaccggagctctgacgaggaaacaatatcagttcctggttttgttaactccgttccggttaaagttttgccaccgggtttgttcacgactgagtcttacgaagcttgggtcgaaatggcggaaaggttccctgaagccaagggtattttggtcaattcatttgaatctctagaacgtaacgcttttgattatttcgatcgtcgtccggataattacccacccgtttacccaatcgggccaattctatgctccaacgatcgtccgaatttggatttatcggaacgagaccggatcttgaaatggctcgatgaccaacccgagtcatctgttgtgtttctctgcttcgggagcttgaagagtctcgctgcgtctcagattaaagagatcgctcaagccttagagctcgtcggaatcagattcctctggtcgattcgaacggacccgaaggagtacgcgagcccgaacgagattttaccggacgggtttatgaaccgagtcatgggtttgggccttgtttgtggttgggctcctcaagttgaaattctggcccataaagcaattggagggttcgtgtcacactgcggttggaactcgatattggagagtttgcgtttcggagttccaattgccacgtggccaatgtacgcggaacaacaactaaacgcgttcacgattgtgaaggagcttggtttggcgttggagatgcggttggattacgtgtcggaatatggagaaatcgtgaaagctgatgaaatcgcaggagccgtacgatctttgatggacggtgaggatgtgccgaggaggaaactgaaggagattgcggaggcgggaaaagaggctgtgatggacggtggatcttcgtttgttgcggttaaaagattcatagatgggctttga

SEQ ID NO: 9 - DNA, 1431 bp, Arabidopsis thaliana
atgaaagcagaagcagagatcatcttcgttacatatccatcccctggtcatcttcttgtctccattgaattcgctaaatctctcatcaaacgtgatgatcgcatccacaccatcaccatcctctactgggctttacctctcgctcctcaagcccaccttttcgctaagtccctcgttgcttcacagcctcgaatccgtctccttgcgttgcctgatgttcaaaaccctccaccattggaactcttctttaaagctcccgaagcttatattcttgagtccaccaagaaaacagttcctttagtcagagacgctctctccactctagtttcttcacgtaaagaatccggttcggttcgtgtagtcggtttggttatcgattttttttgtgttccaatgatcgaagtggcaaacgagcttaaccttccttcttacatcttcctaacgtgtaacgctgggtttttaagtatgatgaagtatctccctgagagacatcgcataaccacttctgagctagatttaagctccggcaacgtagaacatccaattcctggctacgtctgctccgtgccgacgaaggttttgcctccaggtctattcgtgagagagtcctacgaggcttgggtcgagattgcagagaagttccctggagccaagggcattttggtaaactcagtcacatgtcttgagcagaatgcatttgattacttcgctcgtcttgatgagaactatcctccggtttacccggtcggaccggttcttagtttgaaggatcgtccgtctccaaatctggacgcatcggaccgggatcggatcatgagatggctcgaggaccagccggagtcgtcaattgtgtatatctgcttcggaagcctcggaatcattggcaagctgcagattgaagagatagctgaagccttggaactcaccggccacaggtttctttggtcaatacgtacaaatccgacggagaaagcgagcccgtacgatctgttgccggagggatttctcgatcggacggccagtaagggattggtgtgtgattgggccccgcaagtagaagttctggcccataaagcgctcggaggattcgtgtctcactgcggttggaactctgtactggagagcttatggttcggtgttccgatcgccacgtggccaatgtacgctgagcaacagttaaacgcattctcgatggtgaaggagttagggttagccgtggagctgcgtttagactacgtttcggcgtacggagagatagtaaaagctgaggagatcgcgggagccatacgatcattgatggacggtgaggatacgccgaggaagagagtgaaggagatggcggaagcggcgaggaatgctttgatggacggaggatcttcgtttgttgcggttaaacgatttctcgacgagttgatcggcggagatgtttag

SEQ ID NO: 10 - DNA, 1440 bp, Arabidopsis thaliana
atggtgaaggaaacagagctaatcttcattccagttccatccacaggtcatattctcgtccatattgaattcgccaagcgtctcatcaatctcgaccatcggatccacaccatcactattctcaacttatcctcaccctcttctcctcacgcctccgtcttcgccagatctctcatcgcttcccagcccaaaatccgtctccacgaccttccccctatccaagatcctcctccattcgatctttaccaaagagctcccgaagcttacatagtaaaactcatcaagaaaaatactcctctgataaaagacgccgtctccagcatcgtcgcgtcgcgtcgtggaggctcagattcggttcaagtcgccggtttggttctcgatttattctgcaattcattggtaaaagatgttggcaacgagcttaatcttccttcttacatataccttacgtgtaacgctagatacttggggatgatgaaatatattccggatcggcatcggaaaatcgcatctgagttcgatttgagctccggcgatgaagaattgccggttccgggattcataaacgctattccgacgaaatttatgccgcctggattgttcaataaggaagcttacgaggcttacgtagagctagcgccgagattcgcagatgcgaagggtattttggttaattccttcacggagcttgagccgcacccgtttgactatttctctcacctggagaaattccctccggtttacccggtcggaccgattctcagcttgaaagatcgagcgagtccgaacgaagaagcagtcgatcgggatcagatcgttgggtggctcgatgatcagccggagtcatcggtggtgttcctctgtttcgggagcagaggaagcgttgatgagccgcaagtgaaggagatagctcgagctttggaactcgtcggctgcagatttctttggtcaattagaacaagcggcgacgtcgagacgaatcctaacgatgtgttgccggaggggttcatgggccgagtagcaggccgaggtttggtatgtggttgggctccacaagtggaagtgttggcccataaagcaataggaggatttgtgtctcactgtggttggaactccacgcttgaaagcttatggttcggggttcctgtcgcaacgtggccgatgtacgcagagcaacagcttaacgccttcacgctggtgaaagagcttgggcttgcggtggacctgcggatggattacgtgtcgagtcgtgggggtttggtgacttgtgatgagatagccagagccgtacgatctttgatggacggtggagatgagaagagaaaaaaggttaaggagatggctgatgcggcaaggaaggctttgatggatggaggatcgtcttctttggcaactgctcgattcatcgcagaattgtttgaagatggttcgtcgtgctaa

SEQ ID NO: 11 - DNA, 1443 bp, Arabidopsis thaliana
atgaagacagcagagctcatattcgttcctctgccggagaccggccatctcttgtcaacgatcgagtttggaaagcgtctactcaatctagaccgtcggatttctatgattacaatcctctccatgaatcttccttacgctcctcacgccgacgcttctcttgcttcgctaacagcctccgagcctggtatccgaatcatcagtctcccggagatccacgatccacctccgatcaagcttcttgacacttcctccgagacttacatcctcgatttcatccataaaaacataccttgtctcagaaaaaccatccaagatttagtctcatcatcatcatcttccggaggtggtagtagtcatgtcgccggcttgattcttgatttcttctgcgttggtttgatcgacatcggccgtgaggtaaaccttccttcctatatcttcatgacttccaactttggtttcttaggggttctacagtatctcccggaacgacaacgtttgactccgtcggagttcgatgagagctccggcgaggaagagttacatattccggcgtttgtgaaccgtgttcccgccaaggttctgccgccaggtgtgttcgataaactctcttacgggtctctggtcaaaatcggcgagcgattacatgaagccaagggtattttggttaattcatttacccaagtggagccttatgctgctgaacatttttctcaaggacgagattaccctcacgtgtatcctgttgggccggttctcaacttaacgggccgtacaaatccgggtctagcttcggcccaatataaagagatgatgaagtggcttgacgagcaaccagactcgtcggttttgttcctgtgtttcgggagcatgggagtcttccctgcacctcagatcacagagattgctcacgcgctcgagcttatcgggtgcaggttcatctgggcgatccgtacgaacatggcgggagatggcgatcctcaggagccgcttccagaaggatttgtcgatcgaacaatgggccgtggaattgtgtgtagttgggctccacaagtggatatcttggcccacaaggcaacaggtggattcgtttctcactgcgggtggaattccgtccaagagagtctatggtacggtgtacctattgcaacgtggccaatgtatgcggagcaacaactgaacgcatttgagatggtgaaggagttgggcttagcagtggagataaggcttgactacgtggcggatggtgatagggttactttggagatcgtgtcagccgatgaaatagccacagccgtccgatcattgatggatagtgataaccccgtgagaaagaaggttatagaaaaatcttcagtggcgaggaaagctgttggtgatggtgggtcttctacggtggccacatgtaattttatcaaagatattcttggggatcacttttga

SEQ ID NO: 12 - DNA, 1404 bp, Arabidopsis thaliana
atgcggaatgtagagctcatcttcatccccacaccaaccgttggtcatcttgttccgtttcttgaatttgctaggcgtctcattgagcaagatgataggatccgtatcacaatcctcttgatgaaactacaaggtcagtctcatctagacacttatgttaaatcaattgcctcctctcaaccgtttgttagattcattgatgtccctgagttagaggagaaacctacacttggtagtacacaatctgtggaagcttatgtgtatgatgttattgagagaaatatccctcttgtgaggaatatagtcatggatattttaacttctcttgcattggatggagttaaggtcaagggattagttgttgactttttctgtctccctatgattgacgttgctaaagatataagtctccctttctatgtgttcttgactacaaattccgggttcttagctatgatgcagtatctagcagatcgacatagtagagatacatcggtttttgtaagaaactcggaagaaatgttgtcgatacctggatttgtaaaccctgtcccagccaatgttctgccgtcagctctgtttgttgaagatggttatgatgcttacgttaagctggccatattgtttacaaaggccaatggaatcctagtgaatagctcctttgatattgagccttactctgtgaatcattttcttcaagaacagaattatccttctgtttatgctgttggccccatatttgacttgaaagcccagcctcatccagagcaggacctaacccgtcgtgacgagttgatgaaatggcttgatgatcaacccgaggcatcggttgtattcctttgttttgggagtatggcaaggttaagaggttctctagtgaaggaaatagctcatggacttgagctatgtcaatatagattcctctggtcactccgtaaagaagaggtgacaaaggatgatttgccagaggggttccttgaccgtgtcgatggacgtggaatgatatgtggttggtctcctcaggtagaaatactggcccataaggcagtgggaggctttgtttctcactgtggatggaactcaatagtagagagtttgtggtttggcgtgccaattgtgacatggccaatgtatgcagagcaacaactcaatgcgtttctgatggtgaaggaactgaagctagctgtggagctgaagcttgattacagggtacatagtgatgagatagtaaacgcaaacgagatagagaccgctattcgttatgtaatggacacggataataatgttgtgaggaaacgagtgatggatatctcgcagatgatccagagagctacgaagaatggtggatcttcgtttgccgcaattgagaaattcatatatgacgtgataggaattaagccctag

SEQ ID NO: 13 - DNA, 1404 bp, Arabidopsis thaliana
atgaggaatgcagagctcatcttcatcccaacaccaactgttggtcatcttgttccgtttcttgaatttgctaggcgtctcattgagcaggatgatagaatccgtatcaccttcctcttgatgaagcaacaaggtcagtctcatctggattcctatgttaagacaatttcctcgtctctgccgtttgttagatttattgatgtccctgagttagaggagaaaccaacacttggtacacagtctgtggaagcctatgtgtacgattttattgaaacaaatgtccctcttgtgcaaaatataatcatgggtatcctatcttctcctgcatttgatggagttacggtcaagggattcgttgctgattttttctgtctcccgatgattgatgttgcaaaagatgcaagtcttcctttttatgtgttcttgacttcaaattccggattcctagctatgatgcagtatctggcatatggacataagaaagatacctcagtttttgcaagaaactctgaagaaatgttgtcaattcctggatttgtaaaccctgtcccagccaaagtactgccgtcagctctgtttattgaggatggttatgatgctgacgttaaactggctatattgtttacaaaggctaatggaatcctagtgaatacctcctttgatattgagcctacctctctgaatcattttcttggagaagagaattacccttctgtttatgctgttggccccatatttaacccgaaggcccatcctcatccagatcaagacctcgcctgttgtgacgagtcgatgaaatggcttgatgctcaacccgaggcatcagttgtattcctttgttttgggagtatgggtagcttaagaggtcctctagtgaaggaaatagcacatggacttgagctatgtcagtatagattcctctggtcactccgcacagaagaagtgacaaatgatgatcttttgccagagggattcatggaccgtgtcagtggacggggaatgatatgcggttggtctcctcaggtggaaatactggcccataaagcagtgggaggttttgtttctcattgtggatggaactcaatagtagagagtttatggtttggtgtgccaattgtgacatggccaatgtatgcagagcaacagctcaatgcgtttctgatggtgaaggaactgaagctcgcagtggagctgaaactcgattatagtgtacatagtggtgagattgtaagtgcaaacgagatagagacagcgatttcttgtgtaatgaacaaggataataatgttgtgaggaaacgagtgatggatatctcgcagatgatccagagagctacgaagaatggtggatcttcgtttgccgcaattgagaaattcatacatgacgtgataggaaccaggacttag

SEQ ID NO: 14 - DNA, 1443 bp, Arabidopsis thaliana
atggaggaatccaaaacacctcacgttgcgatcataccaagtccgggaatgggtcatctcataccactcgtcgagtttgctaaacgactcgtccatcttcacggcctcaccgttaccttcgtcatcgccggcgaaggtccaccatcaaaagctcagagaaccgtcctcgactctctcccttcttcaatctcctccgtctttctccctcctgttgatctcaccgatctctcttcgtccactcgcatcgaatctcggatctccctcaccgtgactcgttcaaacccggagctccggaaagtcttcgactcgttcgtggagggaggtcgtttgccaacggcgctcgtcgtcgatctcttcggtacggacgctttcgacgtggccgtagaatttcacgtgccaccgtatattttctacccaacaacggccaacgtcttgtcgttttttctccatttgcctaaactagacgaaacggtgtcgtgtgagttcagggaattaaccgaaccgcttatgcttcctggatgtgtaccggttgccgggaaagatttccttgacccggcccaagaccggaaagacgatgcatacaaatggcttctccataacaccaagaggtacaaagaagccgaaggtattcttgtgaataccttctttgagctagagccaaatgctataaaggccttgcaagaaccgggtcttgataaaccaccggtttatccggttggaccgttggttaacattggtaagcaagaggctaagcaaaccgaagagtctgaatgtttaaagtggttggataaccagccgctcggttcggttttatatgtgtcctttggtagtggcggtaccctcacatgtgagcagctcaatgagcttgctcttggtcttgcagatagtgagcaacggtttctttgggtcatacgaagtcctagtgggatcgctaattcgtcgtattttgattcacatagccaaacagatccattgacatttttaccaccgggatttttagagcggactaaaaaaagaggttttgtgatccctttttgggctccacaagcccaagtcttggcgcatccatccacgggaggatttttaactcattgtggatggaattcgactctagagagtgtagtaagcggtattccacttatagcatggccattatacgcagaacagaagatgaatgcggttttgttgagtgaagatattcgtgcggcacttaggccgcgtgccggggacgatgggttagttagaagagaagaggtggctagagtggtaaaaggattgatggaaggtgaagaaggcaaaggagtgaggaacaagatgaaggagttgaaggaagcagcttgtagggtgttgaaggatgatgggacttcgacaaaagcacttagtcttgtggccttaaagtggaaagcccacaaaaaagagttagagcaaaatggcaaccactaa

SEQ ID NO: 15 - DNA, 1455 bp, Arabidopsis thaliana
atgcaaaaaatggcagatggaaacactccacatgtagcaatcataccaagtcccggtataggtcacctcatcccactcgtcgagttagcaaagcgactccttgacaatcacggtttcaccgtcactttcatcatccccggcgattctcctccgtctaaggctcaaagatccgttctcaactctctcccttcctccatagcctccgtcttcctccctcccgccgatctttccgacgttccttcgacagctcgaatcgaaactcggatatcgctcaccgtgactcgttccaacccggcgctccgggagctttttggctcgttatcggcggagaaacgtctcccggcggttctcgtcgtcgatctatttggtacggatgcgttcgacgtggctgctgagttccacgtgtcgccatacattttctatgcatcaaatgccaacgtcctcacgtttctgcttcacttgccgaagctagacgaaacggtgtcgtgtgagtttagggaattaaccgaaccggttattattcccggttgtgtccccataaccggtaaggatttcgtcgatccgtgtcaagaccgaaaagatgaatcatacaaatggcttctacacaacgtcaagagattcaaagaagctgaagggattctagtgaattccttcgtcgatttagagccaaacactataaagattgtacaagaaccggctcctgataaaccaccggtttacctgattgggccgttggttaactcgggttcacacgatgctgacgtgaacgatgagtacaaatgtttaaattggctagacaaccaaccattcgggtcggttctatacgtatcctttggaagcggcggaacactcacgtttgagcagttcattgagctggctcttggcctagcggagagtggaaaacggtttctttgggtcatacgaagtccgagtgggatagctagttcatcgtatttcaatccacaaagccgaaatgatccattttcgtttttaccacaaggcttcttagaccgaaccaaagaaaaaggtctagtggttgggtcatgggctccacaggctcaaattctgactcatacatctataggtggatttttaactcattgtggatggaattcgagtctagaaagtattgtaaacggtgtaccgctcatagcatggccgttatacgcggagcaaaagatgaacgcattgctactcgtggatgttggtgcggctctaagagcacgactgggtgaagacggggtcgtaggaagggaagaagtggcgagagtggtaaaaggattgatagaaggagaagaagggaatgcggtaaggaaaaaaatgaaagagttgaaagaaggatctgttagagtcttaagggacgatggattctctaccaaatcgcttaatgaagtttcgttgaagtggaaagcccaccaacgaaagatcgaccaagaacaggaatcatttctatga

SEQ ID NO: 16 - DNA, 1494 bp, Arabidopsis thaliana
atgagcatagatatttttcaagaaataagaataaagaaaattctactcttaatggcggaagcaaacactccacacatagcaatcatgccgagtcccggtatgggtcaccttatcccattcgtcgagttagcaaagcgactcgttcagcacgactgtttcaccgtcacaatgatcatctccggtgaaacttcgccgtctaaggcacaaagatccgttctcaactctctcccttcctccatagcctccgtatttctccctcccgccgatctttccgatgttccctccacagcgcgaatcgaaactcgggccatgctcaccatgactcgttccaatccggcgctccgggagctttttggctctttatcaacgaagaaaagtctcccggcggttctcgtcgtcgatatgtttggtgcggatgcgttcgacgtggccgttgacttccacgtgtcaccatacattttctatgcatccaatgcaaacgtcttgtcgttttttcttcacttgccgaaactagacaaaacggtgtcgtgtgagtttaggtacttaaccgaaccgcttaagattcccggctgtgtcccgataaccggtaaggactttcttgatacggttcaagaccgaaacgacgacgcatacaaattgcttctccataacaccaagaggtacaaagaagctaaagggattctagtgaattccttcgttgatttagagtcgaatgcaataaaggccttacaagaaccggctcctgataaaccaacggtatacccgattgggccgctggttaacacaagttcatctaatgttaacttggaagacaagttcggatgtttaagttggctagacaaccaaccattcggctcggttctatacatatcatttggaagcggcggaacacttacatgtgagcagtttaatgagcttgctattggtcttgcggagagcggaaaacggtttatttgggtcatacgaagtccaagcgagatagttagttcgtcgtatttcaatccacacagcgagacagaccccttttcgtttttaccaattgggttcttagaccgaaccaaagagaaaggtttggtggttccatcatgggctccacaggttcaaatcctggctcatccatccacatgcgggtttttaacacactgtggatggaattcgaccttagaaagcattgtaaacggtgtaccactcatagcgtggcctttattcgcggagcaaaagatgaatacattgctactcgtggaggatgttggagcggctctaagaatccatgcgggtgaagatgggattgtacggagggaagaagtggtgagagtggtgaaggcactgatggaaggtgaagagggaaaagccataggaaataaagtgaaggagttgaaagaaggagttgttagagtcttgggtgacgatggattgtccagcaagtcatttggtgaagttttgttaaagtggaaaacgcaccagcgagatatcaaccaagagacgtcccactaa

SEQ ID NO: 17 - DNA, 1374 bp, Arabidopsis thaliana
atggaacttcacggagctctagtggctagtccgggcatgggacatgccgtacccatcttagaactcggtaaacatctcctgaaccaccacgggttcgaccgtgtcactgtcttcctagtcacagacgatgtctcacgttcgaaatccctaattggaaaaacgttgatggaagaagatccaaaatttgtgatcaggtttattccactcgatgtttcgggtcaagatctgagtggttcactattgactaaactagcagagatgatgaggaaggcattaccagagatcaagtcttcagtcatggagttagaaccgcggcctagggttttcgtagttgacttgttgggcacggaagctttagaggtggctaaggagcttgggatcatgagaaaacatgttctggttactaccagtgcttggtttctagcttttacggtttatatggcgagtcttgacaaacaggagttgtataagcagttgagtagcataggagcattgcttatacccggatgcagcccggttaagtttgagcgggctcaagatccgagaaaatatattcgggaactcgctgagtctcagcgtattggggatgaggtgataaccgcagatggggtgtttgtgaatacgtggcacagtctggagcaagtgaccatcgggtctttcttggatccagagaatctcggtcgggttatgagaggagtgccggtttatcctgttggaccgctggttagaccagcagaaccaggtttgaaacatggcgtgctggactggcttgacttacaacccaaagagtcagtggtttatgttcttttgggagtggtgggggcactaaccttcgagcagacaaacgagctggcttacggtttggagctgactggccacagatttgtttgggtagtcagaccaccggctgaagacgacccatcggcatcaatgttcgacaagaccaagaatgagacagaacctctcgatttcttacccaacgggtttctagaccgaaccaaagacatcggtttggtggtccgtacatgggcaccacaagaagagattctggcacacaagtcaacaggagggtttgtgactcactgcggatggaactcagttttggagagtattgtgaatggtgtgccaatggtagcttggccgttgtactcagagcagaagatgaacgcgaggatggtttctggggagctaaagattgcgttgcagattaatgttgcagatgggattgtaaagaaggaggtgatagctgaaatggtgaagagagtgatggatgaagaagaaggaaaagagatgagaaagaatgttaaggaactgaagaagacagcagaagaagctctcaacatgactcacattccatctgcttacttcacctaa

SEQ ID NO: 18 - DNA, 1413 bp, Arabidopsis thaliana
atggaccagcctcacgcgcttctagtggctagccctggcttgggtcacctcatccctatcctggagctcggcaaccgtctctcctccgtcctaaacatccacgtcaccattctcgcggtcacctccggctcctcttcaccgacagaaaccgaagccatacatgcagccgcggctagaacaatctgtcaaattacggaaattccctcggtggatgtagacaacctcgtggagccagatgctacaattttcactaagatggtggtgaagatgcgagccatgaagcccgcggtacgagatgccgtgaaattaatgaaacgaaaaccaacggtcatgattgttgactttttgggtacggaactgatgtccgtagccgatgacgtaggcatgacggctaaatacgtttacgttccaactcatgcgtggttcttggcagtcatggtgtacttgccggtgttagatacggtagtggaaggtgagtatgttgatattaaggagcctttgaagataccgggttgtaaaccggtcggaccgaaggagctgatggaaacgatgttagaccggtcgggccagcaatataaagagtgtgtacgagctggcttagaggtacctatgagcgatggtgttttggtaaatacttgggaggagttacaaggaaacactctcgctgcgcttagagaggacgaagaattgagccgggtcatgaaagtaccggtttatcctattgggccaattgttaggactaaccagcatgtagacaaacccaatagtatattcgagtggctagacgagcaacgggaaaggtcagtggtgtttgtgtgtttagggagcggtggaacgttgacgtttgagcaaacagtggaactcgctttgggtttagagttaagtggtcaaaggttcgtttgggttctacgtaggcccgcttcatatctcggggcgatctccagcgatgatgaacaggtaagtgccagtctacctgaaggtttcttggaccgcacgcgtggtgtggggattgtggttacgcaatgggcaccacaagttgagatcttgagccatagatcgatcggtgggttcttgtctcactgcggttggagttcggctttggaaagtttgactaaaggagttccgatcatcgcttggcctctttatgcggagcagtggatgaatgccacgttattgactgaggagatcggtgtggccgttcgtacatcggagttaccgtcggagagagtcatcggaagggaagaagtggcatctctggtgagaaagattatggcggaagaggatgaagaaggacagaaaattagggctaaagctgaggaggtgagggttagctccgaacgagcttggagtaaagacgggtcatcttataattctctattcgaatgggcaaaacgatgttatcttgtaccgtga

SEQ ID NO: 19 - DNA, 1464 bp, Arabidopsis thaliana
atgaagattacaaaaccacatgtggccatgttcgctagccccggaatgggccacatcatcccggtgatcgagctcggaaaacgcttagctggttcccacggcttcgatgtcaccattttcgtccttgaaaccgacgcagcctcagctcaatctcaattccttaactcaccaggctgcgacgcggcccttgttgatatcgttggcctcccaacgcccgatatctccggtttagtcgacccatcagccttttttgggatcaagctcttggtcatgatgcgtgagaccattcctaccatccggtcaaagatagaggagatgcaacacaaaccaacggctctgatcgtagacttgtttggtttggacgcgataccgctcggtggtgagttcaacatgttgacttatatcttcatcgcttcaaacgcacgttttctcgcggtggctttgtttttcccaacgttggacaaagacatggaagaagagcacataatcaagaagcaacctatggttatgcctggatgtgaaccggttcggtttgaagatacacttgaaacattccttgacccaaacagccaactctaccgggaatttgttcctttcggttcggttttcccaacgtgtgatggtattattgtgaatacatgggatgatatggagcccaaaactttgaaatctcttcaagacccaaagctcttgggtcgaattgctggtgtaccggtttatccaattggtcctttgtctagaccggttgatccatctaaaactaatcatccggttttggattggttaaacaaacagccggacgagtcggtactttacatttcatttggaagcggtggctctctctcggctaaacaactaaccgaattggcttggggacttgagatgagtcagcaacggttcgtttgggtggttcgacccccggtggacggttcagcttgcagtgcatatttatccgctaacagtggtaaaatacgagacggtacacctgattatctcccggaaggttttgttagccggactcatgagagaggctttatggtctcttcttgggctccccaagcggagatcttggcccaccaagccgtaggtgggtttctaactcactgcggttggaattcgattctcgagagcgtcgttggtggcgttccgatgatcgcgtggccactttttgcggagcagatgatgaacgcgacactcctcaacgaagagcttggcgttgccgtccgctctaagaaactaccgtcggagggagtgattacgagggcggagatcgaggcgttggtgagaaagatcatggtggaggaggaaggtgctgagatgagaaagaagataaagaagctgaaagagaccgctgccgaatcgctgagttgcgacggtggagtggcgcatgaatcgttgtcaagaatcgccgacgagagcgagcatcttttggagcgtgtcaggtgcatggcacgtggtgcctag

SEQ ID NO: 20 - DNA, 1446 bp, Arabidopsis thaliana
atgcatatcacaaaaccacacgccgccatgttttccagtcccggaatgggccatgtcatcccggtgatcgagcttggaaagcgtctctccgctaacaacggcttccacgtcaccgtcttcgtcctcgaaaccgacgcagcctccgctcaatccaagttcctaaactcaaccggcgtcgacatcgtcaaacttccatcgccggacatttatggtttagtggaccccgacgaccatgtagtgaccaagatcggagtcattatgcgtgcagcagttccagccctccgatccaagatcgctgccatgcatcaaaagccaacggctctgatcgttgacttgtttggcacagatgcgttatgtctcgcaaaggaatttaacatgttgagttatgtgtttatccctaccaacgcacgttttctcggagtttcgatttattatccaaatttggacaaagatatcaaggaagagcacacagtgcaaagaaacccactcgctataccggggtgtgaaccggttaggttcgaagatactctggatgcatatctggttcccgacgaaccggtgtaccgggattttgttcgtcatggtctggcttacccaaaagccgatggaattttggtaaatacatgggaagagatggagcccaaatcattgaagtcccttctaaacccaaagctcttgggccgggttgctcgtgtaccggtctatccaatcggtcccttatgcagaccgatacaatcatccgaaaccgatcacccggttttggattggttaaacgaacaaccgaacgagtcggttctctatatctccttcgggagtggtggttgtctatcggcgaaacagttaactgaattggcgtggggactcgagcagagccagcaacggttcgtatgggtggttcgaccaccggtcgacggttcgtgttgtagcgagtatgtctcggctaacggtggtggaaccgaagacaacacgccagagtatctaccggaagggttcgtgagtcgtactagtgatagaggtttcgtggtcccctcatgggccccacaagctgaaatcctgtcccatcgggccgttggtgggtttttgacccattgcggttggagctcgacgttggaaagcgtcgttggcggcgttccgatgatcgcatggccactttttgccgagcagaatatgaatgcggcgttgctcagcgacgaactgggaatcgcagtcagattggatgatccaaaggaggatatttctaggtggaagattgaggcgttggtgaggaaggttatgactgagaaggaaggtgaagcgatgagaaggaaagtgaagaagttgagagactcggcggagatgtcactgagcattgacggtggtggtttggcgcacgagtcgctttgcagagtcaccaaggagtgtcaacggtttttggaacgtgtcgtggacttgtcacgtggtgcttag

SEQ ID NO: 21 - DNA, 1446 bp, Arabidopsis thaliana
atgcatatcacaaaaccacacgccgccatgttttccagtcccggaatgggccatgtcctcccggtgatcgagctagctaagcgtctctccgctaaccacggcttccacgtcaccgtcttcgtccttgaaactgacgcagcctccgttcagtccaagctccttaactcaaccggtgttgacatcgtcaaccttccatcgcccgacatttctggcttggtagaccccaacgcccatgtggtgaccaagatcggagtcattatgcgtgaagctgttccaaccctccgatccaagatcgttgccatgcatcaaaacccaacggctctgatcattgacttgtttggcacagatgcgttatgtcttgcagcggagttaaacatgttgacttatgtctttatcgcttccaacgcgcgttatctcggagtttcgatatattatccaactttggacgaagttatcaaagaagagcacacagtgcaacgaaaaccgctcactataccggggtgtgaaccggttagatttgaagatattatggatgcatatctggttccggacgaaccggtgtaccacgatttggttcgtcactgtctggcctacccaaaagcggatggaatcttggtgaatacatgggaagagatggagcccaaatcattaaagtcccttcaagacccgaaacttttgggccgggtcgctcgtgtaccggtttatccggttggtccgttatgcagaccgatacaatcatccacgaccgatcacccggtttttgattggttaaacaaacaaccaaacgagtcggttctctacatttccttcgggagtggtggttctctaacggctcaacagttaaccgaattggcgtgggggctcgaggagagccagcaacggtttatatgggtggttcgaccgcccgttgacggctcgtcttgcagtgattatttctcggctaaaggcggtgtaaccaaagacaacacgccagagtatctaccagaagggttcgtgactcgtacttgcgatagaggtttcatgatcccatcatgggcaccgcaagctgaaatcctagcccatcaggccgttggtgggtttttaacacattgtggttggagctcgacgttggaaagcgtcctttgcggcgttccaatgatagcgtggccgcttttcgccgagcagaatatgaacgcggcgttgcttagcgatgaactgggaatctctgttagagtggatgatccaaaggaggcgatttctaggtcgaagattgaggcgatggtgaggaaggttatggctgaggacgaaggtgaagagatgagaaggaaagtgaagaagttgagagacacggcggagatgtcacttagtattcacggtggtggttcggcgcatgagtcgctttgcagagtcacgaaggagtgtcaacggtttttggaatgtgtcggggacttgggacgtggtgcttag

SEQ ID NO: 22 - DNA, 1467 bp, Arabidopsis thaliana
atgggaactcctgtcgaagtctctaagctccatttcttgctcttccctttcatggctcatggccatatgataccaactctagacatggctaagctctttgccaccaaaggagctaaatccactatcctcactacacctctcaatgccaagctcttcttcgagaaacccatcaaatcattcaaccaagacaacccgggactcgaagacatcaccatccagatccttaatttcccttgcacagagcttggtttgcctgatggctgtgagaatactgatttcatcttctccacacctgacctaaacgtaggtgacttgagtcaaaagtttttactcgcaatgaaatatttcgaagagccactagaggagctcctcgtgacaatgagaccagactgtcttgtcggtaacatgttcttcccttggtccactaaagttgctgagaagttcggagtaccgagacttgtgttccacggcacaggctacttctctttatgtgcttctcattgcataaggctccctaagaatgtggcaacaagttctgagccctttgtgattcctgatctcccgggagacattttgattacagaggaacaggtcatggagacagaagaagagtctgtaatggggaggtttatgaaggcaataagagactcagagagagatagctttggcgtgttggtgaacagcttctacgagcttgaacaggcttactcagattatttcaagagctttgtggcgaaaagagcgtggcatatcggtccgctttccttaggaaatagaaagttcgaggagaaagcagaaagaggcaaaaaggcaagcattgatgagcatgaatgtttgaaatggctcgactccaagaaatgtgattcagtgatttacatggcctttggaaccatgtctagctttaaaaacgagcagctgatagagattgcagctggtttagatatgtcaggacatgattttgtctgggtggttaacagaaaaggcagccaagttgagaaggaagattggttaccagaggggtttgaagagaagaccaagggaaaaggattgataatccgagggtgggcgccacaagtgctgatacttgagcacaaagcaattggcggatttttgacgcattgtggatggaactcgttattagaaggggtggcagcgggcctgccaatggtgacatggcccgtgggagccgagcagttctacaacgagaaattggtgacacaagtgttgaaaacaggagtgagtgtgggagtgaagaagatgatgcaagtagttggagacttcattagcagagagaaagtggagggagcggtgagggaagtgatggttggagaagagaggaggaaacgggccaaggagttagcagaaatggcgaaaaatgcggtgaaagaaggaggatcttcagatctagaggtagataggttgatggaagagcttacgttagttaaactgcaaaaagagaaggtataa

SEQ ID NO: 23 - DNA, 1451 bp, Arabidopsis thaliana
atgggtagtgatcatcatcatcgaaagctccacgttatgttcttccctttcatggcttatggtcacatgataccaactctagacatggctaagcttttctctagcagaggagccaaatccacaatcctcaccacatctctcaactccaagatcctccaaaaacccatcgacacattcaagaatctgaatccgggtctcgaaatcgacatccagatcttcaatttcccttgcgtggagctggggttaccagaaggatgtgaaaacgttgatttcttcacttcaaacaacaatgatgataaaaacgagatgatcgtgaaattctttttctcgacaaggtttttcaaagaccagcttgagaaactcctcgggacaacgagaccagactgtcttatcgccgacatgttcttcccctgggctactgaagctgctgggaagttcaatgtgccaagacttgtgttccacggcactggctacttctctttatgcgctggttattgcatcggagtgcataaaccacagaagagagtggcttcaagctctgagccatttgtgattcccgagctccctgggaacattgtgataactgaagaacagatcatagatggcgatggagaatccgacatgggaaagtttatgactgaagttagggaatcggaagtgaagagctcaggagttgttttgaatagtttctacgagctagaacatgattacgccgatttttacaaaagttgtgtacaaaagagagcgtggcatatcggtccgctatcggtttacaacaggggatttgaggagaaggctgagagaggaaagaaagcgaacattgatgaggctgaatgcctcaaatggcttgactccaagaaaccaaattcagtcatttatgtttcctttgggagcgtggctttcttcaagaatgaacagttattcgagatcgctgcagggttagaagcttccggtacaagtttcatttgggttgttaggaaaaccaaagtgatagagaagaatggttaccagaagggttcgaagagagggtgaaagggaaaggtatgataataagaggatgggcaccacaggtgctgatacttgaccaccaagcaaccggtgggtttgtgacccattgcggctggaactcgcttcttgaaggagtggctgcagggctaccaatggtgacatggcctgtaggagcggagcaattctacaatgagaaattggttacgcaagtgctcagaacaggagtgagcgtgggagcgagcaagcatatgaaagttatgatgggagatttcattagcagagagaaagtggataaagcggtgagggaggttttggctggggaagcagcagaggagaggcggagacgggcaaagaagctagcggcgatggctaaagctgccgtggaagaaggagggtcttccttcaacgatctaaacagcttcatggaagagtttagttcataa

SEQ ID NO: 24 - DNA, 1446 bp, Arabidopsis thaliana
atgagtagtgatcctcatcgtaagctccatgttgtgttcttccctttcatggcttatggtcacatgataccaactctagacatggctaagcttttctctagcagaggagccaaatctacaatcctcaccacacctctcaactccaagatcttccaaaaacccatcgaaagattcaagaacctgaatccgagtttcgaaatcgacatccagatcttcgatttcccttgcgtggatctcgggttaccagaaggatgcgaaaacgtcgatttcttcacctcaaacaacaatgatgatagacagtatctgaccttgaagttctttaagtcgacaaggtttttcaaagatcagcttgagaagctcctcgagacaacgagaccagactgtcttatcgccgacatgttcttcccctgggctacggaagctgctgagaagttcaatgtgccaagacttgtgttccacggtactggctacttttctttatgctctgaatattgcatcagagtgcataacccacaaaacatagtagcttcaaggtacgagccatttgtgattcctgatctcccggggaacatagtgataactcaagaacagatagcagaccgtgacgaagaaagcgagatggggaagtttatgattgaggtcaaagaatctgatgtgaagagctcaggtgttattgtaaacagcttctacgagcttgaacctgattacgccgacttttacaagagtgttgtactgaagagagcgtggcatatcggtccgctttcggtttacaacagaggatttgaggagaaggctgagagaggaaagaaagcaagcattaatgaggttgaatgcctcaaatggcttgactccaagaaaccagattcagtcatttacatttcttttgggagcgtggcttgcttcaagaacgagcagctattcgagatcgctgcaggattagaaacttctggagcaaatttcatctgggttgttaggaaaaacataggtattgaaaaagaagaatggttaccagaagggttcgaagagagggtgaaaggaaaagggatgattataagaggatgggcaccacaggtgctcatacttgatcatcaagcaacttgtgggtttgtgacccattgcggctggaactcgcttctggaaggagtggctgcagggctaccaatggtgacatggcctgtagcagcggagcaattctacaatgagaaattggttacgcaagtgctcagaacaggagtgagcgtgggagcgaaaaagaatgtaagaactacgggagatttcattagcagagagaaagtggttaaagcggtgagggaggtgttggttggggaagaggcggatgagaggcgggagagggcaaagaagttggcagagatggctaaagctgccgtggaaggagggtcttctttcaacgatctaaacagcttcatagaagagtttacctcgtaa

SEQ ID NO: 25 - DNA, 1446 bp, Arabidopsis thaliana
atgaacagagagcaaattcatattttgttcttccccttcatggctcatggccacatgattccactcttagacatggccaagcttttcgctagaagaggagccaaatcaactctcctcacaaccccaataaatgctaagatcttggagaaacccattgaagcattcaaagttcaaaatcctgatctcgaaatcggaatcaagatcctcaatttcccttgtgtagagcttggattgccagaaggatgcgagaaccgtgacttcattaactcataccaaaaatctgactcatttgacttgttcttgaagtttcttttctctaccaagtatatgaaacagcagttggagagtttcattgaaacaaccaaaccgagtgctcttgtagccgatatgttcttcccttgggcaacagaatccgcggagaagatcggtgttccaagacttgtgttccacggcacatcatcctttgccttgtgttgttcgtataacatgaggattcataagccacacaagaaagtcgcttcgagttctactccatttgtaatccctggtctccctggagacatagttattacagaagaccaagccaatgtcaccaacgaagaaactccattcggaaagttttggaaagaagtcagggaatcagagaccagtagctttggtgttttggtgaatagcttctacgagctggaatcatcttatgctgatttttaccgtagttttgtggcgaaaaaagcgtggcatataggtccactttcactatccaacagagggattgcagagaaagccggaagagggaaaaaggcaaacattgatgagcaagaatgcctcaaatggcttgactctaagacacctggctcagtagtttacttgtcctttggtagcggaaccggcttacccaacgaacagctgttagagattgctttcggccttgaaggctctggacaaaatttcatttgggtggttagcaaaaatgaaaaccaaggtgaaaatgaagattggttgcctaaagggtttgaagagaggaataaaggaaaagggctgataatacgcggatgggccccgcaagtgctgatacttgaccacaaagcaatcggaggatttgtgacgcattgcggatggaactcgactttggagggcattgccgcagggctgcctatggtgacttggccgatgggggcagaacagttctacaacgagaagttattgacaaaagtgttgagaataggagtgaacgttggagctaccgagttggtgaaaaaaggaaagttgattagtagagcacaagtggagaaggcagtaagggaagtgattggtggtgagaaggcagaggaaaggcggctaagggctaaggagctgggcgagatggctaaagccgctgtggaagaaggagggtcttcttataatgatgtgaacaagtttatggaagagctgaatggtagaaagtag

SEQ ID NO: 26 - DNA, 1455 bp, Arabidopsis thaliana
atgaacagagaagtctctgagagaattcatattttgttcttccccttcatggctcaaggccacatgattccaattttggacatggccaagcttttctcgaggagaggagccaagtcaacccttctcacaaccccaatcaacgctaagatcttcgagaaacctattgaagcattcaaaaatcaaaaccctgatctcgaaatcggaatcaagatcttcaatttcccttgtgtagagcttggattgcctgaaggatgcgagaacgctgactttatcaactcataccaaaaatctgactcaggtgacttgttcttgaagtttcttttctctaccaagtatatgaaacaacagttggagagtttcattgaaacaaccaaaccaagtgctcttgttgccgatatgttcttcccttgggcgacagaatctgctgagaagctcggtgtaccaagacttgtgttccacggtacatctttcttttctttgtgttgttcgtataacatgaggattcataagccacacaagaaagtcgctacgagttctactccttttgtaatccctggtctcccaggagacatagttattacagaagaccaagccaatgttgccaaagaagaaacgccaatgggaaagtttatgaaagaggttagggaatcagagaccaatagctttggtgtattggttaatagcttctacgagctggaatcagcttatgctgatttttatcgtagttttgtggcgaaaagagcttggcatatcggtccgctttcgctatctaacagagagttaggagagaaagccagaagagggaaaaaggctaacattgatgagcaagaatgcctaaaatggctggactctaagacacctggttcagtagtttacttgtcctttgggagcggaactaatttcaccaacgaccagctgttagagatcgcttttggtcttgaaggttctggacaaagtttcatctgggtggttaggaaaaatgaaaaccaaggtgacaatgaagagtggttgcctgaagggtttaaagagaggacaacagggaaagggctaataatacctggatgggcgccgcaagtgctgatacttgaccataaagcaattggaggatttgtgactcattgcggatggaactcggctatagagggcattgccgcggggctgcctatggtaacatggccaatgggggcagaacagttctacaatgagaagctattgacaaaagtgttgagaataggagtgaacgttggagctaccgagttggtgaaaaaaggaaagttgattagtagagcacaagtggagaaggcagtaagggaagtgattggtggtgagaaggcagaggaaaggcggctatgggctaagaagctgggcgagatggctaaagccgctgtggaagaaggagggtcctcttataatgatgtgaacaagtttatggaagagctgaatggtagaaagtag

SEQ ID NO: 27 - DNA, 1476 bp, Arabidopsis thaliana
atggcatcggaatttcgtcctcctcttcattttgttctcttccctttcatggctcaaggccacatgatcccaatggtagatattgcaaggctcctggctcagcgcggggtgactataaccattgtcactacacctcaaaacgcaggccggttcaagaacgttcttagccgggctatccaatccggcttgcccatcaatctcgtgcaagtaaagtttccatctcaagaatcgggttcaccggaaggacaggagaatttggacttgctcgattcattgggggcttcattaaccttcttcaaagcatttagcctgctcgaggaaccagtcgagaagctcttgaaagagattcaacctaggccaaactgcataatcgctgacatgtgtttgccttatacaaacagaattgccaagaatcttggtataccaaaaatcatctttcatggcatgtgttgcttcaatcttctttgtacgcacataatgcaccaaaaccacgagttcttggaaactatagagtctgacaaggaatacttccccattcctaatttccctgacagagttgagttcacaaaatctcagcttccaatggtattagttgctggagattggaaagacttccttgacggaatgacagaaggggataacacttcttatggtgtgattgttaacacgtttgaagagctcgagccagcttatgttagagactacaagaaggttaaagcgggtaagatatggagcatcggaccggtttccttgtgcaacaagttaggagaagaccaagctgagaggggaaacaaggcggacattgatcaagacgagtgtattaaatggcttgattctaaagaagaagggtcggtgctatatgtttgccttggaagtatatgcaatcttcctctgtctcagctcaaagagctcggcttaggcctcgaggaatcccaaagacctttcatttgggtcataagaggttgggagaagtataacgagttacttgaatggatctcagagagcggttataaggaaagaatcaaagaaagaggccttctcataacaggatggtcgcctcaaatgcttatccttacacatcctgccgttggaggattcttgacacattgtggatggaactctactcttgaaggaatcacttcaggcgttccattactcacgtggccactgtttggagaccaattctgcaatgagaaattggcggtgcagatactaaaagccggtgtgagagctggggttgaagagtccatgagatggggagaagaggagaaaataggagtactggtggataaagaaggagtaaagaaggcagtggaggaattgatgggtgatagtaatgatgctaaggagagaagaaaaagagtgaaagagcttggagaattagctcacaaggctgtggaagaaggaggctcttctcattccaacatcacattcttgctacaagacataatgcaattagaacaacccaagaaatga

SEQ ID NO: 28 - DNA, 1491 bp, Arabidopsis thaliana
atggctttcgagaagacccgccaatttcttcctccgcttcactttgttctcttccctttcatggctcaaggccacatgatccccatggtggatattgcaaggatcttggctcagcgcggggtgactattaccattgtcacgacgcctcacaacgcagccaggttcaaagatgtcctaaaccgggccatccagtcaggcttgcacattagggttgagcatgtgaagtttccttttcaagaagctggtttgcaagaaggacaagagaatgttgattttcttgactcaatggagttaatggtacatttctttaaagcggttaacatgcttgaaaatccggtcatgaagctcatggaagagatgaaacctaaaccaagctgcctaatttctgatttttgtttgccttatacaagcaaaatcgctaagaggttcaatatcccaaagatcgttttccatggcgtgtcttgcttttgtcttttgagtatgcatattctacaccgaaaccacaatatcttacatgctttaaagtcggacaaagagtatttcttggttcctagttttccagatagagttgaatttacaaagcttcaagttactgtgaaaacaaactttagtggagattggaaagagatcatggacgaacaggtggatgctgatgacacgtcctatggtgtaattgtcaacacatttcaggatttggagtctgcctatgtgaaaaactacacggaggctagggctggtaaagtatggagcatcggtccggtttccttgtgcaacaaggtaggagaagacaaagctgagaggggaaacaaggcagccattgatcaagacgagtgtattaaatggcttgattctaaagatgtagagtcggtgctgtatgtttgccttggaagtatatgcaatcttcctctggctcagcttagagagctcgggctaggcctcgaggcaactaaaagaccattcatttgggtcataagaggtgggggaaagtatcatgaactagctgagtggatcttagagagcggttttgaagaaagaaccaaagagagaagccttctcataaaaggatggtcgcctcaaatgcttatcctttcacaccctgccgttggaggattcctgacacattgtggatggaactcaactttagaaggaatcacctcaggggttccattgatcacttggccattatttggagaccaattctgcaaccagaaactgatcgtgcaggtgctaaaagcaggtgtaagtgttggggttgaagaggtcatgaaatggggagaagaggagagtattggagtgttagtggataaagaaggagtgaagaaggcagtggacgaaataatgggcgagagtgatgaagcaaaagagagaagaaaaagagtcagagagcttggagaattagctcacaaggctgtggaagaaggaggctcttctcattctaatatcatatttttgctacaagatataatgcaacaagtagaatccaagagttga

SEQ ID NO: 29 - DNA, 1491 bp, Arabidopsis thaliana
atggctacggaaaaaacccaccaatttcatccttctcttcactttgtcctcttccctttcatggctcaaggccacatgattcccatgattgatattgcaagactcttggctcagcgtggtgtgaccataacaattgtcacgacacctcacaacgcagcaaggtttaagaatgtcctaaaccgagcgatcgagtctggcttggccatcaacatactgcatgtgaagtttccatatcaagagtttggtttgccagaaggaaaagagaatatagattcgttagactcaacggagttgatggtacctttcttcaaagcggtgaacttgcttgaagatccggtcatgaagctcatggaagagatgaaacctagacctagctgtctaatttctgattggtgtttgccttatacaagcataatcgccaagaacttcaatataccaaagatagttttccacggcatgggttgctttaatcttttgtgtatgcatgttctacgcagaaacttagagatcctagagaatgtaaagtcggatgaagagtatttcttggttcctagttttcctgatagagttgaatttacaaagcttcaacttcctgtgaaagcaaatgcaagtggagattggaaagagataatggatgaaatggtaaaagcagaatacacatcctatggtgtgatcgtcaacacatttcaggagttggagccaccttatgtcaaagactacaaagaggcaatggatggaaaagtatggtccattggacccgtttccttgtgtaacaaggcaggtgcagacaaagctgagaggggaagcaaggccgccattgatcaagatgagtgtcttcaatggcttgattctaaagaagaaggttcggtgctctatgtttgccttggaagtatatgtaatcttcctttgtctcagctcaaggagctggggctaggccttgaggaatctcgaagatcttttatttgggtcataagaggttcggaaaagtataaagaactatttgagtggatgttggagagcggttttgaagaaagaatcaaagagagaggacttctcattaaagggtgggcacctcaagtccttatcctttcacatccttccgttggaggattcctgacacactgtggatggaactcgactctcgaaggaatcacctcaggcattccactgatcacttggccgctgtttggagaccaattctgcaaccaaaaactggtcgttcaagtactaaaagccggtgtaagtgccggggttgaagaagtcatgaaatggggagaagaagataaaataggagtgttagtggataaagaaggagtgaaaaaggctgtggaagaattgatgggtgatagtgatgatgcaaaagagaggagaagaagagtcaaagagcttggagaattagctcacaaagctgtggaaaaaggaggctcttctcattctaacatcacactcttgctacaagacataatgcaactagcacaattcaagaattga

SEQ ID NO: 30 - DNA, 1491 bp, Arabidopsis thaliana
atggcttccgaaaaatcccacaaagttcatcctcctcttcactttattcttttccctttcatggctcagggccacatgattcccatgattgatatagcaaggctcttggctcagcgcggtgcgacagtaactattgtcacgacacgttataatgcagggaggttcgagaatgtcttaagtcgtgccatggagtctggtttacccatcaacatagtgcatgtgaattttccatatcaagaatttggtttgccagaaggaaaagagaatatagattcgtatgactcaatggagctgatggtacctttctttcaagcagttaacatgctcgaagatccggtcatgaagctcatggaagagatgaaacctagacctagctgtattatttctgatttgctcttgccttatacaagcaaaatcgcaaggaaattcagtataccaaagatagttttccacggcacgggttgctttaatcttttgtgtatgcatgttctacgcagaaacctcgagatcttgaagaacttaaagtcggataaagattatttcctggttcctagttttcctgatagagttgaatttacaaagcctcaagttccagtggaaacaactgcaagtggagattggaaagcgttcttggacgaaatggtagaagcagaatacacatcctatggtgtgatcgtcaacacatttcaggagttggagcctgcttatgtcaaagactacacgaaggctagggctggaaaagtatggtccattggacctgtttccttgtgcaacaaggcaggtgctgataaagctgagaggggaaaccaggccgccattgatcaagatgagtgtcttcaatggcttgattctaaagaagatggttcggtgttatatgtttgccttggaagtatctgtaatctacctttgtctcagctcaaggagctggggctaggccttgaaaaatcccaaagatcttttatttgggtcataagaggttgggaaaagtataatgaactatatgagtggatgatggagagcggttttgaagaaagaatcaaagagagaggacttcttattaaagggtggtcacctcaagtccttatcctttcacatccttccgttggaggattcctgacacactgtggatggaactcgactctcgaaggaatcacctcaggcattccactgatcacttggccgctgtttggagaccaattctgcaaccaaaaactggtcgttcaagtactaaaagccggtgtaagtgccggggttgaagaagtcatgaaatggggagaagaggagaaaataggagtgttagtggataaagaaggagtaaagaaggcagtggaagagttaatgggtgcgagtgatgatgcaaaagagaggagaagaagagtcaaagagcttggagaatcagctcacaaggctgtggaagaaggaggctcttctcattctaacatcacatacttgctacaagacataatgcaacaagtgaaatccaagaactga

SEQ ID NO: 31 - DNA, 1488 bp, Arabidopsis thaliana
atggtttccgaaacaaccaaatcttctccacttcactttgttctcttccctttcatggctcaaggccacatgattcccatggttgatattgcaaggctcttggctcagcgtggtgtgatcataacaattgtcacgacgcctcacaatgcagcgaggttcaagaatgtcctaaaccgtgccattgagtctggcttgcccatcaacttagtgcaagtcaagtttccatatctagaagctggtttgcaagaaggacaagagaatatcgattctcttgacacaatggagcggatgatacctttctttaaagcggttaactttctcgaagaaccagtccagaagctcattgaagagatgaaccctcgaccaagctgtctaatttctgatttttgtttgccttatacaagcaaaatcgccaagaagttcaatatcccaaagatcctcttccatggcatgggttgcttttgtcttctgtgtatgcatgttttacgcaagaaccgtgagatcttggacaatttaaagtcagataaggagcttttcactgttcctgattttcctgatagagttgaattcacaagaacgcaagttccggtagaaacatatgttccagctggagactggaaagatatctttgatggtatggtagaagcgaatgagacatcttatggtgtgatcgtcaactcatttcaagagctcgagcctgcttatgccaaagactacaaggaggtaaggtccggtaaagcatggaccattggacccgtttccttgtgcaacaaggtaggagccgacaaagcagagaggggaaacaaatcagacattgatcaagatgagtgccttaaatggctcgattctaagaaacatggctcggtgctttacgtttgtcttggaagtatctgtaatcttcctttgtctcaactcaaggagctgggactaggcctagaggaatcccaaagacctttcatttgggtcataagaggttgggagaagtacaaagagttagttgagtggttctcggaaagcggctttgaagatagaatccaagatagaggacttctcatcaaaggatggtcccctcaaatgcttatcctttcacatccatcagttggagggttcctaacacactgtggttggaactcgactcttgaggggataactgctggtctaccgctacttacatggccgctattcgcagaccaattctgcaatgagaaattggtcgttgaggtactaaaagccggtgtaagatccggggttgaacagcctatgaaatggggagaagaggagaaaataggagtgttggtggataaagaaggagtgaagaaggcagtggaagaattaatgggtgagagtgatgatgcaaaagagagaagaagaagagccaaagagcttggagattcagctcacaaggctgtggaagaaggaggctcttctcattctaacatctctttcttgctacaagacataatggaactggcagaacccaataattga

SEQ ID NO: 32 - DNA, 1488 bp, Arabidopsis thaliana
atggctttcgaaaaaaacaacgaaccttttcctcttcactttgttctcttccctttcatggctcaaggccacatgattcccatggttgatattgcaaggctcttggctcagcgaggtgtgcttataacaattgtcacgacgcctcacaatgcagcaaggttcaagaatgtcctaaaccgtgccattgagtctggtttgcccatcaacctagtgcaagtcaagtttccatatcaagaagctggtctgcaagaaggacaagaaaatatggatttgcttaccacgatggagcagataacatctttctttaaagcggttaacttactcaaagaaccagtccagaaccttattgaagagatgagcccgcgaccaagctgtctaatctctgatatgtgtttgtcgtatacaagcgaaatcgccaagaagttcaaaataccaaagatcctcttccatggcatgggttgcttttgtcttctgtgtgttaacgttctgcgcaagaaccgtgagatcttggacaatttaaagtctgataaggagtacttcattgttccttattttcctgatagagttgaattcacaagacctcaagttccggtggaaacatatgttcctgcaggctggaaagagatcttggaggatatggtagaagcggataagacatcttatggtgttatagtcaactcatttcaagagctcgaacctgcgtatgccaaagacttcaaggaggcaaggtctggtaaagcatggaccattggacctgtttccttgtgcaacaaggtaggagtagacaaagcagagaggggaaacaaatcagatattgatcaagatgagtgccttgaatggctcgattctaaggaaccgggatctgtgctctacgtttgccttggaagtatttgtaatcttcctctgtctcagctccttgagctgggactaggcctagaggaatcccaaagacctttcatctgggtcataagaggttgggagaaatacaaagagttagttgagtggttctcggaaagcggctttgaagatagaatccaagatagaggacttctcatcaaaggatggtcccctcaaatgcttatcctttcacatccttctgttggagggttcttaacgcactgcggatggaactcgactcttgaggggataactgctggtctaccaatgcttacatggccactatttgcagaccaattctgcaacgagaaactggtcgtacaaatactaaaagtcggtgtaagtgccgaggttaaagaggtcatgaaatggggagaagaagagaagataggagtgttggtggataaagaaggagtgaagaaggcagtggaagaactaatgggtgagagtgatgatgcaaaagagagaagaagaagagccaaagagcttggagaatcagctcacaaggctgtggaagaaggaggctcctctcattctaatatcactttcttgctacaagacataatgcaactagcacagtccaataattga

SEQ ID NO: 33 - DNA, 1473 bp, Arabidopsis thaliana
atgtgttctcatgatcctcttcacttcgtcgtaataccctttatggcccaaggccatatgatcccattggtcgacatctctaggctcttgtcccagcgccaaggcgtgactgtctgcatcatcacaactactcaaaatgtagccaagatcaagacttcactctcattttcctctttgtttgcgactatcaacatcgttgaagttaagtttctgtctcaacaaacgggtttgccagaagggtgcgagagtttagatatgttggcttcaatgggcgatatggtgaagttctttgatgctgccaactcacttgaggagcaagttgagaaagctatggaagagatggttcagccgcggccaagctgcatcattggagacatgagccttcctttcacttcaagacttgccaagaaattcaagatccccaaacttatcttccatgggttttcttgtttcagcctcatgtctatacaagtggttcgagaaagcgggatcttgaaaatgatagaatcaaacgacgagtattttgatttgcccggcttgcctgacaaagttgagttcacgaaacctcaggtctctgtgttgcaacctgttgaaggaaatatgaaagagagtacggccaagattattgaagctgataatgactcttatggtgttattgtgaacacttttgaagagttagaggttgattatgcaagagaatataggaaagcaagggctggaaaagtttggtgcgttggacctgtttccttgtgcaataggttagggttagacaaagctaaaagaggagataaggcttctattggtcaagaccaatgtcttcaatggcttgactctcaagaaactggttcagtgctctacgtttgccttggaagtctatgtaatcttcccttggctcagctcaaagagctgggactaggccttgaggcatctaataaacctttcatatgggttataagagaatggggaaaatatggagatttagcaaattggatgcaacaaagcggatttgaagagcggatcaaagatagaggactggtgatcaaaggttgggcgccgcaagttttcatcctctcacacgcatccattggagggtttttgactcactgtggatggaactcgacactagaaggaattactgcaggagttccattattgacatggcctttgtttgctgaacaattcttgaatgagaagttagttgtgcagatactaaaagcagggttaaagataggagtagagaaattgatgaaatatggaaaagaagaggagataggagcgatggtgagcagagaatgtgtgagaaaagctgtggatgagctaatgggtgatagtgaagaagcagaagagagaagaagaaaagttacagaacttagtgacttggcaaataaggctttggaaaaaggaggatcttcagattctaatatcacattgctcattcaagatattatggagcaatcacaaaatcaattttaa

SEQ ID NO: 34 - DNA, 1524 bp, Arabidopsis thaliana
atggaatcaaaaatagtttcaaaagccaaaagacttcactttgttttgatccctctcatggctcaagggcatctgatccccatggtcgacatctccaagattcttgcacgacaaggcaacatcgttaccatagttacaacccctcaaaatgcttctaggtttgcgaagacagttgaccgagcaagattagagtcgggtctcgaaatcaatgtcgttaaatttccaattccttacaaagaattcggtcttcccaaagattgtgagactctggacactttgccctccaaagacctcctacgaagattctatgacgctgtggataaactccaagagcccatggaacggtttcttgagcaacaagatatccctccaagttgcataatctccgataaatgccttttttggacgtcaagaaccgcaaagaggttcaaaatcccgaggatcgtgttccatggaatgtgttgcttctctcttttgagttcgcacaatatccatcttcatagcccgcacctctcggtttcttcggccgtagagccattccctataccaggaatgccacataggattgagatagctagagctcagttacctggtgcttttgagaagttagcaaatatggatgacgttcgcgagaagatgcgtgaatctgaatcagaagcctttggggttattgttaatagcttccaggaattggagcctggctatgcagaggcctacgctgaggccatcaataagaaggtatggttcgttggacccgtttctttatgcaacgaccgtatggctgacctattcgatagaggaagtaatggtaacatcgcaataagcgagaccgaatgcttgcagtttcttgactcgatgagaccaaggtcagtcttatatgtttctcttggtagcctctgtcgactaatacctaatcaattgatagaactaggtttagggttagaagaatcgggaaaaccctttatttgggtgataaagaccgaggaaaaacacatgattgagctagacgaatggctaaaacgcgaaaattttgaagagcgagttagaggaagagggatagtaataaagggttggagtcctcaggctatgatactctcacatggttcaaccggcgggttcttgactcattgcggttggaattctacaatagaagcgatatgttttggtgtaccaatgatcacatggccgttgttcgctgaacaatttctcaatgagaaactcatcgtggaggttttgaacatcggggttagggttggggtggagattccggtgagatggggagacgaggagagacttggagtgttggtcaagaaaccgagtgttgtgaaagctataaagcttttgatggaccaagattgtcaacgtgtagacgaaaatgatgatgataatgaattcgtgagacgaaggagacgtattcaagaacttgcagtaatggcgaaaaaggctgtggaagaaaagggatcttcgagtattaacgtttcaattttaatccaagatgttttggagcaattgagtctcgtgtag

SEQ ID NO: 35 - DNA, 1383 bp, Arabidopsis thaliana
atggcggaaacaactcccaaagtgaaaggccacgtcgtaatcttaccatacccagttcaaggccacctaaacccaatggttcaattcgctaaacgtctagtctccaaaaacgtcaaagtcacaatcgccaccactacctacaccgcctcctcaatcacaacaccatcactctccgtcgaaccaatctccgatggattcgatttcatccccataggtatccccggtttcagcgtcgatacttactcagaatccttcaagctcaacggatccgaaaccctaactctcctaatcgagaaattcaaatccacagattcaccaatcgattgcttaatctacgattcgtttcttccttggggacttgaagttgctagatctatggaactttcagctgcttctttcttcactaataatctcactgtttgttctgtgttgcgtaaattctctaacggtgactttcctcttcccgctgatcctaattcggcgccgtttcgtatccgtggcttaccgtctttgagctacgatgagttaccttcgtttgtgggacgtcattggttgactcatcctgagcatggcagagttcttctgaatcagtttcctaaccatgaaaatgctgattggttattcgttaatggctttgaagggttagaagaaacacaagattgtgaaaatggtgagtctgatgcaatgaaggcgacgttgatcggaccgatgattccatcggcttatcttgatgatcggatggaagatgataaagactatggtgcgagtctgttgaaaccgatatcgaaggagtgtatggagtggcttgagactaagcaggctcagtcagtagcatttgtttcgtttggttcgtttgggattctctttgagaagcaacttgcagaggtagctattgcgctacaagaatcggatttgaacttcttgtgggtgattaaagaagctcatatagcgaaattgcctgaagggtttgtggaatcgactaaagatagagccttgttggtttcttggtgtaaccagcttgaggttttagctcatgaatcgataggttgctttttgactcattgtggttggaactctacgttggaagggttgagtttgggagttccgatggttggtgtgcctcagtggagtgatcagatgaatgatgctaagtttgtggaggaagtttggaaagttgggtatagagcgaaagaggaagctggggaagtaatcgtgaagagtgaagaattggtgaggtgtttgaaaggagtgatggaaggagagagtagtgtgaagattagagagagttcgaagaagtggaaagatttggctgtgaaggcaatgagtgaaggaggaagctctgatcgaagcattaacgagtttatagagagtttagggaagtaa

SEQ ID NO: 36 - DNA, 1374 bp, Arabidopsis thaliana
atgagtgaagcaaagaagggtcacgtactgttttttccatatccattacaaggccacattaacccaatgatccaactcgctaaacgcttatccaaaaagggcatcaccagcacactcatcatcgcctccaaagaccaccgtgaaccttacacctccgacgactactccatcaccgtccacaccatccacgacggtttctttccacatgaacaccctcacgccaagttcgtagatcttgaccgtttccacaactctacttctcgaagcctgaccgatttcatctctagtgcgaagttgtcggacaatcctccaaaagctctgatctatgatccatttatgccctttgcattggacatagccaaggacttggatctatacgtagtggcatatttcactcaaccatggttggctagtcttgtttactaccatatcaacgaaggcacctacgatgttcccgttgatagacacgagaacccaacacttgcatcgtttcctggtttcccattgttaagccaagatgatctgccttcgttcgcctgcgaaaaagggtcgtaccctcttctacacgagtttgtggttaggcaattctctaatttattgcaagctgattgcattctctgcaacacttttgatcaacttgaaccaaaggtagtgaaatggatgaatgatcaatggccggtgaagaacattggaccggtggttccatcgaagttcttggataaccggttgccagaagacaaagattacgaactcgagaactccaagacagagccagacgagtctgttttgaagtggttgggaaacaggccggcgaagtcggtggtttacgtggcgtttgggacattggtggctttgagcgaaaaacagatgaaggaaattgcaatggcgattagccaaaccggatatcacttcttgtggtctgttagagaatccgagagaagcaaactaccctctggttttatcgaagaggcagaggagaaagactctggacttgtggctaagtgggttcctcagctagaggttttagcacatgaatcaatcgggtgtttcgtgtcacactgtggatggaactcgacattggaggcactatgcttaggggttccaatggtgggcgtgcctcagtggactgatcagcccacaaatgctaagtttatagaggatgtgtggaagattggggttagagtgaggaccgatggagaagggctttcgagtaaagaagagattgcgagatgcattgttgaggtcatggaaggagagagagggaaagagataaggaagaatgttgagaagcttaaggtgttggctcgcgaagctatctctgaaggaggtagttccgacaagaagattgatgagtttgttgctcttttgacttaa

SEQ ID NO: 37 - DNA, 1371 bp, Arabidopsis thaliana
atgggagagaaagcgaaagcaaatgtgttagtcttctcatttccgatacaaggtcacataaaccctctcctccaattctcaaaacgcctactctctaaaaacgtcaacgtcacattcctcaccacttcctccacccacaactccatcctccgccgtgccatcaccggcggagccactgctcttcctctctcttttgtccccattgacgatggattcgaggaagatcacccatctacggacacatctcccgactacttcgcaaagttccaagaaaacgtatctcgaagcctctcagagcttatctcctcgatggacccaaaaccaaacgccgtcgtttacgactcgtgcctgccttatgtcctcgacgtttgccggaaacatcctggcgttgctgcggcgtcgtttttcactcagtcctccaccgtgaacgcgacctatattcatttcttgcgtggagagtttaaggagtttcaaaatgatgtcgttttgcctgcaatgcctccgctgaagggtaatgacttaccggtgtttctgtacgataacaatctctgccggccgttgtttgagctcattagtagccagttcgtgaatgttgacgacattgacttcttcttggttaactctttcgacgaactcgaagtcgaggtgctacaatggatgaaaaaccaatggccggtcaagaacataggaccgatgattccatcaatgtacttagacaaacgattagcaggtgacaaagactacggaatcaacctcttcaatgcccaagtcaacgaatgccttgattggcttgactcaaaaccgcccggttcagtgatctacgtgtcttttggaagcttggccgtcttaaaagacgatcaaatgatagaagtcgcggctggtctaaaacaaactggccataacttcttatgggttgttagagaaactgaaacaaagaagcttccaagcaattacatagaggacatttgtgacaagggattgatagtgaattggagtcctcaattacaagttcttgcacataaatcaatcggttgtttcatgactcattgcgggtggaattcgactttagaggcattgagcttaggagttgctttgataggaatgccggcttatagcgaccagccgactaatgctaagtttattgaagatgtgtggaaggttggggttagggttaaggcagatcaaaatgggtttgttccgaaggaagagattgtgagatgtgttggagaagttatggaagatatgtcggagaaagggaaggagattagaaaaaatgctcggaggttgatggagtttgcaagggaagctttgtctgatggaggaaattctgataagaatattgatgagtttgttgctaaaattgtgaggtaa

SEQ ID NO: 38 - DNA, 1362 bp, Arabidopsis thaliana
atgagagaaggatctcatgttattgttttgcctttcccagcacaaggccacataactccaatgtcccaattctgtaaacgcttagcctcaaaaagtcttaagatcactcttgtcctcgtctccgacaagccctctccgccgtacaaaacagagcacgacacaatcactgtcgtccccatctccaatggtttccaagaaggccaggaacgatcagaagacctagatgagtacatggaaagagtagaatccagcatcaaaaaccgcttaccgaagttgatagaagacatgaaactatcgggaaatcctcctagggctcttgtgtacgactccaccatgccgtggcttctggatgtagctcatagttatggtttgagcggtgccgtgtttttcacgcagccttggcttgtctcagctatttactatcatgtattcaagggctcgttctctgtaccgtctacaaagtatggtcactcgacgttagcatctttcccttcgttaccgattctgaatgcgaatgatttgccgtctttcctctgtgaatcttcctcttacccatatattctaaggactgtgatcgatcagctctcaaacattgatcgagttgatatagttttgtgcaacactttcgataaattggaagaaaagttgctgaaatggattaaaagcgtgtggcctgtcctgaacataggaccaactgttccatcaatgtatttagataagcgactggctgaagacaaaaactacggattcagcctcttcggtgcgaaaatcgctgaatgcatggagtggctcaactcaaagcagcctagttcagttgtttatgtatcatttgggagcttggtggttctaaaaaaagatcaactgatagaactagcggcgggtctgaaacagagcggacatttctttttgtgggttgtgagagagacggagagaagaaaacttccagaaaactatatagaggaaattggtgagaaaggactgaccgtgagctggagtccacaacttgaagttcttacacataaatcgatcggttgtttcgtgacacattgtggatggaactcgacgttagagggattgagtttgggagttccaatgattggtatgcctcattgggcagatcagcctacaaatgctaagttcatggaggatgtgtggaaagttggagttagggttaaagcagacagtgatgggttcgtgagaagagaagagtttgtgagacgtgtggaagaagttatggaggcagagcaaggtaaagagattagaaagaatgctgagaaatggaaagtgttggctcaagaggctgtttctgaaggaggtagttctgataagaacatcaatgagtttgtttctatgttttgttga

SEQ ID NO: 39 - DNA, 1362 bp, Arabidopsis thaliana
atgagagaaggatctcatcttatcgtcttgcctttcccaggacaaggccacataactccaatgtcccagttctgcaaacgcttagcctcaaaaggtcttaagctcactctggtcctcgtctccgacaaaccctctcctccatacaaaacagagcacgactcaatcactgtcttccccatctccaacggcttccaagaaggcgaggaaccattacaagacctcgatgattacatggaaagagtagaaaccagcatcaaaaacaccttaccgaagttggttgaagacatgaaactgtcgggaaatccacctagggctatcgtgtacgactccaccatgccatggcttcttgatgtagctcatagttatggattgagcggtgccgtgtttttcacgcaaccttggcttgtcacagctatttactaccatgttttcaagggttcgttctctgtaccgtctacaaagtacggtcactcgacattagcatctttcccttcgttcccgatgctgactgcaaatgatttgccgtctttcctctgcgaatcgtcctcatacccgaatatactgaggattgtggtggatcagctctcaaacattgatcgagtcgacatagtgttgtgcaacactttcgataaattggaggaaaagttgttgaaatgggtccaaagcttgtggccagtcttgaatattggaccaacggttccatcgatgtatttagacaaacgactgtctgaagacaagaactacggttttagcctcttcaatgcgaaagtcgctgaatgcatggagtggctaaactcaaaggagcctaattctgttgtctatttatcattcggaagtttggtgattctaaaagaagatcaaatgttggaactcgctgcgggtctgaaacagagcggacgtttctttctgtgggttgtgagagagacagagacacacaaacttccaagaaactatgtcgaggaaatcggtgaaaaaggacttattgtaagctggagtcctcagcttgacgtacttgcacataaatcaatcggttgtttcttgacacactgtggatggaactcgacgttagagggattgagtttgggagttccaatgattggtatgccacactggactgatcagcccacgaatgctaagttcatgcaggatgtgtggaaggttggggtaagggttaaggcagaaggtgatgggtttgtgagaagagaagagattatgagaagtgtggaagaagttatggagggagagaaagggaaagagattagaaagaatgctgagaaatggaaagtgttggctcaagaggcagtttctgaaggaggtagctctgataagagcatcaatgagtttgtttctatgttttgttga

SEQ ID NO: 40 - DNA, 1350 bp, Arabidopsis thaliana
atggagaagatgagaggacatgtattagcagtgccatttccaagccaaggacacatcaccccgattcgccaattctgcaaacgacttcactccaaaggtttcaaaaccactcacactctcaccacttttatcttcaacacaatccacctcgacccatctagtcctatctccatagccacaatctccgatggctatgaccagggagggttctcatcagccggttctgtcccggagtacctacaaaacttcaaaaccttcggctccaaaaccgtcgctgatatcatccgcaaacaccagagtactgataaccctattacttgtatcgtctatgattctttcatgccttgggcgcttgaccttgcaatggattttggtctagctgcggctcctttcttcacgcagtcttgcgccgttaactatatcaattatctttcttacataaacaatggtagcttgacacttcccatcaaggatttgcctcttcttgagctccaagatttgcctactttcgtcactcctactggttcacaccttgcttactttgagatggtgcttcaacagttcaccaacttcgacaaagctgatttcgtactcgttaattccttccatgacctcgaccttcatgaagaggagttgttgtcgaaagtatgtcctgtgttgacaattggtccaactgttccatcaatgtacttagaccaacagatcaaatcagacaacgactatgatctgaacctctttgacttaaaagaagctgccttatgcactgactggctagacaagaggccagaaggatcggtagtatatatagcttttgggagcatggctaaactgagtagtgagcagatggaagagattgcttcggcgataagcaacttcagctacctctgggttgtcagagcttcagaggagtcaaagctcccaccagggtttcttgaaacagtggataaagacaagagcttggtcttgaagtggagtcctcagcttcaagttctgtcaaacaaagccatcggttgtttcatgactcactgtggctggaactcaaccatggagggtttgagtttaggggttcccatggtggctatgcctcaatggactgatcaaccaatgaatgcaaagtatatacaagatgtatggaaggttggggttcgtgtgaaagcagagaaagaaagtggcatttgcaaaagagaggagattgagtttagcatcaaggaagtgatggaaggagagaagagcaaagagatgaaagagaatgcgggaaaatggagagacttggctgtgaagtcactcagtgaaggaggttctacagatatcaacattaacgaatttgtatcaaaaattcaaatcaaataa

SEQ ID NO: 41 - DNA, 1350 bp, Arabidopsis thaliana
atggagcataagagaggacatgtattagcagtgccgtacccaacgcaaggacacatcacaccattccgccaattctgcaaacgacttcacttcaaaggtctcaaaaccactctcgctctcaccactttcgtcttcaactccatcaatcctgacctatccggtccaatctccatagccaccatctccgatggctatgaccatgggggtttcgagacagctgactccatcgacgactacctcaaagactttaaaacttccggctcgaaaaccattgcagacatcatccaaaaacaccagactagtgataaccccatcacttgtatcgtctatgatgctttcctgccttgggcacttgacgttgctagagagtttggtttagttgcgactcctttctttacgcagccttgtgctgttaactatgtttattatctttcttacataaacaatggaagcttgcaacttcccattgaggaattgccttttcttgagctccaagatttgccttctttcttctctgtttctggctcttatcctgcttactttgagatggtgcttcaacagttcataaatttcgaaaaagctgatttcgttctcgttaatagcttccaagagttggaactgcatgagaatgaattgtggtcgaaagcttgtcctgtgttgacaattggtccaactattccatcaatttacttagaccaacgtatcaaatcagacaccggctatgatcttaatctctttgaatcgaaagatgattccttctgcattaactggctcgacacaaggccacaagggtcggtggtgtacgtagcattcggaagcatggctcagctgactaatgtgcagatggaggagcttgcttcagcagtaagcaacttcagcttcctgtgggtggtcagatcttcagaggaggaaaaactcccatcagggtttcttgagacagtgaataaagaaaagagcttggtcttgaaatggagtcctcagcttcaagttctgtcaaacaaagccatcggttgtttcttgactcactgtggctggaactcaaccatggaggctttgaccttcggggttcccatggtggcaatgccccaatggactgatcaaccgatgaacgcaaagtacatacaagatgtgtggaaggctggagttcgtgtgaagacagagaaggagagtgggattgccaagagagaggagattgagtttagcattaaggaagtgatggaaggagagaggagcaaagagatgaagaagaacgtgaagaaatggagagacttggctgtcaagtcactcaatgaaggaggttctacggatactaacattgatacatttgtatcaagggttcagagcaaatag

SEQ ID NO: 42 - DNA, 1410 bp, Arabidopsis thaliana
atggcgccaccgcattttctactggtaacgtttccggcgcaaggtcacgtgaacccatctctccgttttgctcgtcggctcatcaaaagaaccggcgcacgtgtcactttcgtcacttgtgtctccgtcttccacaactccatgatcgcaaaccacaacaaagtcgaaaatctctctttccttactttctccgacggtttcgacgatggaggcatttccacctacgaagaccgtcagaaaaggtcggtgaatctcaaggttaacggcgataaggcactatcggatttcatcgaagctactaagaatggtgactctcccgtgacttgcttgatctacacgattcttctcaattgggctccaaaagtagcacgtagatttcaacttccctccgctcttctctggatccaaccggctttggttttcaacatctattacactcatttcatgggaaacaagtccgttttcgagttacctaatctgtcttctctggaaatcagagatcttccatctttcctcacaccttccaacacaaacaaaggcgcatacgatgcgtttcaagaaatgatggagtttctcataaaagaaaccaaaccgaaaattctcatcaacactttcgattcgctggaaccagaggccttaacggctttcccgaatatcgatatggtggcggttggtcctttacttcccacggagattttctcaggaagcaccaacaaatcagttaaagatcaaagtagtagttatacactttggctagactcgaaaacagagtcctctgttatttacgtttcctttggaacaatggttgagttgtccaagaaacagatagaggaactagcgagagcactcatagaagggaaacgaccgtttttgtgggttataactgataaatccaacagagaaacgaaaacagaaggagaagaagagacagagattgagaagatagctggattcagacacgagcttgaagaggttgggatgattgtgtcgtggtgttcgcagatagaggttttaagtcaccgagccgtaggttgttttgtgactcattgtgggtggagctcgacgctggagagtttggttcttggcgttccggttgtggcgtttccgatgtggtcggatcaaccgacgaacgcgaagctactggaagaaagttggaagactggtgtgagggtaagagagaacaaggatggtttggtggagagaggagagatcaggaggtgtttggaagccgtgatggaggagaagtcggtggagttgagggaaaacgcaaagaaatggaagcgtttagcgatggaagcgggtagagaaggaggatcttcggataagaacatggaggcttttgtggaggatatttgtggagaatctcttattcaaaacttgtgtgaagcagaggaggtaaaagtaaagtaa

SEQ ID NO: 43 - DNA, 1368 bp, Arabidopsis thaliana
atggcgcaaccgcattttctactggtaacgtttccggcgcaaggtcacgtgaacccatctctccgttttgctcgtcggctcatcaaaacaactggcgcacgtgtaactttcgccacgtgtctctctgtcattcaccgctctatgatcccaaaccacaacaacgtcgaaaatctctctttccttactttctccgacggattcgacgacggagtcatctccaacaccgacgacgtccaaaaccggttggtacacttcgaacgtaatggcgataaagctctatcggatttcatcgaagctaatcagaatggtgactctcccgtaagttgcttgatctacacgattcttcccaactgggttccaaaagtggcgcgtagatttcatcttccctctgttcatctctggatccaaccagccttcgctttcgacatttattacaattactctacaggaaacaactccgttttcgagttcccgaatctaccttctctcgaaatccgcgatctgccttctttcctctcaccttccaacacgaacaaagccgcacaagcagtatatcaagaactgatggattttctcaaagaagaatctaacccgaaaattctcgtcaacacattcgattcgctggagccagagttcttaacagctattccgaatatagaaatggtggcagttggtcctttacttcctgcggagattttcactggaagcgaatcaggtaaagatttatcaagagatcatcaaagtagtagttatacactttggttagactcgaaaacagagtcctctgttatttatgtttcttttggaacaatggttgagttgtcgaagaaacagatagaggaactagcgagagcactcatagaagggggaagaccgttcttgtgggttataactgataaactcaacagagaagcgaaaatagaaggagaagaagagacagagattgagaagatagctggttttagacacgagcttgaagaggttgggatgattgtctcgtggtgttcgcagatagaggttttgagacaccgagccataggttgttttttgactcattgtgggtggagctcatcactggagagtttggttctcggcgttccagtggtggcgtttccgatgtggtcggatcagccagcaaatgcgaagcttttggaagaaatatggaagacaggtgtgagggtgagagagaactcggaaggtttagtagagagaggagagataatgcggtgtttggaagcagtgatggaggcgaaatcggtggagctgagggaaaacgcagagaaatggaagcgtttagcgactgaagcgggtagagaaggaggatcttcggacaagaatgtggaagcttttgtgaagagtctgttttga

SEQ ID NO: 44 - DNA, 1371 bp, Arabidopsis thaliana
atggccacttccgtcaatggttcccatcgtcgtccacattacttgcttgtaacattcccagcgcaaggtcacatcaacccggcgcttcaactagccaaccgcctcatccaccacggtgcaaccgtcacatactccaccgcagtctctgctcaccgacgtatgggcgagccaccttccacaaaaggtctatccttcgcttggttcaccgatggattcgacgacggtctcaagtcattcgaagaccagaaaatctacatgtccgaactcaaacgatgtggttcaaacgccctgagagacatcatcaaagccaatcttgacgccaccaccgaaacagagcctatcaccggggtaatctactctgttctcgtcccgtgggtttctacggtagcgcgtgagtttcacctcccaactacacttctctggattgaaccagctactgtactagacatctactactactacttcaacacctcttacaaacatctcttcgacgttgaaccgattaaattaccgaaactgccactgatcaccaccggtgacctcccgtcgtttcttcaaccttcgaaggcattaccgtcagctcttgtgactctaagagaacatatcgaagctctcgaaacggaatcaaaccctaagattcttgttaacacattctctgctttggaacacgatgctttaacctctgttgagaaactcaagatgatcccaatcggaccgttggtttcttcctccgagggtaaaaccgatcttttcaaatcttccgacgaggattacacgaaatggttagactcgaagctcgagagatcagtgatttacatttccttaggcacacacgccgatgatttaccagagaaacacatggaagcgcttactcacggcgtgttagctacaaacagaccgtttttatggatcgtgagggagaaaaatccagaagagaagaagaagaatcggtttcttgaattgatcagaggaagtgatcgaggattggtggtgggatggtgttctcagacagctgttttggcgcattgtgctgtgggatgttttgtgactcattgtggttggaattcgacgttggagagtttagagagtggtgttccggtggttgcgtttccgcagtttgctgatcagtgtacaacggcgaagcttgtggaggatacgtggaggattggagtgaaggtgaaggttggggaggaaggagatgtggatggggaggagattagaaggtgtttggagaaggtgatgagtggtggagaagaggcggaggagatgagagagaatgcagagaagtggaaggcgatggctgttgatgcggcagcggaaggtggaccgtcggatttgaatcttaaaggttttgtggacgaggatgagtag

SEQ ID NO: 45 - DNA, 1425 bp, Arabidopsis thaliana
atggccaacaacaattccaactctcccaccggtccacactttctattcgtaacatttccagcccaaggtcacatcaacccatctctcgagctagccaaacgcctcgccggaacaatctctggtgctcgagtcaccttcgccgcctcaatctctgcctacaaccgccgcatgttctctacagaaaacgtccccgaaaccctaatcttcgctacctactccgatggccacgacgacggtttcaaatcctctgcttactccgacaaatctcgtcaagacgccactggaaacttcatgtctgagatgagacgacgtggcaaagagacactaaccgaactaatcgaagataaccggaaacaaaacaggccttttacttgcgtggtttacacgattctcctcacttgggtcgctgagctagcgcgtgagtttcatcttccttctgctcttctttgggtccaaccagtaacagtcttctccattttttaccattacttcaatggctacgaagatgcaatctcagagatggctaataccccctctagttctattaaattaccttctctgccactgcttactgtccgtgatattccttctttcattgtctcttccaatgtctacgcgtttcttctacccgcgtttcgagaacagattgattcactgaaggaagaaataaaccctaagatcctcatcaacactttccaagagcttgagccagaagccatgagctcggttccagataatttcaagattgtccctgtcggtccgttactaacgttgagaacggatttttcgagtcgcggtgaatacatagagtggttggatactaaagcggattcgtctgtgctttatgtttcgttcgggacgcttgccgtgttgagcaagaaacagcttgtggagctttgtaaagcgttgatacaaagtcggagaccattcttgtgggtgattacggataagtcgtacagaaataaagaagatgagcaagagaaggaagaagattgcataagtagtttcagagaagagctcgatgagataggaatggtggtttcatggtgtgatcagtttagggttttgaatcatagatcgataggttgtttcgtgacgcattgcgggtggaactctacgctggagagcttggtttcaggagttccggtggtggcgtttccgcaatggaatgatcagatgatgaacgcgaagcttttagaagattgttggaaaacaggtgtaagagtgatggagaagaaggaagaagaaggagttgtggtggtggatagtgaggagatacggcggtgcattgaggaagttatggaagacaaggcggaggagtttagaggaaatgccacgaggtggaaggatttagcggcggaggctgtgagagaaggaggctcttcctttaatcatctcaaagcttttgtcgatgagcacatgtga

SEQ ID NO: 46 - DNA, 1344 bp, Arabidopsis thaliana
atggagactagagaaacaaaaccagtgatctttctcttccctttccctttacaaggtcacttaaacccaatgtttcagctcgccaacatcttcttcaacagaggcttctccatcactgtgatccacactgagttcaactctccaaactcttccaatttccctcatttcactttcgtatccatccccgatagcttgtctgaacctgaatcctatcccgatgtcatcgagattctccatgacctcaattccaagtgtgttgctccttttggtgattgcttaaagaagcttatatctgaagaaccaacagcagcttgtgtgattgttgacgctctttggtacttcactcacgatttaaccgagaaattcaatttcccgaggattgttctccgaaccgttaacctctcagctttcgtcgctttctcaaagtttcatgttttacgagagaaagggtatctttctttacaagagactaaggcagactcaccggttccggagcttccgtatcttagaatgaaggatcttccatggttccagacagaagatccaagatcaggggataagttacagataggtgtgatgaagtcactaaagtcttcctcaggaatcatattcaacgccattgaagatcttgaaacagatcagcttgatgaagcccgcatagaattcccagttccactcttctgtattggaccctttcacaggtacgtttcagcttcatccagtagcttacttgcacacgacatgacttgtctctcctggttagacaagcaagcaacaaattccgtaatctacgcaagtcttggaagcattgcttcgatcgatgaatctgaattcttggagattgcttggggtctaagaaacagcaaccaaccttttctatgggtggttagacccggtttaatccacgggaaagaatggatcgagattctgcctaaagggttcatcgaaaatctcgagggccggggtaaaatagtgaaatgggcacctcagcctgaagttttagctcaccgtgcaacaggcggattcttaacacattgtggatggaactcaacacttgagggcatatgtgaagctataccaatgatatgcagaccatcttttggggaccagagggtgaatgctagatacattaacgatgtttggaagatcggattgcatttggaaaacaaggtagagagactagtgatcgaaaacgcggttagaacactaatgacgagctcggaaggggaagagatccgcaagaggattatgcccatgaaggaaactgttgaacaatgccttaagcttggaggttcatcatttcggaatctcgaaaacttaattgcttatatattgtctttctaa

SEQ ID NO: 47 - DNA, 1395 bp, Arabidopsis thaliana
atggagaagagaaacgagagacaagtgattctttttcctctaccattacaaggttgcataaaccctatgcttcagctagcaaagatcctttactcaagaggtttttcgatcaccatcatccacacgcgcttcaacgcgcccaaatcttcagaccatcctctcttcactttcttacaaatccgcgacggcttgtctgaatctcagactcaatctcgtgatcttttgcttcaactcacgcttctcaacaacaattgtcagatcccatttcgagagtgtttggctaaactcattaaacctagttcagattcaggaacagaggataggaaaattagctgtgtgatcgatgattccggttgggttttcacacaatccgtggcggagagttttaatcttcctcgatttgtcctctgtgcttataagttctctttctttctcggacattttcttgttcctcagattcgtcgtgaagggtttcttccagtaccagattcggaggcagatgatctagttcctgagtttccaccgcttcgaaagaaagatctttcgagaattatgggaaccagcgctcagagtaagcctctagatgcttacttgcttaagatactcgacgcgacgaagccagcttcagggattatagttatgtcctgcaaagagcttgaccatgattcacttgctgagtccaacaaagttttcagcattccgatatttcccattggcccttttcacattcatgacgtcccagcctcgtctagcagcttgttagaaccggaccagagttgcattccatggttagatatgcgtgaaacgagatcagtagtctacgtgagcttagggagcattgcgagtcttaacgagtctgacttcttggagattgcttgtggactaagaaacaccaaccaatccttcttgtgggttgtccggcctggttcagtccatggcagagattggatcgaatcattaccttcagggttcatggaaagtctcgatggtaaaggaaagatagtgagatgggcaccgcagctagacgttcttgcgcatagagccacgggagggtttttgactcataatggatggaactcgacattagagagtatatgcgaaggagtacctatgatctgcttgccttgtaagtgggaccaatttgtaaacgcgagattcataagcgaagtttggagggttgggattcacttggaaggtcggatagagcgaagagaaatcgagagagctgttataagactaatggttgagtcgaaaggagaagagattcgaggtagaatcaaagtcttgcgagacgaagtaagaaggtcagttaaacaaggaggttcgtcatatcgatctttagatgagttggttgatcgtatatcaatcatcatcgagccactagtgcctacgtga

SEQ ID NO: 48 - DNA, 1353 bp, Arabidopsis thaliana
atggaggagaagagaaatggtctgcgtgtgattctcttccctcttccattacaaggttgcatcaaccctatgcttcagctcgccaacatccttcacgtaagaggcttctccattaccgtgatccacacgcgcttcaacgcgccaaaagcttcaagccatcctctcttcactttcttacagattcctgatggtttgtctgaaacggagattcaagatggtgttatgtctttgctcgcgcaaatcaaccttaacgctgagtctccgtttcgtgattgcttgcgtaaagtgttgctggaatcaaaagagtcagagagggttacttgtttgatcgatgactgtggatggctcttcacacaatctgtttcagagagtttgaagcttccgaggctcgttctctgtacttttaaagccactttcttcaatgcttatccgagtcttccacttatccgaaccaagggatatcttccagtttcagaatcggaagcagaggactctgttcctgagttcccgccgcttcaaaagagagatctttcaaaggttttcggggagttcggagagaaactcgatccgttcttacatgctgtagtcgaaacgacaataagatcttcagggttaatatacatgtcctgcgaagagcttgagaaagattcgttgactctttctaacgaaatttttaaagttccggtttttgcaattggtccgtttcacagctacttctctgcttcgtcaagcagcttgttcacacaagacgagacttgcattctgtggttagatgatcaagaagataaatctgtgatctacgttagtctaggaagcgttgtgaacataacggaaacagagttcttggagattgcgtgtggtttaagcaatagcaaacagcctttcttgtgggtagtacgacccggttcagtactcggcgcgaaatggatcgaaccgctctctgaagggctggttagtagccttgaagagaaaggaaagattgtgaaatgggcaccacaacaggaggttcttgcgcatcgtgccacaggagggtttttgacacacaatggttggaactcaacgctagagagtatatgcgaaggggttcctatgatctgcctaccaggaggttgggatcaaatgctgaattcaagatttgttagcgatatttggaagattggaattcacttggaaggtcggattgaaaaaaaggagattgagaaagctgtgagggtgttaatggaggaaagtgaaggaaataagattcgtgagagaatgaaagttctgaaagatgaggtcgagaaatcggtcaaacaaggaggctcatcttttcaatctattgagactctagctaatcatatactattgttgtaa

SEQ ID NO: 49 - DNA, 1353 bp, Arabidopsis thaliana
atggataagagtaatggcctacgagtgattctgtttccacttccattacaaggatgcatcaaccccatgattcagctagcgaagatcctccactcaagaggtttctccatcactgtgatccacacgcgcttcaatgcgccaaaagcttcaaaccaccctctgttcaccttcttacagatcccagatggcttgtctgaaacagagacaagaactcacgatatcacacttctcctaacgcttctcaaccgaagctgtgagtctccatttcgtgaatgtttgactaaacttttgcagtctgcagattcagaaacaggggaagagaaacagaggattagctgtttgatcgatgattctggatggatattcacacagcccgttgctcagagtttcaatctcccgagattggtccttaacacctacaaagtctccttctttcgggaccattttgttcttcctcaactccgtcgtgaaatgtatcttccattacaagattcagaacaaggtgatgatccagttgaggagtttccaccccttcgaaagaaagatcttttacaaattcttgatcaagaatcggagcaactagactcgtactccaatatgattttggaaacaacaaaagcgtcttcaggtcttatatttgtatccacatgtgaagagttggaccaagactcactgagtcaagcacgtgaagattatcaagtcccaatctttacgataggaccttctcatagctacttcccaggctcatctagtagcttgttcacagtggacgagacttgcattccatggttagacaagcaagaagacaaatccgtgatttacgtgagttttgggagcatctcgaccattggcgaagcagaattcatggagattgcttgggctctaagaaacagcgaccaaccgttcttgtgggtcgtacggggtggttcggtagtccatggtgcagaatggatcgaacagcttcatgagaaaggaaagatagtgaattgggccccacaacaagaggttctaaagcatcaagccattggaggattcttgacacacaatggttggaactcgacggttgagagtgtttttgaaggcgtccctatgatatgtatgccttttgtatgggaccaattgcttaatgcaagatttgttagtgatgtatggatggttgggctgcatctagagggtcggattgagaggaatgtgattgagggaatgataagaagattattttcggaaactgaaggaaaagcgatccgagagaggatggaaattcttaaggagaatgtaggaagatccgttaaaccaaaaggttcggcgtatcgatcgttacaacatttgattgattatataacatatttctag

SEQ ID NO: 50 - DNA, 1356 bp, Arabidopsis thaliana
atggagaagagtaatggcctgcgagtgattctgtttccacttccattacaaggctgcatcaaccctatgattcagctcgccaagatcctccactcaagaggtttttcaatcactgtgatccacacttgcttcaacgcgccaaaagcttcaagccatccactcttcaccttcatacagatccaagatggcttgtctgaaacagagacaagaactcgcgacgtcaaacttctcataacacttctcaaccaaaattgcgagtctccggttcgtgaatgtttgcgtaaactgttgcaatctgccaaggaagagaaacagaggattagctgtttgatcaatgattctggttggatcttcactcaacacttagccaagagtttgaatctcatgagattggcctttaatacctataagatctccttctttcgaagccattttgttcttcctcagctccggcgtgaaatgtttcttccattacaagattcagaacaagatgatccagttgagaagtttccaccgcttagaaagaaagatcttttacggattcttgaagcagattcggtgcagggagactcgtactcggatatgattttggaaaagacaaaggcgtcttcaggtcttatattcatgtcctgtgaagagttggaccaagactcactgagtcaatcacgtgaagattttaaggttccgatatttgcgataggaccttctcatagccattttcctgcttcttctagtagcttgttcacaccggacgagacttgcatcccatggttagacagacaagaagacaaatccgtaatatacgtgagtattgggagcctcgtgaccatcaacgaaacagagctaatggagattgcttggggtctaagtaacagcgaccaaccatttttatgggtcgtccgggttggttcagtcaatggcacggaatggattgaagcaatcccggaatatttcatcaaaaggcttaatgagaagggaaagatagtgaaatgggctccacaacaagaggttctaaagcatcgagctattggaggtttcttgacacataatggttggaactcgacggttgagagtgtttgtgaaggcgtccctatgatctgtttgccttttcgttgggaccaattgttaaatgcaagatttgttagtgatgtatggatggttgggatacatctcgagggtcggattgagagggatgagatcgagagagcgataaggagattattgttggaaactgaaggagaagccatccgagagaggatacaacttcttaaggaaaaagtaggaagatcagttaaacaaaacggttcggcatatcaatctctacaaaatttgattaattatatatcatctttctag

SEQ ID NO: 51 - DNA, 1368 bp, Arabidopsis thaliana
atggagaagagtaatggccttcgagtgattctgtttccacttccattacaaggctgcatcaaccccatgattcagctcgccaagatcctccactcaagaggtttctccatcactgtgatccacacgtgcttcaacgcgccaaaagcttcaagccatcctctcttcaccttcttagagatcccagatggcttgtccgaaacagagaaaagaactaacaataccaaacttctcctaacgcttctcaaccggaactgtgagtctccgtttcgtgaatgtttgagtaaactgttgcagtctgcagattcagaaacaggggaagagaaacagaggattagctgtttgatcgctgattctggatggatgttcacacaacccattgctcagagtttgaaactcccaatattggtcctcagtgtgtttacagtctccttctttcgctgccaatttgttcttcctaagcttcggcgtgaagtgtatcttccacttcaagattcagaacaggaggatctagttcaagagtttccgccgcttcgaaagaaggatattgtacgtattcttgatgtagaaacagatatactagatccattcttggacaaagttctacaaatgacaaaggcgtcttcaggtcttatattcatgtcatgtgaagagttggaccacgactcagtgagtcaggcacgtgaagatttcaaaattcctatctttgggattggaccatctcacagccactttccagctacctctagtagcttgtccacacccgacgagacttgcattccatggttagacaaacaagaagacaaatccgtgatttacgtcagttacgggagcatcgtgaccatcagcgaatcagatttaatagagattgcttggggtctaagaaacagcgaccaacccttcttgttggtcgtacgggttggttcagtccgtggcagagaatggatcgagacaatcccggaagagatcatggaaaagcttaatgagaagggaaagatagtgaaatgggctccgcaacaagacgttctaaagcatcgagccattgggggattcctgacacataatggttggagctcgactgttgagagtgtttgtgaagcagtccctatgatctgtttgccttttcgttgggaccaaatgctaaatgcaagatttgttagcgatgtatggatggtcgggataaacctagaggatcgggttgaaaggaatgagatcgagggagcgataaggagattattggtggaacctgaaggagaagccatccgagagaggatagaacatcttaaggagaaagtaggacgatcgtttcaacaaaacggttccgcatatcaatcgttacaaaatttgattgattatatatcatctttttag

SEQ ID NO: 52 - DNA, 1359 bp, Arabidopsis thaliana
atggcagagattcgccagagaagagtgttgatggtcccagcaccgttccaaggccatttaccttcgatgatgaatctagcgtcctacctttcttcccaaggcttttcaatcacaatcgttagaaacgaattcaatttcaaagatatctcccataatttccctggtataaaattcttcaccatcaaggacggcttgtcagaatctgacgtgaagtctctgggtctccttgaatttgtcctggagcttaactctgtctgtgaacccctattgaaagagtttctaaccaaccatgatgatgttgttgactttatcatttatgatgaatttgtttacttccctcgacgtgttgcggaagatatgaatctgccaaagatggtctttagcccttcttccgccgctacctcgatcagccggtgtgtgcttatggagaaccaatcaaatgggttacttcctccacaagacgcaagatctcaactagaagaaacggtgccagagtttcatccctttcgtttcaaagatctgccttttacagcttatggatctatggagagattaatgatactttacgagaatgtaagcaatagagcctcatcttctggcataatacacaactcttcggattgcttagagaactcattcataacaactgcacaagagaaatggggagttccggtatacccggttggtccactccatatgaccaattccgcaatgtcatgtccaagtttatttgaagaagaaagaaactgtcttgaatggcttgagaagcaagaaacaagctcagtgatctacataagcatggggagcttggcgatgacacaagatatagaggctgtggagatggccatgggatttgtccagagtaatcaacccttcttgtgggtgatccgaccaggctctataaacggacaagaatctttagacttcttaccggaacagttcaaccaaacggtgaccgatggaagaggttttgttgtgaaatgggccccacaaaaagaggtattaaggcatagagcagtgggagggttttggaaccatggtggatggaactcgtgcttggagagcataagcagtggtgtaccaatgatttgtaggccgtattctggtgatcagagggtgaatactcgacttatgtcacatgtttggcaaaccgcgtatgagatcgaaggtgaattggaaagaggagctgttgagatggccgtgaggaggctcattgtggatcaagaaggtcaggagatgagaatgagagccaccatattgaaggaagaggttgaagcctctgtcacaaccgaaggctcttctcacaattctttaaacaatttggtccatgcaataatgatgcaaattgacgaacaatga

SEQ ID NO: 53 - DNA, 1362 bp, Arabidopsis thaliana
atggaagaactaggagtgaagagaaggatagtattggttccagttccagcacaaggtcatgtaactccgattatgcaactcgggaaggctctttactccaagggcttctccatcactgttgttctcacacagtataatcgagttagctcatccaaggacttctctgattttcatttcctcaccatcccaggcagcttgaccgagtctgatctcaaaaaccttggaccattcaagtttctcttcaagctcaatcaaatttgcgaggcaagcttcaagcaatgtattggtcaactattgcaggagcaaggtaatgatatcgcttgtgtcgtctacgatgagtacatgtacttctcccaagctgcagttaaagagtttcaacttcctagcgtcctcttcagcacgacaagtgctactgcctttgtctgtcgctctgttttgtctagagtcaacgcagagtcattcttgcttgacatgaaagatcccaaagtgtcagacaaggaatttccagggttgcatccgctaaggtacaaggacctgccaacttcagcatttgggccattagagagtatactcaaggtttacagtgagactgtcaacattcgaacagcttcggcagttatcatcaactcaacaagctgtctagagagctcatctttggcatggttacaaaaacaactgcaagttccagtgtatcctataggcccacttcacattgcagcttcagcgccttctagtttacttgaagaggacaggagttgccttgagtggttgaacaagcaaaaaataggctcagtgatttacataagtttgggaagcttggctctaatggaaactaaagacatgttggagatggcttggggtttacgtaatagcaaccaacctttcttatgggtgatccgaccgggttctattcccggctcggaatggacagagtctttaccggaggaattcagtaggttggtttcagaaagaggttacattgtgaaatgggcaccacagatagaagttctcagacatcctgcagtgggagggttttggagtcactgcggatggaactcgaccctagagagcatcggggaaggagttccgatgatctgtaggccttttacgggagatcagaaagtcaatgcgaggtacttagagagagtttggagaattggggttcaattggaaggagagctggataaaggaacagtggagagagctgtagagagattgattatggatgaagaaggagcagaaatgaggaagagagttatcaacttgaaagagaagcttcaagcctctgtcaagagtagaggttcctcattcagctcattagacaactttgtcaattccttaaaaatgatgaatttcatgtag

SEQ ID NO: 54 - DNA, 1356 bp, Arabidopsis thaliana
atggaggaaaagccggcgggcagaagagtagtgttggttgcagttccagctcaaggacatatctctccaataatgcaacttgcaaaaacacttcacttgaagggtttctcaatcacaatcgctcagacaaagttcaattactttagcccttcagatgacttcactgattttcagtttgtcaccattccagaaagcttaccagagtctgattttgaggatctcgggccaatagagtttctgcataagctcaacaaagagtgtcaggtgagcttcaaagactgtttgggtcagttgttgctgcaacaaggtaatgagatagcctgtgttgtctacgacgagttcatgtactttgctgaagctgcagccaaagagtttaagcttccaaacgtcattttcagcaccacaagtgccacggcttttgtttgccgctctgcattcgacaaactttatgcaaacagtatcctgactcccttgaaagaacccaaaggacaacaaaacgagctagtgccagagtttcatcccctgagatgcaaagactttccggtttcacattgggcatcattagaaagcatgatggagctgtataggaatacagttgacaaacggacagcttcctcggtgataatcaacacagcgagctgtctagagagctcatctctgtctcgtctgcagcaacagctacaaattccagtttatcctataggccctcttcacctggtggcatcagcttctacgagtcttcttgaagagaacaagagctgtattgaatggttgaacaaacaaaagaaaaactctgtgatattcgtaagcttgggaagcttagctttgatggaaatcaatgaggtgatagaaactgctttgggattggatagtagcaagcaacagttcttgtgggtcattcggccagggtcagtacgtggttcggaatggatagagaacttgcctaaggagtttagtaagataatttcgggtcgaggttacattgtgaaatgggctccacagaaggaagtactttctcatcctgcagtaggaggattttggagccattgcggatggaactcgacactagagagcatcggggaaggagttccaatgatttgcaagccgttttccagtgatcaaatggtgaatgcgagatacttggagtgtgtatggaaaattgggattcaagttgagggtgatctagacagaggagcggtcgagagagctgtgaggaggttaatggtggaggaagaaggggaggggatgaggaagagagctatcagtttgaaagagcaacttagagcctctgttataagtggaggttcttcacacaactcgctagaggagtttgtacactacatgaggactctatga

SEQ ID NO: 55 - DNA, 1362 bp, Arabidopsis thaliana
atggaggaaaagcctgcaaggagaagcgtagtgttggttccatttccagcacaaggacatatatctccaatgatgcaacttgccaaaacccttcacttaaagggtttctcgatcacagttgttcagactaagttcaattactttagcccttcagatgacttcactcatgattttcagttcgtcaccattccagaaagcttaccagagtctgatttcaagaatctcggaccaatacagtttctgtttaagctcaacaaagagtgtaaggtgagcttcaaggactgtttgggtcagttggtgctgcaacaaagtaatgagatctcatgtgtcatctacgatgagttcatgtactttgctgaagctgcagccaaagagtgtaagcttccaaacatcattttcagcacaacaagtgccacggctttcgcttgccgctctgtatttgacaaactatatgcaaacaatgtccaagctcccttgaaagaaactaaaggacaacaagaagagctagttccggagttttatcccttgagatataaagactttccagtttcacggtttgcatcattagagagcataatggaggtgtataggaatacagttgacaaacggacagcttcctcggtgataatcaacactgcgagctgtctagagagctcatctctgtcttttctgcaacaacaacagctacaaattccagtgtatcctataggccctcttcacatggtggcctcagctcctacaagtctgcttgaagagaacaagagctgcatcgaatggttgaacaaacaaaaggtaaactcggtgatatacataagcatgggaagcatagctttaatggaaatcaacgagataatggaagtcgcgtcaggattggctgctagcaaccaacacttcttatgggtgatccgaccagggtcaatacctggttccgagtggatagagtccatgcctgaagagtttagtaagatggttttggaccgaggttacattgtgaaatgggctccacagaaggaagtactttctcatcctgcagtaggagggttttggagccattgtggatggaactcgacactagaaagcatcggccaaggagttccaatgatctgcaggccattttcgggtgatcaaaaggtgaacgctagatacttggagtgtgtatggaaaattgggattcaagtggagggtgagctagacagaggagtggtcgagagagctgtgaagaggttaatggttgacgaagaaggagaggagatgaggaagagagctttcagtttaaaagagcaacttagagcctctgttaaaagtggaggctcttcacacaactcgctagaagagtttgtacacttcataaggactctatga

SEQ ID NO: 56 - DNA, 1350 bp, Arabidopsis thaliana
atggaggaaaagcaagtgaaggagacaaggatagtgttggttccagttccagctcaaggtcatgtaactccgatgatgcaactaggaaaagctcttcactcaaagggtttctccatcactgttgttctgacacagtctaatcgagttagctcttccaaagacttctctgatttccatttcctcaccatcccaggcagcttaactgagtctgatctccaaaacctaggaccacaaaagtttgtgctcaagctcaatcaaatttgtgaggcaagcttcaagcagtgtataggtcaactattgcatgaacaatgtaataatgatattgcttgtgtcgtctacgatgagtacatgtacttctctcatgctgcagtaaaagagtttcaacttcctagtgtcgtctttagcacgacaagtgctactgcttttgtctgtcgctctgttttgtctagagtcaacgcagagtcgttcttgatcgacatgaaagatcctgaaacacaagacaaagtatttccagggttgcatcctctgaggtacaaggatctaccaacttcagtatttgggccaatagagagtacgctcaaggtttacagtgagactgtgaacactcgaacagcttccgctgttatcatcaactcagcaagctgtttagagagctcatctttggcaaggttgcaacaacaactgcaagttccggtgtatcctataggcccacttcatattacagcttcagcgccttctagtttactagaagaagacaggagttgcgttgagtggttgaacaagcaaaaatcaaattcagttatttacataagcttgggaagcttggctctaatggacaccaaagacatgttggagatggcttggggattaagtaatagcaaccaacctttcttatgggtggtcagaccgggctctattccggggtcagaatggacagagtccttaccagaggaattcaataggttggtttcagaaagaggttacattgtgaaatgggctccgcagatggaagttctcagacatcctgcagtaggagggttttggagtcactgtggatggaactcaacagtagagagcatcggggaaggagttccgatgatatgtaggcctttcaccggggatcagaaagtcaatgcgaggtacttagagagagtttggagaattggggttcaattggagggagatctggataaagaaactgtggagagagctgtagagtggttgcttgtggatgaagaaggagcagaaatgaggaagagagccattgacttgaaagaaaagattgaaacctctgttagaagtggaggttcctcatgcagctcactagacgactttgttaattccatgtga

SEQ ID NO: 57 - DNA, 1344 bp, Arabidopsis thaliana
atggagaaaagagtagagaagagaaggatagtgttggttccacttccattactaggacatttcactccgatgatgcaactcggccaagcccttatcttgaagggattctcaattatagttcctcagggagaattcaatcgagtaaactcttcgcagaagttccctggttttcaatttatcaccataccagattctgaactcgaggcaaatggaccagtcgggtctctaacacagctcaacaaaattatggaggcaagcttcaaggactgtataaggcagttgttgaaacaacaaggcaatgatattgcatgtatcatctacgacgagttcatgtatttttgtggagccgtagctgaggagttgaagcttcccaatttcatcttcagtactcaaactgctacacataaagtttgctgcaatgttttaagcaaacttaatgccaagaagtacttgatcgacatggaagagcatgacgtgcaaaacaaggtagtggaaaatatgcatccattaagatacaaagacttaccaactgcaacatttggagaactagaaccttttttggagctctgtagagatgtagtcaacaaaagaacagcctctgctgttatcatcaacaccgtgacctgtctagagagctcgtctctcacaaggctgcaacaagaactccaaattccggtgtatccattaggccctcttcacattacagattcatcgacaggatttactgtgctgcaagaggataggagctgcgttgaatggctgaacaagcagaaaccaaggtctgtcatatacataagtttaggaagcatggttctcatggaaaccaaggagatgttagagatggcttggggaatgttgaatagcaaccaacctttcttatgggtcatccgacctggatctgtctcaggctccgaggggatagagtcattgccagaggaagtcagtaagatggttttagagaaaggatacattgtgaaatgggcaccacaaatagaagtactaggacatccctcagtgggaggcttttggagccactgtggatggaactcaacactcgagagcattgtggaaggagttccaatgatttgcaggccttatcaaggcgagcagatgttaaatgcaatatatctagagagtgtatggagaatagggattcaggtaggaggtgaactggaaagaggagccgtcgagagagctgtgaagaggttgattgtggataaagaaggtgcaagcatgagggagagaacccttgttttaaaagagaagctcaaagcctctattagaggtggaggctcctcatgcaatgcattagatgagcttgtcaagcacttgaagacagagtga

SEQ ID NO: 58 - DNA, 1359 bp, Arabidopsis thaliana
atggagaaaagggtagagaagagaaggattgtgttagttccggttgctgcacaaggacatgtaaccccaatgatgcagcttgggaaagcccttcaatcaaagggcttcttaattactgttgctcagagacagttcaatcaaataggctcatcattgcaacactttcctggttttgactttgtcaccataccagaaagcttacctcagtctgaatctaagaaactaggaccagctgagtatcttatgaatctcaacaaaacaagcgaggcaagcttcaaggagtgtataagtcagttatcgatgcaacaaggcaatgatatagcatgtatcatctatgacaagcttatgtacttctgtgaagcagcagctaaggagtttaagattcctagtgttatcttcagcactagcagtgctacaattcaagtttgctactgtgttttaagtgaactcagtgccgagaagttcttgatcgacatgaaagatcctgaaaagcaagataaggtgttggaaggtttgcatcctttaaggtacaaagacctaccaacttcaggatttggaccattagagccacttttggagatgtgtagggaagtagttaacaaaagaacagcttccgctgttatcatcaacacggcgagctgtctagagagcttgtctctgtcatggctgcaacaagaacttggaattccagtgtatccattaggccctcttcacattacagcttcatcgccgggacctagtttactgcaagaggacatgagctgcattgaatggctgaacaagcagaaaccaaggtcagtcatatacataagcttgggaaccaaagctcacatggagaccaaggagatgttagagatggcctggggattgttgaatagcaaccaacctttcttatgggtcatccgacctggctctgttgcaggcttcgagtggatagagttattaccagaggaagtcattaagatggtaacagaaagaggatacatagcgaaatgggcaccgcagatagaagtacttggacatcctgcagtgggaggattctggagccactgtggatggaactcaacactcgagagtattgtggaaggagtcccaatgatttgcaggcctttacaaggcgaacaaaagttaaatgcgatgtatatagaaagtgtttggaaaatagggattcaacttgaaggtgaagtggaaagggaaggtgtagagagagctgtgaagaggttgatcatagatgaagaaggtgcagccatgagggagagggctcttgatttaaaagagaagctcaatgcctcggtaagaagtggaggctcctcatacaacgcactggatgagcttgtcaagttcttgaatacagagtga

SEQ ID NO: 59 - DNA, 1344 bp, Arabidopsis thaliana
atggagaaaaatgcagagaagaaaagaatagtgttggttccatttccattacaaggacatatcactccaatgatgcaacttggtcaagcacttaacctgaaaggcttctcgattaccgttgctcttggagattccaatcgagtaagttctacgcaacacttccctggttttcaatttgtcacaatacctgaaaccataccactatctcaacacgaggcactcggagttgtcgagtttgtggttacgctcaacaaaacaagcgagacaagtttcaaggactgtatagctcatttgttgctgcaacatggaaatgatattgcttgtatcatttacgacgagctcatgtacttctctgaagctacagctaaggatttaaggattcctagtgtcatattcaccactggtagtgctacaaatcatgtttgttcttgtattttaagcaaactcaacgccgagaagttcttgatcgacatgaaagatcctgaagtgcaaaacatggtggtggaaaatttacatccactaaaatacaaagacttaccaacttcaggaatggggccgctagagcgatttttggagatttgtgccgaagttgtcaacaaaagaacagcttccgctgttataatcaatacgtcaagttgtctagagagctcgtctctgtcatggctgaaacaagaactcagtattccagtgtatccattaggccctcttcacattacaacttcagcaaattttagtttacttgaagaggacaggagctgcattgaatggctgaacaagcagaaactgaggtcagttatatacataagcgtaggaagcatagctcacatggaaaccaaggaagtattggagatggcttggggattgtataatagcaaccaaccttttctatgggtaatccgacccggtacagagtcaatgccagtggaagtcagtaagattgtctcggaaagaggatgcattgtgaaatgggcgccacagaatgaagtacttgtgcatcctgcagtgggaggtttctggagccactgtggatggaactcaacactcgagagtattgtggaaggagttccaatgatttgcagaccgtttaacggtgagcagaagttaaacgcgatgtatatagaaagtgtttggagagtaggggttctgcttcaaggagaagtggagagaggatgtgtagagagagctgtgaagaggttgattgtggatgatgaaggtgtaggaatgagggagagagcccttgttttaaaagagaagctcaatgcctctgtaagaagtggaggctcttcatacaatgcattggatgagctcgtccattacttggaggcagagtatagaaatacttga

SEQ ID NO: 60 - DNA, 1352 bp, Arabidopsis thaliana
atggagaaaatggaagagaagaaaaggatagtgttagttccggttccagcacaaagacatgtaactccaatgatgcagcttggcacagccctaaacatgaagggcttctctattactgttgttgaaggacagttcaataaagtaagctcatctcaaaactttcctggttttcaatttgtaaccataccagatacagagagcttgccagagtctgtgctcgagagactcggaccggtcgagtttttattcgagatcaacaaaaccagtgaggcaagcttcaaggactgtataaggcagtcgttgctgcaacaaggcaatgatatagcatgtatcatctacgacgagtatatgtacttctgtggagctgcagctaaggagttcaaccttcctagtgtaatattcagcacacaaagtgctactaatcaagtttcccgttgcgttttaagaaaactcagtgccgagaagttcttggtggacatggaaggtatcctgaagtgcaggaaacgttggtggaaaatttgcatccattaagatacaaagacctaccaacttcaggagttgggccactagatcgattatttgagctctgtagggaaatagtcaacaaaagaacagcttccgctgttatcatcaacacagtgagatgtctagagagctcgtctctgaaacgtctgcaacatgaactcgggattccggtgtacgcattaggccctcttcacattacagtttcagcagcttctagtttactggaagaggacaggagctgcgttgaatggttgaacaagcaaaaaccgaggtcagtcgtttacataagcttggggagcgtagttcaaatggaaaccaaagaagtgttagagatggctcggggtttatttaatagcaaccagcctttcttatgggtcattcggcctggctctatcgcaggctccgaatggatagagtcactgccagaggaagtcattaagatggtctccgaaagagggtatattgtgaaatgggcaccacagatagaagtacttggacatcctgcagtgggaggattctggagccactgtggatggaactcaacgcttgaaagcattgtggaaggagttccaatgatatgcaggccctttcatggcgagcaaaagttaaacgcactgtgtttagagagtatttggagaatagggtttcaggtgcaaggtaaggtagagaggggaggggtcgagagagctgtgaagaggttgatagtggatgaagaaggtgcagacatgagagagagagcccttgttttaaaagagaatctcaaagcctctgtaagaaatggaggctcctcatacaacgcattggaggagatcgttaacctcatgtag

SEQ ID NO: 61 - DNA, 1350 bp, Arabidopsis thaliana
atggaggagaagctctcgaggagaagaagagtagtgttggttccagttccagctcaaggacatataactccaatgatacaacttgcaaaagcacttcactcaaaaggcttctctattacagttgttcaaaccaagttcaactacttaaacccttcaaatgatttgtctgattttcagtttgtaaccatcccagagaacttaccagtgtctgatcttaagaatctaggaccaggacggtttctgattaagctagctaatgagtgttatgttagctttaaggatttgttaggtcagttgttggttaatgaagaagaagagatcgcttgtgttatctacgacgagttcatgtactttgttgaagtagcagttaaagagtttaagcttcgtaatgttattttaagtactacaagtgcaacggcttttgtttgtcgctttgttatgtgtgaactctatgctaaagatggtttggctcaacttaaagaaggcggtgagcgagaagtggagttagtaccggagttgtatcctatacggtacaaagatttaccaagttcggtatttgcatctgtagaatcttcagtggagttgtttaagaatacatgttataaagggacagcttcctctgtgataatcaacacagtgaggtgtctagagatgtcatctttggagtggcttcaacaagaacttgaaatcccggtgtattctataggcccgcttcatatggtggtgtcagctcctcctacgagtcttttagaagagaacgagagctgtatagaatggttgaacaaacaaaagccgagctcggtgatatacataagcttgggaagttttactttgatggaaactaaagaaatgttggagatggcttatgggtttgttagtagtaaccaacacttcttgtgggtgattcgaccgggatctatatgtggttctgaaatctctgaggaagagttgttgaagaagatggtaattacggatcgaggttacattgtgaaatgggcgccgcaaaaacaagtgcttgcacattctgcggttggagcgttctggagtcattgtggatggaactcgactttagaaagtcttggtgaaggagttccattgatatgtaggccttttactactgatcaaaaggggaatgcaaggtacttggagtgtgtgtggaaagtaggaattcaagtggagggtgagctagagagaggcgcaatcgagagagctgtgaagaggttaatggtggatgaagaaggagaagagatgaagagaagagctctaagtttaaaagagaaactcaaagcctctgttttagctcaaggttcttcacataaatcactagatgacttcatcaagactctgtga

SEQ ID NO: 62 - DNA, 1362 bp, Arabidopsis thaliana
atggaggaaaagcaagagaggaggagaaggatcgtgttgattcccgctccagcacaaggacacatatctccgatgatgcaacttgcaagagcccttcacttaaagggcttctccattacagttgctcaaaccaagttcaattacttgaagccttcaaaagacttagctgattttcagtttatcaccatcccagagagcttaccagcctcggatcttaagaatctaggaccagtttggtttcttcttaaactcaataaagagtgtgagtttagcttcaaggagtgtttaggtcaattgttgctgcaaaaacaacttataccggaagaagagatcgcttgtgtcatctacgacgagttcatgtactttgctgaagctgcagccaaagagtttaaccttcccaaagttattttcagtaccgaaaatgcgacggcttttgcttgtcgctctgccatgtgcaaactctatgcaaaagatggtttggctccccttaaagaaggatgtgggcgagaagaggagctagtgccaaagttgcatccccttagatacaaagacctaccaacttcagcatttgcaccagtagaagcctcagtggaagtgtttaaaagttcatgtgataaagggacagcttccgctatgataatcaacacagtgaggtgtctagagatatcatccttggagtggcttcaacaagaacttaagattccgatatatcctataggccctcttcacatggtttcttcagctcctcctacgagtctactagacgagaatgagagttgcattgattggctgaacaaacaaaagccgagctcggtgatttacataagtttgggaagctttactttgttggaaactaaagaagtgttggaaatggcttcgggcttggttagtagtaaccaacacttcttgtgggtgattcgacccgggtccatacttggttctgaattgactaatgaggaattattgagtatgatggaaataccggatcgaggctacattgtgaaatgggctccacaaaagcaagtgcttgcacattctgcggttggagcattttggagtcattgtggatggaactcgactctagagagcatgggtgaaggagttccgatgatttgtaggccttttactactgatcaaaaggtaaatgcgcggtatgtggagtgtgtctggagagttggggttcaagtggagggtgaactaaagagaggagtagtcgagagagctgtgaagaggttactggtggatgaagaaggagaagagatgaagttgagagctctcagtttgaaagagaaactcaaagtttctgttctaccgggaggttcttcacacagttcactagatgacttaatcaagactctatga

SEQ ID NO: 63 - DNA, 1395 bp, Arabidopsis thaliana
atggaagagagaaaagtgaagagaattatcatgttccctctaccgtttacaggacacttcaaccctatgatcgagcttgctggaatattccacaaccgtggcttctccgtcacgatactccacacttctttcaacttcccggatccttctcgccatccacagtttacttttcgaactatcactcacaaaaacgaaggagaagaagaccctctctctcaatcagaaacttcttcgggtaaggacctcgtcgtccttattagtctgctgaaacaatactacaccgagccgtctcttgcagaggaagtaggcgaaggagggacggtgtgttgtttggtctccgacgctctatgggggaggaacacggagattgtagcgaaagagattggagtgtgtacaatggtgatgaggactagtggtgcggcaacgttttgtgcttatacagctttccctctccttatagataagggttaccttcctatacaaggttctagattagatgagctagtgacagagcttccacctttgaaagtgaaggatcttcctgtaataaaaacgaaagagcctgagggactaaaccgaatacttaacgacatggtggaaggagccaagttatcttccggagtcgtatggaacacatttgaagatcttgaaagacattcactcatggattgtcgcagcaagttacaagttccgttgttcccaatcggaccgtttcacaaacatagaaccgatcttccaccgaagccaaagaacaaggacaaggacgatgatgaaatattaaccgattggcttaacaagcaagctccgcagtctgtggtctatgtgagttttggaagccttgcagctatagaagagaatgagtttttcgaaattgcttggggtctaagaaacagcgaactaccattcttgtgggtggttaggcccgggatggtccggggaaccgagtggcttgagtcattgccttgtgggtttttggaaaatattggtcatcagggaaaaattgtgaaatgggtgaatcaactagagacattggcccatcctgcggttggagcgttttggacgcactgtggatggaactcaacaatagagagcatatgtgaaggtgttccaatgatatgtacgccgtgtttctcggaccagcatgtgaacgcgaggtacatcgttgatgtatggcgagtcgggatgatgttagagagatgtaagatggaaaggacggagattgagaaggtagtaacaagtgtaatgatggagaatggagctggattgacagagatgtgtttggagttgaaagagaaagctaatgtttgcttaagtgaagatgggtcttcttccaagtatctagacaaacttgtcagtcatgtcctgtcttttgattcctcggcttttgcaagttaa

SEQ ID NO: 64 - DNA, 1383 bp, Arabidopsis thaliana
atggaagagagaaaagggaggagaataatcatgttccctcttccatttccagggcacttcaaccccatgatcgagctcgctggaatattccaccaccgtggcttctccgtgacgatcctccacacttcctacaacttccccgatccttctcgccacccacacttcacttttcgaaccatctctcacaacaaagaaggagaagaagatcctctgtctcagtcagaaacttcgagtatggacctaatcgttctcgttcgtcggctgaaacaacgctacgccgaaccgtttcgtaagtctgtggcggcggaagtaggtggaggagagacggtgtgttgtttggtctccgacgctatatgggggaagaacacggaggttgtagcggaagagattggagttcgtagggtggtgttgaggacaggtggtgcgtcgtcgttttgtgcttttgccgctttccctctccttagggataagggttacctccctatacaagattctagattagatgagccagtgacagagcttccacctttgaaagtgaaggatcttccggtaatggaaacgaatgagccggaggaactttaccgggtagttaacgacatggtggaaggagccaagtcttcttcaggagtcatatggaacacatttgaagatcttgaaagactatcacttatgaattgtagcagcaaattacaagttccatttttcccgatcggaccgtttcacaaatatagcgaagatcctacaccgaagacagagaacaaggaagataccgattggctcgacaagcaagacccacagtcggtggtctatgcgagtttcggaagccttgcagctatagaagagaaggagtttctcgagattgcttggggtctaagaaacagtgaacgaccgtttttgtgggtggttaggccggggtctgtcagggggaccgagtggctcgagtcattgcctttagggtttatggaaaacattggagataagggaaaaatcgtgaaatgggcgaatcagttagaggtattggcgcatcctgccattggagcgttttggacacattgtggatggaactcgacactagagagcatatgtgaaggtgttcctatgatatgtacgtcatgtttcacggaccagcatgtgaacgcgagatacatcgttgatgtatggcgagtcgggatgttgttagagagaagtaagatggaaaagaaggagattgaaaaggtgctaagaagtgtaatgatggagaagggagatggattgagggaaaggagtttgaagttgaaagagagagctgatttttgcttaagtaaagatgggtcttcttccaagtatttagacaaacttgtgagtcatgtcctgtcttttgattcttatgcttttgcaagttaa

SEQ ID NO: 65 - DNA, 1362 bp, Arabidopsis thaliana
atgaccaaattctccgagccaatcagagactcccacgtggcagttctcgcgtttttccccgttggcgctcatgccggtcctctcttagccgtcactcgccgtctcgccgccgcttctccctccaccatcttttctttcttcaacaccgcaagatcaaacgcgtcgttgttctcctctgatcatcccgagaacatcaaggtccacgacgtctctgacggtgttccggagggaaccatgctcgggaatccactggagatggtcgagctgtttctcgaagcggctccacgtattttccggagcgaaatcgcggcggcagagatagaagttggaaagaaagtgacatgcatgctaacagatgccttcttctggttcgcagcggacatagcggctgagctgaacgcgacttgggttgccttctgggccggcggagcaaactcactctgtgctcatctctacactgatctcatcagagaaaccatcggtctcaaagatgtgagtatggaagagacattagggtttataccaggaatggagaattacagagttaaagatataccagaggaagttgtatttgaagatttggactctgttttcccaaaggctttataccaaatgagtcttgctttacctcgtgcctctgctgttttcatcagttcctttgaagagttagaacctacattgaactataacctaagatccaaacttaaacgtttcttgaacatcgcccctctcacgttattatcttctacatcggagaaagagatgcgtgatcctcatggctgctttgcttggatggggaagagatcagctgcttctgtagcgtacattagcttcggcaccgtcatggaacctcctcctgaagagcttgtggcgatagcacaagggttggaatcaagcaaagtgccgtttgtttggtcgctgaaggagaagaacatggttcatctaccaaaagggtttttggatcggacaagagagcaagggatagtggttccttgggctccacaagtggaactgctgaaacacgaggcaatgggtgtgaatgtgacacattgtggatggaactcagtgttggagagtgtgtcggcaggtgtaccgatgatcggcagaccgattttggcggataataggctcaacggaagagcagtggaggttgtgtggaaggttggagtgatgatggataatggagtcttcacgaaagaaggatttgagaagtgtttgaatgatgtttttgttcatgatgatggtaagacgatgaaggctaatgccaagaagcttaaagaaaaactccaagaagatttctccatgaaaggaagctctttagagaatttcaaaatattgttggacgaaattgtgaaagtttag

SEQ ID NO: 66 - DNA, 1383 bp, Arabidopsis thaliana
atgaccaaaccctccgacccaaccagagactcccacgtggcagttctcgcttttcctttcggcactcatgcagctcctctcctcaccgtcacgcgccgcctcgcctccgcctctccttccaccgtcttctctttcttcaacaccgcacaatccaactcttcgttattttcctccggtgacgaagcagatcgtccggcgaacatcagagtatacgatattgccgacggtgttccggagggatacgtgtttagcgggagaccacaggaggcgatcgagctgtttcttcaagctgcgccggagaatttccggagagaaatcgcgaaggcggagacggaggttggtacggaagtgaaatgtttgatgactgatgcgttcttctggttcgcggctgatatggcgacggagataaatgcgtcgtggattgcgttttggaccgccggagcaaactcactctctgctcatctctacacagatctcatcagagaaaccatcggtgtcaaagaagtaggtgagcgtatggaggagacaataggggttatctcaggaatggagaagatcagagtcaaagatacaccagaaggagttgtgtttgggaatttagactctgttttctcaaagatgcttcatcaaatgggtcttgctttgcctcgtgccactgctgttttcatcaattcttttgaagatttggatcctacattgacgaataacctcagatcgagatttaaacgatatctgaacatcggtcctctcgggttattatcttctacattgcaacaactagtgcaagatcctcacggttgtttggcttggatggagaagagatcttctggttctgtggcgtacattagctttggtacggtcatgacaccgcctcctggagagcttgcggcgatagcagaagggttggaatcgagtaaagtgccgtttgtttggtcgcttaaggagaagagcttggttcagttaccaaaagggtttttggataggacaagagagcaagggatagtggttccatgggcaccgcaagtggaactgctgaaacacgaagcaacgggtgtgtttgtgacgcattgtggatggaactcggtgttggagagtgtatcgggtggtgtaccgatgatttgcaggccattttttggggatcagagattgaacggaagagcggtggaggttgtgtgggagattggaatgacgattatcaatggagtcttcacgaaagatgggtttgagaagtgtttggataaagttttagttcaagatgatggtaagaagatgaaatgtaatgctaagaaacttaaagaactagcttacgaagctgtctcttctaaaggaaggtcctctgagaatttcagaggattgttggatgcagttgtaaacattatttga

SEQ ID NO: 67 - DNA, 1378 bp, Arabidopsis thaliana
atggccaaaccctcgcagccaacgcgagactcccacgtggcagttctcgttttccccttcggcactcatgcagctcctctcctcgccgtcacgtgccgtctcgccaccgctgctccctccaccgtcttctccttcttcagcaccgcacgatccaactcgtcgttactctcctccgatatccccacaaacattcgtgtccacaacgtcgatgacggtgttcctgagggattcgtgttgacggggaatccacagcacgctgttgagctgtttcttgaagcggcgccagagattttccgaagagaaatcaaggcggccgagaccgaagttggtaggaagttcaagtgcatccttacggatgcgttcctctggttagcagcggagacggcggctgcggagatgaaagcgtcgtgggttgcgtactatggaggcggagcaacctcgctcactgctcatctctacacagatgccatcagagaaaacgtcggtgtcaaaagtaggtgagcgtatggaggagacaatagggtttatctcaggaatggagaagatcagagtcaaagacacacaagaaggcgttgtgtttgggaacttagactctgttttctctaaaacgttgcaccaaatgggtcttgctttacctcgtgccactgctgttttcatcaattcctttgaagaattggatcctacgtttacaaatgatttcagatcggaattcaaacgttacctaaacatcggtcctctcgctttattatcttctccatcgcaaacatcaacgctagtgcacgatcctcacggttgcttggcttggatcgagaagcggtccactgcttctgtagcgtacattgcctttggtagagtcgcgacaccgcctcctgtagagcttgtggcgatagcacaaggattggaatcgagtaaagtgccttttgtttggtcgctacaagagatgaaaatgactcatttaccagaaggctttttggatcggaccagagagcaagggatggtggttccatgggcaccacaagtggagctgctaaaccatgaagcaatgggtgtgtttgtttcgcatggtgggtggaactcagtgttggagagtgtgtcggcaggtgtaccgatgatttgtagaccgattttcggggatcatgcaatcaatgcaagatctgtggaagctgtgtgggagatcggagtgacgattagtagtggagtcttcacgaaggatggatttgaggagagtttggatcgggttttggttcaagatgatggcaagaagatgaaggttaatgctaaaaagcttgaagaactagcacaagaagctgtctctaccaaaggaagctcctttgagaattttggaggattgttggacgaagttgtgaactttggataa

SEQ ID NO: 68 - DNA, 1407 bp, Arabidopsis thaliana
atgggtgtttttggatcgaatgaatcgtcaagcatgagtattgtgatgtatccgtggttagcctttggtcacatgactccttttcttcacctatccaacaagctcgcagagaaaggtcacaagattgttttcttgcttcccaagaaagcactaaaccagcttgaacctcttaatctctacccaaatctcatcactttccacaccatctctatccctcaggtcaaagggctccctccgggtgcggagacaaactccgacgtccctttcttcttgacacatttgcttgcagttgcaatggaccaaacccggccagaggtcgagaccattttccgtacaatcaaaccggacttggttttctatgattctgcccattggataccggaaattgctaaaccgatcggtgctaaaaccgtttgcttcaacatcgttagcgctgcgtcaatcgcactgtctcttgtcccttctgcggagagagaggtcattgatggcaaggaaatgtcaggggaggagttagctaagacgcctctaggttacccatcttcgaaagtagtcttacgtccgcacgaagcaaaatccctgagtttcgtgtggaggaagcacgaggcgattggctctttctttgatgggaaagttaccgcgatgagaaactgcgacgcaatcgctataaggacttgccgtgagacagaaggcaaattctgcgattacataagtaggcagtacagtaaaccggtttacctaacaggaccggttctccctggatcccaacctaatcagccctccttagatcctcaatgggcggagtggctagccaaattcaaccacggttcggttgtgttctgcgctttcggtagccaacccgttgtaaacaagatagatcagtttcaagaactctgtttaggtctagaatcaactggttttccgtttctggttgccattaagcctccttcgggtgtatcaaccgtcgaggaagccttaccggaaggattcaaagagagggttcaaggacgtggcgttgtgtttggaggttggattcagcaaccgttggtgttgaaccatccttcagtgggttgttttgttagccattgcgggtttgggtcgatgtgggagtcgttgatgagtgattgtcagatcgttttggttccgcagcacggagaacagattttgaacgcaaggctgatgacggaggagatggaggtggcggttgaagtggagagggaaaagaaagggtggttctcgcggcaaagcttggagaatgctgtgaagagtgtgatggaggaaggtagtgagatcggtgagaaagtgaggaagaatcatgacaagtggagatgtgttttgactgactctggtttttcagatggttatattgataagtttgaacaaaatttaattgaacttgtgaagtcatga

SEQ ID NO: 69 - DNA, 1344 bp, Arabidopsis thaliana
atgggccaaacgtttcacgcctttatgttcccatggttcgcttttggtcatatgactccatacttgcatttagccaacaagttagctgagagaggtcacagaatcactttcttgatccccaagaaagctcagaagcagcttgaacatctcaatctgtttccagacagcatcgtctttcactctcttactattcctcatgttgatggtctccccgctggagccgagactttctcggatatccctatgccattgtggaagttcttgcccccagctatagatctcacacgcgatcaagttgaagcagcggttagtgccttgagtccggacctgatcttgttcgatattgcttcatgggttccagaagtggctaaagagtatagagtcaagagtatgttgtacaacatcatatcagctacttctatagctcatgactttgtcccaggtggtgaacttggagttcctccacctggttatccttcctcaaagttgttgtaccgcaaacacgatgctcacgccttgttgtccttctccgtctactacaagaggttttctcatcggctcatcacaggtcttatgaattgtgatttcatttcgataaggacatgcaaagaaatcgagggtaaattctgcgagtatcttgagcgtcaataccataaaaaggttttcttgacgggtccaatgcttcctgagccaaacaaaggtaaaccactggaagatcgatggagtcattggctgaacgggtttgaacaaggctctgtagtgttctgtgcattgggaagtcaagtcactctagagaaggaccagttccaagaactttgtttaggaatagagcttacaggtttaccgttttttgtagctgtaacaccaccaaaaggcgcaaagacgattcaagatgcgttaccagaagggttcgaggagagggtgaaagatcgtggagtggttttgggagaatgggtgcaacaaccgttattattggctcatccatcagtaggctgcttcttgagtcattgcggattcgggtcaatgtgggaatctataatgagtgattgccaaatagttttgcttccatttttggctgatcaagttctcaacacaagattgatgaccgaagaactcaaggtttcggttgaagtgcaaagagaagaaacaggatggttctcgaaggagagcttgagtgttgctatcacatctgtgatggaccaagctagtgagatcgggaatctggtgagaaggaaccattccaaattgaaggaggttttggttagtgatggattattaaccggttacaccgataaatttgttgacactttggagaatcttgtcagcgagacaaagcgtgaatga

SEQ ID NO: 70 - DNA, 1359 bp, Arabidopsis thaliana
atgggccaaaagattcacgcttttatgttcccctggtttgcttttggtcatatgactccgtacttgcatctaggcaacaagttagccgagaaaggtcatagggttactttcttgctacctaagaaagctcagaaacaattggaacatcagaatctatttccacacggtatcgtctttcatcctcttgttattcctcatgttgatggcctccctgctggtgccgagacagcctcggatatccccatctcgttggtgaagttcttgtctatagccatggatcttacacgcgatcagatcgaagccgcgattggtgccttgagaccggacctaatcttgttcgatttagctcactgggttccggaaatggctaaagcgcttaaagtcaagagtatgttgtataacgtgatgtcagctacctctatagctcacgaccttgtcccaggtggtgaacttggagttgctccacctggttatccttcatcaaaggcgttgtaccgcgaacacgatgctcacgccttgttaaccttctccggcttctacaagaggttttatcaccggttcaccacaggtcttatgaattgcgatttcatttcgattcggacatgtgaagaaatcgaaggtaaattttgtgactatattgagagtcaatacaagaagaaggttcttttaaccggtccaatgcttcccgagcctgacaagagtaaaccacttgaagatcaatggagtcattggctgagtgggtttggacaaggctctgtagtgttctgtgcattgggaagtcaaaccattctagagaaaaaccaattccaagaactctgtttaggaatagagcttacgggtttaccatttcttgtcgcggttaagccaccaaaaggcgcaaacacaattcatgaagcgttaccagaagggttcgaggaaagggtgaagggtcgtggaatagtttggggagaatgggtgcagcaaccatcctggcaaccattgatattggctcatccatcagtaggttgctttgtgagccattgcggattcgggtcaatgtgggaatctttaatgagtgattgtcaaatagtctttattccagttttgaatgatcaagttctcaccacgagagtaatgacggaggaactcgaggtctccgttgaggtacagagagaagaaacaggatggttctcaaaagaaaacttgagtggtgcaatcatgtctttgatggaccaagacagcgagatagggaaccaagtgaggaggaaccattctaaattgaaggagactttggctagtcctggattattaaccggttacaccgataaatttgttgacactttggagaatctagtcaacgaacaaggatacatatcttga

SEQ ID NO: 71 - DNA, 1368 bp, Arabidopsis thaliana
atgggtggtttgaagtttcatgtacttatgtatccatggttcgcaacaggccatatgaccccgttcctttttcttgccaacaaattggctgagaaaggtcatacggtcacttttttgattcccaagaaagctctgaaacagttggaaaatctcaatctgtttccacacaacattgtctttcgctctgtcaccgtccctcatgtggatggtctccccgttggcacagagacagtctctgagatccccgtgacatcagctgatctcttgatgtctgctatggatctcacacgtgatcaagttgaaggtgtggtccgagccgtggaaccggacctgatcttctttgacttcgctcattggattccagaggtagctagagactttggccttaagactgtaaagtacgtcgtggtatctgcatcgactatagctagtatgcttgttccaggtggtgagttaggtgttcctccgccgggatatccttcatcgaaggtgctgcttcgtaaacaagatgcttacaccatgaagaatctggagtctacaaatacaatcaatgtcggaccaaacttattggaaagagtcactacaagtcttatgaactctgatgtcattgcgataaggacagccagagaaatcgaaggaaacttttgcgactatatcgaaaaacattgcaggaaaaaggttctcttgacaggtccggtgttccctgagccagacaagactagagagctagaggaacgatgggttaagtggctaagtgggtatgaaccagactcagtggtgttttgtgcgttgggctcacaagtcattttagagaaagatcaattccaagaactctgcttaggaatggagctaacaggttcaccgtttcttgtagcggttaagccacctagaggctcatcaacgattcaagaagcacttcctgaaggattcgaggagagggttaaaggaagaggagttgtttggggagaatgggttcaacaaccattgctattgtctcatccatcagtcgggtgctttgtgagccattgtgggtttggatcaatgtgggagtctttgctgagtgattgtcagatagtcttggtaccacagttgggtgatcaggtcctcaacacaagattgctgagtgacgaactcaaggtttcggttgaagtggcaagagaggaaacaggatggttctcgaaagagagcttgttcgatgctatcaatagtgtgatgaaaagggacagtgagatcgggaatctggtgaagaagaatcacaccaagtggagggagacactaactagtcctggacttgtgaccggttatgtcgataatttcatagagtcattgcaggatcttgtctctgggaccaaccatgtttcgaagtag

SEQ ID NO: 72 - DNA, 1362 bp, Arabidopsis thaliana
atgggtggtttgaagtttcatgtacttatgtatccatggttcgcaacaggccatatgaccccgttcctttttcttgccaacaaattggctgagaaaggtcatacggtcactttcttgcttcccaagaaatctctgaaacagttggaacatttcaatctgtttccacacaacattgtctttcgctctgtcaccgtccctcatgtggatggtctccccgttggcacagagacagcctctgagatccctgtgacatcaactgatctcttgatgtctgctatggatctcacacgtgatcaagttgaagctgtggtccgagccgttgaaccggacctgatcttctttgactttgctcattggattccagaagtagctagggacttcggccttaagactgtaaagtacgtcgtggtgtctgcatcgactatagctagtatgcttgtcccaggtggtgagttaggtgttcctccaccgggatatccatcatcaaaggtgctgcttcgtaaacaagatgcttacactatgaagaaactggagcctacaaatacaatcgatgtcggaccaaacctcttggaacgagtcactacaagtcttatgaactctgatgtcattgcgataaggacagccagagaaatcgaaggaaacttttgcgactatatagaaaaacattgcaggaaaaaggttctcttgacaggtccggtgttccctgagccagacaagactagagagctagaggaacgatgggttaagtggctaagtgggtatgaaccagactcagtggtgttttgtgcactgggctcacaagtcattttagagaaagatcaattccaagaactctgcttaggaatggagctaacaggttcaccgtttcttgtagcggttaagccccctagaggctcatcaacgattcaagaagcacttcctgaaggattcgaagagcgggttaaaggaagaggccttgtttggggaggatgggttcaacaaccattgatattgtctcatccatcagtcgggtgctttgtgagccattgtgggtttggatcaatgtgggagtctttgctgagtgattgtcagatagtcttagtaccacagttgggtgatcaagtcctgaacacaagattgctgagtgacgaactcaaggtttcggttgaagtggcaagagaggaaacaggatggttctcgaaagagagcttgtgcgatgctgtcaatagtgtgatgaaaagggacagcgagctcgggaacctggtgaggaagaatcacaccaagtggagggagacagtagctagtcctggactaatgactggttatgtcgatgctttcgtagagtcattgcaggatcttgtctctgggaccacccatgactga

SEQ ID NO: 73 - DNA, 1347 bp, Arabidopsis thaliana
atggggtcaaagtttcatgcttttctttatccatggtttggttttggtcatatgattccgtatcttcatctagctaacaaattagctgaaaaaggtcatagggttactttcttggctcccaagaaagctcagaaacaactcgaacctctcaacttgttcccaaacagcattcacttcgagaatgttactcttcctcatgttgatggtctccctgttggcgcagagacaaccgcggatctcccgaactcatctaagagagtcctcgctgatgccatggatcttctacgcgaacagattgaagttaagattcgttctttgaaacctgacctaattttcttcgattttgttgattggattccacaaatggcaaaagaattaggaatcaaaagtgtaagttaccagatcatatcggcagcttttatagctatgtttttcgctcctcgtgctgaattaggttctcctccacctgggtttccttcatcaaaagtagcattacgtggacatgacgctaacatctattcactcttcgcaaacacccgcaaatttctctttgatcgagtcaccacaggccttaagaactgcgacgtcattgccataaggacatgtgcagaaatcgaaggtaacttatgtgatttcatcgaaagacaatgtcagagaaaagttctcttaaccggtccaatgttccttgatccacaagggaagagtggtaagccgctagaagatcgatggaataattggttaaacggatttgaaccaagctcggtagtgtactgtgcgtttggcacccatttctttttcgagatagatcaatttcaagaactctgtttaggaatggagctcacgggtctaccttttttggtagcggttatgccaccgagagggtcttcaacgattcaagaagcattaccagaagggttcgaagaacggattaaagggcgtggaattgtttggggaggatgggtggaacaacctttgatattgtctcatccatcaataggttgctttgtgaaccattgcgggttcggttcaatgtgggagtctttggttagtgattgccagattgtgtttattccacaattggttgatcaagttctcacaacgagattgttgaccgaagaactcgaggtctccgtgaaagtaaagagagatgaaattactggttggttttcgaaggagagcttgagggatacggtcaaatctgtgatggataaaaatagtgagattgggaatctagtgaggaggaatcataagaaactgaaggaaactttggttagtcctggattgttgagtagttatgctgataagtttgttgacgaattagagaatcatatccacagtaagaattga

SEQ ID NO: 74 - DNA, 1347 bp, Arabidopsis thaliana
atgggatcaaaatttcatgcttttatgtatccatggtttggttttggtcatatgattccatatcttcatttagccaacaaactagctgagaaaggtcatagggtcactttcttcctccccaagaaagctcataagcagctccaacctctcaatctgttcccagacagcattgtctttgagcctcttactctccctcctgtcgatggtctcccttttggcgccgagacagcctcggatctcccaaactcaactaagaaacccatattcgttgccatggatctcttacgcgatcagatcgaagcaaaggtccgtgctttgaaaccagatctaatctttttcgattttgttcattgggttccagaaatggcagaagagtttggaataaagagtgtcaattaccagatcatatcggcagcttgtgtagctatggttcttgcacctagggctgaattagggtttcctccgccggattatcctttatccaaagtggcgttacgtggacatgaagctaacgtctgttctctctttgcgaattcccatgagcttttcggtctgatcaccaaaggccttaagaactgtgacgtcgtttccataaggacctgcgtggaacttgaaggtaagctatgcggtttcatcgaaaaagaatgtcaaaagaaacttctcttaaccggtccaatgctccctgaaccgcaaaataagagtggtaaatttctagaagaccgatggaatcactggttaaacggatttgaaccagggtcggtagtgttttgtgcgtttggcactcaattctttttcgagaaggatcaatttcaagaattctgtttaggaatggagctaatgggtctaccgtttttaatatcggttatgccgccaaaaggctcaccaacggttcaagaagcgttaccaaaaggattcgaagaacgggttaaaaagcatggaatcgtttgggaaggatggttggaacaacctttgatattgtctcatccatcagtaggttgctttgtgaaccattgtggctttggttcaatgtgggagtctttggttagtgattgtcagattgtgtttattccacaattggcagatcaagttctcatcacaagattgttgactgaagaactcgaagtctctgtgaaagtgcagagagaagattccggatggttctcgaaagaggacttgagagatactgttaaatctgtgatggatatagatagtgagattgggaacttagtgaagaggaatcataagaaattgaaagagactttagttagtcctggattgttaagtggttatgctgataagtttgtagaagcattggagattgaagtcaacaacaccaaattttcttga

SEQ ID NO: 75 - DNA, 1362 bp, Arabidopsis thaliana
atggggtcaaagtttcatgcttttatgttcccatggtttggttttggtcacatgactgcatttttgcatctggctaacaaactagcggagaaagaccacaaaataactttcttgctccccaagaaagctcgaaagcaacttgaatctctcaatctcttcccagactgcattgtctttcagactcttaccatcccatctgtagatggcctccctgatggtgctgagacaacctcggatatcccgatctcgttaggcagttttctcgcctcggctatggatcggacacgcattcaggtcaaagaagcagtttctgttggtaaaccggatctgattttcttcgattttgctcactggattccggaaatagctagagagtatggagtcaagagtgtcaatttcataacgatttctgcagcatgtgtagctatttcgttcgtccctggtcgtagtcaagatgacttgggtagtactccaccgggatacccttcctccaaggtgttgcttcggggacacgaaaccaacagtttgtcgttcctctcctatccgtttggagatggaactagtttttacgaacggatcatgataggacttaagaactgcgatgtcatttcgataaggacatgccaagaaatggaaggaaagttctgcgatttcatcgaaaaccaatttcaaagaaaagttctcttgacaggtccaatgcttcctgagccggacaatagcaaaccgctagaagatcaatggcgtcagtggcttagcaagttcgatccgggatcagtaatatattgtgcattgggcagccaaatcattcttgaaaaggatcaattccaagaactctgtttaggaatggagctgacaggtttaccatttcttgtagcggtaaagccaccaaaaggttcatcgacaatccaagaagccttaccaaaagggtttgaagagagggttaaagcacgtggagtggtttggggaggatgggtgcagcaaccattgatattagctcatccatcaataggctgctttgtgagccattgtggtttcgggtcaatgtgggaggctctagtgaatgactgccaaatagtgtttattccacatttgggtgagcaaatattgaacacaagactgatgagcgaggaactcaaggtctcggtagaggtgaaaagagaggaaacgggatggttttcgaaggagagcttgagcggtgcggtcaggtctgtgatggacagagatagcgagctcgggaattgggcgaggaggaaccacgtaaagtggaaggagtctctgcttcgtcatggactaatgagtggttatcttaataagttcgtagaagcattggagaaactagtccaaaatataaatcttgaatga

SEQ ID NO: 76 - DNA, 1329 bp, Arabidopsis thaliana
atggagccaaagtttcatgcttttatgtttccatggtttgcttttggtcatatgattccatttctacatcttgcaaacaaactagctgaaaaaggtcaccgagttactttcttgctacctaagaaagcacaaaaacagttggaacatcacaacttgttcccagacagtattgtctttcaccctctcacagttcctcctgtcaatggcctccctgctggtgccgagacaacctcggatatccccatctcgttggacaacctcttgtccaaagccttggatctcactcgcgatcaggttgaagctgcggttcgtgctttgagacctgacttgatctttttcgattttgctcaatggattccagatatggctaaagaacatatgatcaagagtgtgagttacatcattgtatctgcgacaacaatagctcatacacatgtccctggaggtaaattaggtgttcgcccaccgggttatccgtcatcaaaggtgatgttccgtgaaaacgatgttcatgccttagcaaccttatcgatattttacaagagactgtatcatcagatcactacaggtcttaagagctgtgatgtcattgcattgaggacttgcaaagaagtcgaaggtatgttctgcgactttatatcgcgtcaataccataagaaggttctcttgactggtccaatgttccctgagccagacacaagtaaaccactagaagaacgctggaatcattttctaagcgggttcgcgccgaagtcagtagtgttttgttcacctggcagccaagtaattcttgagaaagatcaattccaagaactctgtttagggatggagctaacaggtttaccatttcttttagcggtaaagccaccaagaggatcatcaacggtccaagaagggttaccagaagggttcgaggagcgggtgaaagatcgtggtgttgtttggggaggatgggtgcaacaacctttgatattggctcatccatcaataggttgctttgtgaaccattgtggtcccggaacaatatgggagtctttggtgagtgattgccaaatggttttgattccatttttaagtgatcaagttctcttcacaagattgatgaccgaggaattcgaggtctctgtagaagtgccgagggaaaaaacaggatggttttcaaaggagagcttgagcaatgctatcaaatctgtgatggataaagacagtgacattgggaagttagtgaggagtaaccacaccaaattgaaggagattttagttagtcctggattattgactggttacgttgatcactttgtagagggattgcaagagaatttgatttga

SEQ ID NO: 77 - DNA, 1329 bp, Arabidopsis thaliana
atggagccaacgttccatgcttttatgtttccctggtttgcttttggtcatatgattccttttctacatcttgcaaacaaactagctgagaaaggtcatcaaatcactttcttgctacctaagaaagcccaaaaacagttggaacatcacaatctgttcccagacagtattgtctttcaccctctcacaatccctcatgtcaatggcctccctgctggtgctgagacaacctcggatatctcaatctcgatggacaacttactgtcggaagccttggatctcactcgcgatcaggttgaagctgcggttcgtgctctgagaccggacttgatcttttttgattttgctcattggattccagaaattgccaaagagcatatgatcaagagtgtgagttacatgatagtatctgcaacaacaatagcttatacatttgcccctggtggtgtattaggtgttcccccaccaggttatccttcatcaaaggtgttgtaccgtgaaaacgatgctcatgccttagcaaccttatctatcttctacaagagactttatcatcagatcactacaggttttaagagctgtgacatcattgcattgaggacatgtaatgaaatcgaaggtaaattctgcgactatatatcaagtcaataccataagaaggttctcttgactggtccaatgctccctgagcaagacacaagtaaaccactagaagaacagttgagtcattttctgagcaggttcccaccgaggtcagtggtgttttgtgcacttggtagccagatcgttcttgaaaaggatcaattccaagaactctgcttagggatggagctgacaggtttaccgtttcttatagcggtaaagccaccgagaggatcatcgacggtcgaagaagggttaccagaagggttccaggagcgggtgaaagggcgtggtgtggtttggggaggatgggtgcaacaaccattgatattggatcatccgtcaataggctgctttgtgaaccattgtggtccgggaacaatatgggagtgtcttatgactgattgtcaaatggttttgcttccatttttaggtgatcaagttctcttcacaagattgatgaccgaggaattcaaggtgtctgtagaagtgtcgagagaaaaaacaggatggttttcaaaggagagcttgagcgatgcgatcaagtctgtgatggataaagatagcgacctcggaaagctagtgaggagtaaccacgccaaattgaaggagactcttggtagtcatggattattaactggttacgtggataaatttgtagaggaattgcaagagtatttgatttga

SEQ ID NO: 78 - DNA, 1344 bp, Arabidopsis thaliana
atgggccaaaattttcacgcttttatgttcccatggttcgcttttggtcatatgactccatacttgcatctagccaacaagctagctgctaaaggtcatagggttactttcttgctgcctaagaaagctcaaaaacagttggaacatcacaatctgtttccagacaggatcatctttcattctcttactattccccatgttgatggcctacctgctggcgcggagaccgcctcggacatccccatctcgttggggaagtttcttaccgcagccatggatctcactcgcgatcaggtcgaagccgcggttcgtgctttgagaccagacctgatctttttcgatactgcttattgggttccggaaatggcgaaagaacacagagtcaagagtgtgatatactttgtgatatcagctaactccatagctcatgaacttgtaccaggtggtgaattaggagttcctccacctggctatccttcgtcaaaagtgttgtaccgtggacacgatgctcacgctttgttgactttttccatcttctacgagaggcttcattaccggataacaacaggtctaaagaattgtgattttatctcaattaggacttgtaaagaaatcgaaggtaaattctgcgactatatagagcgtcaataccagaggaaggttcttttgacaggtccaatgcttccagagccagataacagtagaccactcgaagatcgatggaatcactggctgaatcagttcaaacccggctcggtaatatattgtgcattgggaagtcaaatcactctagagaaggatcaattccaagaactctgtttaggaatggagctcactggtttaccgtttctcgtagcggtaaaaccaccaaaaggcgcaaagacgatccaagaagcgttgccagaagggtttgaggagagggtgaagaatcatggagtagtttggggagaatgggtgcagcaaccattgatattggctcatccatcagtaggctgctttgtgacccattgtgggtttggatcaatgtgggagtctctagtgagtgattgtcaaatagtcttgcttccatatttgtgtgatcaaattctcaacactagattgatgagtgaggaactcgaggtttcggtggaagtgaaaagagaagaaacaggatggttctcgaaagagagcttaagtgttgcgatcacctcggtgatggacaaagatagtgagttagggaatctggtgaggaggaaccacgctaaattaaaggaggttttggttagtcctggattattaaccggttacaccgatgaatttgttgaaactttgcagaatatagtcaacgatacaaatcttgaatga

SEQ ID NO: 79 - DNA, 1386 bp, Arabidopsis thaliana
atgaaagtaacacaaaagccaaagataatattcatcccttatccggcgcaaggccacgtcactccgatgcttcaccttgcatcggctttcctcagccgtggattctcccctgtcgttatgactcccgagtctatccaccgtaggatctcggctactaacgaggatcttgggatcacgttcttggccttatctgacggtcaagatcgtccggacgcacctccctcggacttcttctcgatagagaactcaatggagaacatcatgccaccacagctcgaacggctcctactagaagaagacttggatgtggcttgtgttgtggttgatttgctggcttcgtgggctataggagtggctgatcggtgtggagttccggtcgccggattctggccggtgatgttcgctgcttaccgtttgatccaagcaataccggagctagtccgaacaggcttagtttcccaaaaaggttgtcctcgtcaactagaaaaaacaatagtccagccagagcaaccgctcctatccgcagaagatctaccgtggctgatcggaactcccaaagctcagaaaaaacgattcaagttctggcaaagaactctagaacgaacaaaaagtctccgttggatcttgacaagctcctttaaagatgaatatgaagatgtcgacaaccacaaagcatcctacaaaaaatctaacgatttaaacaaagaaaacaatggtcaaaaccctcaaatccttcatttaggtccattgcataaccaagaagcaacaaataatataactataaccaagactagtttttgggaagaagacatgtcttgtctaggttggcttcaagaacaaaacccgaactcagtcatttatatctcatttggaagttgggtttctcctataggagaatcaaatattcaaacgttggcattggcgttggaagcgtcagggagacctttcctttgggcgttaaaccgagtgtggcaagagggactaccaccaggttttgtgcatagagtcacaattaccaaaaaccaaggaaggatcgtctcatgggctccgcaacttgaagttcttagaaacgattctgtgggatgttacgtgactcattgtggctggaactcgactatggaggcagtggcaagttcccggaggctactatgttatccggtggccggagaccagtttgttaactgtaaatacatcgtggacgtttggaagattggagtgagattgagcgggtttggagagaaggaggttgaagatggactaaggaaagtaatggaggatcaagatatgggtgagagattgaggaagttaagagacagagcaatggggaatgaagctcgtttgagttcggaaatgaattttacatttttaaaaaacgagcttaattag

SEQ ID NO: 80 - DNA, 1395 bp, Arabidopsis thaliana
atggataataactcaaataaaagaatgggaaggccacatgttgtggtcataccttaccctgcacaaggtcatgttcttcctctaataagtttctcacgttaccttgcgaaacaaggaatccaaattacattcataaacaccgagtttaaccataaccgcatcatcagttccttacccaattcacctcatgaagattatgttggggatcagatcaatcttgtttcaatccctgacggtttagaagattcaccagaagagaggaacattccagggaagttgtcggagtctgttttgcgttttatgcctaaaaaagtagaggaattgatcgagaggatgatggcagaaactagcggtggtacgatcattagctgcgttgtagcggatcagagcttgggatgggcaattgaagttgcagctaagtttgggatcagacgcaccgcgttttgtcctgctgcagctgcgtctatggttcttggatttagtattcaaaaacttatcgatgatggtctcatagattctgatgggactgtgagagtaaataagacaattcaactatctcccgggatgccaaagatggaaacagacaagtttgtgtgggtttgtctgaagaacaaagaatctcagaaaaacatattccaacttatgcttcaaaacaataactcgatcgagtcaacggattggttgttgtgtaactctgtccatgaacttgaaactgcagcatttggattgggcccgaatatagtaccaattgggcccattggttgggctcatagtcttgaagagggatccacgtcactaggaagctttttacctcatgaccgggattgtctagattggttggaccggcagattcccggttcggttatatatgttgcctttgggagttttggggtcatgggcaaccctcagttagaagagctagcaattggtctagagcttaccaagaggccagttttgtgggtcactggtgatcaacaaccaatcaaacttgggtcggatcgagtcaaagtggtgagatgggctccacaacgggaggtcctttcttctggagccattgggtgttttgtgagccattgtggatggaattcaactctggaaggagcccaaaatggcataccatttctatgcatcccttattttgcagaccaatttatcaacaaagcatatatatgcgatgtgtggaagattggattaggacttgaaagagacgcacgaggagtggttccgaggttagaggttaagaagaagatcgatgagatcatgagagacggtggagagtatgaagaacgagctatgaaggttaaagagattgtgatgaaaagtgttgcaaaagatggaatatcttgtgagaatcttaataaatttgtcaactggatcaaatcacaagtgaattga

SEQ ID NO: 81 - DNA, 1455 bp, Arabidopsis thaliana
atggtgttcgaaacttgtccatctccaaacccaattcatgtaatgctcgtctcgtttcaaggacaaggccacgtcaaccctcttcttcgtctcggcaagttaattgcttcaaagggtttactcgttaccttcgttacaacggagctttggggcaagaaaatgagacaagccaacaaaatcgttgacggtgaacttaaaccggttggttccggttcaatccggtttgagttctttgatgaagaatgggcagaggatgatgaccggagagctgatttctctttgtacattgctcacctagagagcgttgggatacgagaagtgtctaagcttgtgagaagatacgaggaagcgaacgagcctgtctcgtgtcttatcaataacccgtttatcccatgggtctgccacgtggcggaagagttcaacattccttgtgcggttctctgggttcagtcttgtgcttgtttctctgcttattaccattaccaagatggctctgtttcattccctacggaaacagagcctgagctcgatgtgaagcttccttgtgttcctgtcttgaagaacgacgagattcctagctttctccatccttcttctaggttcacgggttttcgacaagcgattcttgggcaattcaagaatctgagcaagtccttctgtgttctaatcgattcttttgactcattggaacaagaagttatcgattacatgtcaagtctttgtccggttaaaaccgttggaccgcttttcaaagttgctaggacagttacttctgacgtaagcggtgacatttgcaaatcaacagataaatgcctcgagtggttagactcgaggcctaaatcgtcagttgtctacatttcgttcgggacagttgcatatttgaagcaagaacagatcgaagagatcgctcacggagttttgaagtcgggtttatcgttcttgtgggtgattagacctccaccacacgatctgaaggtcgagacacatgtcttgcctcaagaacttaaagagagtagtgctaaaggtaaagggatgattgtggattggtgcccacaagagcaagtcttgtctcatccttcagtggcatgcttcgtgactcattgtggatggaactcgacaatggaatctttgtcttcaggtgttccggtggtttgttgtccgcaatggggagatcaagtgactgatgcagtgtatttgatcgatgttttcaagaccggggttagactaggccgtggagcgaccgaggagagggtagtgccaagggaggaagtggcggagaagcttttggaagcgacagttggggagaaggcagaggagttgagaaagaacgctttgaaatggaaggcggaggcggaagcagcggtggctccaggaggttcgtcggataagaattttagggagtttgtggagaagttaggtgcgggagtaacgaagactaaagataatggatactag

SEQ ID NO: 82 - DNA, 1491 bp, Arabidopsis thaliana
atggagctagaatcttctcctcctctacctcctcatgtgatgctcgtatcttttccagggcaaggccacgttaatccacttcttcgtcttggtaagctcttagcttcaaagggtttgctcataaccttcgtcaccactgagtcatggggcaaaaagatgcgaatctccaacaaaatccaagaccgtgtcctcaaaccggttggtaaaggctatctccggtatgatttcttcgacgacgggcttcctgaagacgacgaagctagcagaaccaacttaaccatcctccgaccacatctagagctggtcggcaaaagagagatcaagaaccttgtgaaacgttacaaggaagtaacgaaacagcccgtgacatgtcttatcaacaaccctttcgtctcttgggtctgtgacgtggcagaagatcttcaaatcccttgtgctgttctttgggttcaatcttgtgcctgcttagctgcttattactattaccaccacaacctagttgacttcccgaccaaaacagaacccgagatcgatgtccaaatctctggcatgcctctcttgaaacatgacgagatcccttctttcattcacccttcaagtcctcactccgctttgcgagaagtgatcatagatcagattaaacggcttcacaagactttctccattttcatcgacactttcaactcattggagaaagacatcattgaccacatgtcgacgctctctctccccggtgttatcagaccgctaggaccactctacaaaatggctaaaaccgtagcttatgatgtcgttaaagtaaacatctctgagccaacggatccttgcatggagtggttagactcgcagccagtttcctccgttgtttacatctcattcgggaccgttgcttacttgaaacaagaacaaatagacgagatcgcttacggtgtgttaaacgccgacgttacgttcttgtgggtgattagacaacaagagttaggtttcaacaaagagaaacatgttttgccggaagaagttaaagggaaagggaagatcgttgaatggtgttcacaagagaaagtattatctcatccttcagtggcatgtttcgtgactcactgtggatggaactcaacgatggaagctgtgtcttccggagtcccgacggtttgttttcctcaatggggagatcaagtcacggacgccgtttacatgatcgatgtttggaagacgggagtgaggctaagccgtggagaggcggaggagaggttagtgccgagggaggaagttgcggagaggttgagagaggttactaaaggagagaaagcgatcgagttgaaaaagaatgctttgaagtggaaggaagaggcggaggcggcggttgctcgcggtggttcgtcggataggaatcttgaaaagtttgtggagaagttgggtgccaaacctgtggggaaagtacaaaacgggagtcataatcatgtcttggctggatcaatcaaaagcttttaa

SEQ ID NO: 83 - DNA, 1440 bp, Arabidopsis thaliana
atggacccgtctcgtcatactcatgtgatgctcgtatctttccccggccaaggtcacgtaaaccctctacttcgtctcggaaagctcatagcctctaaaggcttactcgtcacctttgtcaccacagagaagccatggggcaagaagatgcgtcaagccaacaagattcaagacggtgtgctcaaaccggtcggtctaggtttcatccggtttgagttcttctctgacggcttcgccgacgacgatgaaaaaagattcgacttcgatgccttccgaccacaccttgaagctgtcggaaaacaagagatcaagaatctcgttaagagatataacaaggagccggtgacgtgtctcataaacaacgcttttgtcccatgggtatgtgatgtcgccgaggagcttcacatcccttcggctgttctatgggtccagtcttgtgcttgtctcacggcttattactattaccaccaccggttagttaagttcccgaccaaaaccgagccggacatcagcgttgaaatcccttgcttgccattgttaaagcatgacgagatcccaagctttcttcacccttcgtctccgtatacagcttttggagatatcattttagaccagttaaagagattcgaaaaccacaagtctttctatcttttcatcgacacttttcgcgaactagaaaaagacatcatggaccacatgtcacaactttgtcctcaagccatcatcagtcctgtcggtccgctcttcaagatggctcaaaccttgagttctgacgttaagggagatatatccgagccagcgagtgactgcatggaatggcttgactcaagagaaccatcctcagtcgtttacatctcctttgggactatagccaacttgaagcaagagcagatggaggagatcgctcatggcgttttgagctctggcttgtcggtcttatgggtggttcggcctcccatggaagggacatttgtagaaccacatgttttgcctcgagagctcgaagaaaagggtaaaatcgtggaatggtgtccccaagagagagtcttggctcatcctgcgattgcttgtttcttaagtcactgcggatggaactcgacaatggaggctttaactgccggagtccccgttgtttgttttccgcaatggggagatcaagtgactgatgcggtgtacttggctgatgttttcaagacaggagtgagactaggccgcggagccgctgaggagatgattgtttcgagggaggttgtagcagagaagctgcttgaggccacagttggggaaaaggcggtggagctgagagaaaacgctcggaggtggaaggcggaggccgaggccgccgtggcggacggtggatcatctgatatgaactttaaagagtttgtggacaagttggttacgaaacatgtgacgagagaagacaacggagaacactag

SEQ ID NO: 84 - DNA, 1428 bp, Arabidopsis thaliana
atggagatggaatcgtcgttacctcatgtgatgctcgtatcattcccagggcaaggtcacataagccctcttcttcgtctcggaaagatcattgcctctaaaggcttaatcgtcacctttgtaaccacagaggaaccattgggcaagaagatgcgtcaagccaacaatattcaagacggtgtgctcaaaccggtcgggctaggttttctccggttcgagttcttcgaggatggatttgtctacaaagaagactttgatttgttacaaaaatcacttgaagtttccggaaaacgagagatcaagaatcttgtcaagaaatatgagaagcaaccagtgagatgtctcataaataatgcctttgttccatgggtttgtgacatagccgaggagcttcaaatcccatcagctgttctttgggtccagtcttgtgcttgcctcgccgcttattactattaccaccaccagttagttaagtttccgaccgaaaccgagccggaaataaccgttgacgtccctttcaagccattaacattgaagcatgacgagatccctagctttcttcacccttcctctccgctgtcctctataggaggtaccattttagagcagatcaagcgacttcacaagcctttctctgttctcatcgaaacttttcaagaacttgaaaaagataccattgaccacatgtcccagctctgccctcaagtcaacttcaaccccatcggtccgctttttactatggctaaaaccataaggtctgacatcaagggagacatctccaagccagatagtgactgcatagagtggcttgactcgagagaaccatcctccgttgtttacatctcttttgggactttggctttcttgaagcaaaaccagatcgacgagattgctcacggcattctcaactccgggttgtcctgcttatgggttttgcggcctcccttagaaggcttagccatagaaccgcatgtcttgcctctagagcttgaagagaaagggaagattgtggaatggtgtcaacaagagaaagttttggctcatcctgcggttgcttgcttcttaagtcactgtggatggaactcaaccatggaggctttaacttcaggagttcccgttatttgtttcccgcagtggggagatcaggtgacaaatgcggtgtacatgattgatgttttcaagacaggattgagactcagccgtggagcttccgatgagaggattgttccaagggaggaggttgctgagcgactgcttgaggccaccgttggagagaaggcggtggagctgagagaaaacgctcggaggtggaaggaggaggcggagtctgccgtggcttacggtggaacatcggaaaggaattttcaagagtttgttgacaagttggttgatgtcaagacaatgacaaacattaataatgtcgtgtaa

SEQ ID NO: 85 - DNA, 1371 bp, Arabidopsis thaliana
atgggcagtagtgagggtcaagaaacacatgtcctaatggtaacactaccattccaaggtcacatcaatccaatgctcaaactcgcaaaacatctctcgttatcatcaaagaacctacacatcaatctcgccactattgagtcagcccgtgatctcctctccaccgtagaaaaacctcgttatccggtggacctcgtgttcttctccgatggtctacctaaagaagatccaaaggcccctgaaactcttttgaagtcattgaataaagtcggagccatgaacttgtctaaaatcatcgaagaaaagagatactcttgtatcatctcttcgccttttactccatgggttccagctgttgcagcctctcataacatctcttgtgcaatactttggatccaagcttgtggagcttactcggtttattaccgttactacatgaagacaaactctttccctgatcttgaagatctgaatcaaacggtggagttaccagctttaccattgttggaagttcgagatcttccatcgtttatgttaccttctggtggtgctcacttctataatctaatggcggaatttgcagattgtttgaggtatgtgaaatgggttttggttaattcattctatgaactcgaatcagagataatcgaatcgatggctgatttaaaacctgtaattccaattggtcctctggtttctccatttctgttgggcgatggtgaggaggaaaccctagacggtaaaaacctagatttttgtaaatctgatgattgttgtatggagtggcttgacaagcaagctaggtcttctgttgtgtacatatctttcggaagtatgctcgaaacattggagaatcaggtcgagaccatagcgaaggcgctgaagaacagaggacttccatttctttgggtgataaggccaaaggagaaagcccaaaacgttgctgttttgcaggagatggtgaaagaaggacaaggggttgttctcgagtggagtccacaagagaagattttgagccacgaggcaatctcttgttttgtcacgcattgcggctggaactcgactatggagacggtggtggctggtgttcctgtggtagcgtaccctagctggacggatcagcccattgacgcgcggttgcttgttgatgtgtttggaatcggagtaaggatgaggaatgacagtgtcgatggcgagcttaaggtcgaagaagtagaaagatgcattgaggccgtgacggagggacccgctgccgtggatataagaaggagagcggcggagctaaagcgcgtggcgagattggcgttggcacctggtggatcttcgacacggaatttagacttgttcattagtgatatcacaatcgcctaa

SEQ ID NO: 86 - DNA, 1353 bp, Arabidopsis thaliana
atgggaagtaatgagggtcaagaaacacatgtcctaatggtagcattagcattccaaggtcatctcaatccaatgctcaaattcgcaaaacatctcgcacgaaccaatctacacttcactctcgccaccactgagcaagcccgtgacctcctctcttccaccgctgacgaacctcatagaccggtggacctcgctttcttctcagacggtctacctaaagacgatccaagagatcccgacactctcgcaaagtcattgaaaaaagatggagccaagaacttgtcaaaaatcatcgaagaaaagagatttgattgcatcatctctgtgccttttactccctgggttccagctgttgcagctgcacataacattccttgtgcaatcctctggatccaagcttgtggagctttttctgtttattaccgttattacatgaagacaaatcctttccccgaccttgaagatctgaatcaaacagtggagttaccagctttaccattgttggaagtccgagatctcccgtcattgatgttaccttctcaaggagctaatgtcaataccctaatggcggaatttgcagattgtttgaaagatgtgaaatgggttttggttaactcgttttacgaactcgaatcagagatcatcgagtctatgtctgatttaaaacctataatcccaattggtcctcttgtttctccattcctgttgggaaatgatgaagaaaaaaccctagatatgtggaaagttgatgattattgtatggagtggcttgacaagcaagctaggtcttcagttgtttacatatctttcggaagcatactcaaatcattggagaatcaagttgagaccatagcaacggcattaaaaaacagaggagttccatttctttgggtgatacggccgaaggagaaaggcgaaaacgtccaggttttgcaggagatggttaaagaaggtaaaggggttgtaactgaatggggtcaacaagaaaagatattgagccacatggcgatttcttgcttcatcacgcattgtggatggaactcgacgatcgagacggtggtgactggtgttcccgtggtggcgtatccgacttggatagatcagccgcttgatgcgagactgcttgtggatgtgtttggaatcggagtaaggatgaagaacgacgctatcgatggagagcttaaggttgcagaggtggagagatgcattgaggccgtgacagagggacctgccgccgcggatatgaggaggagagcgacggagctgaagcacgccgcaagatcggcgatgtcacctggtggatcttccgctcagaatttagactcgttcattagtgatatcccaatcacttga

SEQ ID NO: 87 - DNA, 1470 bp, Arabidopsis thaliana
atgggatctcagatcattcataactcacaaaaaccacatgtagtttgtgttccatatccggctcaaggccacatcaaccctatgatgagagtggctaaactcctccacgccagaggcttctacgtcaccttcgtcaacaccgtctacaaccacaatcgtttccttcgttctcgtgggtccaatgccctagatggacttccttcgttccgatttgagtccattgctgacggtctaccagagacagacatggatgccacgcaggacatcacagctctttgcgagtccaccatgaagaactgtctcgctccgttcagagagcttctccagcggatcaacgctggagataatgttcctccggtaagctgtattgtatctgacggttgtatgagctttactcttgatgttgcggaggagcttggagtcccggaggttcttttttggacaaccagtggctgtgcgttcctggcttatctacacttttatctcttcatcgagaagggcttatgtccgctaaaagatgagagttacttgacgaaggagtacttagaagacacggttatagattttataccaaccatgaagaatgtgaaactaaaggatattcctagcttcatacgtaccactaatcctgatgatgttatgattagtttcgccctccgcgagaccgagcgagccaaacgtgcttctgctatcattctaaacacatttgatgaccttgagcatgatgttgttcatgctatgcaatctatcttacctccggtttattcagttggaccgcttcatctcttagcaaaccgggagattgaagaaggtagtgagattggaatgatgagttcgaatttatggaaagaggagatggagtgtttggattggcttgatactaagactcaaaatagtgtcatttatatcaactttgggagcataacggttttgagtgtgaagcagcttgtggagtttgcttggggtttggcgggaagtgggaaagagtttttatgggtgatccggccagatttagtagcgggagaggaggctatggttccgccggactttttaatggagactaaagaccgcagtatgctagcgagttggtgtcctcaagagaaagtactttctcatcctgctattggagggtttttgacgcattgcgggtggaactcgatattggaaagtctttcgtgtggagttccgatggtgtgttggccattttttgctgaccagcaaatgaattgtaagttttgttgtgacgagtgggatgttgggattgagataggtggagatgtgaagagagaggaagttgaggcggtggttagagagctcatggatggagagaagggaaagaaaatgagagaaaaggcggtagagtggcagcgcttagccgagaaagcgacggaacataaacttggttcttccgttatgaattttgagacggttgttagcaagtttcttttgggacaaaaatcacaggattaa

SEQ ID NO: 88 - DNA, 1446 bp, Arabidopsis thaliana
atgggatctcatgtcgcacaaaaacaacacgtagtttgcgttccttatccggctcaaggccacatcaacccaatgatgaaagtggctaaactcctttacgccaaaggcttccatattaccttcgtcaacaccgtctacaaccacaaccgtctcctccggtcccgtgggcctaacgccgttgacgggcttccttctttccggtttgagtccatccctgacggtctacccgagactgacgtggacgtcactcaggacatccctactctttgcgagtccacaatgaagcactgtctcgctccattcaaggagcttctccggcagatcaacgcaagggatgatgttcctcctgtgagctgtatcgtatccgacggttgtatgagcttcacacttgatgctgcggaggagctcggtgtcccggaggttcttttttggacaactagtgcttgtggcttcttggcttacctttactactatcgcttcatcgagaagggattatcaccaataaaagatgagagttacttaaccaaggaacacttggacacaaaaatagactggataccatcgatgaagaacctaagactaaaagacatccctagcttcatccgaacgactaatcctgacgacatcatgctcaactttatcatccgtgaggctgaccgagccaaacgcgcttcagctatcattctcaacacgtttgatgatctcgaacacgacgttatccaatctatgaaatccattgtacctccggtttattctattggaccgttacatttactagagaaacaagagagcggcgagtatagtgaaatcggacggacaggatcgaatctttggagagaggagactgagtgtctggactggctaaacacgaaagctagaaacagtgttgtgtacgttaacttcgggagtataactgttttgagcgcaaaacagcttgtggagtttgcatggggtttggctgcaacggggaaagagtttttgtgggtgatccggccggatttagtagccggggatgaggcaatggttccaccggagtttttaacggctacggcggaccggaggatgttggcaagttggtgtcctcaagagaaagtcctttctcatccggccattggagggttcttgacgcattgcgggtggaactcgacgttggaaagtctatgcggtggagttccaatggtgtgttggccgttttttgcagagcaacaaactaattgtaagttttctcgtgacgaatgggaggttgggattgagattggtggagatgtgaagagagaagaggttgaggcggtggttagggagttgatggatgaagagaagggaaagaatatgagagagaaggcggaagagtggcggcgcttggcgaatgaagcgacggagcataagcatggttcttctaaattgaactttgagatgctcgttaataaggttcttttaggggagtag

SEQ ID NO: 89 - DNA, 1467 bp, Arabidopsis thaliana
atgggatcccgttttgtttctaacgaacaaaaaccacacgtagtttgcgtgccttacccagctcaaggccacattaaccctatgatgaaagtggctaaactcctccacgtcaaaggcttccacgtcaccttcgtcaacaccgtctacaaccacaaccgtctactccgatcccgtggggccaacgcactcgatggacttccttccttccagttcgagtcaatacctgacggtcttccggagactggcgtggacgccacgcaggacatccctgccctttccgagtccacaacgaaaaactgtctcgttccgttcaagaagcttctccagcggattgtcacgagagaggatgtccctccggtgagctgtattgtatcagatggttcgatgagctttactcttgacgtagcggaagagcttggtgttccggagattcatttttggaccactagtgcttgtggcttcatggcttatctacacttttatctcttcatcgagaagggtttatgtccagtaaaagatgcgagttgcttgacgaaggaatacttggacacagttatagattggataccgtcaatgaacaatgtaaaactaaaagacattcctagttttatacgtaccactaatcctaacgacataatgctcaacttcgttgtccgtgaggcatgtcgaaccaaacgtgcctctgctatcattctgaacacgtttgatgaccttgaacatgacataatccagtctatgcaatccattttaccaccggtttatccaatcggaccgcttcatctcttagtaaacagggagattgaagaagatagtgagattggaaggatgggatcaaatctatggaaagaggagactgagtgcttgggatggcttaatactaagtctcgaaatagcgttgtttatgttaactttgggagcataacaataatgaccacggcacagcttttggagtttgcttggggtttggcggcaacgggaaaggagtttctatgggtgatgcggccggattcagtagccggagaggaggcagtgattccaaaagagtttttagcggagacagctgatcgaagaatgctgacaagttggtgtcctcaggagaaagttctttctcatccggcggtcggagggttcttgacccattgcgggtggaattcgacgttagaaagtctttcatgcggagttccaatggtatgttggccattttttgctgagcaacaaacaaattgtaagttttcttgtgatgaatgggaggttggtattgagatcggtggagatgtcaagaggggagaggttgaggcggtggttagagagctcatggatggagagaaaggaaagaaaatgagagagaaggctgtagagtggcggcgcttggccgagaaagctacaaagcttccgtgtggttcgtcggtgataaattttgagacgattgtcaacaaggttctcttgggaaagatccctaacacgtaa

SEQ ID NO: 90 - DNA, 1470 bp, Arabidopsis thaliana
atggaacaacatggcggttctagctcacagaaacctcacgcaatgtgcataccttatccagcacaaggccacatcaacccaatgctgaaactagccaagctcctccacgctagaggcttccacgtcactttcgtcaacaccgactacaaccaccgccgtatcctccaatcacgtggccctcacgctctcaacggtctcccctcgtttcgcttcgagactatccccgacggtcttccttggacagacgtcgacgctaagcaagacatgctcaagcttattgactccacaataaacaactgtttagctccattcaaagacctcatcctccggttaaactccggttctgatataccaccggttagctgtatcatctccgacgcttcaatgagcttcacaattgacgcagcggaggagcttaaaattccggtagttctcctctggaccaacagtgctactgctttaatcttgtatctccattaccaaaaactcatcgagaaagagataattcccctcaaagattcgagtgacttgaagaagcatttagagacggagattgattggataccgtcgatgaagaagattaagcttaaggattttccagatttcgtcaccacgacgaatcctcaagatccgatgattagtttcatccttcatgtaaccggaagaatcaaaagagcttctgcgatcttcatcaacactttcgaaaaactcgagcataacgttctcttatctctgcgatctcttctccctcagatctactccgttggaccgttccagattctggagaatcgcgaaatcgataagaacagcgaaatcagaaagctaggattgaatctctgggaagaagagacggagtctttggattggctagatactaaagctgagaaagctgtgatttacgtcaacttcgggagtctaacggttttgactagtgagcagatcttagagttcgcttggggtttagcgaggagcgggaaagagtttctctgggtggtgagatctggtatggtcgacggagatgattcgattcttccggcggagtttttatcggagacgaagaatcgaggaatgttaattaaaggatggtgttctcaggagaaggtactttcgcatccggcgattggaggatttttgactcactgtggatggaattcgacgttggagagtttgtacgccggtgttccgatgatctgttggccattttttgctgatcagttgacgaatcgaaagttctgttgcgaggattgggggattgggatggagatcggcgaggaggtgaagagggagagagtggagacggtggttaaagagctcatggacggagagaagggaaagaggttaagagagaaggtggtggagtggcggcgcttggcggaagaagcttcggcgccaccgttgggatcatcgtacgtgaattttgaaacggtggttaataaagtccttacatgtcacacgattagatcgacctaa

SEQ ID NO: 91 - DNA, 1440 bp, Arabidopsis thaliana
atggcgtctcatgctgttacaagcggacaaaaaccacacgtagtttgcatacctttcccggctcaaggccacatcaatccgatgctcaaagtggctaaactcctctatgccagaggcttccatgttaccttcgtcaacactaactacaaccataaccgtctcatccggtcacgtggtcccaactcccttgatgggcttccttcttttcggttcgagtccatccctgacggtctaccggaggaaaacaaggacgtcatgcaggatgtccctaccctttgtgagtccaccatgaaaaactgtctagctcctttcaaggagcttctccggcggatcaacaccacaaaggatgttcctccggtaagctgtattgtatccgacggtgtgatgagctttactcttgatgctgcagaggagcttggagtcccggatgttcttttttggacaccaagtgcttgtggcttcttggcttatctacacttctatcgcttcatcgagaaggggttatcaccaataaaagatgaaagttctttggacacaaaaataaattggataccatcgatgaaaaacctaggacttaaagacatcccaagctttatccgtgcaactaatactgaagacataatgcttaacttttttgtccatgaggctgaccgagccaaacgcgcttccgctatcattctcaacacattcgatagtcttgagcatgatgtcgtccgttctattcaatctatcatacctcaagtgtacactattggaccgcttcatctatttgtgaatcgggatatcgacgaggaaagtgacatcggacagataggaacgaatatgtggagagaggagatggagtgtttggattggcttgatactaagtctccaaacagtgtcgtttatgttaatttcggtagcataacagtgatgagtgcgaaacaactcgtggagtttgcttggggtttagcagcgaccaaaaaagattttttgtgggtgattaggccggatttagtagccggtgatgtgccaatgcttccgccggactttctaatagagacggctaaccgaaggatgctagcgagttggtgtcctcaagaaaaagttctttctcatccggcagttggagggttcttaacgcatagtggatggaattcgactttggagagtctctccggtggagttccaatggtgtgttggccgttctttgcggaacagcaaacaaattgtaaatattgttgtgatgaatgggaagtggggatggagatcggtggagatgtgaggagggaggaggttgaggagttggttagagaactcatggacggagacaaaggaaagaaaatgaggcaaaaggccgaagagtggcagcgcttggctgaggaagcgacgaagcctatttatggttcgtcggaactaaattttcagatggtcgttgacaaggttcttttaggggagtag

SEQ ID NO: 92 - DNA, 1464 bp, Arabidopsis thaliana
atggaatctcatgttgttcataacgcacaaaagccacacgtagtttgcgtgccttacccggctcaaggccacatcaatccgatgctgaaagtggctaaactcctctacgctaaaggctttcacgtcaccttcgttaacactctctacaaccacaaccgtctcctccggtcccgtggtcccaacgcgctcgacgggtttccttcattccggttcgagtccatccctgacggtctaccggagactgatggcgataggacgcagcatactcctaccgtttgcatgtccattgagaaaaactgtctcgctccattcaaagagattctgcgccggatcaacgataaagatgatgttcctccagtgagttgtattgtatcggacggtgtgatgagttttactcttgacgcagccgaggaactaggtgtcccagaggttattttttggaccaatagtgcttgtggtttcatgactattctacacttttatcttttcatcgagaagggtctatctccttttaaagacgaaagttacatgtcaaaggagcatctagacacagttatagattggataccatcaatgaagaatcttaggttaaaggacatccctagctatatacgtaccacaaatcctgacaacataatgcttaatttcctcattcgagaagttgagcgatctaaacgcgctagtgctatcattctcaacacgtttgatgaactcgagcatgatgttatccagtctatgcaatctattttacctccggtttattctattgggccactccatctccttgtgaaggaagaaataaacgaggctagtgaaataggacagatgggattaaatttgtggagagaggagatggaatgtttggattggctcgatacaaaaactccaaacagtgttctttttgttaactttggatgcataacggtgatgagtgcaaaacagcttgaagaatttgcttggggtttggcggcaagtaggaaagagtttttatgggtgatccgtcctaatttagtggtgggagaggcgatggtggttcttccacaagagtttttagcggagacgatagaccggagaatgttagctagttggtgtcctcaggagaaagttctttctcatcccgcgataggagggttcttgacgcattgcgggtggaactcaacattggagagtctcgctggtggtgttccgatgatatgttggccatgtttttcggagcaaccgacgaattgtaagttttgttgtgatgagtggggagtgggtatagagattggtaaagatgtgaagagagaggaggtcgagacggtggttagagaacttatggatggagaaaaggggaaaaagctgagagaaaaggcggaagagtggcggcggttggccgaggaagcgacgaggtataaacatggttcgtcggtcatgaatcttgagacgcttatacataaagttttcttagaaaatcttagatga

SEQ ID NO: 93 - DNA, 1473 bp, Arabidopsis thaliana
atggagagagcaaagtcgaggaagcctcatatcatgatgataccatacccacttcaaggtcacgttatcccttttgtccacttagccatcaaacttgcttctcatggcttcaccatcactttcgtcaacaccgactccatccaccaccacatctccaccgctcaccaagatgacgccggtgacatcttctccgccgctcgcagctccggccagcacgacatacgttacaccaccgtgagcgacggcttccctttagactttgaccggtcactgaaccatgaccagtttttcgaaggcattctccacgtcttctctgcccacgtggatgatctcatcgccaaactctcccgccgtgatgatcctcccgtgacttgcttgatcgccgacacgttttatgtttggtcatctatgatttgcgacaagcacaaccttgtaaatgtctcgttttggaccgaacctgccttggtcctcaatctctattatcacatggatctcctcatatctaacggtcatttcaaatctcttgataatcgtaaagacgtgatcgattacgtaccaggggttaaagcaatagaaccaaaggacttgatgtcatatcttcaagtaagcgacaaagacgtagacacaaatacagtagtatacagaatattattcaaggcctttaaagacgtcaagagagccgacttcgtcgtatgcaacacggtgcaagagctcgaaccagactctctctcggctctacaagccaaacaaccggtttacgctatcggtccggttttctcaactgattcggtagttcccacaagcttatgggccgagtcagactgtaccgagtggcttaagggccggcccactgggtcagttctctacgtctcgtttggtagctatgcacatgttggtaagaaggagattgttgagatagctcatgggcttttgcttagtgggattagtttcatttgggttttacgtccggatatagttggatccaacgtaccagattttcttccagccgggtttgtggaccaagcccaagatcgaggtcttgtggtccaatggtgctgccagatggaagttatttcaaatccggccgtgggagggtttttcacacattgtggatggaattcaattctagagagcgtttggtgtggtttgcctttgttgtgttatccacttttgacagatcagttcacgaataggaagcttgtggtcgatgattggtgcattgggattaatctttgtgagaagaagacaatcacaagggaccaagtctcagcgaatgttaaaagattgatgaatggagaaacttcaagtgagctaagaaacaatgttgaaaaggttaaacgtcatctcaaagatgcggttacaaccgttggatcttcggagacgaattttaacttgtttgttagtgaggtccgaaatagaatagaaactaaattgtgtaatgtaaatggactagaaataagtccatcaaactaa

SEQ ID NO: 94 - DNA, 1449 bp, Arabidopsis thaliana
atggcggacgttagaaaccctacaaaaaatcatcatggtcatcatcatcttcatgctctcttgatcccatatccatttcaagggcatgtaaacccatttgtacacttagccatcaagctcgcgtcacaggggatcaccgtcactttcgtcaacactcattacatccaccaccagatcacaaacggctccgatggagatattttcgctggagttaggtcagagtctggccttgacataaggtacgcgacggtttccgatggtttaccggtcggatttgaccggtcgttgaaccatgacacgtaccaatcgtcgctgttgcacgtgttctatgcgcatgtggaagagcttgtggcgagtcttgttggaggagacggcggtgtgaatgtgatgatcgccgacacattctttgtttggccgtctgtggtggctaggaagtttggtttggtttgtgtctcgttttggaccgaagctgctttagtattttcactttattaccatatggatctgcttcggattcatggccattttggtgctcaagaaacccgcagcgatctaatcgactacattcccggagtcgccgcaattaacccaaaagacacggcgtcgtatcttcaagaaaccgacacgtcatcagtagttcatcaaatcatcttcaaagcattcgaagacgtgaaaaaagtcgattttgtactctgcaacacaattcagcaattcgaagacaaaacaatcaaagccctaaacacaaaaatcccattttacgcaatcggaccaatcataccattcaataaccaaaccggttcagtcacaacctcactctggtctgaatcagattgtacacaatggctcaacactaaaccaaaaagctccgtactttatatctcctttggtagttacgctcatgtcacaaagaaggatcttgttgagatagctcacgggattttgttgagtaaagttaatttcgtttgggtggtgagaccagacattgttagttcagacgaaaccaatccattaccagaagggtttgaaacagaagctggagatcgtgggattgtaataccatggtgttgtcaaatgacggttttgtcacatgagagtgttggtgggtttttgacacattgtggttggaactcgatattggagacgatttggtgtgaggttcctgtgttgtgttttccattgttgactgatcaggttacgaataggaagcttgtggttgatgattgggagattgggattaatctttgtgaagataagagtgattttggtagagatgaagttgggaggaatattaaccgtttgatgtgtggtgtttcgaaagagaagatcggacgggttaaaatgagtttggaaggtgcggtgagaaacagtggatcttcttcggagatgaatttaggtttgtttattgatggacttttgtctaaggttggtttatctaatgggaaagcttaa

SEQ ID NO: 95 - DNA, 1371 bp, Arabidopsis thaliana
atgaatccaatcaaacctcagccactcggagtccgccacgtggtggccatgccttggccaggaagaggccacatcaacccaatgttaaacctctgcaaaagcctcgtccggcgagacccaaacctcaccgtcacattcgtcgtcaccgaagaatggctcgggttcatcgggtccgacccgaaacctaaccggatccatttcgccactctccccaacatcattccctccgagctcgtccgagccaacgacttcatcgccttcatcgacgccgtcctcaccagattagaagagccgttcgaacagctacttgaccgtctaaactctcctcccaccgcaatcatcgccgatacttacatcatttgggcagtacgtgtaggcacaaaaaggaatattccggtggcttctttctggactacgtcagccacgattctctccctcttcattaactccgatcttctcgcaagtcacggccattttccgatcgaaccatcagaatcaaaactagacgagattgttgattacatccccggtttatctccgacaagactcagtgacttacagatcttacacggctatagtcatcaagtcttcaatatattcaaaaagtctttcggtgagctttataaagctaagtatcttctcttcccttctgcttatgagctcgaaccaaaagccattgactttttcacttccaagtttgatttcccggtttactccactggtccgttaatacccttggaagaactatccgttggaaatgagaatagagaacttgattactttaagtggcttgatgagcaacctgaaagctctgttctttacatatctcaagggagttttctttcagtctccgaagctcagatggaggagattgttgtaggagttagagaggctggagttaagttcttttgggtggctcgtgggggtgagttaaagcttaaggaggctcttgaaggtagcttgggtgttgtggtgagctggtgtgatcagctacgtgttttgtgtcatgcggctataggcgggttttggacgcattgcgggtataactcgacattggaagggatatgttcgggagtaccgttgcttacatttcctgttttttgggatcagtttctgaatgctaagatgattgttgaggagtggagagttggaatggggatcgagaggaagaagcagatggagttgttgatagtgagtgatgagatcaaggaattggtaaaaaggtttatggatggagagagtgaagaagggaaagagatgagaagaaggacttgtgatctcagtgagatatgtcgtggagcggttgcgaaaggtggttcttctgatgctaacatcgatgctttcattaaagatattactaagatcgtgtga

SEQ ID NO: 96 - DNA, 1368 bp, Arabidopsis thaliana
atggatccaaatgaatctccaccaaaccaatttcgccacgtggtggccatgccttatccaggtcgaggacacatcaaccctatgatgaacctctgcaaacgccttgtccgtcgataccctaaccttcacgtcaccttcgtcgtcacagaagaatggctcgggtttattggacccgacccgaaacccgaccggatccatttctccactctccctaatctcatcccttccgagcttgtcagggccaaagacttcataggcttcattgatgccgtctacacaagattggaagaaccattcgagaagcttcttgacagcctcaattcaccacctccgagtgtaatattcgccgacacttacgtcatttgggctgtgcgagtcggcagaaaaaggaatattccggtggtttctctctggaccatgtcagccacgattctctccttcttcctccactctgatctactcataagtcatggccatgctctgttcgaaccatcagaagaagaggttgttgattacgtccccggtttatctccgacgaaactccgagatttgccgccgatatttgacggttacagcgaccgagtcttcaagacagctaagttgtgtttcgatgaactaccaggagctaggtctttactcttcaccaccgcctatgagcttgaacacaaagctattgacgctttcacctccaagctcgatatcccggtctacgctattggtcctttaataccttttgaagaactttctgttcaaaatgataacaaggaacctaattacatccagtggcttgaggaacaaccggaaggctctgttctttacatatctcagggaagttttctttcggtctcggaagctcagatggaggaaatagtgaaaggactgagagaaagtggagtccggtttctttgggtggctcgtgggggcgagttaaagcttaaggaggctcttgaaggtagcttaggtgtagtggtgagctggtgtgatcagcttcgggtgctgtgtcacaaagctgtaggcgggttttggactcattgcgggtttaactcgacattggaagggatatattcaggagtaccaatgctagcgtttccgttgttttgggatcagattctgaacgctaagatgattgttgaggactggagagtcggaatgaggatcgagaggacgaaaaagaatgagttgttgatagggagagaggagatcaaggaagtagtgaagaggtttatggatagagagagtgaagaagggaaagagatgagaagaagggcttgtgaccttagtgaaatcagtcgaggagctgttgcgaaaagcggttcgtctaatgtaaacatcgatgagttcgttcggcatattaccaatacaaattaa

SEQ ID NO: 97 - DNA, 1389 bp, Arabidopsis thaliana
atgggtgaagaagctatagttctgtatcctgcaccaccaataggtcacttagtgtccatggttgagttaggtaaaaccatcctctccaaaaacccatctctctccatccacattatcttagttccaccgccttatcagccggaatcaaccgccacttacatctcctccgtctcctcctccttcccttcaataaccttccaccatcttcccgccgtcacaccgtactcctcctcctccacctctcgccaccaccacgaatctctcctcctagagatcctctgttttagcaacccaagtgtccaccgaactcttttctcactctctcggaatttcaatgtccgagcaatgatcatcgatttcttctgcaccgccgttttagacatcaccgctgacttcacgttcccggtttacttcttctacacctctggagccgcatgtctcgccttttccttctatctcccgaccatcgacgaaacaacccccggaaaaaacctcaaagacattcctacagttcatatccccggcgttcctccgatgaagggctccgatatgcctaaggcggtgctcgaacgagacgatgaggtctacgatgtttttataatgttcggtaaacagctctcgaagtcgtcagggattattatcaatacgtttgatgctttagaaaacagagccatcaaggccataacagaggagctctgttttcgcaatatttatccaattggaccgctcattgtaaacggaagaatcgaagatagaaacgacaacaaggcagtttcttgtctcaattggctggattcgcagccggaaaagagtgttgtgtttctctgttttggaagcttaggtttgttctcaaaagaacaggtgatagagattgctgttggtttagagaaaagtgggcagagattcttgtgggtggtccgtaatccacccgagttagaaaagacagaactggatttgaaatcactcttaccagaaggattcttaagccgaaccgaagacaaagggatggtcgtgaaatcatgggctccgcaagttccggttctgaatcataaagcagtcgggggattcgtcactcattgcggttggaattcaattcttgaagctgtttgtgctggtgtgccgatggtggcttggccgttgtacgctgagcagaggtttaatagagtgatgattgtggatgagatcaagattgcgatttcgatgaatgaatcagagacgggtttcgtgagctctacagaggtggagaaacgagtccaagagataattggggagtgtccggttagggagcgaaccatggctatgaagaacgcagccgaattagccttgacagaaactggttcgtctcataccgcattaactactttactccagtcgtggagcccaaagtga

SEQ ID NO: 98 - DNA, 1398 bp, Arabidopsis thaliana
atgacggaagtgttattgttgccgggaactaaatcggagaattcaaaaccaccgcacatagtggtgtttccattcccagcacaaggccacttacttcctctacttgacttaactcaccaactctgcctccgtggattcaacgtctccgtcatcgttactcccggtaaccttacttacctctctcctcttctctccgctcatccctcctccgtcacctccgtcgttttccctttccctcctcatccttcactctctcccggcgtcgaaaacgttaaagacgtcggaaattcaggaaatctcccgatcatggcttctcttcgtcagctacgagaaccaatcatcaactggttccaatctcatccgaatccgcctatcgctctcatctccgatttcttcctcggatggactcacgatctctgcaatcaaatcggtatccccagattcgctttcttctccatcagcttcttcttagtttccgttcttcaattttgcttcgagaacatcgatctaatcaaatcaacggatccgattcatctccttgatcttcctcgcgctccgattttcaaagaagagcatcttccgtctatagtccgacgaagtctccaaactccgtcaccggatctcgaatcaatcaaagatttctccatgaatttgttgagctacggatctgttttcaattcttctgagattctggaagatgattatcttcagtacgtgaaacagaggatgggtcatgatcgggtttatgttattggcccgctttgttcaatcgggtcgggtcttaaatcgaattcgggttctgtagacccgagtttgctgagttggttagacggatccccaaacgggtcagttctatacgtttgtttcggaagtcaaaaggcgttgactaaagaccagtgtgatgctttggctctaggcttagagaaaagcatgacccggtttgtttgggtggttaagaaagatccgatacccgacgggtttgaggatcgggtttccggaaggggattggtggtaagaggatgggtctcccagctggcggtgttgcgacacgtggcggttggtggatttttgagccattgtggatggaactcagtgcttgaagggataacgagtggggctgtgatcttgggctggcccatggaggcggaccagtttgtgaacgcgaggttgcttgtggagcatttgggtgttgcggttagggtttgcgaaggtggtgaaactgtgcctgactcggatgagttgggtcgggtcatagcggaaacgatgggtgagggaggacgcgaggtggctgctcgggctgaggagatacggcggaagaccgaggctgccgtgacggaggcaaatggaagctccgttgaaaatgtacaaagacttgtcaaagaatttgaaaaagtctaa

SEQ ID NO: 99 - DNA, 1422 bp, Arabidopsis thaliana
atgaaagtgaacgaggaaaacaacaagccgacaaagacccatgtcttaatcttcccatttccggcgcaaggtcacatgattcccctcctcgacttcacccaccgccttgctctccgcggcggcgccgccttaaaaataaccgtcctagtcactccaaaaaaccttccttttctctctccgcttctctccgccgtagttaacatcgaaccacttatcctcccttttccctcccacccttcaatcccctccggcgtcgaaaacgtccaagacttacctccttcaggcttccctttaatgatccacgcgcttggtaatctccacgcgccgcttatctcttggattacttctcacccttctcctccagtagccatcgtatctgatttcttccttggttggaccaaaaacctcggaatccctcgtttcgatttctctccctccgctgctatcacttgctgcatactcaatactctctggatcgaaatgcccaccaagatcaacgaagatgacgataacgagatcctccactttcccaagatcccgaattgtccaaaataccgttttgatcagatctcctctctttacagaagttacgttcacggagatccagcttgggagttcataagagactcctttagagataacgtggcgagttggggactcgtcgtgaactcgttcaccgccatggaaggtgtttatctcgaacatcttaagcgagagatgggccatgatcgtgtatgggctgtaggcccaattattccgttatctggggataaccgtggtggcccgacttctgtttctgttgatcacgtgatgtcgtggcttgacgcacgtgaggataaccacgtggtgtacgtgtgctttggaagtcaagtagttttgactaaagagcagactcttgcactcgcctctgggcttgagaaaagcggcgtccatttcatatgggccgtaaaggagcccgttgagaaagactcaacacgtggcaacatcctggacggtttcgacgatcgcgtggctgggagaggtctggtgatcagaggatgggctccacaagtagctgtgctacgtcaccgagccgttggcgcgtttttaacgcactgtggttggaactctgtggtggaggcggttgtcgccggcgttttgatgctgacgtggccgatgagagctgaccagtacactgacgcgtctctggtggttgatgagttgaaagtaggtgtgcgtgcttgcgaaggacctgacacggtgcctgacccggacgagttagctcgagttttcgctgattccgtgaccggaaatcaaacggagaggatcaaagccgtggagctgaggaaagcagcgttggatgcgattcaagaacgtgggagctcagtgaatgatttagatggatttatccaacatgtcgttagtttaggactaaacaaatga

SEQ ID NO: 100 - DNA, 1308 bp, Arabidopsis thaliana
atgacaacaacaacaacgaagaagccgcacgttctggtgataccgtttccacaatccggtcacatggttccacatcttgacctcacgcatcagattcttctccgtggagccaccgtcactgtcctcgtcacacccaaaaactcttcctatctcgatgctctccgttctcttcactccccggaacacttcaaaaccctaatccttccttttccttctcacccttgtataccttccggtgtcgaatctctccagcaacttcctctcgaagctatagttcacatgtttgatgctctctctcgtctccacgaccctctcgttgactttctcagccgtcaaccaccgtcggatctccccgacgccatcctaggaagctcatttctcagcccttggattaacaaagtagctgatgctttctctattaagtccattagtttcttacccatcaatgctcattcgatctccgtcatgtgggctcaagaagatagaagcttcttcaacgatctcgagactgccacaacggaaagctacgggctcgtcatcaacagtttctacgacctcgagcctgagtttgtagaaactgttaaaacacgtttcctgaatcaccaccgtatatggaccgtcggaccgttgctcccctttaaagctggcgttgaccgtggcggacaaagctcaatcccgccggcgaaagtctcggcttggttagattcgtgccccgaggataactccgtcgtatacgtcggttttggaagccagatccggctcacggcggagcaaacagctgctttagcggcggcgttggagaaaagcagtgtgcgtttcatatgggcggtgagagacgcagctaagaaggtgaactccagcgataactccgttgaggaagatgtgatcccggcgggatttgaagagagagtgaaggagaaaggactcgtgataagaggatgggccccacaaactatgattcttgagcatcgagccgttggatcttacctaactcatttgggttggggttcggttctggaaggaatggtcggaggagttatgttgctagcgtggccgatgcaagcagaccatttctttaacacgacgctcatcgttgataaactaagagccgcagtgcgagttggagagaacagagactcggttcctgactcggacaagctcgctaggattttggctgagtcggcgagagaggacttgccggagagagttacgttgatgaagctgagggagaaagctatggaggccattaaagaaggtgggagctcttacaagaacttggatgagctcgttgcagagatgtgtttgtaa

SEQ ID NO: 101 - DNA, 1437 bp, Arabidopsis thaliana
atgtccgtttcaacacatcaccaccacgtggtcctcttccctttcatgtcaaaaggccacatcatccctctcctccaattcggtcgtctcctcctccgtcaccaccgcaaagaaccaaccatcaccgtcaccgttttcaccactcccaagaaccaacctttcatctcagacttcctctcggatacgccggagatcaaagtcatctctctccctttcccggaaaacatcaccggaatccctcccggcgtcgagaacaccgaaaagctcccatccatgtcacttttcgtccccttcacacgcgccacgaagcttctccaacctttcttcgaagaaacactcaagactcttccaaaagtttcgttcatggtctctgatggattcctctggtggacatcggagtctgcagctaagttcaacattccaagatttgtctcctacggcatgaactcttactccgccgctgtctccatctctgttttcaaacacgaactctttaccgaaccggaaagtaaatctgataccgaaccggtcactgtaccagactttccatggatcaaggtcaagaagtgtgatttcgaccatggcactaccgagccggaagaatcaggtgcagccctcgaactatctatggaccaaatcaagtcgaccaccacaagccatgggtttttagtcaatagcttctacgagctcgagtcagcatttgttgattacaacaacaactctggtgataaaccaaagtcgtggtgtgttgggccactgtgtttgacagatcctcctaaacaggggagtgctaaaccggcttggattcattggttggatcagaagcgagaggaagggcgtccggttttgtacgtggcgtttggaacgcaggcagagatatcgaacaagcagcttatggaactagctttcggcttggaagattcaaaggtgaactttctgtgggtcacaagaaaagatgtggaggagattattggagaaggattcaacgatagaataagagagagtgggatgatagtgagagattgggtggaccaatgggagatattgtcacatgaaagtgtcaaaggatttttgagccattgtgggtggaactcagcacaagagagcatatgtgtcggggtcccattgttggcttggccgatgatggccgagcaaccgctcaatgcgaagatggttgtggaggagataaaggtgggagtaagagttgaaacggaagatgggagtgtaaaaggttttgtgacaagagaagaactaagtggaaagattaaagaactgatggaaggagaaacggggaaaaccgcaagaaagaatgtaaaagaatattcgaaaatggcgaaagcggctttggtcgaagggactggttcgtcatggaagaatttagatatgattcttaaggagttatgtaagagtagagattcaaacggtgctagtgagtag

SEQ ID NO: 102 - DNA, 1406 bp, Arabidopsis thaliana
atggagttagaaaaagttcacgtggttttgttcccatacttgtccaaagggcacatgattcctatgctccaattagctcgtctcctcttatcccactccttcgccggagacatctccgtcaccgtcttcaccactcctttgaaccgtcctttcatcgttgactcactctccggcaccaaagcgaccatcgtcgacgtacctttccctgataacgtcccggagatcccacccggcgtcgagtgcactgacaaactccctgctttgtcgtcctccctcttcgttcctttcacaagagccaccaagtcaatgcaggcagactttgagcgagagctcatgtcactgccacgtgtcagtttcatggtctcagacggtttcttgtggtggacgcaagagtcagctcgaaagctagggtttcctcggcttgttttctttggtatgaattgcgcttccaccgttatatgtgacagtgtttttcaaaaccagcttctatctaatgttaagtccgagacggagccagtttctgtaccggagtttccgtggattaaggttaggaaatgtgatttcgttaaagatatgtttgatccaaaaaccaccacagatcctggattcaagcttatcctagatcaagtcacgtctatgaatcaaagccaaggtatcatattcaatacatttgacgaccttgaacccgtgtttattgatttctacaagcgtaaacgcaaactcaagctttgggcagttggaccgctttgttacgtaaataacttggcttggatgatgaagtagaagagaaggtcaaacctagttggatgaaatggctagatgaaaagcgagacaagggatgcaatgttctgtatgtggctttcgggtcacaagccgagatctcgagagaacaactagaggagattgcgttagggttggaagaatcgaaggtgaacttcttgtgggtggtcaaaggaaatgaaataggaaaagggtttgaagagagagtgggagaaagaggaatgatggtgagagatgaatgggttgatcagaggaagatattagagcacgagagtgttagagggttcttgagccattgtgggtggaattctctgacggagagcatttgctcggaggttccaatcttggcgtttcctttagcagcggagcaacctctgaatgcgattttggtggtggaagagctgagagtggcggagagagtggtggcggcgagtgaaggggttgtgagaagagaagagattgcagagaaagtgaaggagttgatggagggagagaaagggaaagagctgaggaggaatgtcgaggcatatggtaagatggcgaagaaggctttggaggaaggtattggttcgtctaggaagaatttagacaaccttatcaacgagttttgtaacaatggaacatga

SEQ ID NO: 103 - DNA, 1479 bp, Arabidopsis thaliana
atggccgtttcatcgtcgcatcatgcggttctcttcccttacatgtcaaaaggccacacgattcctctcctccaattcgcccgtctcctcctccgtcaccgccgtatcgtctccgtagacgacgaagaaccaaccatttccgtcaccgtcttcaccaccccaaaaaaccaaccattcgtctcaaacttcctctctgacgtcgcatcatctatcaaagtaatctccctccctttccctgaaaacatcgccggaatccctcccggcgtcgagagcaccgacatgctcccttccatatcactttacgtgcccttcacgcgcgcaaccaaatctctccagcctttcttcgaagcagaactcaagaatcttgagaaagtttctttcatggtctccgatggattcttatggtggacatcggaatccgccgctaaatttgagatcccgagacttgccttctacggcatgaactcctacgcatcggctatgtgctccgccatttcggtacacgagctctttaccaaaccggaaagtgttaaatctgatactgaaccggttactgtaccggattttccatggatatgtgttaagaagtgtgagttcgatccggttttgaccgaaccggatcaatcggatccagcgttcgagctactcattgaccatcttatgtccaccaagaaaagccgtggagttatagtgaacagcttttacgagctcgagtcaacgttcgttgactaccggctccgtgataacgatgaaccaaaaccgtggtgtgttgggcctttgtgtttggtaaatcctccaaaaccggagagtgataaaccggattggattcattggttggaccggaaactagaggaaagatgtccggttatgtatgtggcgtttggaacgcaggctgagatatcgaacgagcagctcaaggaaatagcattagggttggaagattccaaggtcaatttcttgtgggtcacgagaaaggacttggaagaagtaactggaggattagggttcgaaaagagagtgaaagagcatgggatgattgtgagagattgggtagaccaatgggagatattgtcacataaaagtgtcaaagggtttttgagtcattgtggatggaactcggcgcaagagagtatttgcgctggggttccactactcgcttggccaatgatggcagagcagccactcaatgcgaagttggtagtggaggagctaaagatcggagtaagaatcgaaacagaagatgtaagtgtgaaaggattcgtgacaagagaagaacttagtcgaaaggttaaacaattgatggagggagagatggggaagacaacgatgaagaatgtaaaagagtatgcgaaaatggcgaaaaaagctatggctcaagggactggttcgtcttggaagagtttggattcgcttctggaagagctttgtaagagtagagagccagacggtgttaataagttgtcaagttctgatgcttag

SEQ ID NO: 104 - DNA, 1413 bp, Arabidopsis thaliana
atgacaaacttcaaagacaacgatggagatggaaccaaactccacgtggtaatgtttccatggttagcctttggtcacatggttccatacttggagctctctaaactcatagctcaaaagggtcacaaagtctctttcatttccactccacgtaacatcgaccgtctcctcccatggttaccggaaaatctctcctccgtcattaacttcgtcaagctatcacttcccgtcggcgacaacaaactcccggaagacggtgaagctaccacagacgtccctttcgaactcataccttacttaaaaatcgcttacgacgggttaaaagttccggtgacggagtttcttgaatcttcgaaacccgattgggttcttcaagatttcgcggggttttggcttcctccaatctctcgtcgtctcggaatcaaaaccggattctttagcgctttcaacggcgcgacgctcggtattcttaaaccgccggggttcgaagagtaccgtacttcgccggcggattttatgaagccgcctaagtgggttccgtttgaaacttcggtagctttcaagttatttgaatgcaggttcattttcaaaggatttatggcggaaaccaccgaagggaatgttcccgacatccaccgtgtcggcggcgtaattgacggctgtgacgtcatcttcgtacggagctgttacgagtatgaagcggagtggttaggacttacacaagaacttcaccggaaaccggttataccggtcggagttttgcctccaaaaccggacgaaaagtttgaagataccgacacgtggctgtctgttaaaaaatggttggactcacggaaaagtaagtccattgtctacgtagcttttggttcagaagctaaaccgagtcaaacggagctaaatgagatcgctctcggtttagagctttctggtttacctttcttttgggtgttaaagactcgtcgtggtccgtgggataccgaaccggtcgagcttccggaaggattcgaagagcgtacagcggatagagggatggtgtggagaggttgggttgagcaattgcgtacattgagccatgactcgatcggtttggttctgactcatcccggttggggaacgataattgaagctatccggtttgctaaaccgatggcaatgctggtttttgtgtatgaccaaggattgaatgcgagagtcattgaagagaagaaaattgggtatatgatccctcgagacgagacagaaggtttctttactaaagaaagtgttgcgaattcgctaagattggtaatggtggaagaagaaggaaaggtttatagagagaatgtgaaggagatgaaaggagtgtttggagatatggatagacaagatcgttatgtggattcattcttggaatatcttgttactaatcgttaa

SEQ ID NO: 105 - DNA, 1401 bp, Arabidopsis thaliana
atggccgagccaaaaccgaagcttcatgttgcagtgttcccatggttagctttaggtcacatgattccttacttgcaactctcaaagctcatagcaaggaaaggccatactgtgtccttcatctccacagctcgtaacatttcacgtcttcccaatatatcctccgacctttccgtgaatttcgtttctttgccgttaagtcaaaccgtcgaccatctcccagagaacgctgaggccaccactgatgtcccggagactcacatagcttatctgaagaaagcatttgatgggctttctgaagctttcacagagtttttagaagcttccaaaccaaactggatagtgtatgatatcttgcaccattgggtcccgcctatcgctgagaagctcggcgtgagacgagccatcttctgcacgttcaacgcagcttccatcatcatcatcggtgggccagcatcagtcatgattcaaggtcatgaccctcgaaagactgctgaagatcttatcgtgcctccaccatgggtcccgtttgagaccaacatagtttaccgtctctttgaagctaagaggatcatggagtatcccacggcaggtgtaactggagttgaattgaacgacaactgtagattgggtttggcttacgttggctctgaggttattgtgattagatcatgtatggaactcgaacctgagtggattcaattgctcagtaaactccaaggaaagcctgtgattccaattggtttactcccggctacaccaatggatgatgcagatgacgagggaacatggttagacatcagagaatggctagacagacatcaagcaaagtctgtggtttatgtagccttaggaactgaagtgacaattagtaacgaagagattcaaggtttagctcatgggttggagctttgcaggttacctttcttttggacgctaaggaagaggactagagcttctatgctactacctgatgggttcaaagagagagtcaaagagcgtggagtcatttggaccgagtgggtacctcagaccaagatactgagccatggttcagttggtgggtttgttactcattgtggttggggatcagctgtggaagggcttagctttggtgtccctttgatcatgtttccatgtaacctagaccagccgctagtggctaggttgctcagtgggatgaatataggcttggagattccaaggaatgagcgagacgggctgttcacgagtgcttctgttgcagagacaatcagacatgttgttgtggaagaagaaggaaagatctacaggaacaatgctgcatctcagcaaaagaaaatattcgggaacaagagattgcaagatcagtatgcggatggttttatcgagtttctggagaatcctatagcaggagtgtag

SEQ ID NO: 106 - DNA, 1383 bp, Arabidopsis thaliana
atggtcgacaagagagaagaagttatgcacgtagccatgtttccatggctagctatgggtcatctccttccttttcttcgtctctccaagttactagctcaaaagggtcacaagatctctttcatatcaacaccaagaaacatcgaaagacttcctaaattacaatcaaacctcgcctcctccatcaccttcgtctctttccctctccctcccatctcaggcttgcctccttcttcagaatcatccatggacgttccttacaacaagcaacagtctcttaaagccgcttttgatcttcttcagccaccgttgaaagagtttctccgacggtcttctccggattggatcatatacgactatgcttctcactggcttccttctattgcggccgagcttggaatctctaaggctttctttagtctctttaacgcagctactctctgtttcatgggaccgtcttcgtctttgattgaagaaattagatcaacgccggaagatttcacggtggtgccaccgtgggtcccgttcaagtcaaacatcgtgtttcgttatcatgaagttactagatacgttgagaagacagaggaagatgtaaccggagtctctgactcagttcggtttggttactcgattgacgaaagcgatgcggtttttgtccgtagctgtccggagtttgaaccggaatggtttggtttactaaaagacctgtaccgtaaaccggtatttccaatcgggtttttgcctccggttattgaagacgacgatgccgttgatactacatgggttcgtataaagaagtggctcgacaagcaacggcttaattcagttgtttacgtgtcacttggcaccgaagcgagtcttcgtcatgaggaagtaactgagctagctcttgggttagagaagtcagagacaccgttcttttgggtcctaaggaacgagccaaagattccagatgggttcaaaacacgagtcaagggacgtggaatggttcatgttggttgggttccacaagtgaaaatacttagtcacgagtcagtaggagggttcttgacacattgtggttggaactcagtggtggaagggttagggtttggtaaagttccaatcttttttccggtgttgaatgagcaaggacttaatacgaggttgttgcatgggaaaggacttggtgttgaggtttcaagagatgagagagatgggtcgtttgattctgactcggtcgctgactcgattaggttggtgatgattgatgatgctggcgaggagataagggctaaggctaaagtgatgaaggatttgtttgggaacatggatgagaatattcgttatgttgacgaacttgttaggtttatgagaagtaaaggatcatcatcatcatcatga

SEQ ID NO: 107 - DNA, 1467 bp, Arabidopsis thaliana
atggcggaagctaaacccagaaatctgagaatcgtgatgttccctttcatgggacaaggccatatcatcccgtttgtagctttagcccttcgtttagagaagattatgattatgaacagagccaacaaaaccaccatctctatgatcaatactccttcgaacatccccaaaatacgctccaatcttccacctgaatcctccataagtctcatagagttacctttcaacagctctgatcatggccttcctcacgacggcgagaatttcgattctcttccttactctctcgtcatcagccttcttgaagcttctaggtcgcttcgtgagccctttcgagacttcatgacgaagatcttgaaggaagaagggcagagctcggttatagtgatcggtgatttcttcttgggttggatcggtaaggtttgcaaagaggttggtgtttattcagtgatctttagtgcttctggtgcttttggtttaggttgttatagatccatatggttaaacttgccacataaagaaaccaaacaagatcagtttctcttagatgatttccctgaagcaggggagattgagaaaactcagttgaattctttcatgttagaagctgatggaaccgatgattggtctgttttcatgaagaagattatacctggatggtctgacttcgatggattcttgttcaacacggttgctgaaatcgatcagatgggattatcctacttccgtagaataaccggtgttccggtttggccagttgggccggttttgaagtctccggataagaaggtgggatcgaggtcgacagaggaagcagtgaagtcatggcttgactcaaaaccggaccattcggttgtgtacgtatgtttcggttcaatgaactcgattttgcaaacgcatatgttagaattggctatggcattagagagtagcgagaagaacttcatatgggtggtgaggccgcccataggtgtggaggtgaagagtgagtttgatgtgaaagggtatctaccggaaggatttgaggaaagaataacaagatcggaaagagggttacttgtgaagaaatgggcaccacaagttgatatattgtcacacaaggcaacatgtgtgtttttgagtcattgcggatggaactcgatactcgaatcacttagccacggtgtgccactgctcggatggcccatggcagccgagcagttcttcaattccatattgatggagaaacatattggggtatcggttgaggtggcgcgtgggaagagatgtgagatcaaatgtgatgacattgtttctaagatcaaactggtgatggaggagactgaagtagggaaagagattaggaagaaggctagagaggtgaaggagttagtgaggagagcaatggtagatggagttaaaggttcctccgtcattggtttggaagagtttcttgaccaagcaatggtcaagaaagtggagaattga

SEQ ID NO: 108 - protein, 481 aa, Arabidopsis thaliana
MGKQEDAELVIIPFPFSGHILATIELAKRLISQDNPRIHTITILYWGLPFIPQADTIAFLRSLVKNEPRIRLVTLPEVQDPPPMELFVEFAESYILEYVKKMVPIIREALSTLLSSRDESGSVRVAGLVLDFFCVPMIDVGNEFNLPSYIFLTCSAGFLGMMKYLPERHREIKSEFNRSFNEELNLIPGYVNSVPTKVLPSGLFMKETYEPWVELAERFPEAKGILVNSYTALEPNGFKYFDRCPDNYPTIYPIGPILCSNDRPNLDSSERDRIITWLDDQPESSVVFLCFGSLKNLSATQINEIAQALEIVDCKFIWSFRTNPKEYASPYEALPHGFMDRVMDQGIVCGWAPQVEILAHKAVGGFVSHCGWNSILESLGFGVPIATWPMYAEQQLNAFTMVKELGLALEMRLDYVSEDGDIVKADEIAGTVRSLMDGVDVPKSKVKEIAEAGKEAVDGGSSFLAVKRFIGDLIDGVSISK

SEQ ID NO: 109 - protein, 474 aa, Arabidopsis thaliana
MAKQQEAELIFIPFPIPGHILATIELAKRLISHQPSRIHTITILHWSLPFLPQSDTIAFLKSLIETESRIRLITLPDVQNPPPMELFVKASESYILEYVKKMVPLVRNALSTLLSSRDESDSVHVAGLVLDFFCVPLIDVGNEFNLPSYIFLTCSASFLGMMKYLLERNRETKPELNRSSDEETISVPGFVNSVPVKVLPPGLFTTESYEAWVEMAERFPEAKGILVNSFESLERNAFDYFDRRPDNYPPVYPIGPILCSNDRPNLDLSERDRILKWLDDQPESSVVFLCFGSLKSLAASQIKEIAQALELVGIRFLWSIRTDPKEYASPNEILPDGFMNRVMGLGLVCGWAPQVEILAHKAIGGFVSHCGWNSILESLRFGVPIATWPMYAEQQLNAFTIVKELGLALEMRLDYVSEYGEIVKADEIAGAVRSLMDGEDVPRRKLKEIAEAGKEAVMDGGSSFVAVKRFIDGL

SEQ ID NO: 110 - protein, 479 aa, Arabidopsis thaliana
MVKETELIFIPVPSTGHILVHIEFAKRLINLDHRIHTITILNLSSPSSPHASVFARSLIASQPKIRLHDLPPIQDPPPFDLYQRAPEAYIVKLIKKNTPLIKDAVSSIVASRRGGSDSVQVAGLVLDLFCNSLVKDVGNELNLPSYIYLTCNARYLGMMKYIPDRHRKIASEFDLSSGDEELPVPGFINAIPTKFMPPGLFNKEAYEAYVELAPRFADAKGILVNSFTELEPHPFDYFSHLEKFPPVYPVGPILSLKDRASPNEEAVDRDQIVGWLDDQPESSVVFLCFGSRGSVDEPQVKEIARALELVGCRFLWSIRTSGDVETNPNDVLPEGFMGRVAGRGLVCGWAPQVEVLAHKAIGGFVSHCGWNSTLESLWFGVPVATWPMYAEQQLNAFTLVKELGLAVDLRMDYVSSRGGLVTCDEIARAVRSLMDGGDEKRKKVKEMADAARKALMDGGSSSLATARFIAELFEDGSSC

SEQ ID NO: 111 - protein, 467 aa, Arabidopsis thaliana
MRNVELIFIPTPTVGHLVPFLEFARRLIEQDDRIRITILLMKLQGQSHLDTYVKSIASSQPFVRFIDVPELEEKPTLGSTQSVEAYVYDVIERNIPLVRNIVMDILTSLALDGVKVKGLVVDFFCLPMIDVAKDISLPFYVFLTTNSGFLAMMQYLADRHSRDTSVFVRNSEEMLSIPGFVNPVPANVLPSALFVEDGYDAYVKLAILFTKANGILVNSSFDIEPYSVNHFLQEQNYPSVYAVGPIFDLKAQPHPEQDLTRRDELMKWLDDQPEASVVFLCFGSMARLRGSLVKEIAHGLELCQYRFLWSLRKEEVTKDDLPEGFLDRVDGRGMICGWSPQVEILAHKAVGGFVSHCGWNSIVESLWFGVPIVTWPMYAEQQLNAFLMVKELKLAVELKLDYRVHSDEIVNANEIETAIRYVMDTDNNVVRKRVMDISQMIQRATKNGGSSFAAIEKFIYDVIGIKP

SEQ ID NO: 112 - protein, 480 aa, Arabidopsis thaliana
MEESKTPHVAIIPSPGMGHLIPLVEFAKRLVHLHGLTVTFVIAGEGPPSKAQRTVLDSLPSSISSVFLPPVDLTDLSSSTRIESRISLTVTRSNPELRKVFDSFVEGGRLPTALVVDLFGTDAFDVAVEFHVPPYIFYPTTANVLSFFLHLPKLDETVSCEFRELTEPLMLPGCVPVAGKDFLDPAQDRKDDAYKWLLHNTKRYKEAEGILVNTFFELEPNAIKALQEPGLDKPPVYPVGPLVNIGKQEAKQTEESECLKWLDNQPLGSVLYVSFGSGGTLTCEQLNELALGLADSEQRFLWVIRSPSGIANSSYFDSHSQTDPLTFLPPGFLERTKKRGFVIPFWAPQAQVLAHPSTGGFLTHCGWNSTLESVVSGIPLIAWPLYAEQKMNAVLLSEDIRAALRPRAGDDGLVRREEVARVVKGLMEGEEGKGVRNKMKELKEAACRVLKDDGTSTKALSLVALKWKAHKKELEQNGNH

SEQ ID NO: 113 - protein, 470 aa, Arabidopsis thaliana
MDQPHALLVASPGLGHLIPILELGNRLSSVLNIHVTILAVTSGSSSPTETEAIHAAAARTICQITEIPSVDVDNLVEPDATIFTKMVVKMRAMKPAVRDAVKLMKRKPTVMIVDFLGTELMSVADDVGMTAKYVYVPTHAWFLAVMVYLPVLDTVVEGEYVDIKEPLKIPGCKPVGPKELMETMLDRSGQQYKECVRAGLEVPMSDGVLVNTWEELQGNTLAALREDEELSRVMKVPVYPIGPIVRTNQHVDKPNSIFEWLDEQRERSVVFVCLGSGGTLTFEQTVELALGLELSGQRFVWVLRRPASYLGAISSDDEQVSASLPEGFLDRTRGVGIVVTQWAPQVEILSHRSIGGFLSHCGWSSALESLTKGVPIIAWPLYAEQWMNATLLTEEIGVAVRTSELPSERVIGREEVASLVRKIMAEEDEEGQKIRAKAEEVRVSSERAWSKDGSSYNSLFEWAKRCYLVP

SEQ ID NO: 114 - protein, 488 aa, Arabidopsis thaliana
MGTPVEVSKLHFLLFPFMAHGHMIPTLDMAKLFATKGAKSTILTTPLNAKLFFEKPIKSFNQDNPGLEDITIQILNFPCTELGLPDGCENTDFIFSTPDLNVGDLSQKFLLAMKYFEEPLEELLVTMRPDCLVGNMFFPWSTKVAEKFGVPRLVFHGTGYFSLCASHCIRLPKNVATSSEPFVIPDLPGDILITEEQVMETEEESVMGRFMKAIRDSERDSFGVLVNSFYELEQAYSDYFKSFVAKRAWHIGPLSLGNRKFEEKAERGKKASIDEHECLKWLDSKKCDSVIYMAFGTMSSFKNEQLIEIAAGLDMSGHDFVWVVNRKGSQEEKEDWLPEGFEEKTKGKGLIIRGWAPQVLILEHKAIGGFLTHCGWNSLLEGVAAGLPMVTWPVGAEQFYNEKLVTQVLKTGVSVGVKKMMQVVGDFISREKVEGAVREVMVGEERRKRAKELAEMAKNAVKEGGSSDLEVDRLMEELTLVKLQKEKV

SEQ ID NO: 115 - protein, 483 aa, Arabidopsis thaliana
MGSDHHHRKLHVMFFPFMAYGHMIPTLDMAKLFSSRGAKSTILTTSLNSKILQKPIDTFKNLNPGLEIDIQIFNFPCVELGLPEGCENVDFFTSNNNDDKNEMIVKFFFSTRFFKDQLEKLLGTTRPDCLIADMFFPWATEAAGKFNVPRLVFHGTGYFSLCAGYCIGVHKPQKRVASSSEPFVIPELPGNIVITEEQIIDGDGESDMGKFMTEVRESEVKSSGVVLNSFYELEHDYADFYKSCVQKRAWHIGPLSVYNRGFEEKAERGKKANIDEAECLKWLDSKKPNSVIYVSFGSVAFFKNEQLFEIAAGLEASGTSFIWVVRKTKDDREEWLPEGFEERVKGKGMIIRGWAPQVLILDHQATGGFVTHCGWNSLLEGVAAGLPMVTWPVGAEQFYNEKLVTQVLRTGVSVGASKHMKVMMGDFISREKVDKAVREVLAGEAAEERRRRAKKLAAMAKAAVEEGGSSFNDLNSFMEEFSS

SEQ ID NO: 116 - protein, 481 aa, Arabidopsis thaliana
MSSDPHRKLHVVFFPFMAYGHMIPTLDMAKLFSSRGAKSTILTTPLNSKIFQKPIERFKNLNPSFEIDIQIFDFPCVDLGLPEGCENVDFFTSNNNDDRQYLTLKFFKSTRFFKDQLEKLLETTRPDCLIADMFFPWATEAAEKFNVPRLVFHGTGYFSLCSEYCIRVHNPQNIVASRYEPFVIPDLPGNIVITQEQIADRDEESEMGKFMIEVKESDVKSSGVIVNSFYELEPDYADFYKSVVLKRAWHIGPLSVYNRGFEEKAERGKKASINEVECLKWLDSKKPDSVIYISFGSVACFKNEQLFEIAAGLETSGANFIWVVRKNIGIEKEEWLPEGFEERVKGKGMIIRGWAPQVLILDHQATCGFVTHCGWNSLLEGVAAGLPMVTWPVAAEQFYNEKLVTQVLRTGVSVGAKKNVRTTGDFISREKVVKAVREVLVGEEADERRERAKKLAEMAKAAVEGGSSFNDLNSFIEEFTS

SEQ ID NO: 117 - protein, 481 aa, Arabidopsis thaliana
MNREQIHILFFPFMAHGHMIPLLDMAKLFARRGAKSTLLTTPINAKILEKPIEAFKVQNPDLEIGIKILNFPCVELGLPEGCENRDFINSYQKSDSFDLFLKFLFSTKYMKQQLESFIETTKPSALVADMFFPWATESAEKIGVPRLVFHGTSSFALCCSYNMRIHKPHKKVASSSTPFVIPGLPGDIVITEDQANVTNEETPFGKFWKEVRESETSSFGVLVNSFYELESSYADFYRSFVAKKAWHIGPLSLSNRGIAEKAGRGKKANIDEQECLKWLDSKTPGSVVYLSFGSGTGLPNEQLLEIAFGLEGSGQNFIWVVSKNENQGENEDWLPKGFEERNKGKGLIIRGWAPQVLILDHKAIGGFVTHCGWNSTLEGIAAGLPMVTWPMGAEQFYNEKLLTKVLRIGVNVGATELVKKGKLISRAQVEKAVREVIGGEKAEERRLRAKELGEMAKAAVEEGGSSYNDVNKFMEELNGRK

SEQ ID NO: 118 - protein, 484 aa, Arabidopsis thaliana
MNREVSERIHILFFPFMAQGHMIPILDMAKLFSRRGAKSTLLTTPINAKIFEKPIEAFKNQNPDLEIGIKIFNFPCVELGLPEGCENADFINSYQKSDSGDLFLKFLFSTKYMKQQLESFIETTKPSALVADMFFPWATESAEKLGVPRLVFHGTSFFSLCCSYNMRIHKPHKKVATSSTPFVIPGLPGDIVITEDQANVAKEETPMGKFMKEVRESETNSFGVLVNSFYELESAYADFYRSFVAKRAWHIGPLSLSNRELGEKARRGKKANIDEQECLKWLDSKTPGSVVYLSFGSGTNFTNDQLLEIAFGLEGSGQSFIWVVRKNENQGDNEEWLPEGFKERTTGKGLIIPGWAPQVLILDHKAIGGFVTHCGWNSAIEGIAAGLPMVTWPMGAEQFYNEKLLTKVLRIGVNVGATELVKKGKLISRAQVEKAVREVIGGEKAEERRLWAKKLGEMAKAAVEEGGSSYNDVNKFMEELNGRK

SEQ ID NO: 119 - protein, 491 aa, Arabidopsis thaliana
MASEFRPPLHFVLFPFMAQGHMIPMVDIARLLAQRGVTITIVTTPQNAGRFKNVLSRAIQSGLPINLVQVKFPSQESGSPEGQENLDLLDSLGASLTFFKAFSLLEEPVEKLLKEIQPRPNCIIADMCLPYTNRIAKNLGIPKIIFHGMCCFNLLCTHIMHQNHEFLETIESDKEYFPIPNFPDRVEFTKSQLPMVLVAGDWKDFLDGMTEGDNTSYGVIVNTFEELEPAYVRDYKKVKAGKIWSIGPVSLCNKLGEDQAERGNKADIDQDECIKWLDSKEEGSVLYVCLGSICNLPLSQLKELGLGLEESQRPFIWVIRGWEKYNELLEWISESGYKERIKERGLLITGWSPQMLILTHPAVGGFLTHCGWNSTLEGITSGVPLLTWPLFGDQFCNEKLAVQILKAGVRAGVEESMRWGEEEKIGVLVDKEGVKKAVEELMGDSNDAKERRKRVKELGELAHKAVEEGGSSHSNITFLLQDIMQLEQPKK

SEQ ID NO: 120 - protein, 496 aa, Arabidopsis thaliana
MATEKTHQFHPSLHFVLFPFMAQGHMIPMIDIARLLAQRGVTITIVTTPHNAARFKNVLNRAIESGLAINILHVKFPYQEFGLPEGKENIDSLDSTELMVPFFKAVNLLEDPVMKLMEEMKPRPSCLISDWCLPYTSIIAKNFNIPKIVFHGMGCFNLLCMHVLRRNLEILENVKSDEEYFLVPSFPDRVEFTKLQLPVKANASGDWKEIMDEMVKAEYTSYGVIVNTFQELEPPYVKDYKEAMDGKVWSIGPVSLCNKAGADKAERGSKAAIDQDECLQWLDSKEEGSVLYVCLGSICNLPLSQLKELGLGLEESRRSFIWVIRGSEKYKELFEWMLESGFEERIKERGLLIKGWAPQVLILSHPSVGGFLTHCGWNSTLEGITSGIPLITWPLFGDQFCNQKLVVQVLKAGVSAGVEEVMKWGEEDKIGVLVDKEGVKKAVEELMGDSDDAKERRRRVKELGELAHKAVEKGGSSHSNITLLLQDIMQLAQFKN

SEQ ID NO: 121 - protein, 496 aa, Arabidopsis thaliana
MASEKSHKVHPPLHFILFPFMAQGHMIPMIDIARLLAQRGATVTIVTTRYNAGRFENVLSRAMESGLPINIVHVNFPYQEFGLPEGKENIDSYDSMELMVPFFQAVNMLEDPVMKLMEEMKPRPSCIISDLLLPYTSKIARKFSIPKIVFHGTGCFNLLCMHVLRRNLEILKNLKSDKDYFLVPSFPDRVEFTKPQVPVETTASGDWKAFLDEMVEAEYTSYGVIVNTFQELEPAYVKDYTKARAGKVWSIGPVSLCNKAGADKAERGNQAAIDQDECLQWLDSKEDGSVLYVCLGSICNLPLSQLKELGLGLEKSQRSFIWVIRGWEKYNELYEWMMESGFEERIKERGLLIKGWSPQVLILSHPSVGGFLTHCGWNSTLEGITSGIPLITWPLFGDQFCNQKLVVQVLKAGVSAGVEEVMKWGEEEKIGVLVDKEGVKKAVEELMGASDDAKERRRRVKELGESAHKAVEEGGSSHSNITYLLQDIMQQVKSKN

SEQ ID NO: 122 - protein, 495 aa, Arabidopsis thaliana
MVSETTKSSPLHFVLFPFMAQGHMIPMVDIARLLAQRGVIITIVTTPHNAARFKNVLNRAIESGLPINLVQVKFPYLEAGLQEGQENIDSLDTMERMIPFFKAVNFLEEPVQKLIEEMNPRPSCLISDFCLPYTSKIAKKFNIPKILFHGMGCFCLLCMHVLRKNREILDNLKSDKELFTVPDFPDRVEFTRTQVPVETYVPAGDWKDIFDGMVEANETSYGVIVNSFQELEPAYAKDYKEVRSGKAWTIGPVSLCNKVGADKAERGNKSDIDQDECLKWLDSKKHGSVLYVCLGSICNLPLSQLKELGLGLEESQRPFIWVIRGWEKYKELVEWFSESGFEDRIQDRGLLIKGWSPQMLILSHPSVGGFLTHCGWNSTLEGITAGLPLLTWPLFADQFCNEKLVVEVLKAGVRSGVEQPMKWGEEEKIGVLVDKEGVKKAVEELMGESDDAKERRRRAKELGDSAHKAVEEGGSSHSNISFLLQDIMELAEPNN

SEQ ID NO: 123 - protein, 495 aa, Arabidopsis thaliana
MAFEKNNEPFPLHFVLFPFMAQGHMIPMVDIARLLAQRGVLITIVTTPHNAARFKNVLNRAIESGLPINLVQVKFPYQEAGLQEGQENMDLLTTMEQITSFFKAVNLLKEPVQNLIEEMSPRPSCLISDMCLSYTSEIAKKFKIPKILFHGMGCFCLLCVNVLRKNREILDNLKSDKEYFIVPYFPDRVEFTRPQVPVETYVPAGWKEILEDMVEADKTSYGVIVNSFQELEPAYAKDFKEARSGKAWTIGPVSLCNKVGVDKAERGNKSDIDQDECLEWLDSKEPGSVLYVCLGSICNLPLSQLLELGLGLEESQRPFIWVIRGWEKYKELVEWFSESGFEDRIQDRGLLIKGWSPQMLILSHPSVGGFLTHCGWNSTLEGITAGLPMLTWPLFADQFCNEKLVVQILKVGVSAEVKEVMKWGEEEKIGVLVDKEGVKKAVEELMGESDDAKERRRRAKELGESAHKAVEEGGSSHSNITFLLQDIMQLAQSNN

SEQ ID NO: 124 - protein, 460 aa, Arabidopsis thaliana
MAETTPKVKGHVVILPYPVQGHLNPMVQFAKRLVSKNVKVTIATTTYTASSITTPSLSVEPISDGFDFIPIGIPGFSVDTYSESFKLNGSETLTLLIEKFKSTDSPIDCLIYDSFLPWGLEVARSMELSAASFFTNNLTVCSVLRKFSNGDFPLPADPNSAPFRIRGLPSLSYDELPSFVGRHWLTHPEHGRVLLNQFPNHENADWLFVNGFEGLEETQDCENGESDAMKATLIGPMIPSAYLDDRMEDDKDYGASLLKPISKECMEWLETKQAQSVAFVSFGSFGILFEKQLAEVAIALQESDLNFLWVIKEAHIAKLPEGFVESTKDRALLVSWCNQLEVLAHESIGCFLTHCGWNSTLEGLSLGVPMVGVPQWSDQMNDAKFVEEVWKVGYRAKEEAGEVIVKSEELVRCLKGVMEGESSVKIRESSKKWKDLAVKAMSEGGSSDRSINEFIESLGK

SEQ ID NO: 125 - protein, 453 aa, Arabidopsis thaliana
MREGSHLIVLPFPGQGHITPMSQFCKRLASKGLKLTLVLVSDKPSPPYKTEHDSITVFPISNGFQEGEEPLQDLDDYMERVETSIKNTLPKLVEDMKLSGNPPRAIVYDSTMPWLLDVAHSYGLSGAVFFTQPWLVTAIYYHVFKGSFSVPSTKYGHSTLASFPSFPMLTANDLPSFLCESSSYPNILRIVVDQLSNIDRVDIVLCNTFDKLEEKLLKWVQSLWPVLNIGPTVPSMYLDKRLSEDKNYGFSLFNAKVAECMEWLNSKEPNSVVYLSFGSLVILKEDQMLELAAGLKQSGRFFLWVVRETETHKLPRNYVEEIGEKGLIVSWSPQLDVLAHKSIGCFLTHCGWNSTLEGLSLGVPMIGMPHWTDQPTNAKFMQDVWKVGVRVKAEGDGFVRREEIMRSVEEVMEGEKGKEIRKNAEKWKVLAQEAVSEGGSSDKSINEFVSMFC

SEQ ID NO: 126 - protein, 449 aa, Arabidopsis thaliana
MEKMRGHVLAVPFPSQGHITPIRQFCKRLHSKGFKTTHTLTTFIFNTIHLDPSSPISIATISDGYDQGGFSSAGSVPEYLQNFKTFGSKTVADIIRKHQSTDNPITCIVYDSFMPWALDLAMDFGLAAAPFFTQSCAVNYINYLSYINNGSLTLPIKDLPLLELQDLPTFVTPTGSHLAYFEMVLQQFTNFDKADFVLVNSFHDLDLHEEELLSKVCPVLTIGPTVPSMYLDQQIKSDNDYDLNLFDLKEAALCTDWLDKRPEGSVVYIAFGSMAKLSSEQMEEIASAISNFSYLWVVRASEESKLPPGFLETVDKDKSLVLKWSPQLQVLSNKAIGCFMTHCGWNSTMEGLSLGVPMVAMPQWTDQPMNAKYIQDVWKVGVRVKAEKESGICKREEIEFSIKEVMEGEKSKEMKENAGKWRDLAVKSLSEGGSTDININEFVSKIQIK

SEQ ID NO: 127 - protein, 453 aa, Arabidopsis thaliana
MEELGVKRRIVLVPVPAQGHVTPIMQLGKALYSKGFSITVVLTQYNRVSSSKDFSDFHFLTIPGSLTESDLKNLGPFKFLFKLNQICEASFKQCIGQLLQEQGNDIACVVYDEYMYFSQAAVKEFQLPSVLFSTTSATAFVCRSVLSRVNAESFLLDMKDPKVSDKEFPGLHPLRYKDLPTSAFGPLESILKVYSETVNIRTASAVIINSTSCLESSSLAWLQKQLQVPVYPIGPLHIAASAPSSLLEEDRSCLEWLNKQKIGSVIYISLGSLALMETKDMLEMAWGLRNSNQPFLWVIRPGSIPGSEWTESLPEEFSRLVSERGYIVKWAPQIEVLRHPAVGGFWSHCGWNSTLESIGEGVPMICRPFTGDQKVNARYLERVWRIGVQLEGELDKGTVERAVERLIMDEEGAEMRKRVINLKEKLQASVKSRGSSFSSLDNFVNSLKMMNFM

SEQ ID NO: 128 - protein, 453 aa, Arabidopsis thaliana
MEEKPARRSVVLVPFPAQGHISPMMQLAKTLHLKGFSITVVQTKFNYFSPSDDFTHDFQFVTIPESLPESDFKNLGPIQFLFKLNKECKVSFKDCLGQLVLQQSNEISCVIYDEFMYFAEAAAKECKLPNIIFSTTSATAFACRSVFDKLYANNVQAPLKETKGQQEELVPEFYPLRYKDFPVSRFASLESIMEVYRNTVDKRTASSVIINTASCLESSSLSFLQQQQLQIPVYPIGPLHMVASAPTSLLEENKSCIEWLNKQKVNSVIYISMGSIALMEINEIMEVASGLAASNQHFLWVIRPGSIPGSEWIESMPEEFSKMVLDRGYIVKWAPQKEVLSHPAVGGFWSHCGWNSTLESIGQGVPMICRPFSGDQKVNARYLECVWKIGIQVEGELDRGVVERAVKRLMVDEEGEEMRKRAFSLKEQLRASVKSGGSSHNSLEEFVHFIRTL

SEQ ID NO: 129 - protein, 460 aa, Arabidopsis thaliana
MTKPSDPTRDSHVAVLAFPFGTHAAPLLTVTRRLASASPSTVFSFFNTAQSNSSLFSSGDEADRPANIRVYDIADGVPEGYVFSGRPQEAIELFLQAAPENFRREIAKAETEVGTEVKCLMTDAFFWFAADMATEINASWIAFWTAGANSLSAHLYTDLIRETIGVKEVGERMEETIGVISGMEKIRVKDTPEGVVFGNLDSVFSKMLHQMGLALPRATAVFINSFEDLDPTLTNNLRSRFKRYLNIGPLGLLSSTLQQLVQDPHGCLAWMEKRSSGSVAYISFGTVMTPPPGELAAIAEGLESSKVPFVWSLKEKSLVQLPKGFLDRTREQGIVVPWAPQVELLKHEATGVFVTHCGWNSVLESVSGGVPMICRPFFGDQRLNGRAVEVVWEIGMTIINGVFTKDGFEKCLDKVLVQDDGKKMKCNAKKLKELAYEAVSSKGRSSENFRGLLDAVVNII

SEQ ID NO: 130 - protein, 484 aa, Arabidopsis thaliana
MVFETCPSPNPIHVMLVSFQGQGHVNPLLRLGKLIASKGLLVTFVTTELWGKKMRQANKIVDGELKPVGSGSIRFEFFDEEWAEDDDRRADFSLYIAHLESVGIREVSKLVRRYEEANEPVSCLINNPFIPWVCHVAEEFNIPCAVLWVQSCACFSAYYHYQDGSVSFPTETEPELDVKLPCVPVLKNDEIPSFLHPSSRFTGFRQAILGQFKNLSKSFCVLIDSFDSLEQEVIDYMSSLCPVKTVGPLFKVARTVTSDVSGDICKSTDKCLEWLDSRPKSSVVYISFGTVAYLKQEQIEEIAHGVLKSGLSFLWVIRPPPHDLKVETHVLPQELKESSAKGKGMIVDWCPQEQVLSHPSVACFVTHCGWNSTMESLSSGVPVVCCPQWGDQVTDAVYLIDVFKTGVRLGRGATEERVVPREEVAEKLLEATVGEKAEELRKNALKWKAEAEAAVAPGGSSDKNFREFVEKLGAGVTKTKDNGY

SEQ ID NO: 131 - protein, 456 aa, Arabidopsis thaliana
MGSSEGQETHVLMVTLPFQGHINPMLKLAKHLSLSSKNLHINLATIESARDLLSTVEKPRYPVDLVFFSDGLPKEDPKAPETLLKSLNKVGAMNLSKIIEEKRYSCIISSPFTPWVPAVAASHNISCAILWIQACGAYSVYYRYYMKTNSFPDLEDLNQTVELPALPLLEVRDLPSFMLPSGGAHFYNLMAEFADCLRYVKWVLVNSFYELESEIIESMADLKPVIPIGPLVSPFLLGDGEEETLDGKNLDFCKSDDCCMEWLDKQARSSVVYISFGSMLETLENQVETIAKALKNRGLPFLWVIRPKEKAQNVAVLQEMVKEGQGVVLEWSPQEKILSHEAISCFVTHCGWNSTMETVVAGVPVVAYPSWTDQPIDARLLVDVFGIGVRMRNDSVDGELKVEEVERCIEAVTEGPAAVDIRRRAAELKRVARLALAPGGSSTRNLDLFISDITIA

SEQ ID NO: 132 - protein, 479 aa, Arabidopsis thaliana
MASHAVTSGQKPHVVCIPFPAQGHINPMLKVAKLLYARGFHVTFVNTNYNHNRLIRSRGPNSLDGLPSFRFESIPDGLPEENKDVMQDVPTLCESTMKNCLAPFKELLRRINTTKDVPPVSCIVSDGVMSFTLDAAEELGVPDVLFWTPSACGFLAYLHFYRFIEKGLSPIKDESSLDTKINWIPSMKNLGLKDIPSFIRATNTEDIMLNFFVHEADRAKRASAIILNTFDSLEHDVVRSIQSIIPQVYTIGPLHLFVNRDIDEESDIGQIGTNMWREEMECLDWLDTKSPNSVVYVNFGSITVMSAKQLVEFAWGLAATKKDFLWVIRPDLVAGDVPMLPPDFLIETANRRMLASWCPQEKVLSHPAVGGFLTHSGWNSTLESLSGGVPMVCWPFFAEQQTNCKYCCDEWEVGMEIGGDVRREEVEELVRELMDGDKGKKMRQKAEEWQRLAEEATKPIYGSSELNFQMVVDKVLLGE

SEQ ID NO: 133 - protein, 462 aa, Arabidopsis thaliana
MGEEAIVLYPAPPIGHLVSMVELGKTILSKNPSLSIHIILVPPPYQPESTATYISSVSSSFPSITFHHLPAVTPYSSSSTSRHHHESLLLEILCFSNPSVHRTLFSLSRNFNVRAMIIDFFCTAVLDITADFTFPVYFFYTSGAACLAFSFYLPTIDETTPGKNLKDIPTVHIPGVPPMKGSDMPKAVLERDDEVYDVFIMFGKQLSKSSGIIINTFDALENRAIKAITEELCFRNIYPIGPLIVNGRIEDRNDNKAVSCLNWLDSQPEKSVVFLCFGSLGLFSKEQVIEIAVGLEKSGQRFLWVVRNPPELEKTELDLKSLLPEGFLSRTEDKGMVVKSWAPQVPVLNHKAVGGFVTHCGWNSILEAVCAGVPMVAWPLYAEQRFNRVMIVDEIKIAISMNESETGFVSSTEVEKRVQEIIGECPVRERTMAMKNAAELALTETGSSHTALTTLLQSWSPK

SEQ ID NO: 134 - protein, 473 aa, Arabidopsis thaliana
MKVNEENNKPTKTHVLIFPFPAQGHMIPLLDFTHRLALRGGAALKITVLVTPKNLPFLSPLLSAVVNIEPLILPFPSHPSIPSGVENVQDLPPSGFPLMIHALGNLHAPLISWITSHPSPPVAIVSDFFLGWTKNLGIPRFDFSPSAAITCCILNTLWIEMPTKINEDDDNEILHFPKIPNCPKYRFDQISSLYRSYVHGDPAWEFIRDSFRDNVASWGLVVNSFTAMEGVYLEHLKREMGHDRVWAVGPIIPLSGDNRGGPTSVSVDHVMSWLDAREDNHVVYVCFGSQVVLTKEQTLALASGLEKSGVHFIWAVKEPVEKDSTRGNILDGFDDRVAGRGLVIRGWAPQVAVLRHRAVGAFLTHCGWNSVVEAVVAGVLMLTWPMRADQYTDASLVVDELKVGVRACEGPDTVPDPDELARVFADSVTGNQTERIKAVELRKAALDAIQERGSSVNDLDGFIQHVVSLGLNK